(19) United States
(12) Patent Application Publication
(10) Pub. No.: US 2007/0093440 A1
(43) Pub. Date: Apr. 26, 2007

(54) Medical treatment

(76) Inventors: Brian Robert Champion, Cambridge (GB); Grahame James McKenzie, Cambridge (GB); Yvette Stallwood, Cambridge (GB)

Correspondence Address: FROMMER LAWRENCE & HAUG, 745 FIFTH AVENUE- 10TH FL., NEW YORK, NY, 10151, US

(21) Appl. No.: 11/495,015

(22) Filed: Jul. 27, 2006

Related U.S. Application Data
(63) Continuation-in-part of application No. PCT/GB05/00243, filed on Aug. 11, 2005.

(30) Foreign Application Priority Data
Jan. 28, 2004 (GB) 0401807.3
Jan. 28, 2004 (GB) 0401792.7
Sep. 4, 2004 (GB) 0419703.4

(51) Int. Cl. A61K 48/00; C07H 21/02

(52) U.S. Cl. 514/44; 536/23.1

(57) ABSTRACT

An RNAi agent targets a component of a human Notch signalling pathway other than presenilin1 or presenilin2 by RNA interference to reduce expression of said component.

### CROSS REFERENCE TO RELATED APPLICATIONS

This application is a continuation-in-part of International Application No. PCT/GB2005/000243, filed Jan. 27, 2005, published as WO 2005/073250 on Aug. 11, 2005, and claiming priority to GB Application Serial Nos. 0401807.3 and 0401792.7, filed Jan. 28, 2004, and 0419703.4, filed Sep. 4, 2004.

Reference is made to U.S. application Ser. No. 09/310,685, filed May 4, 1999; Ser. No. 09/870,902, filed May 31, 2001; Ser. No. 10/013,310, filed Dec. 7, 2001; Ser. No. 10/147,354, filed May 16, 2002; Ser. No. 10/357,321, filed Feb. 3, 2002; Ser. No. 10/682,230, filed Oct. 9, 2003; Ser. No. 10/720,896, filed Nov. 24, 2003; Ser. Nos. 10/763,362, 10/764,415 and 10/765,727, all filed Jan. 23, 2004; Ser. No. 10/812,144, filed Mar. 29, 2004; Ser. Nos. 10/845,834 and 10/846,989, both filed May 14, 2004; Ser. No. 10/877,563, filed Jun. 25, 2004; Ser. No. 10/899,422, filed Jul. 26, 2004; Ser. No. 10/958,784, filed Oct. 5, 2004; Ser. No. 11/050,328, filed Feb. 3, 2005; Ser. No. 11/058,066, filed Feb. 14, 2005; Ser. No. 11/071,796, filed Mar. 3, 2005; Ser. No. 11/078,735, filed Mar. 10, 2005; Ser. No. 11/103,077, filed Apr. 11, 2005; Ser. No. 11/178,724, filed Jul. 11, 2005; Ser. No. 11/188,417, filed Jul. 25, 2005, Ser. Nos. 11/231,494 and 11/232,404, both filed Sep. 21, 2005.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates to modulation of the Notch signalling pathway in therapy, and particularly, but not exclusively, in immunotherapy.

### BACKGROUND OF THE INVENTION

International Patent Publication No WO 98/20142 describes how manipulation of the Notch signalling pathway can be used in immunotherapy and in the prevention and/or treatment of T-cell mediated diseases. In particular, allergy, autoimmunity, graft rejection, tumour induced aberrations to the T-cell system and infectious diseases caused, for example, by Plasmodium species, Microfilariae, Helminths, Mycobacteria, HIV, Cytomegalovirus, Pseudomonas, Toxoplasma, Echinococcus, Haemophilus influenza type B, measles, Hepatitis C or Toxicara, may be targeted.

It has also been shown that it is possible to generate a class of regulatory T cells which are able to transmit antigen-specific tolerance to other T cells, a process termed infectious tolerance (WO98/20142). The functional activity of these cells can be mimicked by over-expression of a Notch ligand protein on their cell surfaces or on the surface of antigen presenting cells. In particular, regulatory T cells can be generated by over-expression of a member of the Delta or Serrate family of Notch ligand proteins.

A description of the Notch signalling pathway and conditions affected by it may be found in our published PCT Applications WO 98/20142, WO 00/36089 and WO 0135990. The text of each of PCT/GB97/03058 (WO 98/20142), PCT/GB99/04233 (WO 00/36089) and PCT/GB00/04391 (WO 0135990) is hereby incorporated herein by reference (see also Hoyne G. F. et al (1999) Int Arch Allergy Immunol 118:122-124; Hoyne et al. (2000) Immunology 100:281-288; Hoyne G. F. et al (2000) Intl Immunol 12:177-185; Hoyne, G. et al. (2001) Immunological Reviews 182:215-227).

A description of the Notch signalling pathway and conditions affected by it may be found, for example, in our published PCT Applications as follows:
PCT/GB97/03058 (filed on 6 Nov. 1997 and published as WO 98/20142; claiming priority from GB 9623236.8 filed on 7 Nov. 1996, GB 9715674.9 filed on 24 Jul. 1997 and GB 9719350.2 filed on 11 Sep. 1997);
PCT/GB99/04233 (filed on 15 Dec. 1999 and published as WO 00/36089; claiming priority from GB 9827604.1 filed on 15 Dec. 1999);
PCT/GB00/04391 (filed on 17 Nov. 2000 and published as WO 0135990; claiming priority from GB 9927328.6 filed on 18 Nov. 1999);
PCT/GB01/03503 (filed on 3 Aug. 2001 and published as WO 02/12890; claiming priority from GB 0019242.7 filed on 4 Aug. 2000);
PCT/GB02/02438 (filed on 24 May 2002 and published as WO 02/096952; claiming priority from GB 0112818.0 filed on 25 May 2001);
PCT/GB02/03381 (filed on 25 Jul. 2002 and published as WO 03/012111; claiming priority from GB 0118155.1 filed on 25 Jul. 2001);
PCT/GB02/03397 (filed on 25 Jul. 2002 and published as WO 03/012441; claiming priority from GB0118153.6 filed on 25 Jul. 2001, GB0207930.9 filed on 5 Apr. 2002, GB 0212282.8 filed on 28 May 2002 and GB 0212283.6 filed on 28 May 2002);
PCT/GB02/03426 (filed on 25 Jul. 2002 and published as WO 03/011317; claiming priority from GB0118153.6 filed on 25 Jul. 2001, GB0207930.9 filed on 5 Apr. 2002, GB 0212282.8 filed on 28 May 2002 and GB 0212283.6 filed on 28 May 2002);
PCT/GB02/04390 (filed on 27 Sep. 2002 and published as WO 03/029293; claiming priority from GB 0123379.0 filed on 28 Sep. 2001);
PCT/GB02/05137 (filed on 13 Nov. 2002 and published as WO 03/041735; claiming priority from GB 0127267.3 filed on 14 Nov. 2001, PCT/GB02/03426 filed on 25 Jul. 2002, GB 0220849.4 filed on 7 Sep. 2002, GB 0220913.8 filed on 10 Sep. 2002 and PCT/GB02/004390 filed on 27 Sep. 2002);
PCT/GB02/05133 (filed on 13 Nov. 2002 and published as WO 03/042246; claiming priority from GB 0127271.5 filed on 14 Nov. 2001 and GB 0220913.8 filed on 10 Sep. 2002).

Each of PCT/GB97/03058 (WO 98/20142), PCT/GB99/04233 (WO 00/36089), PCT/GB00/04391 (WO 0135990), PCT/GB01/03503 (WO 02/12890), PCT/GB02/02438 (WO 02/096952), PCT/GB02/03381 (WO 03/012111), PCT/GB02/03397 (WO 03/012441), PCT/GB02/03426 (WO 03/011317), PCT/GB02/04390 (WO 03/029293), PCT/GB02/05137 (WO 03/041735) and PCT/GB02/05133 (WO 03/042246) are hereby incorporated herein by reference.

The present invention seeks to provide further methods of modulating the Notch signalling pathway, and, in particular, for modulating immune responses.

### SUMMARY OF THE INVENTION

According to a first aspect of the invention there is provided an RNAi agent which targets a component of a human Notch signalling pathway (in this general case the target preferably not being presenilin1 or presenilin2) by RNA interference to reduce expression of said component.

According to a further aspect of the invention there is provided a method for treating a disease or disorder (in this general case the target preferably not being presenilin1 or presenilin2) by modulating Notch signalling by RNA interference.

According to a more particular aspect of the invention there is provided a method for treating an immune disease or disorder by modulating Notch signalling by RNA interference.

According to a further aspect of the invention there is provided a method for modulating an immune response by modulating Notch signalling by RNA interference. For example, without wishing to be bound by any theory of mode of action, in one embodiment immune responses may be reduced by increasing Notch signalling to increase production or activity of regulatory T-cells (Tregs). In an alternative embodiment immune responses may be increased by decreasing Notch signalling to reduce or inhibit regulatory T-cells (Tregs).

According to a further aspect of the invention there is provided a method for modulating immune cell activation by modulating Notch signalling by RNA interference.

According to a further aspect of the invention there is provided a method for modulating lymphocyte activation by modulating Notch signalling by RNA interference.

According to a further aspect of the invention there is provided a method for modulating T-cell activation by modulating Notch signalling by RNA interference.

According to a further aspect of the invention there is provided a method for increasing lymphocyte (e.g. T-cell) activation by reducing Notch signalling by RNA interference, e.g. by use of an RNAi agent, e.g. a Notch or Notch ligand RNAi agent, as described herein.

According to a further aspect of the invention there is provided a method for reducing lymphocyte (e.g. T-cell) activation to treat an immune disorder by increasing Notch signalling by RNA interference, e.g. by use of an RNAi agent as described herein.

According to a further aspect of the invention there is provided a method for modulating T-cell to T-cell Notch signalling to treat an immune disorder by use of a Delta protein or nucleic acid or a fragment, derivative, variant, peptidomimetic or antibody thereof.

According to one aspect there is provided a method for increasing T-cell to T-cell Notch signalling to treat an immune disorder such as allergy, autoimmune disease or transplant rejection by use of a Delta agonistic protein or nucleic acid or a fragment, derivative, variant, peptidomimetic or antibody thereof.

Alternatively there is provided a method for decreasing T-cell to T-cell Notch signalling to increase an immune response by use of a Delta antagonistic protein or nucleic acid or a fragment, derivative, variant, peptidomimetic or antibody thereof.

Suitably the Delta nucleic acid may be a Delta RNAi agent as described herein.

In one embodiment the RNAi agent may not target a Jagged Notch ligand.

In one embodiment the RNAi agent may not target a Delta Notch ligand.

In one embodiment the RNAi agent may not target Notch 1 or Notch 2.

In one embodiment the RNAi agent may not target a Notch receptor.

In one embodiment the RNAi agent may not target a Presenilin.

According to a further aspect of the invention there is provided a method for modulating APC/T-cell Notch signalling to treat an immune disorder by use of an RNAi agent which modulates Notch signalling as described herein.

According to a further aspect of the invention there is provided a method for treating a disease or disorder associated with Notch signaling comprising reducing expression of a component of the Notch signaling pathway in a target cell of a mammal, said method comprising administering to said mammal an effective amount of an RNAi agent specific for said component to reduce expression thereof.

According to a further aspect of the invention there is provided a pharmaceutical composition for modulation of Notch signaling comprising an RNAi agent which downregulates expression of a component of the Notch signaling pathway by RNA interference.

According to a further aspect of the invention there is provided a pharmaceutical composition for treatment of an immune disease or disorder comprising an RNAi agent which downregulates expression of a component of the Notch signaling pathway by RNA interference.

According to a further aspect of the invention there is provided a pharmaceutical composition for modulation of an immune response comprising an RNAi agent which downregulates expression of a component of the Notch signaling pathway by RNA interference.

According to a further aspect of the invention there is provided a pharmaceutical composition comprising:
i) an RNAi agent targeting a component of the Notch signaling pathway; and
ii) an antigen or antigenic determinant or a nucleic acid coding for an antigen or antigenic determinant;
   as a combined preparation for simultaneous, separate or sequential administration for the modulation of an immune response.

Generally, use of an RNAi agent which increases Notch signalling will reduce an immune response, which may be useful for example to treat unwanted immune responses for example in autoimmune disease, allergy or graft rejection.

Conversely, use of an RNAi agent which decreases Notch signalling may increase an immune response, which may be useful for example, for vaccination or treatment of cancer or infectious disease.

According to a further aspect of the invention there is provided a pharmaceutical composition comprising:
i) an RNAi agent targeting a component of the Notch signaling pathway; and
ii) an antigen or antigenic determinant or a nucleic acid coding for an antigen or antigenic determinant;
   as a combined preparation for simultaneous, separate or sequential administration for the modulation of an immune response to said antigen or antigenic determinant.

According to a further aspect of the invention there is provided a pharmaceutical composition comprising:
i) an RNAi agent targeting a component of the Notch signaling pathway to increase Notch signalling; and
ii) an antigen or antigenic determinant or a nucleic acid coding for an antigen or antigenic determinant;
   as a combined preparation for simultaneous, separate or sequential administration for reducing an immune response to said antigen or antigenic determinant, such as an allergen, autoantigen, pathogen antigen or graft antigen.

According to a further aspect of the invention there is provided a pharmaceutical composition comprising:
i) an RNAi agent targeting a component of the Notch signaling pathway to decrease Notch signalling; and
ii) an antigen or antigenic determinant or a nucleic acid coding for an antigen or antigenic determinant;
   as a combined preparation for simultaneous, separate or sequential administration for increasing an immune response to said antigen or antigenic determinant, such as a pathogen or cancer antigen.

According to a further aspect of the invention there is provided a cancer vaccine composition comprising an RNAi agent targeting a component of the Notch signalling pathway which is effective to reduce Notch signalling.

According to a further aspect of the invention there is provided a pathogen vaccine composition comprising an RNAi agent targeting a component of the Notch signalling pathway which is effective to reduce Notch signalling.

Suitably the RNAi agent may be in the form of a siNA, such as a siRNA.

Alternatively, the RNAi may be in the form of a shRNA.

Suitably the RNAi agent comprises a transcription template coding for an interfering ribonucleic acid, suitably an shRNA or siRNA. Suitably the transcription template comprises a DNA sequence, which may suitably encode a shRNA.

Suitably the RNAi agent targets a Notch ligand to reduce expression of thereof.

Suitably, the RNAi agent targets Delta to reduce expression thereof.

Suitably the RNAi agent targets Delta1, Delta3 or Delta4 to reduce expression thereof.

Suitably the RNAi agent targets Delta1 to reduce expression thereof.

Suitably the Delta1 target sequence comprises a sequence of about 19-22 nucleic acids of human Delta1.

Suitably the RNAi agent targets Jagged, suitably Jagged1 or Jagged2, to reduce expression thereof.

Suitably the RNAi agent targets expression of Notch, such as Notch1, Notch2, Notch3 or Notch4, to reduce expression thereof. For example, the RNAi agent may target Notch IC to reduce expression thereof.

Alternatively, the RNAi agent may target a Fringe to reduce expression thereof.

Alternatively, the RNAi agent may target a Notch IC protease complex component to reduce expression thereof.

Alternatively, the RNAi agent may target a Notch Ubiquitin ligase to reduce expression thereof.

Alternatively, the RNAi agent may target Deltex to reduce expression thereof.

Alternatively, the RNAi agent may target a member of the HES family of basic helix-loop-helix transcriptional regulators, or a CSL transcriptional cofactor to reduce expression thereof.

Preferably the RNAi agent targets Notch signalling in immune cells, suitably in T-cells, B-cells or APCs.

According to a further aspect of the invention there is provided a vector, preferably an expression vector, coding for an RNAi agent as defined above.

According to a further aspect of the invention there is provided a vector comprising:
i) a first polynucleotide sequence coding for an RNAi agent as defined above; and
ii) a second polynucleotide sequence coding for an antigen or antigenic determinant.

In one embodiment the antigen may be an autoantigen, allergen, pathogen antigen or graft antigen or antigenic determinant thereof.

In an alternative embodiment the antigen may be a pathogen or tumour antigen or antigenic determinant thereof.

According to a further aspect of the invention there is provided the use of an RNAi agent as described herein for the manufacture of a medicament for modulation of expression of a cytokine such as those selected from IL-10, IL-5, IL-2, TNF-alpha, IFN-gamma or IL-13.

Thus in one aspect there is provided the use of an RNAi agent as described herein which activates Notch signalling for the manufacture of a medicament for increasing IL-10 expression in an immune cell. Conversely, there is provided the use of an RNAi agent as described herein which reduces Notch signalling for the manufacture of a medicament for decreasing IL-10 expression in an immune cell.

Alternatively there is provided the use of an RNAi agent as described herein which activates Notch signalling for the manufacture of a medicament for decrease of expression of a cytokine selected from IL-2, IL-5, TNF-alpha, IFN-gamma or IL-13. Conversely there is provided the use of an RNAi agent as described herein which reduces Notch signalling for the manufacture of a medicament for increase of expression of a cytokine selected from IL-2, IL-5, TNF-alpha, IFN-gamma or IL-13.

According to a further aspect of the invention there is provided the use of an RNAi agent as described herein which activates Notch signalling for the manufacture of a medicament for generating an immune modulatory cytokine profile with increased IL-10 expression and reduced IL-5 expression. Conversely there is provided the use of an RNAi agent as described herein which reduces Notch signalling for the manufacture of a medicament for generating an immune modulatory cytokine profile with reduced IL-10 expression and increased IL-5 expression.

According to a further aspect of the invention there is provided the use of an RNAi agent as described herein which activates Notch signalling for the manufacture of a medicament for generating an immune modulatory cytokine profile with increased IL-10 expression and reduced IL-2, IFN-gamma, IL-5, IL-13 and TNF-alpha expression. Conversely there is provided the use of an RNAi agent as described herein which reduces Notch signalling for the manufacture of a medicament for generating an immune modulatory cytokine profile with reduced IL-10 expression and increased IL-2, IFN-gamma, IL-5, IL-13 and TNF-alpha expression.

According to a further aspect of the present invention there is provided a method for detecting immunologically active RNAi agent modulators of Notch signalling comprising the steps of:
(a) contacting a cell of the immune system with a candidate RNAi agent;
(b) monitoring Notch signalling; and
(c) determining whether the candidate modulator modulates Notch signalling.

“Contacting” means bringing together in such a way so as the cell may interact with the candidate modulator. Preferably this will be in an aqueous solvent or buffering solution.

According to a further aspect of the invention there is provided a method for detecting immunologically active RNAi agent modulators of Notch signalling comprising the steps of (in any order):
(a) activating a cell of the immune system;
(b) contacting the cell with a candidate RNAi agent modulator of Notch signalling;
(c) monitoring Notch or immune signalling; and
(d) determining whether the candidate modulator modulates Notch or immune signalling.

According to a further aspect of the invention there is provided a method for detecting immunologically active RNAi agent modulators of Notch or immune signalling comprising the steps of (in any order):
(a) activating a cell of the immune system;
(b) activating Notch signalling in the cell;
(c) contacting the cell with a candidate RNAi agent modulator of Notch or immune signalling;
(d) monitoring Notch or immune signalling; and
(e) determining whether the candidate modulator modulates Notch or immune signalling.

According to a further aspect of the invention there is provided a method for detecting immunologically active RNAi agent modulators of Notch or immune signalling comprising the steps of (in any order):
(a) activating Notch signalling in a cell of the immune system;
(b) contacting the cell with a candidate RNAi agent modulator of Notch signalling;
(c) monitoring Notch or immune signalling; and
(d) determining whether the candidate modulator modulates Notch or immune signalling.

According to a further aspect of the invention there is provided a method for detecting immunologically active RNAi agent modulators of Notch signalling comprising the steps of (in any order):
(a) activating a cell of the immune system;
(b) contacting the cell with a candidate RNAi agent modulator of Notch signalling;
(c) monitoring Notch or immune signalling; and
(d) determining whether the candidate modulator modulates Notch or immune signalling.

According to a further aspect of the invention there is provided a method for detecting immunologically active RNAi agent modulators of Notch signalling comprising the steps of (in any order):
(a) activating a cell of the immune system;
(b) activating Notch signalling in the cell;
(c) contacting the cell with a candidate RNAi agent modulator of Notch signalling;
(d) monitoring Notch or immune signalling; and
(e) determining whether the candidate modulator modulates Notch or immune signalling.

Suitably immune cell activation is at least 20%, preferably at least 70% optimal with respect to Notch or immune signalling.

In a preferred embodiment, the step of monitoring Notch signalling comprises the steps of monitoring levels of expression of at least one target gene. The target gene may be an endogenous target gene of the Notch signalling pathway or a reporter gene.

Known endogenous target genes of the Notch signalling pathway include Deltex, Hes-1, Hes-5, E(spl), Il-10, CD-23, Dlx-1, CTLA4, CD-4, Numb, Mastermind and Dsh.

Many reporter genes are standard in the art and include genes encoding an enzymatic activity, genes comprising a radiolabel or a fluorescent label and genes encoding a predetermined polypeptide epitope.

Preferably at least one target gene is under the transcriptional control of a promoter region sensitive to Notch signalling. Even more preferably, at least one target gene is under the transcriptional control of a promoter region sensitive to Notch signalling and a second signal, and/or a third signal wherein the second and third signals are different.

An example of a signal of use in the present invention is a signal that results from activation of a signalling pathway specific to cells of the immune system, such as a T cell receptor (TCR) signalling pathway, a B cell receptor (BCR) signalling pathway or a Toll-like receptor (TLR) signalling pathway, with or without an accessory signal (known in the art as costimulatory signals for T and B cell receptor signalling).

Another example of a signal of use in the present invention is a costimulus specific to cells of the immune system such as B7 proteins including B7.1-CD80, B7.2-CD86, B7H1, B7H2, B7H3, B7RP1, B7RP2, CTLA4, ICOS, CD2, CD24, CD27, CD28, CD30, CD34, CD38, CD40, CD44, CD45, CD49, CD69, CD70, CD95 (Fas), CD134, CD134L, CD153, CD154, 4-1BB, 4-1BB-L, LFA-1, ICAM-1, ICAM-2, ICAM-3, OX40, OX40L, TRANCE/RANK ligands, Fas ligand, MHC class II, DEC205-CD205, CD204-Scavenger receptor, CD14, CD206 (mannose receptor), Toll-like receptors (TLR) such as TLR 1-9, CD207 (Langerin), CD209 (DC-SIGN), FCγ receptor 2 (CD32), CD64 (FCγ receptor 1), CD68, CD83, CD33, CD54, BDCA-2, BDCA-3, BDCA-4, chemokine receptors, cytokines, growth factors or growth factor receptor agonists, and variants, derivatives, analogues and fragments thereof.

In a preferred embodiment, the method of the present invention is carried out in a T cell or T cell progenitor or an antigen presenting cell (APC). APCs are cells which are capable of expressing MHC class II molecules and able to present antigens to CD4+ T cells. Preferably, the APC will be a myeloid lineage cell such as a dendritic cell, for example a Langerhans cell, a monocyte or macrophage or a primary cell or a B lineage cell.

Levels of expression of at least one target gene can be monitored with a protein or a nucleic acid assay.

In accordance with another aspect of the present invention there is provided a method for detecting RNAi agent modulators of Notch signalling comprising the steps of:
(a) activating a cell of the immune system;
(b) contacting the cell with a candidate RNAi agent modulator;
(c) monitoring Notch signalling;
(wherein steps (a), (b) and (c) can be carried out in any order); and
(d) determining whether the candidate modulator modulates Notch signalling.

Suitably the expression of the at least one target gene is monitored with a protein or nucleic acid assay

Preferably the cell of the immune system is a T-cell or T-cell progenitor.

Preferably the T-cell is activated by activation of the T-cell receptor.

Preferably the T-cell receptor is activated with an antigen or antigenic determinant.

Preferably the T-cell receptor is activated by an anti-CD3 or anti-TCR antibody which are preferably bound to a support. Preferably the anti-CD3 or anti-TCR antibody is bound to a particulate support.

Preferably the T-cell is co-activated, suitably by activation of CD28.

Preferably the T-cell receptor is co-activated by an anti-CD28 antibody or CD28 ligand, such as an active domain of B7.

Preferably the T-cell is activated by an anti-CD3 antibody and co-activated by anti-CD28 antibody.

Alternatively the T-cell may be activated with a calcium ionophore or an activator of protein kinase C or MAP Kinase.

Suitably the immune cell may be transfected with an expression vector coding for Notch, a constitutively active truncated form of Notch or a Notch IC domain, and if desired a Notch reporter construct.

In a preferred embodiment the method comprises the steps of:
i) activating Notch signalling in the immune cell with a further agent; and
ii) determining whether the candidate modulator modulates such Notch signalling activation and/or immune cell activation.

In one embodiment Notch signalling may be activated with a Notch ligand or an active portion of a Notch ligand, for example a Notch ligand EC domain. Suitably the Notch ligand may be bound to a membrane or support.

According to a further aspect of the present invention there is provided a particle comprising an active portion of a Delta ligand bound to a particulate support matrix.

Preferably the particulate support matrix is a bead. The bead may be, for example, a magnetic bead (e.g. as available under the trade name “Dynal”) or a polymeric bead such as a Sepharose bead. Suitably a plurality of active portions of a Delta ligand are bound to the particulate support matrix.

According to a yet further aspect of the present invention there is provided a modulator identifiable or identified by the method of the invention.

According to yet another aspect of the present invention there is provided the use of an RNAi agent modulator according to the present invention in the preparation of a medicament for the treatment of a disease or condition of, or related to the immune system. Preferably, the disease is a T-cell mediated disease.

According to yet another aspect of the present invention there is provided a pharmaceutical composition comprising a therapeutically effective amount of at least one RNAi agent modulator according to the invention and a pharmaceutically acceptable carrier, diluent and/or excipient.

Preferably the Notch signalling pathway is activated with an agent capable of activating a Notch receptor. Suitably the modulator targets a Notch ligand or a biologically active fragment or derivative of a Notch ligand.

### DETAILED DESCRIPTION

Various preferred features and embodiments of the present invention will now be described in more detail by way of non-limiting examples and with reference to the accompanying Figures, in which:

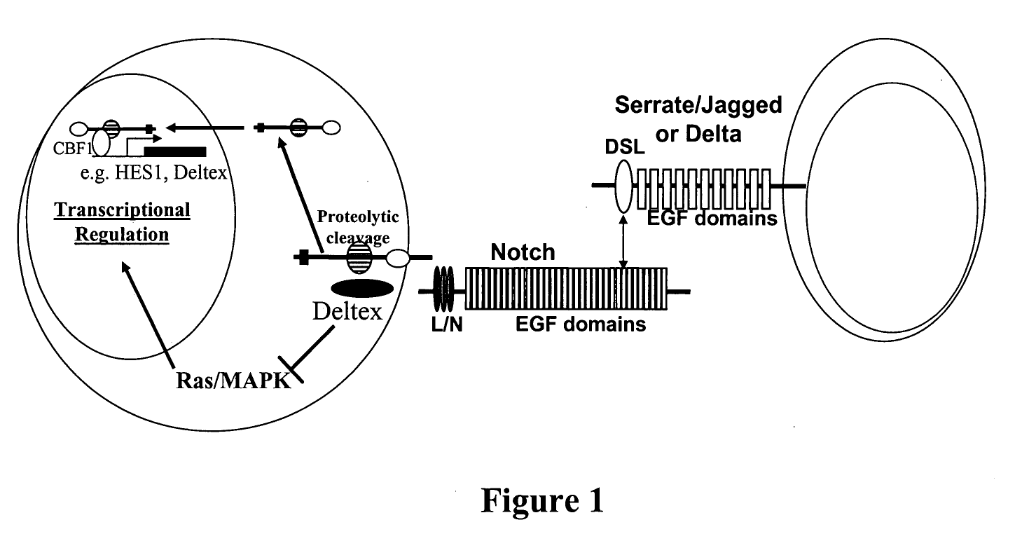

FIG. 1 shows a schematic representation of the Notch signalling pathway.

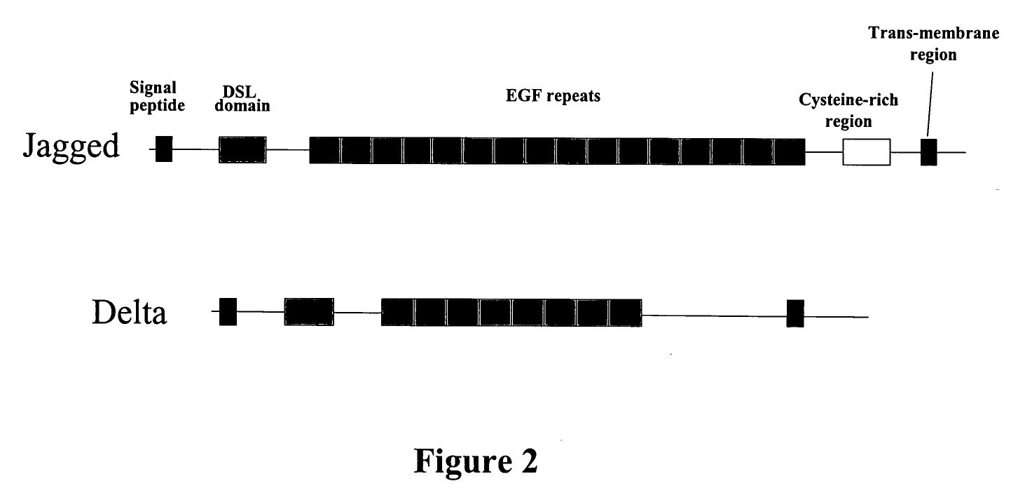

FIG. 2 shows schematic representations of the Notch ligands Jagged and Delta.

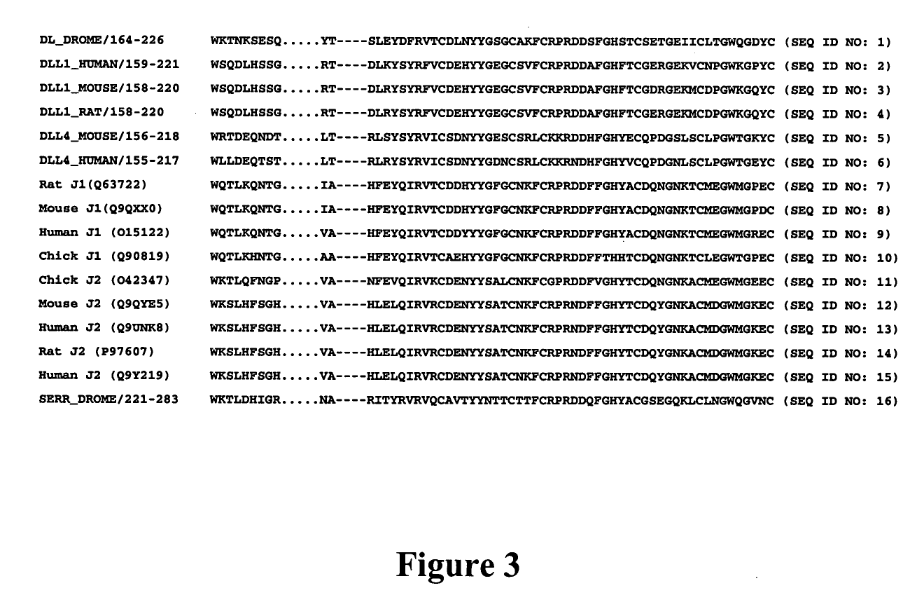

FIG. 3 shows aligned amino acid sequences (SEQ ID NOs: 1-16) of DSL domains from various Drosophila and mammalian Notch ligands.

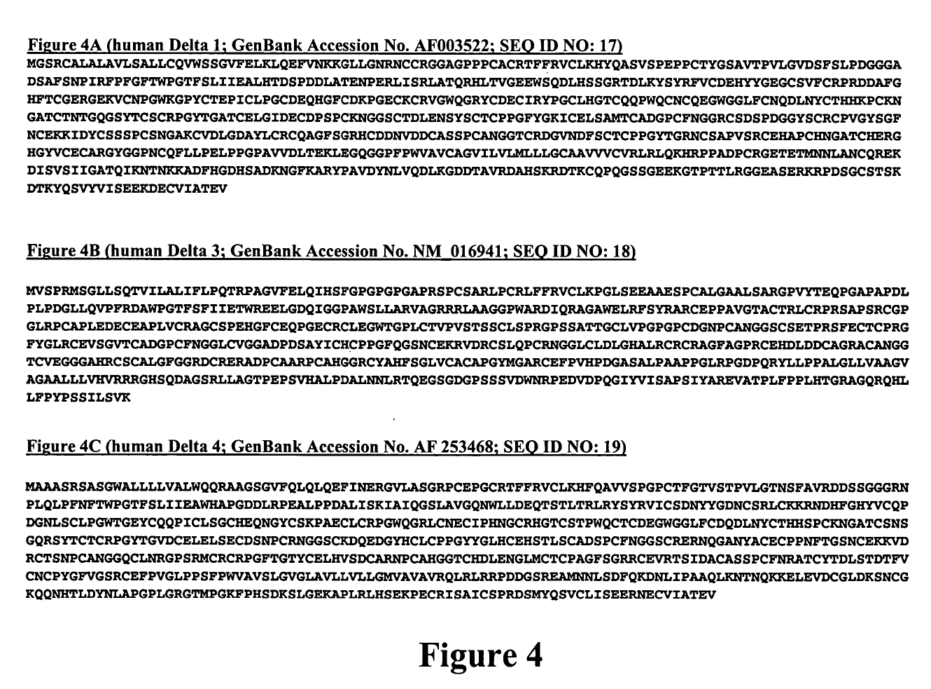

FIG. 4A shows the amino acid sequence of human Delta-1 (SEQ ID NO: 17); FIG. 4B shows the amino acid sequence of human Delta-3 (SEQ ID NO: 18); and FIG. 4C shows the amino acid sequence of human Delta-4 (SEQ ID NO: 19).

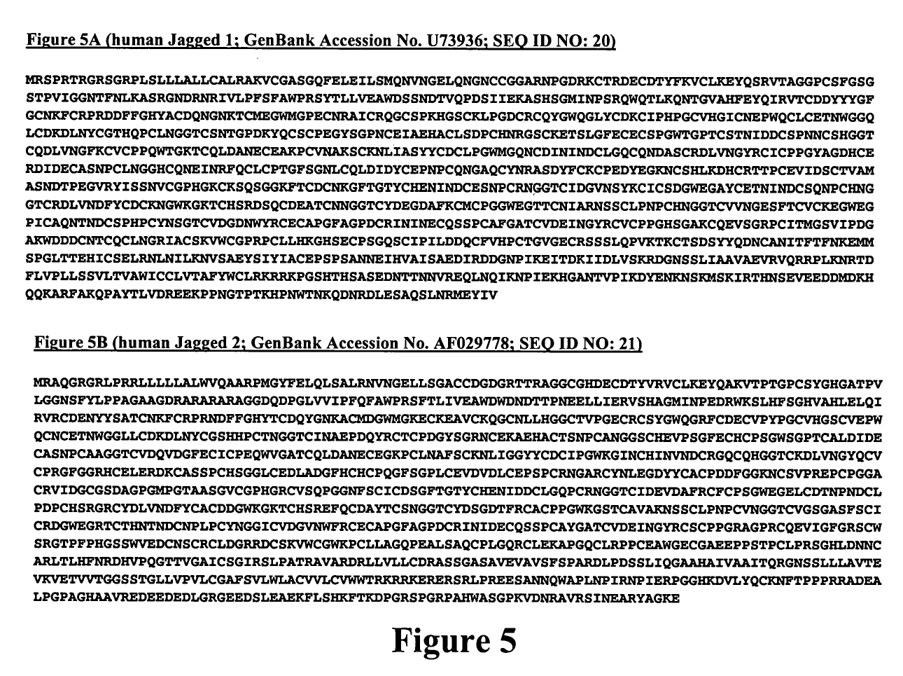

FIG. 5A shows the amino acid sequence of human Jagged-1 (SEQ ID NO: 20); and FIG. 5B shows the amino acid sequence of human Jagged-2 (SEQ ID NO: 21).

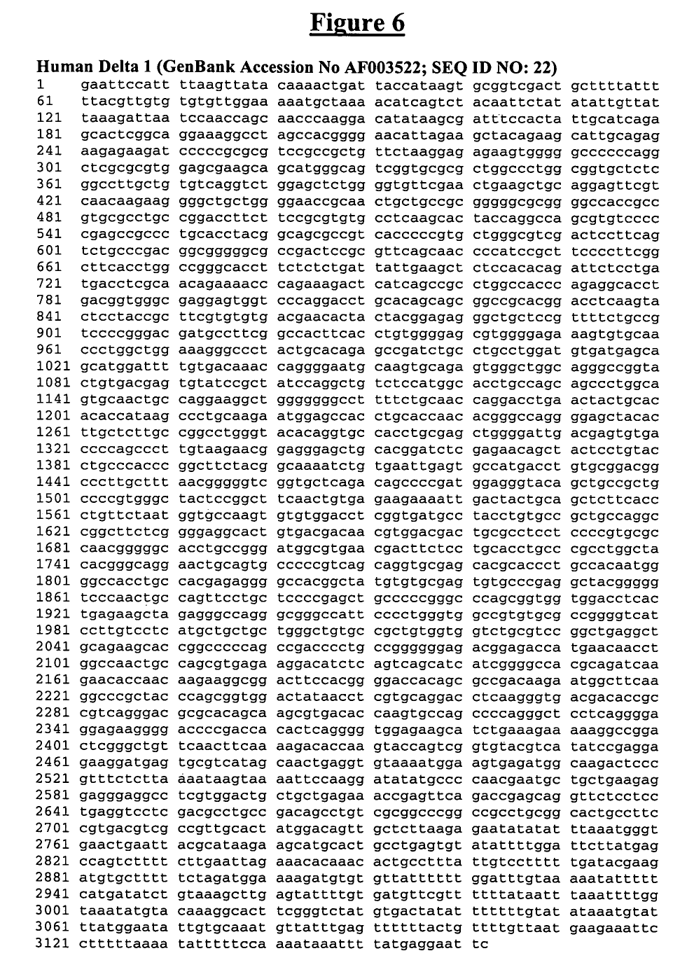

FIG. 6 shows a nucleic acid sequence of human Delta1 (SEQ ID NO: 22).

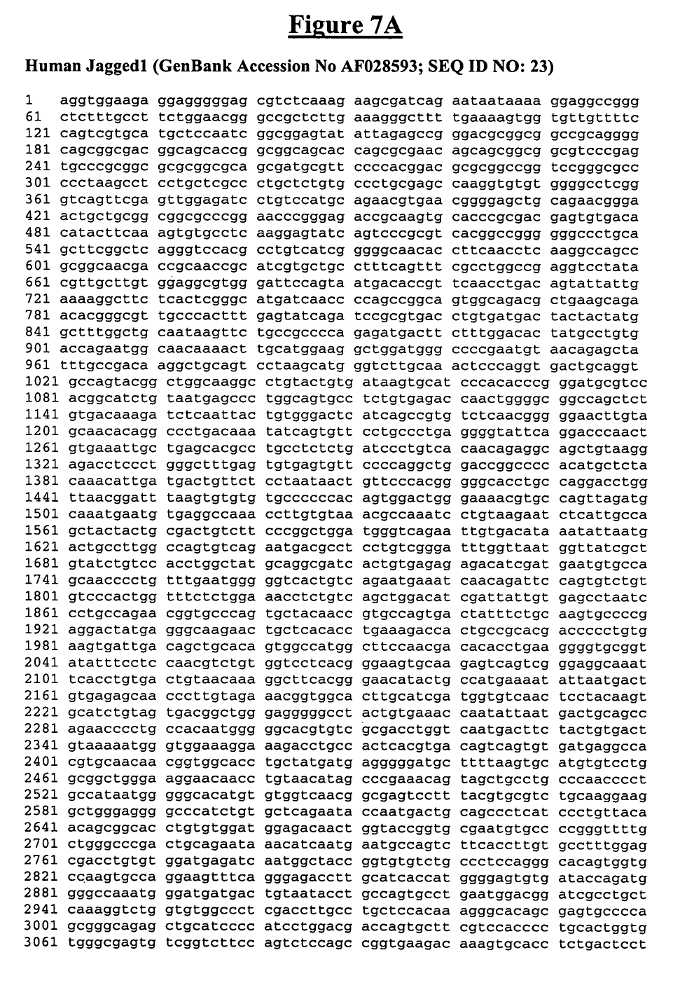

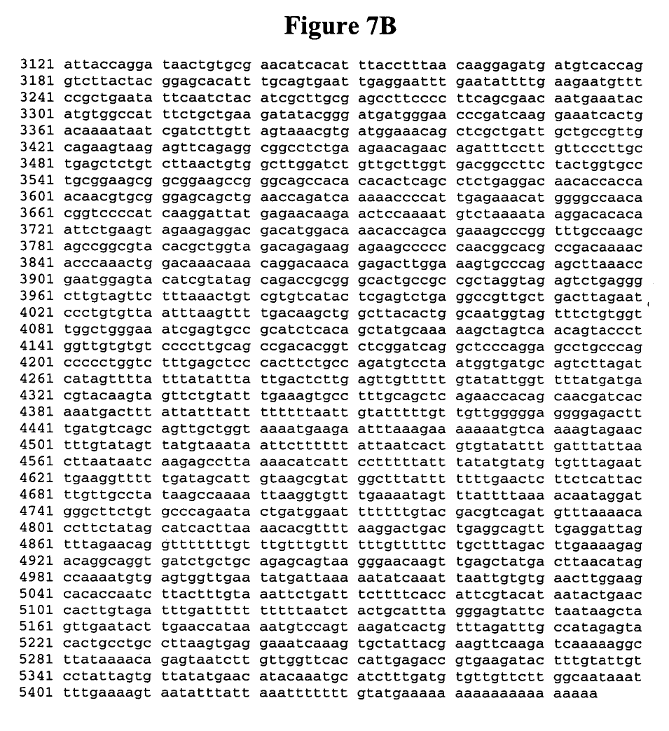

FIGS. 7A and 7B show a nucleic acid sequence of human Jagged1 (SEQ ID NO: 23).

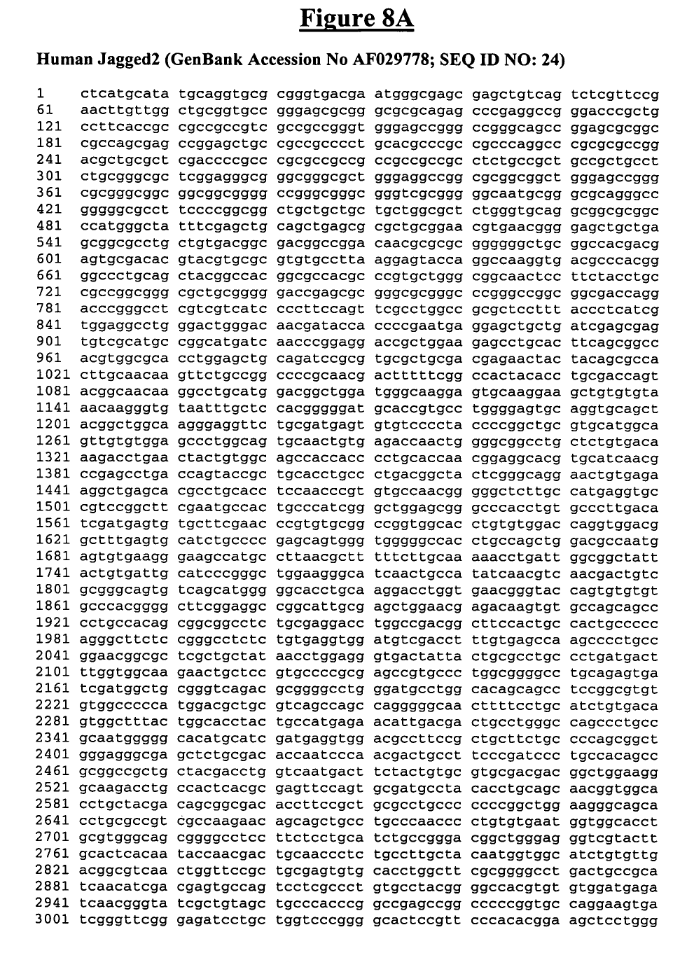

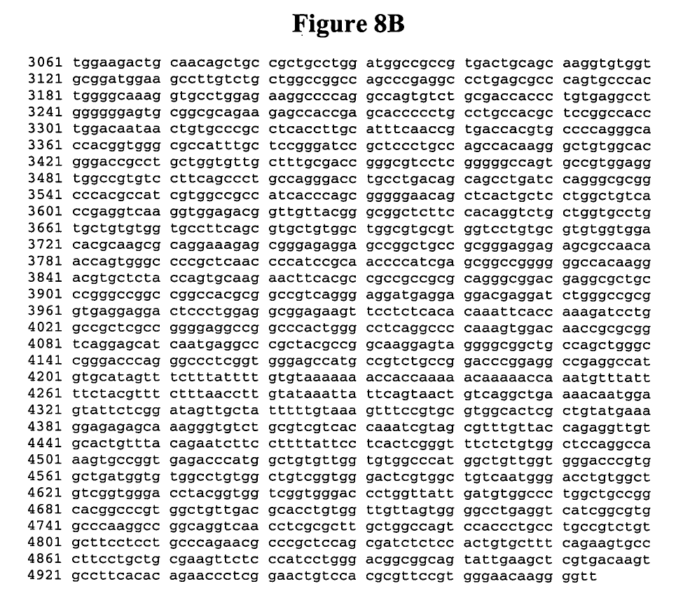

FIGS. 8A and 8B shows a nucleic acid sequence of human Jagged2 (SEQ ID NO: 24).

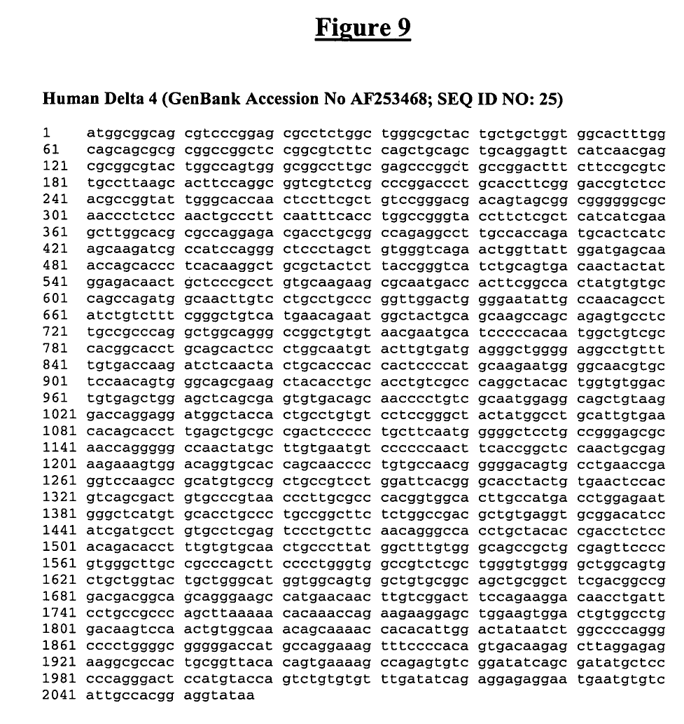

FIG. 9 shows a nucleic acid sequence of human Jagged1 (SEQ ID NO: 25).

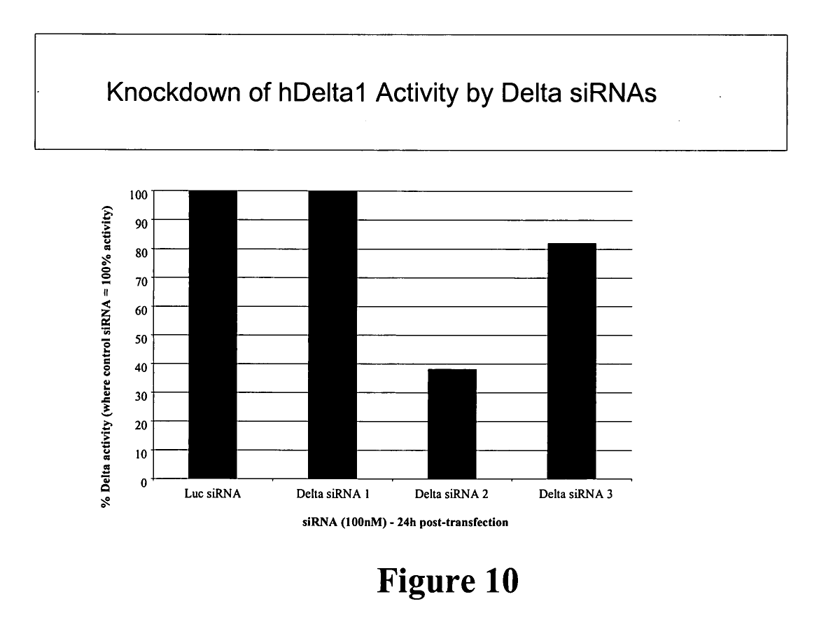

FIG. 10 shows the knockdown of hDelta1 activity by Delta siRNAs in CHO cells transfected with FL Delta V5+Delta siRNA or control Luc siRNA, assayed using CHO-N2 assay, in the presence of 10 mM LiCl.

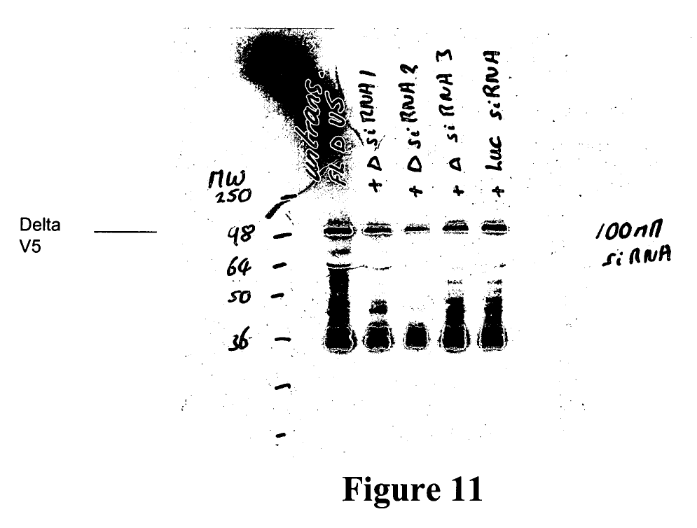

FIG. 11 shows a Western blot of an extract of CHO cells co-transfected with FL hDelta1 V5-His and Delta siRNA, run on a 12% SDS-PAGE gel and Western blotted with anti-V5 HRP.

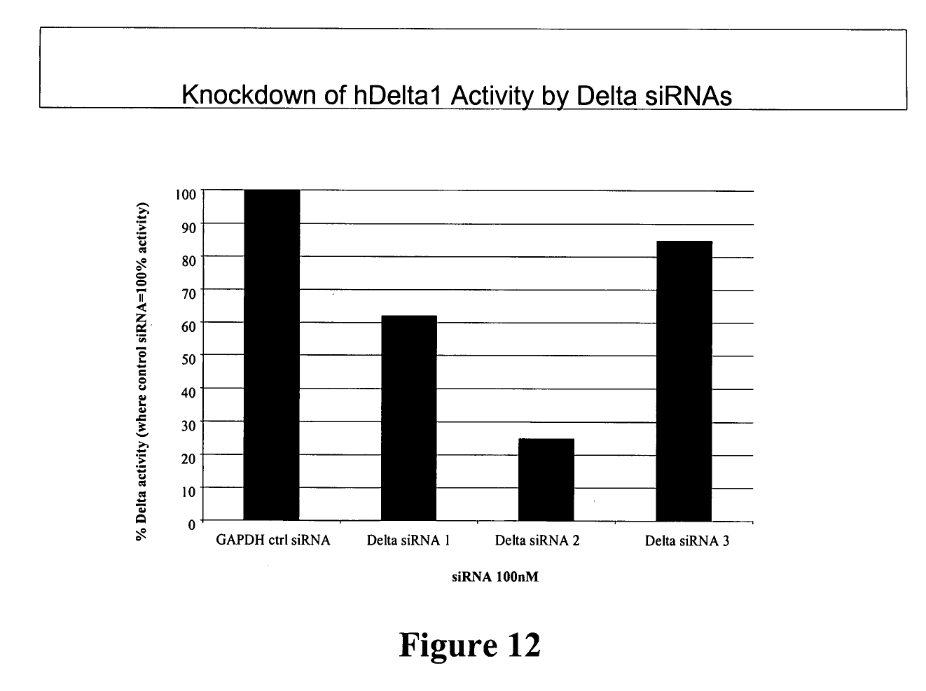

FIG. 12 shows the knockdown of hdelta1 activity by Delta siRNAs in CHO-Delta cells transfected with Delta siRNAs and control GAPDH siRNA, assayed using CHO-N2 assay.

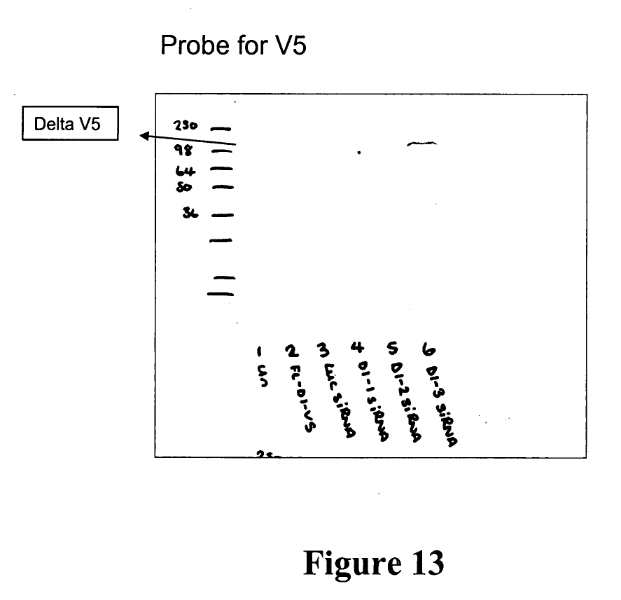

FIG. 13 shows a Western blot of an extract of Jurkat cells co-transfected with FL hDelta1 V5-His and Delta siRNA, run on a 12% SDS-PAGE gel and Western blotted with anti-V5 HRP.

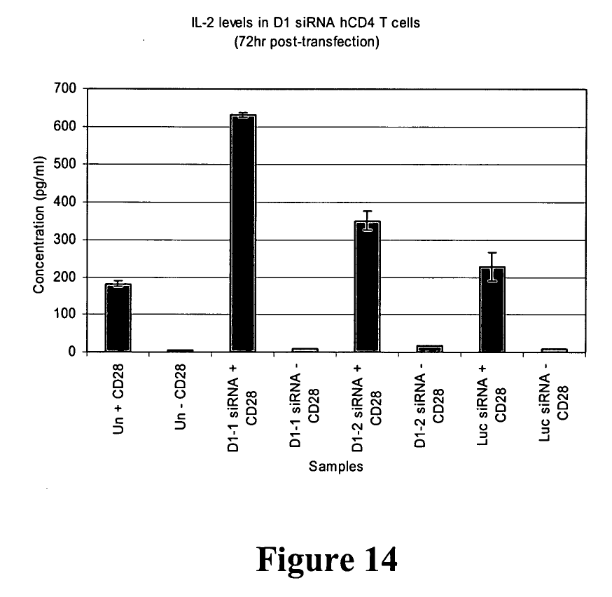

FIG. 14 shows IL-2 levels in D1 siRNA treated hCD4 T cells at 72 hours post transfection.

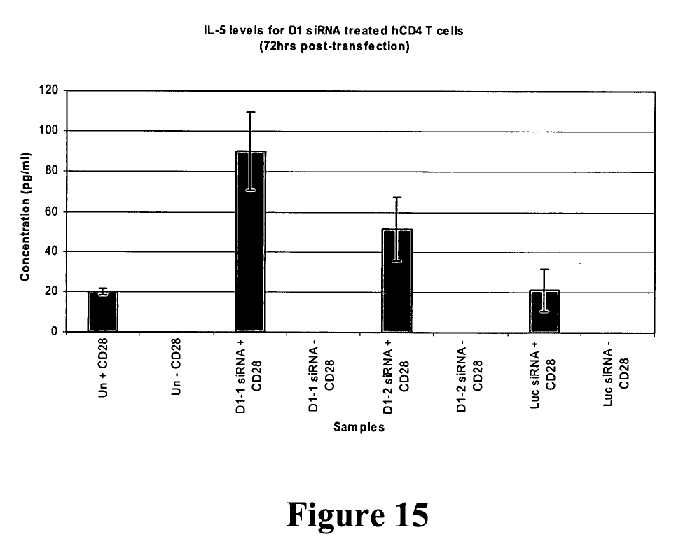

FIG. 15 shows IL-5 levels in D1 siRNA treated hCD4 T cells at 72 hours post transfection.

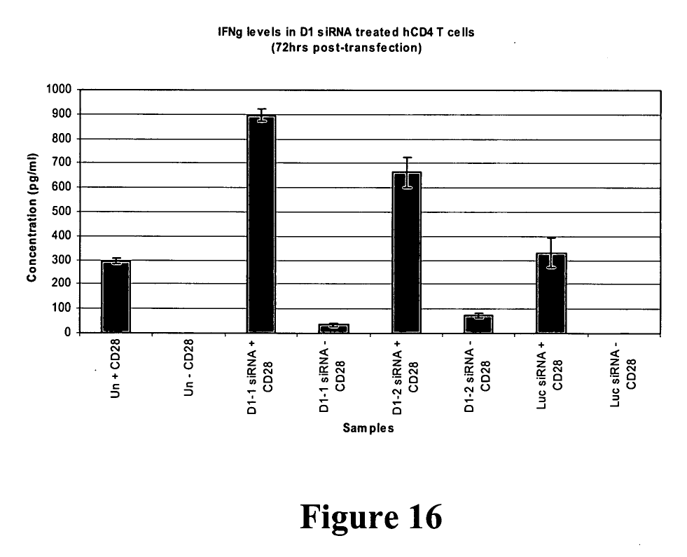

FIG. 16 shows IFN-γ levels in D1 siRNA treated hCD4 T cells at 72 hours post transfection.

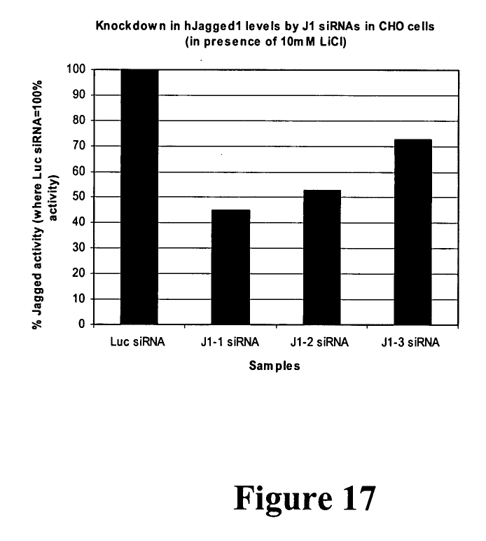

FIG. 17 shows the knockdown of hjagged1 levels by J1 siRNAs in CHO cells in the presence of 10 mM LiCl.

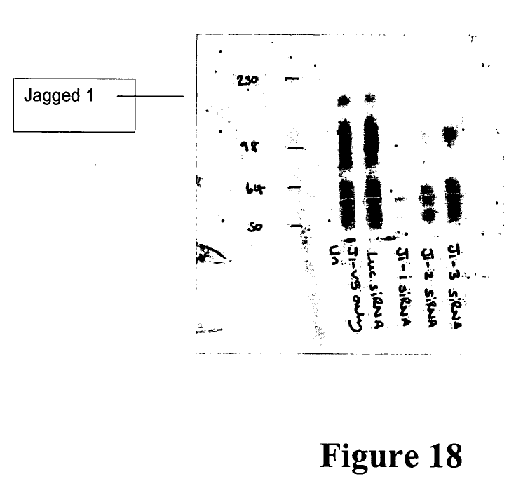

FIG. 18 shows a Western blot of an extract of CHO cells co-transfected with FL hDelta1 V5-His and Jagged1 siRNA, run on an 8% SDS-PAGE gel and Western blotted with anti-V5 HRP.

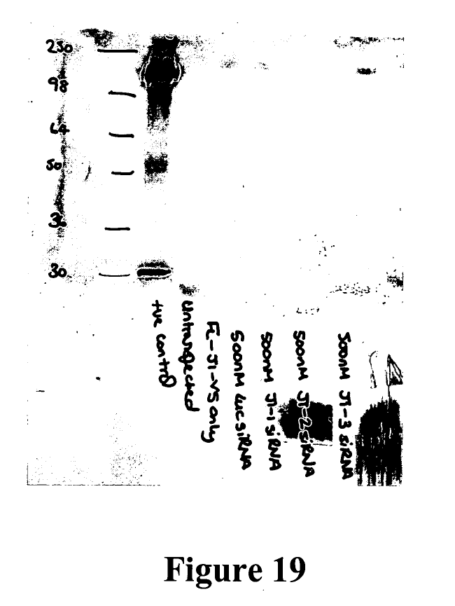

FIG. 19 shows a Western blot of an extract of Jurkat cells co-transfected with FL hDelta1 V5-His and Jagged1 siRNA, run on an 8% SDS-PAGE gel and Western blotted with anti-V5 HRP.

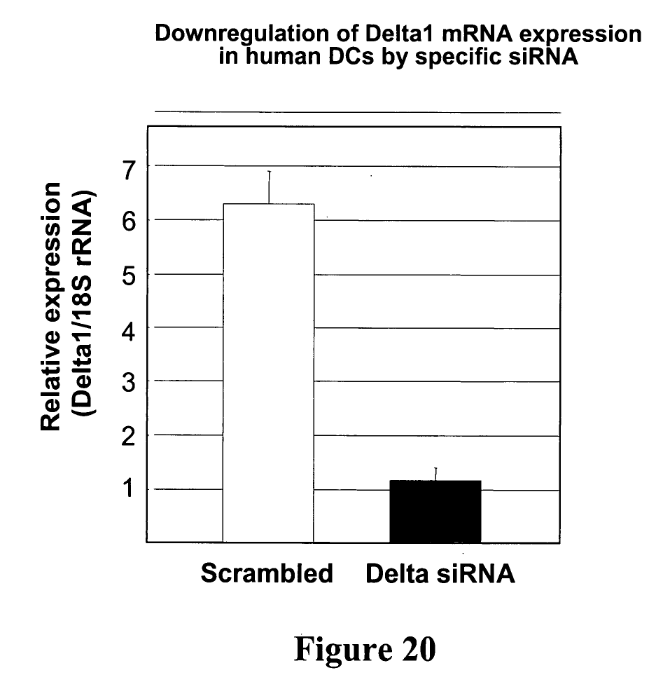

FIG. 20 shows downregulation of Delta1 mRNA expression in human dendritic cells (“DCs”) by specific siRNA. TNF-matured DCs were transfected with 100 nM siRNA, harvested 24 hours post-transfection and assayed for expression at the message level by Lightcycler™.

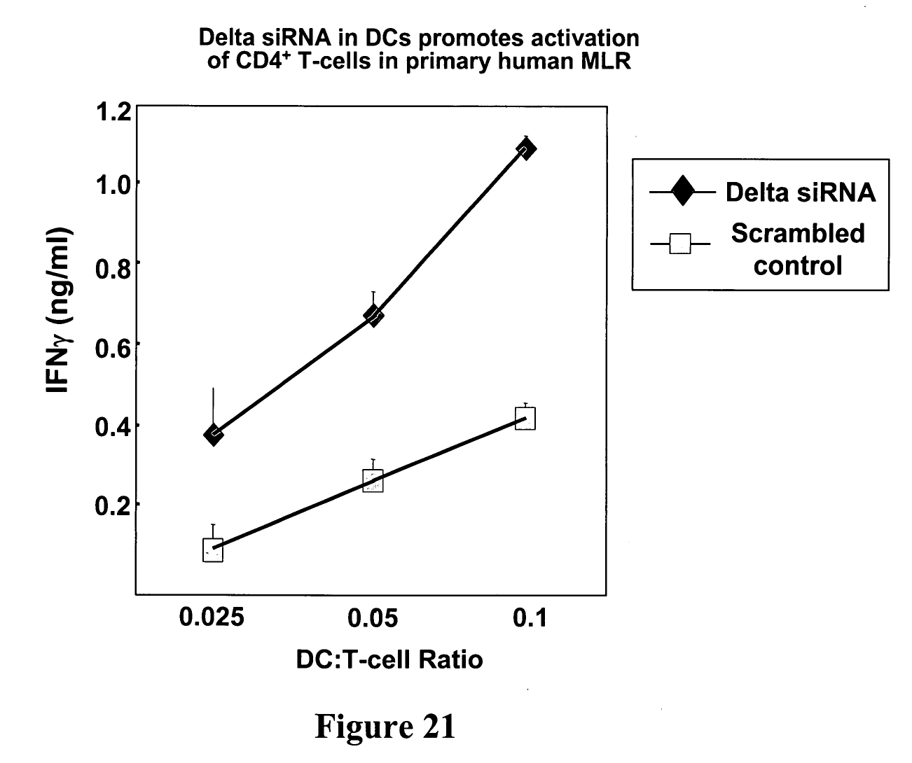

FIG. 21 shows that Delta siRNA in DCs promotes activation of CD4+ T-cells in primary human MLR. DCs derived from CD 14+ PMBCs were matured with TNFα, transfected with siRNA and cultured for five days with allogenic CD4+ T-cells.

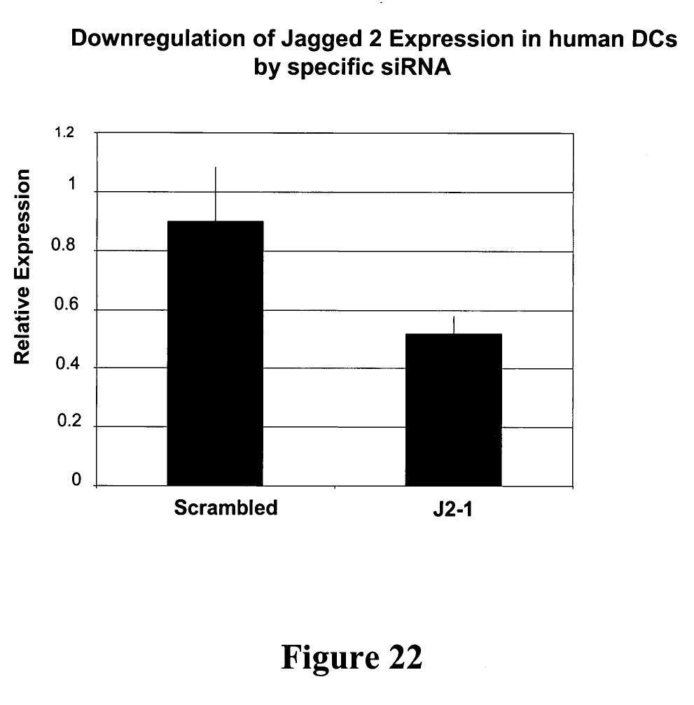

FIG. 22 shows downregulation of Jagged2 expression in human DCs by specific siRNA. TNF-matured DCs were transfected with 100 nM siRNA, harvested 24 hours post-transfection and assayed for expression at the message level by Lightcycler™.

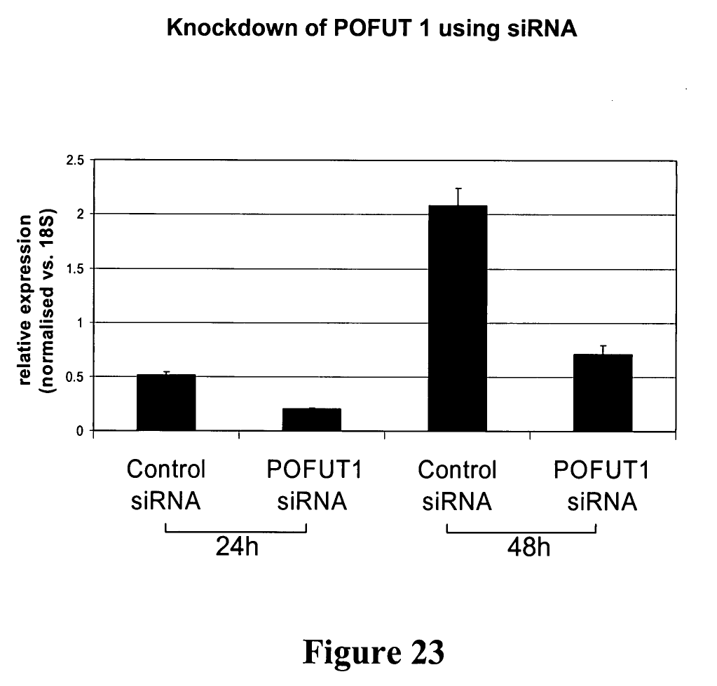

FIG. 23 shows knockdown of POFUT1 in hCD4+ T-cells over a 48 hour period using 100 nM POFUT1 siRNA.

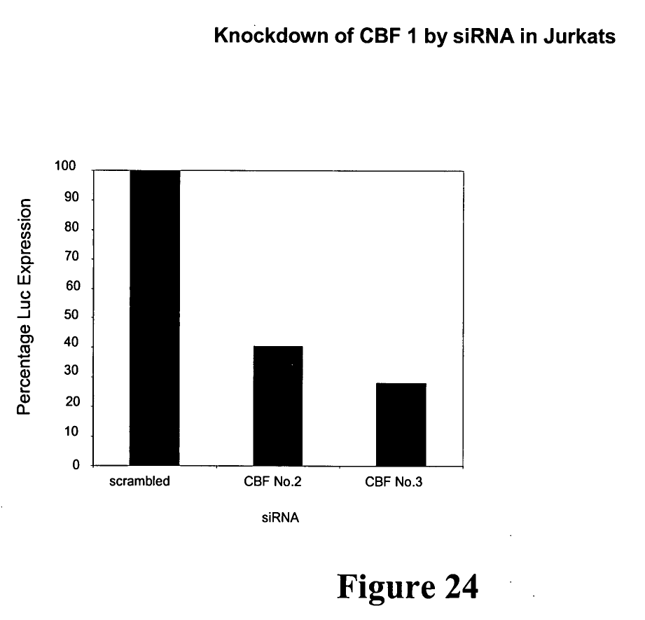

FIG. 24 shows knockdown of CBF 1 by siRNA in Jurkat cells. Jurkats were co-transfected with CBF-VP16, CBF-Luc and 3×CBF specific siRNAs.

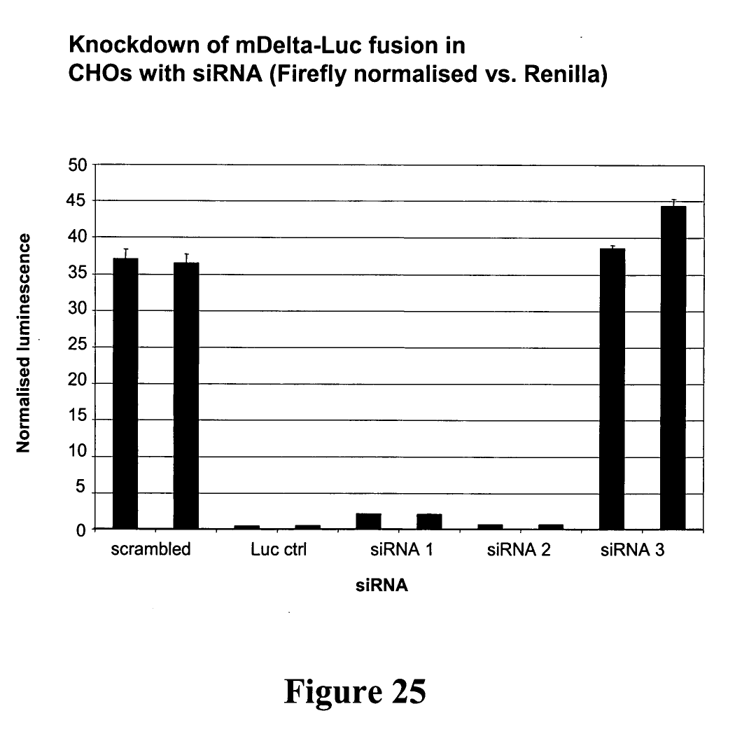

FIG. 25 shows knockdown of mDelta-Luc fusion in CHO cells with siRNA (Firefly normalised versus Renilla).

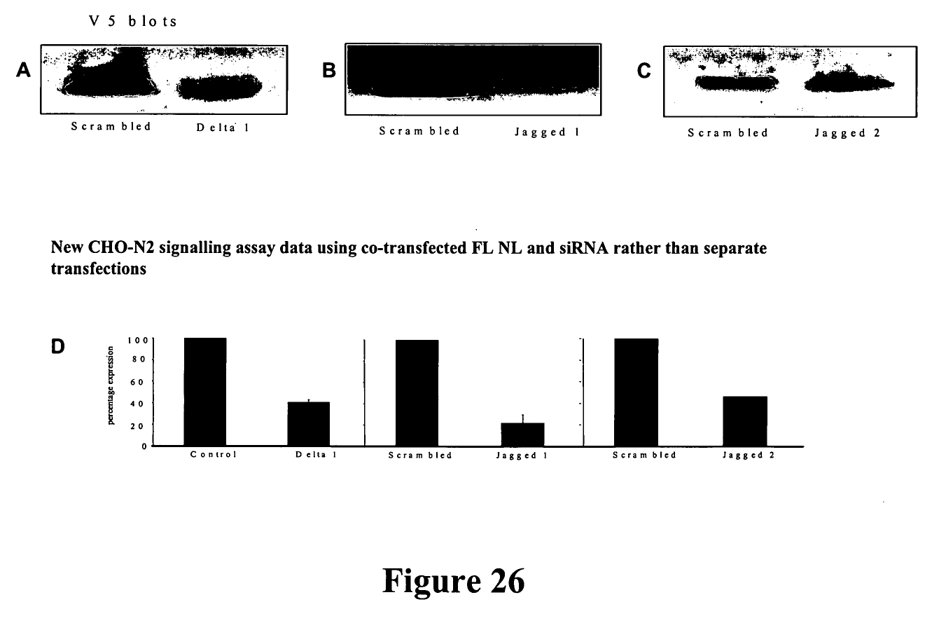

FIGS. 26A-26C show V5 blots using CHO cells co-transfected with FL-NL and siRNAs, rather than separate transfections. FIG. 26D shows CHO-N2 signalling assay data using cells co-transfected with FL-NL and siRNA, rather than separate transfections.

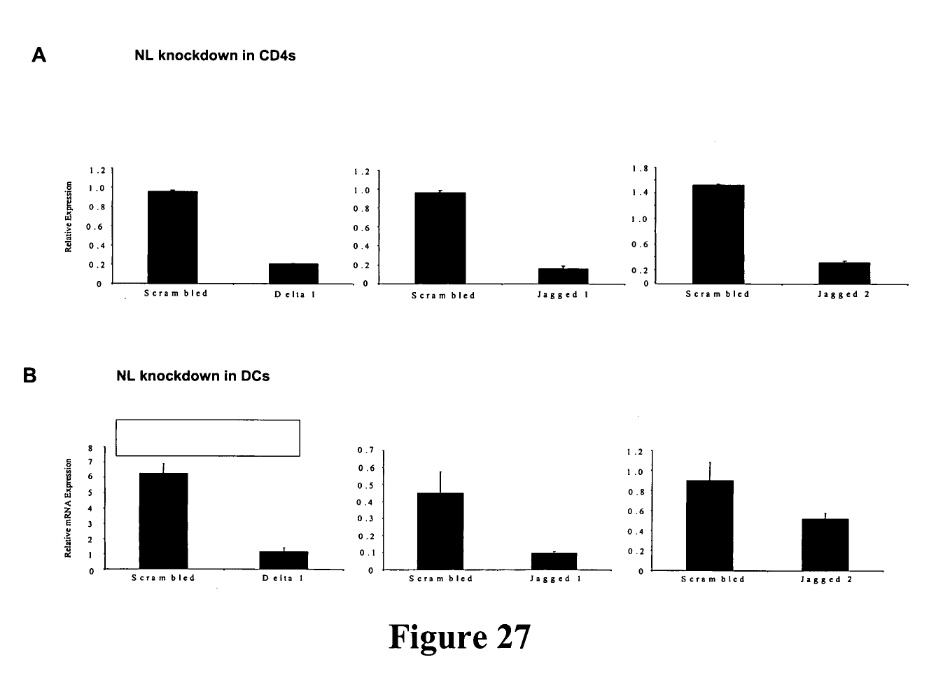

FIG. 27A shows endogenous knockdown in CD4 cells for Delta1, Jagged1 and Jagged2.

FIG. 27B shows endogenous knockdown in hDC cells for Delta1, Jagged1 and Jagged2.

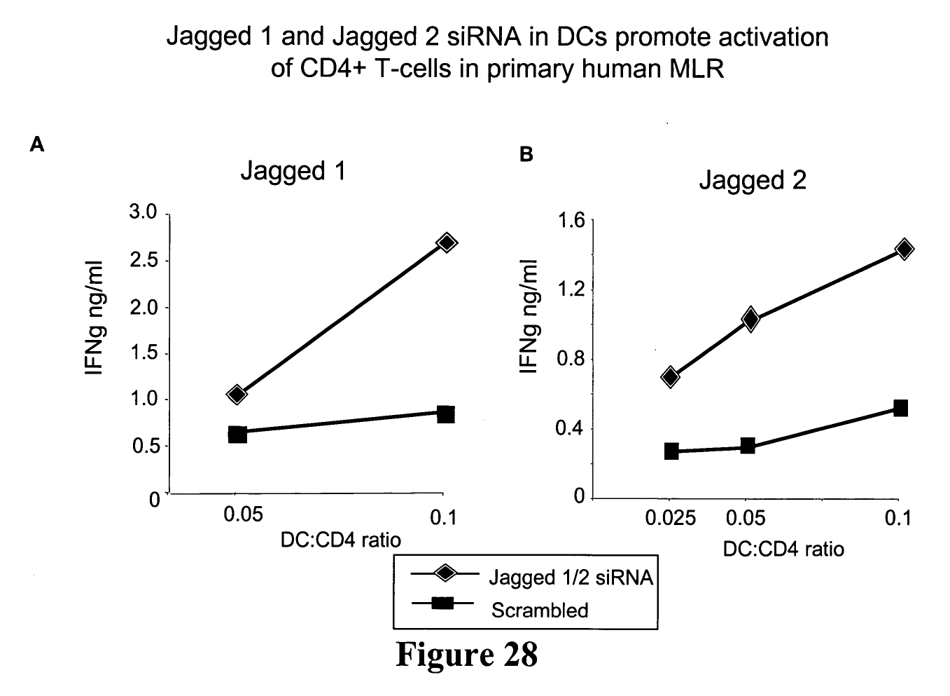

FIG. 28 shows activation by Jagged1 (FIG. 28A) and Jagged2 (FIG. 28B) siRNA in DCs of CD4+ T-cells in primary human MLR.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; and J. E. Coligan, A. M. Kruisbeek, D. H. Margulies, E. M. Shevach and W. Strober (1992 and periodic supplements; Current Protocols in Immunology, John Wiley & Sons, New York, N.Y.). Each of these general texts is hereby incorporated herein by reference.

For the avoidance of doubt, Drosophila and vertebrate names are used interchangeably and all homologues are included within the scope of the invention.

RNA Interference (RNAi)

The discussion that follows discusses the proposed mechanism of RNA interference mediated by short interfering RNA as is presently known, and is not meant to be limiting and is not an admission of prior art. Chemically-modified short interfering nucleic acids may typically possess similar or improved capacity to mediate RNAi as do native siRNA molecules and are expected to possess improved stability and activity in vivo; therefore, this discussion is not meant to be limiting only to siRNA and can be applied to interfering NA as a whole.

As described, for example, in US Patent Publication 20030190635 (McSwiggen), RNA interference refers to the process of sequence-specific post transcriptional gene silencing in animals mediated by short interfering RNAs (siRNA) (Fire et al., 1998, Nature, 391, 806). The corresponding process in plants is commonly referred to as post transcriptional gene silencing or RNA silencing and is also referred to as quelling in fungi. The process is thought to be an evolutionarily conserved cellular defense mechanism used to prevent the expression of foreign genes which is commonly shared by diverse flora and phyla (Fire et al., 1999, Trends Genet., 15, 358). Such protection from foreign gene expression may have evolved in response to the production of double stranded RNAs (dsRNA) derived from viral infection or the random integration of transposon elements into a host genome via a cellular response that specifically destroys homologous single stranded RNA or viral genomic RNA.

As described, for example, in US Patent Publication No 20030203868 (Bushman) in RNA interference as it occurs naturally, during the initiation step, input dsRNA is digested into 21-23 nucleotide small interfering RNAs (siRNAs), which have also been called “guide RNAs” as described in Hammond et al. Nature Rev Gen 2: 110-119 (2001); Sharp, Genes Dev 15: 485-490 (2001); and Hutvagner and Zamore, Curr Opin Genetics & Development 12:225-232 (2002), which are incorporated herein by reference. The siRNAs are produced when an enzyme (DICER) belonging to the RNase III family of dsRNA-specific ribonucleases progressively cleaves dsRNA, which can be introduced directly or via a transgene or vector. Successive cleavage events degrade the RNA to 19-21 base pair duplexes (siRNAs), each with 2-nucleotide 3′ overhangs as described by Hutvagner and Zamore, Curr. Opin. Genetics & Development 12:225-232 (2002); Bernstein et al., Nature 409:363-366 (2001), which are incorporated herein by reference. In the effector step, the siRNA duplexes bind to a nuclease complex to form what is known as the RNA-induced silencing complex, or RISC. The active RISC then targets the homologous transcript by base pairing interactions and cleaves the mRNA approximately 12 nucleotides from the 3′ terminus of the siRNA (Nykanen et al., Cell 107:309-321 (2001), which is incorporated herein by reference in its entirety).

A strand of an siRNA that corresponds to a region on a target gene transcript is often referred to as the sense strand, while the other strand, which is complementary, is frequently termed the antisense strand.

In some host cells use of longer ds RNA (double stranded RNA) provokes a non-specific cytotoxic response. In contrast, the introduction of shorter dsRNAs, in particular siRNAs, appears to suppress gene expression without producing a non-specific cytotoxic response because the small size of the siRNAs, as compared to larger dsDNA, prevents activation of the dsRNA-inducible interferon system in mammalian cells and avoids the non-specific phenotypes that can be observed by introducing larger dsRNA.

As used herein, “long” dsRNAs refer to those which are longer than typical siRNAs, longer than about 23 nucleotides and are processed to be used as primers. Similarly, “short” double-stranded RNAs are siRNAs which can be used as primers for RNAi. Methods for making such “long” or “short” dsRNAs are discussed below, but can be any methods known to one skilled in the art. Therefore, the term “dsRNA” encompasses molecules of the size referred to in the art as siRNAs as well as larger RNA duplexes, as long as functionality with regard to modulation of Notch signalling via target gene knockdown is preserved.

As used herein, a double-stranded RNA corresponding to a target gene refers to a double-stranded RNA copy that, except for possessing Uracil instead of Thymine, preferably has substantially the same nucleic acid sequence as a portion of the DNA duplex that encodes a target gene on its coding strand, which is also referred to as non-template strand, plus strand, or sense strand. Thus, a double-stranded RNA corresponding to a target gene transcript preferably has one strand that has substantially the sequence that would result during mRNA synthesis from the template or anti-sense strand, which corresponds to a portion of the target gene, and its complementary sequence.

A dsRNA corresponding to a target gene can have, for example, between 50 and 100 contiguous base pairs, between 25 and 50 contiguous base pairs, between 14 and 26 contiguous base pairs that correspond to the target gene, between 15 and 25, between 16 and 24, between 17 and 23, between 18 and 22, between 19 and 21 contiguous base pairs, up to the full length of the corresponding DNA duplex, as long as the dsRNA is capable of target gene inhibition. In this regard, the dsRNA corresponding to the target gene can be of any length as long as dsRNA-dependent protein kinase (PKR) is not induced upon formation of the dsRNA. A major component of the mammalian non-specific response to dsRNA is mediated by the dsRNA-dependent protein kinase, PKR, which phosphorylates and inactivates the translation factor eIF2a, leading to a generalized suppression of protein synthesis and cell death via both nonapoptotic and apoptotic pathway. PKR can be one of several kinases in mammalian cells that can mediate this response.

Because siRNAs act as the primers for specific recognition of the RNA to be cleaved, there are structural features which have been identified to produce siRNAs which act most efficiently.

Preferred structural features of siRNAs include a free 3′ hydroxyl group (this allows the siRNA to act as a primer for the RdRP reaction), a 5′ phosphate group, and 3′ overhangs. This most likely corresponds to the cleavage pattern of an RNase III-like enzyme. RNase III makes two staggered cuts in both strands of a dsRNA, leaving a 3′ overhang of 2 nucleotides. The “long” dsRNAs have been found to be processed by the cell into siRNAs. Thus, large dsRNAs can be processed to 21-23 nucleotide siRNAs with a free 3′ hydroxyl group, a 5′ phosphate group, and 3′ overhangs of 2 nucleotides.

Oligos are suitably about 21 nucleotides in length with a GC content close to 50%, Runs of 3 or more Gs or Cs are preferably avoided and target sequences may typically start with 2 adenosines. Suitably theey may have symmetrical 3′ overhands and, preferably, have low homology to other gene sequences which they may come into contact with when administered.

The structural features of “long” double-stranded RNAs would appear to be less stringent since they are not active in the priming reaction but will simply be processed into the active siRNAs with the most advantageous features. However, overhangs of 17-20 nucleotides were less potent than blunt-ended siRNAs. The inhibitory effect of long 3′ ends was particularly pronounced for dsRNAs of less than 100 bp. Interestingly, a 5′ terminal phosphate, although present after dsRNA processing was not required to mediate target RNA cleavage and was absent from the short synthetic RNAs which worked with high efficiency. In addition, the size of a “long” double-stranded RNAs can have an effect on the efficiency.

Preferred lengths for efficient processing of dsRNA into 21 and 22 nucleotide fragments are determined by the fact that short dsRNA (<150 bp) appear to be less effective than longer dsRNAs in degrading target mRNA. Thus, “long” double-stranded RNAs can suitably be from about 38 nucleotides to about full-length, from about 50 base pairs to about 1000 base pairs. The “long” double-stranded RNAs can range in size from about 150 base pairs to about 505 base pairs, including, but not limited to: 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, and 500 base pairs.

The target cleavage site was found to be located near the center of the region covered by the 21 or 22 nucleotide RNAs, 11 or 12 nucleotides downstream of the first nucleotide that is complementary to the 21 or 22 nucleotide guide sequence. Thus, it would be possible to design a pair of 21 or 22 nucleotide RNAs to cleave a target RNA at almost any given position. In addition, the overhangs did not need to be complementary to produce efficient cleavage. The direction of dsRNA processing determined whether a sense or an antisense target RNA was cleaved by the siRNP endonuclease.

General siRNA Design Guidelines

The following design guidelines have been published by Ambion, but it will be appreciated that these represent only one of many approaches, and that many other strategies may also be employed.

1. Find 21 Nucleotide Sequences in the Target mRNA that Begin with an AA Dinucleotide.

Beginning with the AUG start codon of your transcript, scan for AA dinucleotide sequences. Record each AA and the 3′ adjacent 19 nucleotides as potential siRNA target sites.

This strategy for choosing siRNA target sites is based on the observation by Elbashir et al. (Elbashir, et al. (2001) EMBO J 20: 6877-6888) that siRNAs with 3′ overhanging UU dinucleotides are particularly effective. This is also compatible with using RNA pol III to transcribe hairpin siRNAs because RNA pol III terminates transcription at 4-6 nucleotide poly(T) tracts creating RNA molecules with a short poly(U) tail.

In Elbashir's and subsequent publications, siRNAs with other 3′ terminal dinucleotide overhangs have been shown to effectively induce RNAi. If desired, this target site selection strategy may be mdified to design siRNAs with other dinucleotide overhangs, but it is generally recommended to avoid G residues in the overhang because of the potential for the siRNA to be cleaved by RNase at single-stranded G residues.

2. Select 2-4 Target Sequences.

Choose target sites from among the sequences identified in Step 1 based on the following guidelines:
Ambion researchers report that siRNAs with 30-50% GC content are more active than those with a higher G/C content.
Since a 4-6 nucleotide poly(T) tract acts as a termination signal for RNA pol III, avoid stretches of more than 4 T's or A's in the target sequence when designing sequences to be expressed from an RNA pol III promoter.
Since some regions of mRNA may be either highly structured or bound by regulatory proteins, it is recommended to select siRNA target sites at different positions along the length of the gene sequence.
Compare the potential target sites to the appropriate genome database (e.g. human) and eliminate any target sequences with more than 16-17 contiguous base pairs of homology to other coding sequences. It is recommended to use BLAST, which can be found on the National Center for Biotechnology Information website, maintained by the National Institutes of Health.
   Ambion's siRNA Target Finder

A number of software packages are available to assist with design. For example, Ambion's online target finder (available at Ambion's website) can be used to find potential sequences based on the design guidelines described above. Alternatively, the Whitehead Institute of Biomedical Research at MIT has a publicly available siRNA design tool available on its website that incorporates additional selection parameters and integrates BLAST searches of the human and mouse genome databases.

Corresponding siRNAs can then be chemically synthesized, created by in vitro transcription, or expressed from a vector or PCR product.

Specific Guidelines for Designing siRNA Hairpins Encoded by siRNA Expression Vectors and siRNA Expression Cassettes

The following recommendations for siRNA hairpin design and cloning strategy are provided by Ambion and available at Ambion's website.

The first step in designing an appropriate insert is to choose the siRNA target site by following the steps described above. For screening, it is recommended to test about four siRNA sequences per target, spacing the siRNA sequences down the length of the gene sequence to reduce the chances of targeting a region of the mRNA that is either highly structured or bound by regulatory proteins. Because constructing and testing four siRNA expression plasmids per target can be time-consuming, it may be preferred to screen potential siRNA sequences using PCR-derived siRNA expression cassettes (SECs). SECs are PCR products that include promoter and terminator sequences flanking a hairpin siRNA template and can be prepared with Ambion's Silencer™ Express Kits. This screening strategy also permits the rapid identification of the best combination of promoter and siRNA sequence in the experimental system. SECs found to effectively elicit gene silencing can be readily cloned into a vector for long term studies. Ambion scientists have determined that sequences that function well as transfected siRNAs also function well as siRNAs that are expressed in vivo. The only exception is that siRNA sequences to be expressed in vivo should preferably not contain a run of 4 or 5 A's or T's, as these can act as termination sites for Polymerase III.

For traditional cloning into pSilencer vectors, two DNA oligonucleotides that encode the chosen siRNA sequence are designed for insertion into the vector. Suitably, the DNA oligonucleotides consist of a 19-nucleotide sense siRNA sequence linked to its reverse complementary antisense siRNA sequence by a short spacer. Ambion report use of a 9-nucleotide spacer (TTCAAGAGA), although many other suitable spacers can be used. Suitably about 5-6 T's are added to the 3′ end of the oligonucleotide. In addition, for cloning into Ambion's pSilencer 1.0-U6 vector, nucleotide overhangs to the EcoR I and Apa I restriction sites are preferably added to the 5′ and 3′ end of the DNA oligonucleotides, respectively. In contrast, for cloning into Ambion's pSilencer 2.0-U6, 2.1-U6, 3.0-H1, or 3.1-H1 vectors, nucleotide overhangs with BarnH I and Hind III restriction sites are added to the 5′ and 3′ end of the DNA oligonucleotides, respectively. The resulting RNA transcript is expected to fold back and form a stem-loop structure comprising a 19 bp stem and 9 nt loop with 2-3 U's at the 3′ end.

For cloning into Ambion's pSilencer adeno 1.0-CMV vector, DNA oligonucleotides with stem-loop structures are suitably created similar to those of pSilencer 2.0 and 3.0 vectors described above. However, one notable exception is the absence of 5-6 T's from the 3′-end of the oligonucleotides for the CMV-based vector system since the transcription termination signal for the CMV-based vector system is provided by the SV40 polyA terminator. In addition, for cloning into the pSilencer adeno 1.0-CMV vector, nucleotide overhangs containing the Xho I and Spe I restriction sites are preferably added to the 5′ and 3′ end of the DNA oligonucleotides, respectively.

For preparing SECs containing an H1 or U6 promoter by PCR using Ambion's Silencer Express Kits, (Elbashir, et al. (2001)) one or two DNA oligonucleotides encoding the siRNA sequence are designed and ordered, (Editors of Nature Cell Biology (2003) Whither RNAi? Nat Cell Biol. 5:489-490.) the oligonucleotides are used as primers in one or more PCRs with the promoter-containing template included in the kit, and (Brown, D., Jarvis, R., Pallotta, V., Byrom, M., and Ford, L. (2002) RNA interference in mammalian cell culture: design, execution, and analysis of the siRNA effect. Ambion TechNotes 9(1): 3-5.) the resulting PCR product is column purified. Ambion typically recommend a loop sequence of 5′-UUUGUGUAG-3′ for their SECs, although other loop sequences can also be used. As with vector insert design, a 5-6 T termination sequence is added to act as an RNA pol III terminator. For subsequent cloning convenience, EcoRI and HindIII restriction sites are also encoded by the primers. The detailed design parameters for the oligonucleotide primers used with the Silencer Express Kits can be found in the kits' Instruction Manual.

For cloning of functional Silencer Express Kit-derived SECs into vectors, the SEC and destination vector should be restricted with EcoRI and HindIII. Linearized destination vectors with neomycin, hygromycin and puromycin resistance genes, called pSEC Vectors, are available.

Selection of siRNA Targets

In addition to the suggested procedure for selecting siRNA targets by scanning a mRNA sequence for AA dinucleotides and recording the 19 nucleotides immediately downstream of the AA, a number of other methods have been employed by other researchers. In one method, the selection of the siRNA target sequence is purely empirically determined (Sui, G., Soohoo, C., Affar, E. B., Gay, F., Shi, Y., Forrester, W. C., and Shi, Y. (2002) A DNA vector-based RNAi technology to suppress gene expression in mammalian cells. Proc. Natl. Acad. Sci. USA 99(8): 5515-5520.), as long as the target sequence starts with GG and does not share significant sequence homology with other genes as analyzed by BLAST search.

In another approach, a more elaborate method is employed to select the siRNA target sequences. This procedure exploits an observation that any accessible site in endogenous mRNA can be targeted for degradation by the synthetic oligodeoxyribonucleotide/RNase H method (Lee, N. S., Dohjima, T., Bauer, G., Li, H., Li, M.-J., Ehsani, A., Salvaterra, P., and Rossi, J. (2002) Expression of small interfering RNAs targeted against HIV-1 rev transcripts in human cells. Nature Biotechnology 20 : 500-505.). Any accessible site identified in this fashion is then used as insert sequence in the U6 promoter-driven siRNA constructs.

Order of the Sense and Antisense Strands within the Hairpin siRNAs

A hairpin siRNA expression cassette is typically constructed to contain the sense strand of the target, followed by a short spacer, then the antisense strand of the target, in that order. One group of researchers has found that reversal of the order of sense and antisense strands within the siRNA expression constructs did not affect the gene silencing activities of the hairpin siRNA (Yu, J.-Y., DeRuiter, S. L., and Turner, D. L. (2002) RNA interference by expression of short-interfering RNAs and hairpin.RNAs in mammalian cells. Proc. Natl. Acad. Sci. USA 99(9): 6047-6052). In contrast, another group of researchers has found that similar reversal of order in another siRNA expression cassette caused partial reduction in the gene silencing activities of the hairpin siRNA (Paul, C. P., Good, P. D., Winer, I., and Engelke, D. R. (2002) Effective expression of small interfering RNA in human cells. Nature Biotechnology 20 : 505-508). It is not clear what is responsible for this difference in observation. At the present time, it is still preferable to construct the siRNA expression cassette in the order of sense strand, short spacer, and antisense strand.

Length of the siRNA Stem

There appears to be some degree of variation in the length of nucleotide sequence being used as the stem of siRNA expression cassette. Several research groups including Ambion have used 19 nucleotides-long sequences as the stem of siRNA expression cassette. In contrast, other research groups have used siRNA stems ranging from 21 nucleotides-long to 25-29 nucleotides-long. It is found that hairpin siRNAs with these various stem lengths all function well in gene silencing studies.

Length and Sequence of the Loop Linking Sense and Antisense Strands of Hairpin siRNA

Various research groups have reported successful gene silencing results using hairpin siRNA with loop size ranging between 3 to 23 nucleotides The following are examples of loop size specific loop sequences used by various research groups:

| | | |
|---|---|---|
| Loop | | |
| Size | | |
| (# of | Specific | |
| Nucleo- | Loop | |
| tides) | Sequence | Reference |

| | | |
|---|---|---|
| 3 | AUG | Sui, G., Soohoo, C., Affar, E. |
| | | B., Gay, F., Shi, Y., Forrester, |
| | | W. C., and Shi, Y. (2002) A DNA |
| | | vector-based RNAi technology to |
| | | suppress gene expression in mam- |
| | | malian cells. Proc. Natl. Acad. |
| | | Sci. USA 99(8): 5515-5520. |
| 3 | CCC | Paul, C. P., Good, P. D., Winer, |
| | | I., and Engelke, D. R. (2002) |
| | | Effective expression of small |
| | | interfering RNA in human cells. |
| | | Nature Biotechnology 20: |
| | | 505-508. |
| 4 | UUCG | Lee, N. S., Dohjima, T., Bauer, |
| | | G., Li, H., Li, M. -J., Ehsani, |
| | | A., Salvaterra, P., and Rossi, |
| | | J. (2002) Expression of small |
| | | interfering RNAs targeted a- |
| | | gainst HIV-1 rev transcripts in |
| | | human cells. Nature Biotechnol- |
| | | ogy 20: 500-505 |
| 5 | CCACC | Paul, C. P., Good, P. D., Winer, |
| | | I., and Engelke, D. R. (2002) |
| | | Effective expression of small |
| | | interfering RNA in human cells. |
| | | Nature Biotechnology 20: |
| | | 505-508. |
| 6 | CTCGAG | Editors of Nature Cell Biology |
| | | (2003) Whither RNAi? Nat Cell |
| | | Biol. 5: 489-490. |
| 6 | AAGCUU | Editors of Nature Cell Biology |
| | | (2003) Whither RNAi? Nat Cell |
| | | Biol. 5: 489-490. |
| 7 | CCACACC | Paul, C. P., Good, P. D., Winer, |
| | | I., and Engelke, D. R. (2002) |
| | | Effective expression of small |
| | | interfering RNA in human cells. |
| | | Nature Biotechnology 20: |
| | | 505-508. |
| 9 | UUCAAGAGA | Yu, J. -Y., DeRuiter, S. L., and |
| | | Turner, D. L. (2002) RNA inter- |
| | | ference by expression of short- |
| | | interfering RNAs and hairpin |
| | | RNAs in mammalian cells. Proc. |
| | | Natl. Acad. Sci. USA 99(9): |
| | | 6047-6052. |
| 23 | — | Jacque, J. -M., Triques, K., and |
| | | Stevenson, M. (2002) Modulation |
| | | of HIV-1 replication by RNA in- |
| | | terference. Nature 418: 435-438. |

Components of the Notch Signalling Pathway and Target Sequences

According to the present invention, Notch signalling may be either increased or decreased, depending on the target chosen. Thus, on the one hand, Notch signalling will generally be reduced by targeting a component of the pathway which normally promotes Notch signalling. Conversely, Notch signalling will generally be increased by targeting a component of the pathway which normally inhibits Notch signalling.

For example, targets for upregulation of Notch signalling (wherein use as a target of RNAi will generally be expected to increase Notch signalling) include, without limitation, mammalian homologues of the following:
Notch Ubiquitin ligases, especially E3/Nedd4 ubiquitin ligases, such as mammalian homologues of Suppressor of Deltex, SEL-10 (e.g. see GenBank Accession No AY008274; human SEL-10), Itch (e.g. see GenBank Accession No AB056663; human Itch), AIP4 (e.g. see GenBank Accession No AF038564; human AIP4);
Fringes such as Manic Fringe (e.g. see GenBank Accession No U94352; human Manic Fringe), Radical Fringe (e.g. see GenBank Accession No BC014495; human Radical Fringe) and Lunatic Fringe (e.g. see GenBank Accession No BC014851; human Lunatic Fringe) for differential Notch ligand signalling;
Numb (e.g. see GenBank Accession Nos AF171938, AF171939, AF171940, AF171941; human Numb isoforms 1-4);
Ligand proteases such as Kuzbanian and ADAM proteases; and
Mindbomb (e.g. see GenBank Accession No AY147849; human Mindbomb).

Targets for downregulation of Notch signalling (wherein use as a target of RNAi will generally be expected to decrease Notch signalling) include, without limitation, the following:
Notch ligands such as Delta, such as Delta 1 (e.g. see GenBank Accession No AF003522; human Delta1), Delta 3 (e.g. see GenBank Accession No NM_{—}016941; human Delta3), Delta4 (e.g. see GenBank Accession No AF253468; human Delta4);
Jagged such as Jagged 1 (e.g. see GenBank Accession No AF028593; human Jagged1) and Jagged 2 (e.g. see GenBank Accession No AF029778; human Jagged2);
Notch such as Notch1 (e.g. see GenBank Accession No AF308602; human Notchl), Notch 2 (e.g. see GenBank Accession No AF315356; human Notch2), Notch3 (e.g. see GenBank Accession No NM_{—}000435; human Notch3) and Notch4 (e.g. see GenBank Accession No U95299; human Notch4);
Notch IC protease complex components such as gamma secretases, nicastrin (e.g. see GenBank Accession No AF240468; human nicastrin), presenilin (PS) (e.g. see GenBank Accession No AF416717; human PS1—and GenBank Accession No BT006984; human PS2);
CSL transcriptional cofactors such as CBF-1, especially human CBF-1; Members of the HES (Hairy/Enhancer of Split) family of basic helix-loop-helix transcriptional regulators such as HES-1, Hey and HerP;
Fringes such as Manic Fringe (e.g. see GenBank Accession No U94352; human Manic Fringe), Radical Fringe (e.g. see GenBank Accession No BC014495; human Radical Fringe) and Lunatic Fringe (e.g. see GenBank Accession No BC014851; human Lunatic Fringe) for downregulating Jagged signalling; and Deltex (e.g. see GenBank Accession No AF053700; human Deltex).
   Nucleic Acids

The term nucleic acid is a term of art that refers to a polymer containing at least two nucleotides. Natural nucleotides contain a deoxyribose (DNA) or ribose (RNA) group, a phosphate group, and a base. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups on the base such as, but not limited to, amines, alcohols, thiols, carboxylates, and alkylhalides. The term “base” also encompasses any base analog of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil-, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. Nucleotides are the monomeric units of nucleic acid polymers and are linked together through the phosphate groups in natural polynucleotides. Natural polynucleotides have a ribose-phosphate backbone. Artificial or synthetic polynucleotides are polymerized in vitro and contain the same or similar bases but may contain a backbone of a type other than the natural ribose-phosphate backbone. These backbones include, but are not limited to: PNAs (peptide nucleic acids), phosphorothioates, phosphorodiamidates, morpholinos, and other variants of the phosphate backbone of natural polynucleotides.

An RNAi agent such as an interfering RNA (e.g. siRNA) for use with the present invention suitably comprises or codes for a sequence that is preferably identical or nearly identical to a portion of a gene coding for a component of the Notch signalling pathway. RNA may be polymerized in vitro, recombinant RNA, contain chimeric sequences, or derivatives of these groups. The siRNA may contain ribonucleotides, deoxyribonucleotides, synthetic nucleotides, or any suitable combination such that expression of the target gene is inhibited. The RNA is preferably double stranded, but may be single, triple, or quadruple stranded.

Chemical Modification

Chemically synthesizing nucleic acid molecules with modifications (base, sugar and/or phosphate) can prevent their degradation by serum ribonucleases, which can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; Gold et al., U.S. Pat. No. 6,300,074; and Burgin et al., supra; all of which are incorporated by reference). All of the above references describe various chemical modifications that can be made to the base, phosphate and/or sugar moieties of the nucleic acid molecules described herein. Modifications that enhance their efficacy in cells, and removal of bases from nucleic acid molecules to shorten oligonucleotide synthesis times and reduce chemical requirements are desired.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-O-allyl, and/or 2′-H nucleotide base modifications (for a review, see Usman and Cedergren, 1992, TIBS, 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication, PCT No. WO 92/07065; Perrault et al. Nature, 1990, 344, 565-568; Picken et al., Science, 1991, 253, 314-317; Usman and Cedergren, Trends in Biochem. Sci., 1992, 17, 334-339; Usman et al., International Publication PCT No. WO 93/15187; Sproat, U.S. Pat. No. 5,334,711; Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 (filed on Apr. 20, 1998); Karpeisky et al., 1998, Tetrahedron Lett., 39, 1131; Eamshaw and Gait, 1998, Biopolymers (Nucleic Acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated in their totality by reference herein). Such publications describe general methods and strategies to determine the location of incorporation of sugar, base and/or phosphate modifications and the like into nucleic acid molecules without modulating catalysis, and are incorporated by reference herein. In view of such teachings, similar modifications can be used as described herein to modify the siNA nucleic acid molecules of the instant invention so long as the ability of siNA to promote RNAi is cells is not significantly inhibited.

Thus, in one embodiment, the invention provides or uses modified interfering nucleic acid molecules, with phosphate backbone modifications comprising one or more phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications, see Hunziker and Leumann, 1995, Nucleic Acid Analogues: Synthesis and Properties, in Modem Synthetic Methods, VCH, 331-417, and Mesmaeker et al., 1994, Novel Backbone Replacements for Oligonucleotides, in Carbohydrate Modifications in Antisense Research, ACS, 24-39.

Thus, interfering nucleic acid molecules having chemical modifications that maintain or enhance activity are also provided. Such a nucleic acid is also generally more resistant to nucleases than an unmodified nucleic acid. Accordingly, the in vitro and/or in vivo activity should not be significantly lowered. In cases in which modulation is the goal, therapeutic nucleic acid molecules delivered exogenously should optimally be stable within cells until translation of the target RNA has been modulated long enough to reduce the levels of the undesirable protein. This period of time varies between hours to days depending upon the disease state. Improvements in the chemical synthesis of RNA and DNA (Wincott et al., 1995, Nucleic Acids Res. 23, 2677; Caruthers et al., 1992, Methods in Enzymology 211, 3-19 (incorporated by reference herein)) have expanded the ability to modify nucleic acid molecules by introducing nucleotide modifications to enhance their nuclease stability, as described above.

While chemical modification of oligonucleotide internucleotide linkages with phosphorothioate, phosphorothioate, and/or 5′-methylphosphonate linkages improves stability, excessive modifications can sometimes cause some toxicity or decreased activity. Therefore, when designing nucleic acid molecules, the number of such intemucleotide linkages should preferably be minimized for lower toxicity, increased efficacy and higher specificity.

In one embodiment, nucleic acid molecules of the invention may include one or more (e.g., about 1, 2, 3, 4, 5, 6, 7, 8, 9, 10, or more) G-clamp nucleotides. A G-clamp nucleotide is a modified cytosine analog wherein the modifications confer the ability to hydrogen bond both Watson-Crick and Hoogsteen faces of a complementary guanine within a duplex (see for example Lin and Matteucci, 1998, J. Am. Chem. Soc., 120, 8531-8532). A single G-clamp analog substitution within an oligonucleotide can result in substantially enhanced helical thermal stability and mismatch discrimination when hybridized to complementary oligonucleotides. The inclusion of such nucleotides in nucleic acid molecules of the invention may provide both enhanced affinity and specificity to nucleic acid targets, complementary sequences, or template strands. In another embodiment, nucleic acid molecules of the invention may include one or more (e.g., about 1, 2, 3, 4, 5, 6, 7, 8, 9, 10, or more) LNA “locked nucleic acid” nucleotides such as a 2′, 4′-C mythylene bicyclo nucleotide (see for example Wengel et al., International PCT Publication No. WO 00/66604 and WO 99/14226).

In another embodiment, the invention provides conjugates and/or complexes of interfering nucleic acid molecules of the invention. Such conjugates and/or complexes can be used to facilitate delivery of nucleic acid molecules into a biological system, such as a cell. The conjugates and complexes provided by the instant invention can impart therapeutic activity by transferring therapeutic compounds across cellular membranes, altering the pharmacokinetics, and/or modulating the localization of nucleic acid molecules of the invention. The present invention encompasses the design and synthesis of novel conjugates and complexes for the delivery of molecules, including, but not limited to, small molecules, lipids, phospholipids, nucleosides, nucleotides, nucleic acids, antibodies, toxins, negatively charged polymers and other polymers, for example proteins, peptides, hormones, carbohydrates, polyethylene glycols, or polyamines, across cellular membranes. In general, the transporters described are designed to be used either individually or as part of a multi-component system, with or without degradable linkers. These compounds are expected to improve delivery and/or localization of nucleic acid molecules of the invention into a number of cell types originating from different tissues, in the presence or absence of serum (see Sullenger and Cech, U.S. Pat. No. 5,854,038). Conjugates of the molecules described herein can be attached to biologically active molecules via linkers that are biodegradable, such as biodegradable nucleic acid linker molecules.

The term “biodegradable linker” as used herein, refers to a nucleic acid or non-nucleic acid linker molecule that is designed as a biodegradable linker to connect one molecule to another molecule, for example, a biologically active molecule to an interfering nucleic acid molecule of the invention or the sense and antisense strands of a siNA molecule. The biodegradable linker is designed such that its stability can be modulated for a particular purpose, such as delivery to a particular tissue or cell type. The stability of a nucleic acid-based biodegradable linker molecule can be modulated by using various chemistries, for example combinations of ribonucleotides, deoxyribonucleotides, and chemically-modified nucleotides, such as 2′-O-methyl, 2′-fluoro, 2′-amino, 2′-O-amino, 2′-C-allyl, 2′-O-allyl, and other 2′-modified or base-modified nucleotides. The biodegradable nucleic acid linker molecule can be a dimer, trimer, tetramer or longer nucleic acid molecule, for example, an oligonucleotide of about 2, 3, 4, 5, 6, 7, 8, 9, 10, 11, 12, 13, 14, 15, 16, 17, 18, 19, or 20 or more nucleotides in length, orcan comprise a single nucleotide with a phosphorus-based linkage, for example, a phosphoramidate or phosphodiester linkage. The biodegradable nucleic acid linker molecule can also comprise nucleic acid backbone, nucleic acid sugar, or nucleic acid base modifications.

The term “biodegradable” as used herein, refers to degradation in a biological system, for example enzymatic degradation or chemical degradation.

The term “biologically active molecule” as used herein, refers to compounds or molecules that are capable of eliciting or modifying a biological response in a system. Non-limiting examples of biologically active interfering nuclic acid molecules either alone or in combination with othe molecules contemplated by the instant invention include therapeutically active molecules such as antibodies, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, siNA, dsRNA, allozymes, aptamers, decoys and analogs thereof. Biologically active molecules of the invention also include molecules capable of modulating the pharmacokinetics and/or pharmacodynamics of other biologically active molecules, for example, lipids and polymers such as polyamines, polyamides, polyethylene glycol and other polyethers.

The term “phospholipid” as used herein, refers to a hydrophobic molecule comprising at least one phosphorus group. For example, a phospholipid can comprise a phosphorus-containing group and saturated or unsaturated alkyl group, optionally substituted with OH, COOH, oxo, amine, or substituted or unsubstituted aryl groups.

Therapeutic nucleic acid molecules (e.g., small interfering nucleic acid (siNA) molecules) delivered exogenously are preferably stable within cells until reverse transcription of the RNA has been modulated long enough to reduce the levels of the RNA transcript. The nucleic acid molecules are preferably resistant to nucleases in order to function as effective intracellular therapeutic agents.

Use of the nucleic acid-based molecules of the invention may also lead to better treatment of the disease progression by affording the possibility of combination therapies (e.g., multiple siNA molecules targeted to different genes; nucleic acid molecules coupled with known small molecule modulators; or intermittent treatment with combinations of molecules, including different motifs and/or other chemical or biological molecules). The treatment of subjects with siNA molecules can also include combinations of different types of nucleic acid molecules, such as enzymatic nucleic acid molecules (ribozymes), allozymes, antisense molecules, 2,5-A oligoadenylate, decoys, and aptamers.

In another aspect a siNA molecule may for example comprise one or more 5′- and/or a 3′-cap structure, for example on only the sense siNA strand, antisense siNA strand, or both siNA strands.

By “cap structure” is meant a chemical modification, which has been incorporated at either terminus of the oligonucleotide (see, for example, Adamic et al., U.S. Pat. No. 5,998,203, incorporated by reference). Such terminal modifications protect the nucleic acid molecule from exonuclease degradation, and may help in delivery and/or localization within a cell. Such a cap may for example be present at the 5′-terminus (5′-cap) or at the 3′-terminal (3′-cap) or may be present on both termini. In non-limiting examples: a 5′-cap may suitably for example be selected from the group comprising glyceryl, inverted deoxy abasic residue (moiety); 4′,5′-methylene nucleotide; 1-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety.

In yet another embodiment, a 3′-cap may suitably for example be selected from a group comprising glyceryl, inverted deoxy abasic residue (moiety), 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

Various modifications to nucleic acid structure can be made to enhance the utility of these molecules. Such modifications will enhance shelf-life, half-life in vitro, stability, and ease of introduction of such oligonucleotides to the target site, e.g., to enhance penetration of cellular membranes, and confer the ability to recognize and bind to targeted cells.

As described, for example, in US Patent Publication No. 20030206887 (Morrissey), one or more chemically-modified interfering nucleic acid constructs may suitably be employed if desired. Examples of such chemical modifications include without limitation phosphorothioate internucleotide linkages, 2′-deoxyribonucleotides, 2′-O-methyl ribonucleotides, 2′-deoxy-2′-fluoro ribonucleotides, “universal base” nucleotides, “acyclic” nucleotides, 5-C-methyl nucleotides, terminal glyceryl and/or inverted deoxy abasic residue incorporation, and the like. Such chemical modifications, when used in various siNA constructs, may have the advantage of preserving RNAi activity in cells while at the same time, increasing the serum stability of the construct.

For example, the antisense region of an siNA molecule can comprise a phosphorothioate internucleotide linkage at the 3′-end of said antisense region. The antisense region may if desired comprise between about one and about five phosphorothioate internucleotide linkages at the 5′-end of said antisense region. The 3′-terminal nucleotide overhangs of a siNA molecule of the invention may if desired comprise ribonucleotides or deoxyribonucleotides that are chemically modified at a nucleic acid sugar, base, or backbone. The 3′-terminal nucleotide overhangs may if desired comprise one or more universal base ribonucleotides. The 3′-terminal nucleotide overhangs may if desired comprise one or more acyclic nucleotides.

Introduction of chemically-modified nucleotides into nucleic acid may be of use to increase in vivo stability and bioavailability of RNA molecules. For example, the use of chemically-modified nucleic acid molecules may enable use of a lower dose of a particular nucleic acid molecule for a given therapeutic effect since chemically-modified nucleic acid molecules tend to have a longer half-life in serum. Furthermore, certain chemical modifications may improve the bioavailability of nucleic acid molecules by targeting particular cells or tissues and/or improving cellular uptake of the nucleic acid molecule. Therefore, even if the activity of a chemically-modified nucleic acid molecule is reduced as compared to a native nucleic acid molecule, for example when compared to a native RNA nucleic acid molecule, the overall activity of the modified nucleic acid molecule can be greater than the native molecule due to improved stability and/or delivery of the molecule. Chemically-modified siNA can also minimize the possibility of activating interferon activity in humans.

One embodiment of the invention provides an expression vector comprising a nucleic acid sequence encoding at least one siNA molecule of the invention in a manner that allows expression of the nucleic acid molecule. Another embodiment of the invention provides a cell, preferably a mammalian cell, comprising such an expression vector. The mammalian cell can be a human cell. The siNA molecule of the expression vector may suitably comprise a sense region and an antisense region and the antisense region may suitably comprise a sequence complementary to a sequence coding a component of the Notch signalling pathway, and the sense region may suitably comprise a sequence complementary to the antisense region. Such an siNA molecule may for example comprise two distinct strands having complementary sense and antisense regions, or alternatively may comprise a single strand having complementary sense and antisense regions.

### EXAMPLES OF CHEMICAL MODIFICATION OF NUCLEIC ACIDS

It will be appreciated that any appropriate chemical modification may be made within the scope of the present invention. The following non-limiting examples (see for example US Patent Publication No. 20030206887 (Morrissey)) are thus provided for illustrative purposes only.

For example, in one embodiment, chemical modification may comprise one or more (e.g., about 1, 2, 3, 4, 5, 6, 7, 8, 9, 10, or more) nucleotides comprising a backbone modified intemucleotide linkage such as that of Formula I: 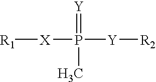
wherein for example each R1 and R2 is independently any nucleotide, non-nucleotide, or polynucleotide which can be naturally occurring or chemically modified, each X and Y is independently O, S, N, alkyl, or substituted alkyl, each Z and W is independently O, S, N, alkyl, substituted alkyl, O-alkyl, S-alkyl, alkaryl, or aralkyl, and wherein W, X, Y, and Z are optionally not all O.

Chemically-modified internucleotide linkages of Formula I, for example wherein any Z, W, X, and/or Y independently comprises a sulphur atom, may for example be present in either or both oligonucleotide strands of an siNA duplex, for example in the sense strand, the antisense strand, or both strands. For example, siNA molecules may if desired comprise one or more chemically-modified internucleotide linkages of Formula I at the 3′-end, the 5′-end, or both of the 3′- and 5′-ends of the sense strand, the antisense strand, or both strands. For example, siNA molecules may if desired comprise between about 1 and about 5 or more chemically-modified intemucleotide linkages of Formula I at the 5′-end of the sense strand, the antisense strand, or both strands.

In one embodiment, a chemically-modified short interfering nucleic acid (siNA) molecule may for example comprise one or more nucleotides or non-nucleotides such as those of Formula II: 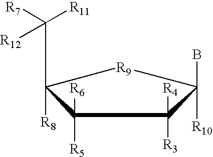
wherein each R3, R4, R5, R6, R7, R8, R10, R11 and R12 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2, and B is a nucleosidic base such as adenine, guanine, uracil, cytosine, thymine, 2-aminoadenosine, 5-methylcytosine, 2,6-diaminopurine, or any other non-naturally occurring base that can be complementary or non-complementary to target RNA or a non-nucleosidic base such as phenyl, naphthyl, 3-nitropyrrole, 5-nitroindole, nebularine, pyridone, pyridinone, or any other non-naturally occurring universal base that can be complementary or non-complementary to target RNA.

The chemically-modified nucleotide or non-nucleotide of Formula II can be present in one or both oligonucleotide strands of the siNA duplex, for example in the sense strand, the antisense strand, or both strands. The siNA molecules of the invention can comprise one or more chemically-modified nucleotide or non-nucleotide of Formula II at the 3′-end, the 5′-end, or both of the 3′- and 5′-ends of the sense strand, the antisense strand, or both strands. For example, an exemplary siNA molecule may if desired comprise between about 1 and about 5 or more chemically-modified nucleotide or non-nucleotide of Formula II at the 5′-end of the sense strand, the antisense strand, or both strands. In anther non-limiting example, an exemplary siNA molecule may comprise between about 1 and about 5 or more chemically-modified nucleotide or non-nucleotide of Formula II at the 3′-end of the sense strand, the antisense strand, or both strands.

In another non-limiting example, a chemically-modified short interfering nucleic acid (siNA) molecule may comprise one or more modified nucleotides or non-nucleotides such as those of Formula III: 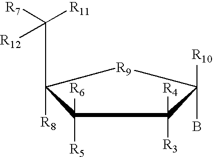
wherein each R3, R4, R5, R6, R7, R8, R10, R11 and R12 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2, and B is a nucleosidic base such as adenine, guanine, uracil, cytosine, thymine, 2-aminoadenosine, 5-methylcytosine, 2,6-diaminopurine, or any other non-naturally occurring base that can be employed to be complementary or non-complementary to target RNA or a non-nucleosidic base such as phenyl, naphthyl, 3-nitropyrrole, 5-nitroindole, nebularine, pyridone, pyridinone, or any other non-naturally occurring universal base that can be complementary or non-complementary to target RNA.

The chemically-modified nucleotide or non-nucleotide of Formula III can be present in one or both oligonucleotide strands of the siNA duplex, for example in the sense strand, the antisense strand, or both strands. Chemically modified siNA molecules may thus comprise one or more chemically-modified nucleotide or non-nucleotide of Formula III at the 3′-end, the 5′-end, or both of the 3′- and 5′-ends of the sense strand, the antisense strand, or both strands. For example, an exemplary siNA molecule may id desired comprise between about 1 and about 5 or more chemically-modified nucleotide or non-nucleotide of Formula III at the 5′-end of the sense strand, the antisense strand, or both strands. In another non-limiting example, an exemplary siNA molecule may comprise between about 1 and about 5 or more chemically-modified nucleotide or non-nucleotide of Formula III at the 3′-end of the sense strand, the antisense strand, or both strands.

In another non-limiting example, a chemically-modified short interfering nucleic acid (siNA) molecule may comprise a nucleotide having Formula II or III, wherein the nucleotide having Formula II or III is in an inverted configuration. For example, the nucleotide having Formula II or III is connected to the siNA construct in a 3′,3′; 3′-2′, 2′-3′; or 5′,5′ configuration, such as at the 3′-end, 5′-end, or both 3′ and 5′-ends of one or both siNA strands.

In another non-limiting example, a chemically-modified short interfering nucleic acid (siNA) molecule may comprise a 5′-terminal phosphate group having Formula IV: 4 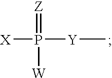
wherein each X and Y is independently O, S, N, alkyl, substituted alkyl, or alkylhalo; each Z and W is independently O, S, N, alkyl, substituted alkyl, O-alkyl, S-alkyl, alkaryl, aralkyl, or alkylhalo; and wherein W, X, Y and Z are not all O.

In one embodiment, a siNA molecule may have a 5′-terminal phosphate group having Formula IV on the target-complementary strand, for example a strand complementary to a target RNA, wherein the siNA molecule comprises an all RNA siNA molecule. In another embodiment, a siNA molecule may have a 5′-terminal phosphate group having Formula IV on the target-complementary strand wherein the siNA molecule also comprises e.g. 1-3 nucleotide 3′-terminal nucleotide overhangs having e.g. between about 1 and about 4 deoxyribonucleotides on the 3′-end of one or both strands. In another embodiment, a 5′-terminal phosphate group having Formula IV may be present on the target-complementary strand of a siNA molecule of the invention, for example a siNA molecule having chemical modifications having any of Formulae I-VII.

In another non-limiting example, a chemically-modified short interfering nucleic acid (siNA) molecule may comprise one or more phosphorothioate intemucleotide linkages. For example, in a non-limiting example, a chemically-modified short interfering nucleic acid (siNA) may have about 1, 2, 3, 4, 5, 6, 7, 8 or more phosphorothioate intemucleotide linkages in one or both siNA strands. The phosphorothioate intemucleotide linkages can be present in one or both oligonucleotide strands of the siNA duplex, for example in the sense strand, the antisense strand, or both strands. The siNA molecules of the invention can comprise one or more phosphorothioate intemucleotide linkages at the 3′-end, the 5′-end, or both of the 3′- and 5′-ends of the sense strand, the antisense strand, or both strands. For example, an exemplary siNA molecule of the invention can comprise between about 1 and about 5 or more consecutive phosphorothioate intemucleotide linkages at the 5′-end of the sense strand, the antisense strand, or both strands. In another non-limiting example, an exemplary siNA molecule of the invention can comprise one or more pyrimidine phosphorothioate intemucleotide linkages in the sense strand, the antisense strand, or both strands. In yet another non-limiting example, an exemplary siNA molecule of the invention can comprise one or more purine phosphorothioate intemucleotide linkages in the sense strand, the antisense strand, or both strands.

In another non-limiting example, a chemically-modified short interfering nucleic acid (siNA) molecule may comprise one or more phosphorothioate intemucleotide linkages, and/or one or more 2′-deoxy, 2′-O-methyl, 2′-deoxy-2′-fluoro, and/or one or more universal base-modified nucleotides, and optionally a terminal cap molecule at the 3′-end, the 5′-end, or both of the 3′- and 5′-ends of the sense strand; and wherein the antisense strand comprises one or more phosphorothioate intemucleotide linkages, and/or one or 2′-deoxy, 2′-O-methyl, 2′-deoxy-2′-fluoro, and/or one or more universal base-modified nucleotides, and optionally a terminal cap molecule at the 3′-end, the 5′-end, or both of the 3′- and 5′-ends of the antisense strand. In another embodiment, one or more pyrimidine nucleotides of the sense and/or antisense siNA stand are chemically modified with 2′-deoxy, 2′-O-methyl and/or 2′-deoxy-2′-fluoro nucleotides, with or without one or more, phosphorothioate intemucleotide linkages and/or a terminal cap molecule at the 3′-end, the 5′-end, or both of the 3′- and 5′-ends, being present in the same or different strand.

In another non-limiting example, a chemically-modified short interfering nucleic acid (siNA) molecule may comprise between about 1 and about 5, phosphorothioate intemucleotide linkages, and/or one or more 2′-deoxy, 2′-O-methyl, 2′-deoxy-2′-fluoro, and/or one or more universal base-modified nucleotides, and optionally a terminal cap molecule at the 3′-end, the 5′-end, or both of the 3′- and 5′-ends of the sense strand; and wherein the antisense strand comprises any of between about 1 and about 5 or more phosphorothioate intemucleotide linkages, and/or one or more 2′-deoxy, 2′-O-methyl, 2′-deoxy-2′-fluoro, and/or one or more universal base-modified nucleotides, and optionally a terminal cap molecule at the 3′-end, the 5′-end, or both of the 3′- and 5′-ends of the antisense strand. In another embodiment, one or more, for example one or more pyrimidine nucleotides of the sense and/or antisense siNA stand are chemically modified with 2′-deoxy, 2′-O-methyl and/or 2′-deoxy-2′-fluoro nucleotides, with or without between about 1 and about 5 or more phosphorothioate intemucleotide linkages and/or a terminal cap molecule at the 3′-end, the 5′-end, or both of the 3′- and 5′-ends, being present in the same or different strand.

In another non-limiting example, a chemically-modified short interfering nucleic acid (siNA) molecule may comprise one or more, phosphorothioate intemucleotide linkages, and/or between one or more 2′-deoxy, 2′-O-methyl, 2′-deoxy-2′-fluoro, and/or one or more universal base-modified nucleotides, and optionally a terminal cap molecule at the 3′-end, the 5′-end, or both of the 3′- and 5′-ends of the sense strand; and wherein the antisense strand comprises any of between about 1 and about 10 or more phosphorothioate intemucleotide linkages, and/or one or more 2′-deoxy, 2′-O-methyl, 2′-deoxy-2′-fluoro, and/or one or more universal base-modified nucleotides, and optionally a terminal cap molecule at the 3′-end, the 5′-end, or both of the 3′- and 5′-ends of the antisense strand. In another embodiment, one or more, pyrimidine nucleotides of the sense and/or antisense siNA stand may be chemically modified with 2′-deoxy, 2′-O-methyl and/or 2′-deoxy-2′-fluoro nucleotides, with or without one or more, phosphorothioate internucleotide linkages and/or a terminal cap molecule at the 3′-end, the 5′-end, or both of the 3′- and 5′-ends, being present in the same or different strand.

In another non-limiting example, a chemically-modified short interfering nucleic acid (siNA) molecule may comprise between about 1 and about 5 or more phosphorothioate intemucleotide linkages, and/or one or more 2′-deoxy, 2′-O-methyl, 2′-deoxy-2′-fluoro, and/or one or more universal base-modified nucleotides, and optionally a terminal cap molecule at the 3′-end, the 5′-end, or both of the 3′- and 5′-ends of the sense strand; and wherein the antisense strand comprises any of between about 1 and about 5 or more phosphorothioate intemucleotide linkages, and/or one or more 2′-deoxy, 2′-O-methyl, 2′-deoxy-2′-fluoro, and/or one or more universal base-modified nucleotides, and optionally a terminal cap molecule at the 3′-end, the 5′-end, or both of the 3′- and 5′-ends of the antisense strand. In another embodiment, one or more pyrimidine nucleotides of the sense and/or antisense siNA stand may be chemically modified with 2′-deoxy, 2′-O-methyl and/or 2′-deoxy-2′-fluoro nucleotides, with or without between about 1 and about 5 or more phosphorothioate intemucleotide linkages and/or a terminal cap molecule at the 3′-end, the 5′-end, or both of the 3′- and 5′-ends, being present in the same or different strand.

In another non-limiting example, a chemically-modified short interfering nucleic acid (siNA) molecule may comprise between about 1 and about 5, specifically about 1, 2, 3, 4, 5 or more phosphorothioate internucleotide linkages in each strand of the siNA molecule.

In another non-limiting example, a chemically-modified short interfering nucleic acid (siNA) molecule may comprise one or more 2′-5′ internucleotide linkages. The 2′-5′ intemucleotide linkage(s) may for example be at 3′-end the 5′-end, the 3′-end, or both of the 5′- and 3′-ends of one or both siNA sequence strands. Alernatively or in addition, 2′-5′ intemucleotide linkage(s) may be present at various other positions within one or both siNA sequence strands.

In another non-limiting example, a short interfering nucleic acid (siNA) molecule may comprise a duplex having two strands, one or both of which can be chemically modified, wherein each strand is for example between about 18 and about 27 (e.g., about 18, 19, 20, 21, 22, 23, 24, 25, 26, or 27) nucleotides in length, wherein the duplex has for example between about 18 and about 23 (e.g., about 18, 19, 20, 21, 22, or 23) base pairs, and wherein the chemical modification may if desired comprise a structure having any of Formulae I-VII. For example, an exemplary chemically-modified siNA molecule of the invention comprises a duplex having two strands, one or both of which can be chemically modified with a chemical modification having any of Formulae I-VII, wherein each strand consists of about 21 nucleotides, each having two 2-nucleotide 3′-terminal nucleotide overhangs, and wherein the duplex has about 19 base pairs.

In another non-limiting example, a short interfering nucleic acid (siNA) molecule may comprise a single-stranded hairpin structure, wherein the siNA is for example between about 36 and about 70 (e.g., about 36, 40, 45, 50, 55, 60, 65, or 70) nucleotides in length having between about 18 and about 23 (e.g., about 18, 19, 20, 21, 22, or 23) base pairs, and wherein the siNA may if desired include a chemical modification comprising a structure having any of Formulae I-VII. For example, an exemplary chemically-modified siNA molecule may comprise a linear oligonucleotide having between about 42 and about 50 (e.g., about 42, 43, 44, 45, 46, 47, 48, 49, or 50) nucleotides that is optionally chemically modified with a chemical modification having any of Formulae I-VII, wherein the linear oligonucleotide may form a hairpin structure having about 19 base pairs and a 2 nucleotide 3′-terminal nucleotide overhang.

In another non-limiting example, a short interfering nucleic acid (siNA) molecule may for example comprise a stem loop motif, wherein the loop portion of the siNA molecule is biodegradable. For example, a linear hairpin siNA may be designed such that degradation of the loop portion of the siNA molecule in vivo can generate a double-stranded siNA molecule with 3′-terminal overhangs, such as 3′-terminal nucleotide overhangs comprising about 2 nucleotides.

In another non-limiting example, a short interfering nucleic acid (siNA) molecule may comprise a circular nucleic acid molecule, wherein the siNA is for example between about 38 and about 70 (e.g., about 38, 40, 45, 50, 55, 60, 65, or 70) nucleotides in length having for example between about 18 and about 23 (e.g., about 18, 19, 20, 21, 22, or 23) base pairs, and wherein the siNA may optionally include a chemical modification, for example a structure having any of Formulae I-VII. For example, an exemplary chemically-modified siNA molecule of the invention may comprise a circular oligonucleotide having between about 42 and about 50 (e.g., about 42, 43, 44, 45, 46, 47, 48, 49, or 50) nucleotides optionally chemically modified with a chemical modification such as, for example, any of Formulae I-VII, wherein the circular oligonucleotide forms a “dumbbell” shaped structure having about 19 base pairs and about 2 loops.

In another non-limiting example, a short interfering nucleic acid (siNA) molecule may comprise two loop motifs, wherein one or both loop portions of the siNA molecule may be biodegradable. For example, a circular siNA molecule may be designed such that degradation of the loop portions of the siNA molecule in vivo can generate a double-stranded siNA molecule with for example 3′-terminal overhangs, such as 3′-terminal nucleotide overhangs comprising about 2 nucleotides.

In another non-limiting example, a chemically-modified short interfering nucleic acid (siNA) molecule may comprise at least one abasic moiety, for example a moiety of Formula V: 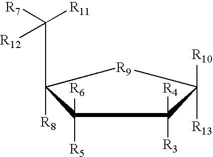
wherein for example each R3, R4, R5, R6, R7, R8, R10, R11, R12, and R13 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2.

In another non-limiting example, a short interfering nucleic acid (siNA) molecule may comprise at least one inverted abasic moiety, for example a moiety of Formula VI:

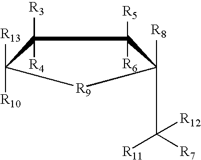

wherein for example each R3, R4, R5, R6, R7, R8, R10, R11, R12, and R13 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2, and either R2, R3, R8 or R13 may serve as points of attachment to the siNA molecule.

In another non-limiting example, a chemically-modified short interfering nucleic acid (siNA) molecule may for example comprise one or more substituted polyalkyl moieties, for example a moiety of Formula VII: 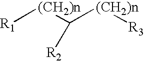
wherein for example each n is independently an integer for example from 1 to 12, each of R1, R2 and R3 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or a group having Formula I, and either R1, R2 or R3 may serve as points of attachment to the siNA molecule. Suitably for example, in Formula VII, R1 and R2 may be hydroxyl (OH) groups, n may be 1, and R3 may comprise O and is the point of attachment to the 3′-end, 5-end, or both 3′ and 5′-ends of one or both strands of a double-stranded siNA molecule or to a single-stranded siNA molecule. This modification may be referred to as “glyceryl”.

In another non-limiting example, a moiety having any of Formula V, VI or VII of the invention may be present at the 3′-end, the 5′-end, or both of the 3′- and 5′-ends of a siNA molecule. For example, a moiety having Formula V, VI or VII can be present at the 3′-end, the 5′-end, or both of the 3′- and 5′-ends of the antisense strand, the sense strand, or both the antisense and sense strands of an siNA molecule. Alternatively or in addition, a moiety of Formula VII may for example be present at the 3′-end or the 5′-end of a hairpin siNA molecule as described herein.

In another embodiment, an siNA molecule may comprise an abasic residue for example having Formula V or VI, wherein the abasic residue having Formula V or VI is connected to the siNA construct in a 3′-3′, 3′-2′, 2′-3′, or 5′-5′ configuration, such as at the 3′-end, 5′-end, or both 3′ and 5′-ends of one or both siNA strands.

In another embodiment, a siNA molecule may for example comprise one or more locked nucleic acid (LNA) nucleotides, for example at the 5′-end, 3′-end, 5′ and 3′-end, or any combination thereof, of the siNA molecule.

In another embodiment, a siNA molecule may for example comprise one or more acyclic nucleotides, for example at the 5′-end, 3′-end, 5′ and 3′-end, or any combination thereof, of the siNA molecule.

In one embodiment, for example, the invention provides a chemically-modified short interfering nucleic acid (siNA) molecule capable of mediating RNA interference (RNAi) against Notch signalling wherein the chemically-modified siNA comprises a sense region, where any (e.g., one or more or all) pyrimidine nucleotides present in the sense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and where any (e.g., one or more or all) purine nucleotides present in the sense region are 2′-deoxy purine nucleotides (e.g., wherein all purine nucleotides are 2′-deoxy purine nucleotides or alternately a plurality of purine nucleotides are 2′-deoxy purine nucleotides).

In one embodiment, for example, the invention provides a chemically-modified short interfering nucleic acid (siNA) molecule capable of mediating RNA interference (RNAi) against Notch signalling wherein the chemically-modified siNA comprises a sense region, where any (e.g., one or more or all) pyrimidine nucleotides present in the sense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and where any (e.g., one or more or all) purine nucleotides present in the sense region are 2′-deoxy purine nucleotides (e.g., wherein all purine nucleotides are 2′-deoxy purine nucleotides or alternately a plurality of purine nucleotides are 2′-deoxy purine nucleotides), wherein any nucleotides comprising a 3′-terminal nucleotide overhang that are present in said sense region are 2′-deoxy nucleotides.

In one embodiment, for example, the invention provides a chemically-modified short interfering nucleic acid (siNA) molecule capable of mediating RNA interference (RNAi) against Notch signalling wherein the chemically-modified siNA comprises an antisense region, where any (e.g., one or more or all) pyrimidine nucleotides present in the antisense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein any (e.g., one or more or all) purine nucleotides present in the antisense region are 2′-O-methyl purine nucleotides (e.g., wherein all purine nucleotides are 2′-O-methyl purine nucleotides or alternately a plurality of purine nucleotides are 2′-O-methyl purine nucleotides).

In one embodiment, for example, the invention provides a chemically-modified short interfering nucleic acid (siNA) molecule capable of mediating RNA interference (RNAi) against Notch signalling wherein the chemically-modified siNA comprises an antisense region, where any (e.g., one or more or all) pyrimidine nucleotides present in the antisense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein any (e.g., one or more or all) purine nucleotides present in the antisense region are 2′-O-methyl purine nucleotides (e.g., wherein all purine nucleotides are 2′-O-methyl purine nucleotides or alternately a plurality of purine nucleotides are 2′-O-methyl purine nucleotides), wherein any nucleotides comprising a 3′-terminal nucleotide overhang that are present in said antisense region are 2′-deoxy nucleotides.

In one embodiment, for example, the invention provides a chemically-modified short interfering nucleic acid (siNA) molecule capable of mediating RNA interference (RNAi) against Notch signalling wherein the chemically-modified siNA comprises a sense region, where one or more pyrimidine nucleotides present in the sense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and where one or more purine nucleotides present in the sense region are 2′-deoxy purine nucleotides (e.g., wherein all purine nucleotides are 2′-deoxy purine nucleotides or alternately a plurality of purine nucleotides are 2′-deoxy purine nucleotides), and inverted deoxy abasic modifications that are optionally present at the 3′-end, the 5′-end, or both of the 3′- and 5′-ends of the sense region, the sense region optionally further comprising a 3′-terminal nucleotide overhang having between about 1 and about 4 or more 2′-deoxyribonucleotides; and wherein the chemically-modified short interfering nucleic acid molecule comprises an antisense region, where one or more pyrimidine nucleotides present in the antisense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein one or more purine nucleotides present in the antisense region are 2′-O-methyl purine nucleotides (e.g., wherein all purine nucleotides are 2′-O-methyl purine nucleotides or alternately a plurality of purine nucleotides are 2′-O-methyl purine nucleotides), and a terminal cap modification that is optionally present at the 3′-end, the 5′-end, or both of the 3′- and 5′-ends of the antisense sequence, the antisense region optionally further comprising a 3′-terminal overhang having between about 1 and about 4 or more 2′-deoxynucleotides, wherein the overhang nucleotides can further comprise one or more phosphorothioate internucleotide linkages.

In one embodiment, for example, the invention provides a chemically-modified short interfering nucleic acid (siNA) molecule capable of mediating RNA interference (RNAi) against Notch signalling wherein the siNA comprises a sense region, where one or more pyrimidine nucleotides present in the sense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and where one or more purine nucleotides present in the sense region are purine ribonucleotides (e.g., wherein all purine nucleotides are purine ribonucleotides or alternately a plurality of purine nucleotides are purine ribonucleotides), and inverted deoxy abasic modifications that are optionally present at the 3′-end, the 5′-end, or both of the 3′- and 5′-ends of the sense region, the sense region optionally further comprising a 3′-terminal nucleotide overhang having between about 1 and about 4 (e.g, about 1, 2, 3, or 4) 2′-deoxyribonucleotides; and wherein the siNA comprises an antisense region, where one or more pyrimidine nucleotides present in the antisense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein any purine nucleotides present in the antisense region are 2′-O-methyl purine nucleotides (e.g., wherein all purine nucleotides are 2′-O-methyl purine nucleotides or alternately a plurality of purine nucleotides are 2′-O-methyl purine nucleotides), and a terminal cap modification that is optionally present at the 3′-end, the 5′-end, or both of the 3′- and 5′-ends of the antisense sequence, the antisense region optionally further comprising a 3′-terminal nucleotide overhang having between about 1 and about 4 (e.g, about 1, 2, 3, or 4) 2′-deoxynucleotides, wherein the overhang nucleotides can further comprise one or more (e.g., 1, 2, 3, or 4) phosphorothioate internucleotide linkages.

In one embodiment, the invention provides a chemically-modified short interfering nucleic acid (siNA) molecule capable of mediating RNA interference (RNAi) against Notch signalling wherein the chemical modification comprises a conjugate covalently attached to the chemically-modified siNA molecule. In another embodiment, the conjugate is covalently attached to the chemically-modified siNA molecule via a biodegradable linker. In one embodiment, the conjugate molecule is attached at the 3′-end of either the sense strand, the antisense strand, or both strands of the chemically-modified siNA molecule. In another embodiment, the conjugate molecule is attached at the 5′-end of either the sense strand, the antisense strand, or both strands of the chemically-modified siNA molecule. In yet another embodiment, the conjugate molecule is attached to both the 3′-end and the 5′-end of the sense strand, the antisense strand, or both strands of the chemically-modified siNA molecule, or any combination thereof. In one embodiment, a conjugate molecule of the invention comprises a molecule that facilitates delivery of a chemically-modified siNA molecule molecule into a biological system such as a cell. In another embodiment, the conjugate molecule attached to the chemically-modified siNA molecule is a poly ethylene glycol, human serum albumin, or a ligand for a cellular receptor that can mediate cellular uptake. Examples of specific conjugate molecules contemplated by the instant invention that can be attached to chemically-modified siNA molecules are described in Vargeese et al., U.S. Ser. No. 60/311,865, incorporated by reference herein.

In one embodiment, the invention provides a chemically-modified short interfering nucleic acid (siNA) molecule capable of mediating RNA interference (RNAi) against Notch signalling wherein one or both strands of the siNA molecule that are assembled from two separate oligonucleotides comprise ribonucleotides at positions within the siNA that are critical for siNA mediated RNAi in a cell. All other positions within the siNA can also include chemically-modified nucleotides and/or non-nucleotides such as nucleotides and or non-nucleotides having any of Formulae I-VII or any combination thereof to the extent that the ability of the siNA molecule to support RNAi activity in a cell is maintained.

In an alternative embodiment, neither of the strands of the siNA molecule that are assembled from two separate oligonucleotides comprise ribonucleotides may be critical for siNA mediated RNAi in a cell. In this case, all the positions within the siNA molecule can include chemically-modified nucleotides and/or non-nucleotides such as nucleotides and or non-nucleotides having any of Formulae I-VII or any combination thereof to the extent that the ability of the siNA molecule to support RNAi activity in a cell is maintained.

In one embodiment, the invention provides a chemically-modified short interfering nucleic acid (siNA) molecule capable of mediating RNA interference (RNAi) against Notch signalling wherein the antisense region and/or the sense region of the siNA molecule comprise ribonucleotides at positions within the siNA that are critical for siNA mediated RNAi in a cell. All other positions within the siNA can include chemically-modified nucleotides and/or non-nucleotides such as nucleotides and/or non-nucleotides having any of Formulae I-VII or any combination thereof to the extent that the ability of the siNA molecule to support RNAi activity in a cell is maintained.

In one embodiment, the invention features a chemically-modified short interfering nucleic acid (siNA) molecule capable of mediating RNA interference (RNAi) against Notch signalling wherein the antisense region and/or the sense region of the siNA molecule are assembled from two separate oligonucleotides that comprise ribonucleotides that are critical for siNA mediated RNAi in a cell. For example, all the positions within the siNA molecule can include chemically-modified nucleotides and/or non-nucleotides such as nucleotides and or non-nucleotides having any of Formulae I-VII or any combination thereof to the extent that the ability of the siNA molecule molecule to support RNAi activity in a cell is maintained.

Vectors, Promoters and Expression

Two approaches are particularly suitable for expressing a double-stranded interfering RNA (although it will be appreciated that others may also be used). In the first, the two nucleic acid sequences constituting the two strands of the RNA duplex are transcribed by individual promoters that drive their expression. In the second, the two strands of complementary nucleic acid sequences are expressed off a single promoter resulting in a fold-back stem-loop or hairpin structure that is processed into the dsRNA. A promoter useful in the present invention can be a promoter of eukaryotic or prokaryotic origin that can provide high levels of constitutive expression across a variety of cell types and will be sufficient to direct the transcription of a distally located sequence, which is a sequence linked to the 5′ end of the promoter sequence in a cell.

An inducible promoter is transcriptionally active when bound to a transcriptional activator that, in turn, is activated under a specific set of conditions, for example, in the presence of a particular combination of chemical signals that affect binding of the transcriptional activator to the inducible promoter and/or affect function of the transcriptional activator itself. Thus, an inducible promoter is a promoter that, either in the absence of an inducer, does not direct expression, or directs low levels of expression, of a nucleic acid sequence to which the inducible promoter is operably linked; or exhibits a low level of expression in the presence of a regulating factor that, when removed, allows high-level expression from the promoter, for example, the tet system. In the presence of an inducer, an inducible promoter directs transcription at an increased level.

It is understood that the function of a promoter can be further modified, if desired, to include appropriate regulatory elements to provide for the desired level of expression or replication in the host cell. For example, appropriate promoter and enhancer elements can be chosen to provide for constitutive, inducible or cell type-specific expression. Useful constitutive promoter and enhancer elements for expression of a target gene transcript include, for example, RSV, CMV, CAG, SV40 and IgH elements. Other constitutive, inducible and cell type-specific regulatory elements are well known in the art. One skilled in the art will be able to select and/or modify the promoter that is most effective for the desired application and cell type so as to optimize target gene silencing.

Thus, promoters that are useful in the invention include those promoters that are sufficient to render promoter-dependent gene expression controllable for cell-type specificity, cell-stage specificity, or tissue-specificity, and those promoters that are inducible by external signals or agents. The promoter sequence can be one that does not occur in nature, so long as it functions in a vertebrate cell.

For the therapeutic and prophylactic applications of the present invention, transient controllable expression of a dsRNA can allow for controlled target inhibition. In this embodiment, the expression of the dsRNA transgene can be induced or suppressed by the simple administration or cessation of administration to an organism, respectively, of an exogenous inducer such as, for example, tetracycline or its derivative doxycycline. In this embodiment, the invention allows for efficient regulation of Notch signalling, a low background level of inhibition in the off state, fast induction kinetics, and large window of regulation by administering the inducer, for example, tetracycline or a tetracycline analogue to the individual. The level of dsRNA expression can be varied depending upon which particular inducer, for example, which tetracycline analogue is used. In addition, the level of dsRNA expression can also be modulated by adjusting the dose of the inducer that is administered to the patient to thereby adjust the concentration achieved in the circulation and in the tissues of interest. The inducer can be administered by any route appropriate for delivery of the particular inducing compound and preferred routes of administration can include oral administration, intravenous administration and topical administration.

A vector useful in the methods of the invention includes any nucleic acid that functions to carry, harbor or express the nucleic acid sequences corresponding to a dsRNA capable of modulating Notch signalling. The structure of the vector can include any desired form that is feasible to make and desirable for a particular application of the invention. Such forms include, for example, circular forms such as plasmids and phagemids, as well as linear or branched forms. A nucleic acid vector can be composed of, for example, DNA or RNA, as well as contain partially or fully, nucleotide derivatives, analogs and mimetics. Such nucleic acid vectors can for example be obtained from natural sources, produced recombinantly or chemically synthesized.

In certain embodiments, a viral vector can be used to practice the invention. As exemplified below, a dsRNA can be encoded on a retroviral vector, for example, a lentiviral vector. Unlike other retroviruses, lentiviruses have the ability to efficiently infect and transduce non-proliferating cells, including for example, terminally differentiated cells. Lentiviruses also have the ability to efficiently infect and transduce proliferating cells. Despite the pathogenesis associated with lentiviruses, it is well known to those skilled in the art that the undesirable properties of lentiviruses can be recombinantly separated so that its beneficial characteristics can be harnessed as a delivery vehicle for therapeutic or diagnostic nucleic acid sequences. Therefore, lentiviral-based vectors can be produced that are safe, replication-defective and self-inactivating, while still maintaining the beneficial ability to transduce non-dividing cells and integrate into the host chromosome for stable expression. A description of the various different modalities of lentiviral vector and packaging systems for vector assembly and gene delivery can be found, for example, in Naldini et al., Science 272:263-267 (1996); Naldini et al., Proc. Natl. Acad. Sci. USA 93:11382-11388 (1996); Zufferey et al., Nature Bio. 15:871-875 (1997); Dull et al., J. Virol. 72:463-8471 (1998); Miyoshi et al., J. Virol. 72:8150-8157 (1998), and Zufferey et al., J. Virol. 72:9873-9880 (1998), all of which are incorporated herein by reference.

As described herein, other modifications to enhance safety and specificity include the use of specific internal promoters that regulate gene expression, either temporally or with tissue or cell specificity as well as the introduction of post-transcriptional regulatory elements that enhance expression of the dsRNA including, for example, the Woodchuck hepatitis virus post-transcriptional regulatory element (WPRE) and the Cana PPT flap, as described, for example, by Zephyr et al., J Viol. 1999. 73(4):2886-92; Zennou et al., Cell 101:173-85 (2000), both of which are incorporated herein by reference.

Packaging cell lines for vector poduction can be chosen that continuously produce high-titer vector. A packaging cell line useful for producing a retroviral vector of the invention further can be one in which the expression of packaging genes and VSV-G, and therefore the production of vector, can be turned on at will as described by Kafri et al., J. Virol. 73(1): 576-84 (1999), which is incorporated herein by reference.

A pseudotyped viral vector that encodes a dsRNA capable of inhibiting a pathogen can be produced by transfecting cells with a viral vector, for example, a retroviral vector. As described herein, exemplary host cells for transfection with the lentiviral vector production system include, for example, mammalian primary cells; established mammalian cell lines, such as COS, CHO, HeLa, NIH3T3, 293T and PC12 cells; amphibian cells, such as Xenopus embryos and oocytes; and other vertebrate cells. Exemplary host cells also include insect cells (for example, Drosophila), yeast cells (for example, S. cerevisiae, S. pombe, or Pichia pastoris) and prokaryotic cells (for example, E. coli).

Methods for introducing a nucleic acid into a host cell are well known in the art and include, for example, various methods of transfection such as calcium phosphate, DEAE-dextran and lipofection methods, electroporation and microinjection. The methods of isolating, cloning and expressing nucleic acid molecules of the invention referred to herein are routine in the art and are described in detail, for example, in Sambrook et al., Molecular Cloning: A Laboratory Manual, Cold Spring Harbor Laboratory, New York (1992) and in Ausubel et al., Current Protocols in Molecular Biology, John Wiley and Sons, Baltimore, Md. (1998), which are incorporated herein by reference.

In providing a patient (or cell) with the RNAi agents, the dosage of administered agent will vary depending on such factors as the patient's age, weight, height, sex, general medical condition, previous medical history, and the like.

One vector useful for both in vivo and ex vivo delivery is a liposome or comparable vesicle-like structure. The liposome can be produced in a solution containing the agent so that the agent is encapsulated during polymerization. Alternatively, the liposomes can be polymerized first, and the agent can be added later by resuspending the polymerized liposomes in a solution of the agent and treating with sonication to effect encapsulation. In one embodiment, the liposome is produced so that in the right pH or under the right conditions, the agent is evulsed. For example, “micromachines” evulse their contents when treated with a specific frequency radio wave. Alternatively the liposomes can be produced to be uncharged which will allow them to be taken up by the cell.

For ex vivo applications cells (such as immune cells) may suitably be isolated and contacted with siRNAs or “long” double-stranded RNAs. The dsRNAs can be induced to be taken up by the cells using any method known to one of skill in the art, including but not limited to transfection, transformation, lipofection, electroporation, microinjection, transduction, infection, use of viral vectors, and using products such as TansMessenger™ Transfection Reagent, PolyFect™ transfection reagent, Effectene™ transfection reagent, and SuperFect™ transfection reagent (all from Qiagen, Inc.), Lipofectamine™ transfection reagent (Gibco) and the Amaxa Nucleofector™ system (Amaxa Inc, MD, US). The cells may then be re-introduced into the mammal.

For in vivo gene therapy, any methods of known in the art can be used. In addition, any gene therapy vector can be used to produce the dsRNA, for example, by encoding an RNA hairpin. Many such vectors are easily obtainable from commercial vendors known to those skilled in the art. However, in one implementation of gene therapy, a replicating virus can be engineered to contain (in the case of a RNA virus) or produce (in the case of a DNA virus) an RNA precursor of the desired siRNA. For example, a replication competent vaccinia virus can be used, which is engineered to encode an RNA hairpin which is subsequently converted into an siRNA. Alternatively, an RNA virus such a picorna virus can be engineered to contain an RNA hairpin as a part of its genome. In either case, the RNA structure can be designed so that the hairpin could be cleaved by Dicer or other nuclease to produce the siRNA. Replication of the virus would thereby seed many tissues with the siRNA.

The term “interfering nucleic acid”, “interfering RNA”, “short interfering nucleic acid” (siNA), “short interfering oligonucleotide”, or “chemically-modified interfering nucleic acid” as used herein refers to any nucleic acid molecule capable of mediating RNA interference (“RNAi”) or gene silencing; see for example Bass, 2001, Nature, 411, 428-429; Elbashir et al., 2001, Nature, 411, 494-498; Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914. For example the molecule may comprise a double-stranded polynucleotide molecule comprising self-complementary sense and antisense regions, wherein the antisense region comprises complementarity to a target nucleic acid molecule coding for a component of the Notch signalling pathway. Alternatively the molecule may also for example comprise a single-stranded hairpin polynucleotide having self-complementary sense and antisense regions, wherein the antisense region comprises complementarity to such a target nucleic acid molecule. The molecule may also comprise a circular single-stranded polynucleotide having two or more loop structures and a stem comprising self-complementary sense and antisense regions, wherein the antisense region comprises complementarity to a target nucleic acid molecule, and wherein the circular polynucleotide can be processed either in vivo or in vitro to generate an active siNA capable of mediating RNAi. As used herein, interfering nucleic acid molecules need not be limited to those molecules containing only RNA, but may also comprise chemically-modified nucleotides and non-nucleotides. In certain embodiments, interfering nucleic acid molecules of the invention lack 2′-hydroxy (2′-OH) containing nucleotides. Applicant describes in certain embodiments short interfering nucleic acids that do not require the presence of nucleotides having a 2′-hydroxy group for mediating RNAi and as such, interfering nucleic acid molecules of the invention optionally do not contain any ribonucleotides (e.g., nucleotides having a 2′-OH group). Modified short interfering nucleic acid molecules for use in the invention can also be referred to as short interfering modified oligonucleotides “siMON.” As used herein, the term siNA includes molecules that are capable of mediating sequence specific RNAi, for example short interfering RNA (siRNA), double-stranded RNA (dsRNA), micro-RNA, short hairpin RNA (shRNA), short interfering oligonucleotide, short interfering nucleic acid, short interfering modified oligonucleotide, chemically-modified siRNA, post-transcriptional gene silencing RNA (ptgsRNA), and others.

The term “RNAi agent” as used herein means any agent capable of mediating RNA interference (“RNAi”). For example the agent may comprise a double-stranded polynucleotide molecule comprising sense and antisense regions, wherein the antisense region comprises complementarity to a target nucleic acid molecule coding for a component of the Notch signalling pathway. Alternatively the molecule may also for example comprise a single-stranded hairpin polynucleotide having sense and antisense regions, wherein the antisense region comprises complementarity to such a target nucleic acid molecule. The molecule may also comprise a circular single-stranded polynucleotide having one or more loop structures and a stem comprising self-complementary sense and antisense regions, wherein the antisense region comprises complementarity to a target nucleic acid molecule, and wherein the circular polynucleotide can be processed either in vivo or in vitro to generate an active siNA capable of mediating RNAi. An RNAi agent may also be a vector, e.g. nucleic acid vector such as a DNA vector, coding for a molecule capable of mediating sequence specific RNAi, for example coding for short interfering RNA (siRNA), double-stranded RNA (dsRNA), micro-RNA, short hairpin RNA (shRNA), short interfering oligonucleotide, short interfering nucleic acid, and precursors and derivatives thereof.

By “modulate” is meant that the expression of the gene, or level of RNA molecule or equivalent RNA molecules encoding one or more proteins or protein subunits, or activity of one or more proteins or protein subunits is up-regulated or down-regulated, such that expression, level, or activity is greater than or less than that observed in the absence of the modulator. For example, the term “modulate” can mean “inhibit” or “reduce” but the use of the word “modulate” is not limited to this definition.

By “inhibition of expression” or “reduction of expression” it is meant that the activity of a gene expression product or level of RNAs or equivalent RNAs encoding one or more gene products is reduced below that observed in the absence of the nucleic acid molecule of the invention. In one embodiment, inhibition with a siNA molecule preferably is below that level observed in the presence of an inactive or attenuated molecule that is unable to mediate an RNAi response. In another embodiment, inhibition of gene expression with the siNA molecule of the instant invention is greater in the presence of the siNA molecule than in its absence.

By “complementarity” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al., 1987, CSH Symp. Quant. Biol. LII pp. 123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9 or 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

By “RNA” is meant a molecule comprising at least one ribonucleotide residue. By “ribonucleotide” is meant a nucleotide with a hydroxyl group at the 2′ position of a beta-D-ribo-furanose moiety. The terms include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siNA or internally, for example at one or more nucleotides of the RNA. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

The term “phosphorothioate” as used herein preferably refers to an internucleotide linkage having Formula I, wherein Z and/or W comprise a sulfur atom. Hence, the term phosphorothioate refers to both phosphorothioate and phosphorodithioate internucleotide linkages.

The term “universal base” as used herein preferably refers to nucleotide base analogs that form base pairs with each of the natural DNA/RNA bases with little discrimination between them. Non-limiting examples of universal bases include C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art (see for example Loakes, 2001, Nucleic Acids Research, 29, 2437-2447).

The term “acyclic nucleotide” as used herein preferably refers to any nucleotide having an acyclic ribose sugar, for example where any of the ribose carbons (C1, C2, C3, C4, or C5), are independently or in combination absent from the nucleotide.

By “improved capacity to mediate RNAi” is meant to include RNAi activity measured in vitro and/or in vivo where the RNAi activity is a reflection of both the ability of the siNA to mediate RNAi and the stability of the siRNAs of the invention. In this invention, the product of these activities can be increased in vitro and/or in vivo compared to an all RNA siRNA or an siNA containing a plurality of ribonucleotides. In some cases, the activity or stability of the siNA molecule can be decreased (i.e., less than ten-fold), but the overall activity of the siNA molecule is enhanced, in vitro and/or in vivo.

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine, and therefore lacks a base at the 1′-position.

An “alkyl” group refers to a saturated aliphatic hydrocarbon, including straight-chain, branched-chain, and cyclic alkyl groups. Preferably, the alkyl group has 1 to 12 carbons. More preferably, it is a lower alkyl of from 1 to 7 carbons, more preferably 1 to 4 carbons. The alkyl group can be substituted or unsubstituted. When substituted the substituted group(s) is preferably, hydroxyl, cyano, alkoxy, ═O, ═S, NO2 or N(CH3)2, amino, or SH. The term also includes alkenyl groups that are unsaturated hydrocarbon groups containing at least one carbon-carbon double bond, including straight-chain, branched-chain, and cyclic groups. Preferably, the alkenyl group has 1 to 12 carbons. More preferably, it is a lower alkenyl of from 1 to 7 carbons, more preferably 1 to 4 carbons. The alkenyl group may be substituted or unsubstituted. When substituted the substituted group(s) is preferably, hydroxyl, cyano, alkoxy, ═O, ═S, NO2, halogen, N(CH3)2, amino, or SH. The term “alkyl” also includes alkynyl groups that have an unsaturated hydrocarbon group containing at least one carbon-carbon triple bond, including straight-chain, branched-chain, and cyclic groups. Preferably, the alkynyl group has 1 to 12 carbons. More preferably, it is a lower alkynyl of from 1 to 7 carbons, more preferably 1 to 4 carbons. The alkynyl group may be substituted or unsubstituted. When substituted the substituted group(s) is preferably, hydroxyl, cyano, alkoxy, ═O, ═S, NO2 or N(CH3)2, amino or SH.

Such alkyl groups can also include aryl, alkylaryl, carbocyclic aryl, heterocyclic aryl, amide and ester groups. An “aryl” group refers to an aromatic group that has at least one ring having a conjugated pi electron system and includes carbocyclic aryl, heterocyclic aryl and biaryl groups, all of which may be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

By “nucleotide” as used herein is as recognized in the art to include natural bases (standard), and modified bases including those known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see, for example, Usman and McSwiggen, supra; Eckstein et al., International PCT Publication No. WO 92/07065; Usman et al., International PCT Publication No. WO 93/15187; Uhlman & Peyman, supra, all are hereby incorporated by reference herein). There are several examples of modified nucleic acid bases known in the art as summarized by Limbach et al., 1994, Nucleic Acids Res. 22, 2183. Some of the non-limiting examples of base modifications that can be introduced into nucleic acid molecules include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents.

By “abasic” is meant sugar moieties lacking a base or having other chemical groups in place of a base at the 1′ position, see for example Adamic et al., U.S. Pat. No. 5,998,203.

By “unmodified nucleoside” is meant one of the bases adenine, cytosine, guanine, thymine, or uracil joined to the 1′ carbon of .beta.-D-ribo-furanose.

By “modified nucleoside” is meant any nucleotide base that contains a modification in the chemical structure of an unmodified nucleotide base, sugar and/or phosphate.

In connection with 2′-modified nucleotides as described for the present invention, by “amino” is meant 2′-NH2 or 2′-O—NH2, which may be modified or unmodified. Such modified groups are described, for example, in Eckstein et al., U.S. Pat. No. 5,672,695 and Matulic-Adamic et al., U.S. Pat. No. 6,248,878, which are both incorporated by reference in their entireties.

Synthesis of Nucleic Acid Molecules

To prepare an RNAi agent useful in a method of the invention standard methods known in the art can suitably be used as described, for example, in Ausubel et al., Current Protocols in Molecular Biology (Supplement 56), John Wiley & Sons, New York (2001); Sambrook and Russel, Molecular Cloning: A Laboratory Manual, 3rd ed., Cold Spring Harbor Press, Cold Spring Harbor (2001); and Dieffenbach and Dveksler, PCR Primer: A Laboratory Manual, Cold Spring Harbor Press (1995), Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 umol scale protocol with a 2.5 min coupling step for 2′-O-methylated nucleotides and a 45 sec coupling step for 2′-deoxy nucleotides or 2′-deoxy-2′-fluoro nucleotides. Alternatively, syntheses at the 0.2 umol scale can be performed on a 96-well plate synthesizer, such as the instrument produced by Protogene (Palo Alto, Calif.) with minimal modification to the cycle. For example, RNA can be transcribed from PCR products, followed by gel purification. Standard procedures known in the art for in vitro transcription of RNA from PCR templates carrying, for example, T7 or SP6 promoter sequences can be used. For example dsRNAs may suitbably be synthesized using a PCR template and an Ambion (Austin, Tex., USA) T7 MegaScript kit, following the Manufacturer's recommendations and the RNA can then be precipitated with LiCl and resuspended in buffer. The specific dsRNAs produced can be tested for resistance to digestion by RNases A and T1. dsRNAs can be produced with 3′ overhangs at one or both termini of preferably 1-10 nucleotides, more preferably 1-3 nucleotides or with blunt ends at one or both termini. Thymidine nucleotide overhangs were found to be well-tolerated in mammalian cells, and the sequence of the overhang appears not to contribute to target recognition. Thus, any type of overhang can be used, however, the use of thymidine has been found to reduce costs and can enhance nuclease resistance of siRNAs in the cell culture medium and within transfected cells.

Thus, a dsRNA, including siRNA, can be both partially or completely double-stranded. Generally, a siRNA encompasses to fragments of at least 18, at least 19, at least 20, at least 21, at least 22, at least 23, at least 24, at least 25, at least 30, at least 35, at least 40, at least 45, at least 50 or more nucleotides per strand, with characteristic 3′ overhangs of at least 1, at least 2, at least 3, or at least 4 nucleotides. As set forth above, an interfering dsRNA can be of any length desired by the user as long as the ability to inhibit target gene expression is preserved.

The 21-23 nucleotide dsRNAs can be chemically synthesized by any method known to one of skill in the art, for example using Expedite RNA phosphoramidites and thymidine phosphoramidite (Proligo, Boulder, Colo.). Synthetic oligonucleotides can be deprotected and gel-purified. dsRNA annealing can be carried out by any method known in the art, for example: a phenol-chloroform extraction, followed by mixing equimolar concentrations of sense and antisense RNA (50 nM to 10 mM, depending on the length and amount available) and incubating in an appropriate buffer (such as 0.3 M NaOAc, pH 6) at 90.degree. C. for 30 sec and then extracting with phenol/chloroform and chloroform. The resulting dsRNA can be precipitated with ethanol and dissolved in an appropriate buffer depending on the intended use of the dsRNA.

Preferably small nucleic acid motifs (“small” in ths context refers to nucleic acid motifs no more than 100 nucleotides in length, preferably no more than 80 nucleotides in length, and most preferably no more than 50 nucleotides in length; e.g., individual siNA oligonucleotide sequences or siNA sequences synthesized in tandem) are used for exogenous delivery. The simple structure of these molecules increases the ability of the nucleic acid to invade targeted regions of protein and/or RNA structure. Exemplary molecules of the instant invention are chemically synthesized, and others can similarly be synthesized.

The method of synthesis used for RNA including certain siNA molecules of the invention follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59,

Alternatively, the nucleic acid molecules of the present invention can be synthesized separately and joined together post-synthetically, for example, by ligation (Moore et al., 1992, Science 256, 9923; Draper et al., International PCT Publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204), or by hybridization following synthesis and/or deprotection.

The siNA molecules of the invention can also suitably be synthesized via a tandem synthesis methodology, wherein both siNA strands are synthesized as a single contiguous oligonucleotide fragment or strand separated by a cleavable linker which is subsequently cleaved to provide separate siNA fragments or strands that hybridize and permit purification of the siNA duplex. The linker can be a polynucleotide linker or a non-nucleotide linker. Tandem synthesis of siNA can be readily adapted to both multiwell/multiplate synthesis platforms such as 96 well or similarly larger multi-well platforms. The tandem synthesis of siNA as described herein can also be readily adapted to large scale synthesis platforms employing batch reactors, synthesis columns and the like.

A siNA molecule can also be assembled from two distinct nucleic acid strands or fragments wherein one fragment includes the sense region and the second fragment includes the antisense region of the RNA molecule.

The nucleic acid molecules provided by and used in the present invention can be modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). siNA constructs can be purified by gel electrophoresis using general methods or can be purified by high pressure liquid chromatography (HPLC; see Wincott et al., supra, the totality of which is hereby incorporated herein by reference) and re-suspended in water.

In another aspect of the invention, siNA molecules of the invention may be expressed from transcription units inserted into DNA or RNA vectors. The recombinant vectors may suitably be DNA plasmids or viral vectors. siNA expressing viral vectors can be constructed based on, but not limited to, adeno-associated virus, retrovirus, adenovirus, or alphavirus. The recombinant vectors capable of expressing the siNA molecules can be delivered as described herein, and persist in target cells. Alternatively, viral vectors can be used that provide for transient expression of siNA molecules.

Administration of Nucleic Acid Molecules

An RNAi agent may be delivered using a delivery vehicle, including liposomes, for administration to a subject, carriers and diluents and their salts, and/or can be present in pharmaceutically acceptable formulations. Methods for the delivery of nucleic acid molecules are described in Akhtar et al., 1992, Trends Cell Bio., 2, 139; Delivery Strategies for Antisense Oligonucleotide Therapeutics, ed. Akhtar, 1995, Maurer et al., 1999, Mol. Membr. Biol., 16, 129-140; Hofland and Huang, 1999, Handb. Exp. Pharmacol., 137, 165-192; and Lee et al., 2000, ACS Symp. Ser., 752, 184-192, all of which are incorporated herein by reference. Beigelman et al., U.S. Pat. No. 6,395,713, and Sullivan et al., PCT WO 94/02595, further describe the general methods for delivery of nucleic acid molecules. These protocols can be utilized for the delivery of virtually any nucleic acid molecule. Nucleic acid molecules can be administered to cells by a variety of methods known to those of skill in the art, including, but not restricted to, encapsulation in liposomes, by iontophoresis, or by incorporation into other vehicles, such as hydrogels, cyclodextrins, biodegradable nanocapsules, and bioadhesive microspheres, or by proteinaceous vectors (O'Hare and Normand, International PCT Publication No. WO 00/53722). Alternatively, the nucleic acid/vehicle combination is locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry et al., 1999, Clin. Cancer Res., 5, 2330-2337 and Barry et al., International PCT Publication No. WO 99/31262. The molecules of the instant invention can be used as pharmaceutical agents. Pharmaceutical agents prevent, modulate the occurrence, or treat (alleviate a symptom to some extent, preferably all of the symptoms) of a disease state in a patient.

Thus, the invention provides a pharmaceutical composition comprising one or more nucleic acid(s) of the invention in an acceptable carrier, such as a stabilizer, buffer, or the like. The polynucleotides of the invention can be administered (e.g., RNA, DNA or protein) and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration, suppositories for rectal administration, sterile solutions, suspensions for injectable administration, and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, including for example a human. Suitable forms, in part, depend upon the use or the route of entry, for example oral, transdermal, or by injection. Such forms should not prevent the composition or formulation from reaching a target cell (i.e., a cell to which the negatively charged nucleic acid is desirable for delivery). For example, pharmacological compositions injected into the blood stream should be soluble. Other factors are known in the art, and include considerations such as toxicity and forms that prevent the composition or formulation from exerting its effect.

If desired, RNAi agents such as siRNAs (or nucleic acids coding for siRNAs, shRNAs and the like) or vectors coding therefor, can be introduced by “GeneGun” as in typical DNA-mediated vaccination. For example, siRNAs can be affixed to particles/beads, and ballistically/biolistically introduced into, for example, skin, muscle or mucosal surfaces using the Gene Gun. RNAi can be initiated at the site of injection, then spread systemically. As an alternative, DNAs can be introduced that encode hairpin structure RNAs in front of a promoter active in human cells. Introduction of the DNA into human cells can be accomplished for example by GeneGun, injection, or other known methods. Transcription suitably yields a hairpin RNA, which can then be cleaved by Dicer or other nuclease in situ to yield the effective siRNA.

By “systemic administration” is meant in vivo systemic absorption or accumulation of drugs in the blood stream followed by distribution throughout the entire body. Administration routes that lead to systemic absorption include, without limitation: intravenous, subcutaneous, intraperitoneal, inhalation, oral, intrapulmonary and intramuscular. Each of these administration routes exposes the siNA molecules of the invention to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

By “pharmaceutically acceptable formulation” is meant, a composition or formulation that allows for the effective distribution of the nucleic acid molecules of the instant invention in the physical location most suitable for their desired activity. Nonlimiting examples of agents suitable for formulation with the nucleic acid molecules of the instant invention include: P-glycoprotein inhibitors (such as Pluronic P85), which can enhance entry of drugs into the CNS (Jolliet-Riant and Tillement, 1999, Fundam. Clin. Pharmacol., 13, 16-26); biodegradable polymers, such as poly (DL-lactide-coglycolide) microspheres for sustained release delivery after intracerebral implantation (Emerich, D F et al., 1999, Cell Transplant, 8, 47-58) (Alkermes, Inc. Cambridge, Mass.); and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies for the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058.

Nucleic acid molecules of the invention can for example be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per patient per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy.

The nucleic acid molecules of the present invention can also be administered to a patient in combination with other therapeutic compounds to increase the overall therapeutic effect. The use of multiple compounds to treat an indication can increase the beneficial effects while reducing the presence of side effects.

In another aspect of the invention, RNA molecules may be expressed from transcription units (see for example Couture et al., 1996, TIG., 12, 510) inserted into DNA or RNA vectors. The recombinant vectors can be, for example, DNA plasmids or viral vectors. siNA expressing viral vectors can be constructed based on, but not limited to, adeno-associated virus, retrovirus, adenovirus, or alphavirus. In another embodiment, pol III based constructs are used to express nucleic acid molecules of the invention (see for example Thompson, U.S. Pat. Nos. 5,902,880 and 6,146,886). The recombinant vectors capable of expressing the siNA molecules can be delivered as described above, and persist in target cells. Alternatively, viral vectors can be used that provide for transient expression of nucleic acid molecules. Such vectors can be repeatedly administered as necessary. Once expressed, the siNA molecule interacts with the target mRNA and generates an RNAi response. Delivery of siNA molecule expressing vectors can be systemic, such as by intravenous or intra-muscular administration, by administration to target cells ex-planted from a patient followed by reintroduction into the patient, or by any other means that would allow for introduction into the desired target cell (for a review see Couture et al., 1996, TIG., 12, 510).

In one aspect the invention features an expression vector comprising a nucleic acid sequence encoding at least one siNA molecule of the instant invention. The expression vector can encode one or both strands of a siNA duplex, or a single self complementary strand that self hybridizes into a siNA duplex. The nucleic acid sequences encoding the siNA molecules of the instant invention can be operably linked in a manner that allows expression of the siNA molecule (see for example Paul et al., 2002, Nature Biotechnology, 19, 505; Miyagishi and Taira, 2002, Nature Biotechnology, 19, 497; Lee et al., 2002, Nature Biotechnology, 19, 500; and Novina et al., 2002, Nature Medicine, advance online publication doi:10.1038/nm725).

In another aspect, the invention features an expression vector comprising: a) a transcription initiation region (e.g., eukaryotic pol I, II or III initiation region); b) a transcription termination region (e.g., eukaryotic pol I, II or III termination region); and c) a nucleic acid sequence encoding at least one of the siNA molecules of the instant invention; wherein said sequence is operably linked to said initiation region and said termination region, in a manner that allows expression and/or delivery of the siNA molecule. The vector can optionally include an open reading frame (ORF) for a protein operably linked on the 5′ side or the 3′-side of the sequence encoding the siNA of the invention; and/or an intron (intervening sequences).

Transcription of the siNA molecule sequences can be driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743-7; Gao and Huang, 1993, Nucleic Acids Res., 21, 2867-72; Lieber et al., 1993, Methods Enzymol., 217, 47-66; Zhou et al., 1990, Mol. Cell. Biol., 10, 4529-37). Several investigators have demonstrated that nucleic acid molecules expressed from such promoters can function in mammalian cells (e.g., Kashani-Sabet et al., 1992, Antisense Res. Dev., 2, 3-15; Ojwang et al., 1992, Proc. Natl. Acad. Sci. USA, 89, 10802-6; Chen et al., 1992, Nucleic Acids Res., 20, 4581-9; Yu et al., 1993, Proc. Natl. Acad. Sci. USA, 90, 6340-4; L'Huillier et al., 1992, EMBO J., 11, 4411-8; Lisziewicz et al., 1993, Proc. Natl. Acad. Sci. U.S.A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as siNA in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736). The above siNA transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors).

In another aspect the invention features an expression vector comprising a nucleic acid sequence encoding at least one of the siNA molecules of the invention, in a manner that allows expression of that siNA molecule. The expression vector comprises in one embodiment; a) a transcription initiation region; b) a transcription termination region; and c) a nucleic acid sequence encoding at least one strand of the siNA molecule; wherein the sequence is operably linked to the initiation region and the termination region, in a manner that allows expression and/or delivery of the siNA molecule.

In another embodiment the expression vector comprises: a) a transcription initiation region; b) a transcription termination region; c) an open reading frame; and d) a nucleic acid sequence encoding at least one strand of a siNA molecule, wherein the sequence is operably linked to the 3′-end of the open reading frame; and wherein the sequence is operably linked to the initiation region, the open reading frame and the termination region, in a manner that allows expression and/or delivery of the siNA molecule. In yet another embodiment the expression vector comprises: a) a transcription initiation region; b) a transcription termination region; c) an intron; and d) a nucleic acid sequence encoding at least one siNA molecule; wherein the sequence is operably linked to the initiation region, the intron and the termination region, in a manner which allows expression and/or delivery of the nucleic acid molecule.

In another embodiment, the expression vector comprises: a) a transcription initiation region; b) a transcription termination region; c) an intron; d) an open reading frame; and e) a nucleic acid sequence encoding at least one strand of a siNA molecule, wherein the sequence is operably linked to the 3′-end of the open reading frame; and wherein the sequence is operably linked to the initiation region, the intron, the open reading frame and the termination region, in a manner which allows expression and/or delivery of the siNA molecule.

In another embodiment, the invention provides a method for identifying a Notch signalling pathway gene sequence that is a target for RNA interference aimed at modulating an immune reponse by selecting a candidate Notch signalling pathway target gene sequence; contacting a host immune cell (e.g. T-cell, B-cell or APC) with a dsRNA that corresponds to the target gene sequence; and identifying whether the dsRNA modulates the immune reponse.

Notch Signalling

As used herein, the expression “Notch signalling” is synonymous with the expression “the Notch signalling pathway” and refers to any one or more of the upstream or downstream events that result in, or from, (and including) activation of the Notch receptor.

Preferably, by “Notch signalling” we refer to any event directly upstream or downstream of Notch receptor activation or inhibition including activation or inhibition of Notch/Notch ligand interactions, upregulation or downregulation of Notch or Notch ligand expression or activity and activation or inhibition of Notch signalling transduction including, for example, proteolytic cleavage of Notch and upregulation or downregulation of the Ras-Jnk signalling pathway.

Thus, by “Notch signalling” we refer to the Notch signalling pathway as a signal tranducing pathway comprising elements which interact, genetically and/or molecularly, with the Notch receptor protein. For example, elements which interact with the Notch protein on both a molecular and genetic basis are, by way of example only, Delta, Serrate and Deltex. Elements which interact with the Notch protein genetically are, by way of example only, Mastermind, Hairless, Su(H) and Presenilin.

In one aspect, Notch signalling includes signalling events taking place extracellularly or at the cell membrane. In a further aspect, it includes signalling events taking place intracellularly, for example within the cell cytoplasm or within the cell nucleus.

Modulation of Notch Signalling

The term “modulate” as used herein refers to a change or alteration in the biological activity of the Notch signalling pathway or a target signalling pathway thereof. The term “modulator” may refer to antagonists or inhibitors of Notch signalling, i.e. compounds which block, at least to some extent, the normal biological activity of the Notch signalling pathway. Conveniently such compounds may be referred to herein as inhibitors or antagonists. Alternatively, the term “modulator” may refer to agonists of Notch signalling, i.e. compounds which stimulate or upregulate, at least to some extent, the normal biological activity of the Notch signalling pathway. Conveniently such compounds may be referred to as upregulators or agonists. Preferably the modulator is an agonist of Notch signalling, and preferably an agonist of the Notch receptor (e.g. an agonist of the Notch1, Notch2, Notch3 and/or Notch4 receptor).

In the present invention Notch signalling preferably means specific signalling, meaning that the signalling results substantially or at least predominantly from the Notch signalling pathway, and preferably from Notch/Notch ligand interaction, rather than any other significant interfering or competing cause, such as cytokine signalling. Thus, in a preferred embodiment, Notch signalling excludes cytokine signalling.

The Notch signalling pathway is described in more detail below.

Key targets for Notch-dependent transcriptional activation are genes of the Enhancer of split complex (E[spl]). Moreover these genes have been shown to be direct targets for binding by the Su(H) protein and to be transcriptionally activated in response to Notch signalling. By analogy with EBNA2, a viral coactivator protein that interacts with a mammalian Su(H) homologue CBF1 to convert it from a transcriptional repressor to a transcriptional activator, the Notch intracellular domain, perhaps in association with other proteins may combine with Su(H) to contribute an activation domain that allows Su(H) to activate the transcription of E(spl) as well as other target genes. It should also be noted that Su(H) is not required for all Notch-dependent decisions, indicating that Notch mediates some cell fate choices by associating with other DNA-binding transcription factors or be employing other mechanisms to transduce extracellular signals.

According to one aspect of the present invention the active agent may be Notch or a fragment thereof which retains the signalling transduction ability of Notch or an analogue of Notch which has the signalling transduction ability of Notch.

As used herein the term “analogue of Notch” includes variants thereof which retain the signalling transduction ability of Notch. By “analogue” we include a protein which has Notch signalling transduction ability, but generally has a different evolutionary origin to Notch. Analogues of Notch include proteins from the Epstein Barr virus (EBV), such as EBNA2, BARFO or LMP2A.

In one embodiment, the active agent may be a Notch ligand, or a polynucleotide encoding a Notch ligand. Notch ligands of use in the present invention include endogenous Notch ligands which are typically capable of binding to a Notch receptor polypeptide present in the membrane of a variety of mammalian cells, for example hemapoietic stem cells.

The term “Notch ligand” as used herein means an agent capable of interacting with a Notch receptor to cause a biological effect. The term as used herein therefore includes naturally occurring protein ligands such as Delta and Serrate/Jagged as well as antibodies to the Notch receptor, peptidomimetics and small molecules which have corresponding biological effects to the natural ligands. Preferably the Notch ligand interacts with the Notch receptor by binding.

Particular examples of mammalian Notch ligands identified to date include the Delta family, for example Delta or Delta-like 1 (Genbank Accession No. AF003522—Homo sapiens), Delta-3 (Genbank Accession No. AF084576—Rattus norvegicus) and Delta-like 3 (Mus musculus) (Genbank Accession No. NM_{—}016941—Homo sapiens) and U.S. Pat. No. 6,121,045 (Millennium), Delta-4 (Genbank Accession Nos. AB043894 and AF 253468—Homo sapiens) and the Serrate family, for example Serrate-1 and Serrate-2 (WO97/01571, WO96/27610 and WO92/19734), Jagged-1 (Genbank Accession No. U73936—Homo sapiens) and Jagged-2 (Genbank Accession No. AF029778—Homo sapiens), and LAG-2. Homology between family members is extensive.

In an alternative embodiment, an activator of Notch signalling will act downstream of the Notch receptor. Thus, for example, the activator of Notch signalling may be a constitutively active Deltex polypeptide or a polynucleotide encoding such a polypeptide. Other downstream components of the Notch signalling pathway of use in the present invention include the polypeptides involved in the Ras/MAPK cascade catalysed by Deltex, polypeptides involved in the proteolytic cleavage of Notch such as Presenilin and polypeptides involved in the transcriptional regulation of Notch target genes, preferably in a constitutively active form.

Notch Signalling Pathway

The Notch signalling pathway directs binary cell fate decisions in the embryo. Notch was first described in Drosophila as a transmembrane protein that functions as a receptor for two different ligands, Delta and Serrate. Vertebrates express multiple Notch receptors and ligands (discussed below). At least four Notch receptors (Notch-1, Notch-2, Notch-3 and Notch-4) have been identified to date in human cells (see for example GenBank Accession Nos. AF308602, AF308601 and U95299—Homo sapiens).

Notch proteins are synthesized as single polypeptide precursors that undergo cleavage via a Furin-like convertase that yields two polypeptide chains that are further processed to form the mature receptor. The Notch receptor present in the plasma membrane comprises a heterodimer of two Notch proteolytic cleavage products, one comprising an N-terminal fragment consisting of a portion of the extracellular domain, the transmembrane domain and the intracellular domain, and the other comprising the majority of the extracellular domain. The proteolytic cleavage step of Notch to activate the receptor occurs in the Golgi apparatus and is mediated by a furin-like convertase.

Notch receptors are inserted into the membrane as heterodimeric molecules consisting of an extracellular domain containing up to 36 epidermal growth factor (EGF)-like repeats [Notch 1/2=36, Notch 3=34 and Notch 4=29], 3 Cysteine Rich Repeats (Lin-Notch (L/N) repeats) and a transmembrane subunit that contains the cytoplasmic domain. The cytoplasmic domain of Notch contains six ankyrin-like repeats, a polyglutamine stretch (OPA) and a PEST sequence. A further domain termed RAM23 lies proximal to the ankyrin repeats and is involved in binding to a transcription factor, known as Suppressor of Hairless [Su(H)] in Drosophila and CBFI in vertebrates (Tamura K, et al. (1995) Curr. Biol. 5:1416-1423 (Tamura)). The Notch ligands also display multiple EGF-like repeats in their extracellular domains together with a cysteine-rich DSL (Delta-Serrate Lag2) domain that is characteristic of all Notch ligands (Artavanis-Tsakomas et al. (1995) Science 268:225-232, Artavanis-Tsakomas et al. (1999) Science 284:770-776).

The Notch receptor is activated by binding of extracellular ligands, such as Delta, Serrate and Scabrous, to the EGF-like repeats of Notch's extracellular domain. Delta requires cleavage for activation. It is cleaved by the ADAM disintegrin metalloprotease Kuzbanian at the cell surface, the cleavage event releasing a soluble and active form of Delta. An oncogenic variant of the human Notch-1 protein, also known as TAN-1, which has a truncated extracellular domain, is constitutively active and has been found to be involved in T-cell lymphoblastic leukemias.

The cdc10/ankyrin intracellular-domain repeats mediate physical interaction with intracellular signal transduction proteins. Most notably, the cdc10/ankyrin repeats interact with Suppressor of Hairless [Su(H)]. Su(H) is the Drosophila homologue of C-promoter binding factor-1 [CBF-1], a mammalian DNA binding protein involved in the Epstein-Barr virus-induced immortalization of B-cells. It has been demonstrated that, at least in cultured cells, Su(H) associates with the cdc10/ankyrin repeats in the cytoplasm and translocates into the nucleus upon the interaction of the Notch receptor with its ligand Delta on adjacent cells. Su(H) includes responsive elements found in the promoters of several genes and has been found to be a critical downstream protein in the Notch signalling pathway. The involvement of Su(H) in transcription is thought to be modulated by Hairless.

The intracellular domain of Notch (NotchIC) also has a direct nuclear function (Lieber et al. (1993) Genes Dev 7(10):1949-65 (Lieber)). Recent studies have indeed shown that Notch activation requires that the six cdc10/ankyrin repeats of the Notch intracellular domain reach the nucleus and participate in transcriptional activation. The site of proteolytic cleavage on the intracellular tail of Notch has been identified between gly1743 and val1744 (termed site 3, or S3) (Schroeter, E. H. et al. (1998) Nature 393(6683):382-6 (Schroeter)). It is thought that the proteolytic cleavage step that releases the cdc10/ankyrin repeats for nuclear entry is dependent on Presenilin activity.

The intracellular domain has been shown to accumulate in the nucleus where it forms a transcriptional activator complex with the CSL family protein CBF1 (suppressor of hairless, Su(H) in Drosophila, Lag-2 in C. elegans) (Schroeter; Struhl, G. et al. (1998) Cell 93(4):649-60 (Struhl)). The NotchIC-CBF1 complexes then activate target genes, such as the bHLH proteins HES (hairy-enhancer of split like) 1 and 5 (Weinmaster G. (2000) Curr. Opin. Genet. Dev. 10:363-369 (Weinmaster)). This nuclear function of Notch has also been shown for the mammalian Notch homologue (Lu, F. M. et al. (1996) Proc Natl Acad Sci 93(11):5663-7 (Lu)).

S3 processing occurs only in response to binding of Notch ligands Delta or Serrate/Jagged. The post-translational modification of the nascent Notch receptor in the Golgi (Munro S, Freeman M. (2000) Curr. Biol. 10:813-820 (Munro); Ju B J, et al. (2000) Nature 405:191-195 (Ju)) appears, at least in part, to control which of the two types of ligand is expressed on a cell surface. The Notch receptor is modified on its extracellular domain by Fringe, a glycosyl transferase enzyme that binds to the Lin/Notch motif. Fringe modifies Notch by adding O-linked fucose groups to the EGF-like repeats (Moloney D J, et al. (2000) Nature 406:369-375 (Moloney), Brucker K, et al. (2000) Nature 406:411-415 (Brucker)). This modification by Fringe does not prevent ligand binding, but may influence ligand induced conformational changes in Notch. Furthermore, recent studies suggest that the action of Fringe modifies Notch to prevent it from interacting functionally with Serrate/Jagged ligands but allow it to preferentially bind Delta (Panin V M, et al. (1997) Nature 387:908-912 (Panin), Hicks C, et al. (2000) Nat. Cell. Biol. 2:515-520 (Hicks)). Although Drosophila has a single Fringe gene, vertebrates are known to express multiple genes (Radical, Manic and Lunatic Fringes) (Irvine K D (1999) Curr. Opin. Genet. Devel. 9:434-441 (Irvine)).

In an alternative embodiment, the activator of Notch signalling may act downstream of the Notch receptor. Thus, for example, the activator of Notch signalling may be a constitutively active Deltex polypeptide or a polynucleotide encoding such a polypeptide. Other downstream components of the Notch signalling pathway of use in the present invention include Deltex-1, Deltex-2, Deltex-3, Suppressor of Deltex (SuDx), Numb and isoforms thereof, Numb associated Kinase (NAK), Notchless, Dishevelled (Dsh), emb5, Fringe genes (such as Radical, Lunatic and Manic), PON, LNX, Disabled, Numblike, Nur77, NFkB2, Mirror, Warthog, Engrailed-1 and Engrailed-2, Lip-1 and homologues thereof, the polypeptides involved in the Ras/MAPK cascade modulated by Deltex, polypeptides involved in the proteolytic cleavage of Notch such as Presenilin and polypeptides involved in the transcriptional regulation of Notch target genes, preferably in a constitutively active form, and analogues, derivatives, variants and fragments thereof.

Signal transduction from the Notch receptor can occur via two different pathways (FIG. 1). The better defined pathway involves proteolytic cleavage of the intracellular domain of Notch (Notch IC) that translocates to the nucleus and forms a transcriptional activator complex with the CSL family protein CBF1 (suppressor of Hairless, Su(H) in Drosophila, Lag-2 in C. elegans). NotchIC-CBF1 complexes then activate target genes, such as the bHLH proteins HES (hairy-enhancer of split like) 1 and 5. Notch can also signal in a CBF1-independent manner that involves the cytoplasmic zinc finger containing protein Deltex. Unlike CBF1, Deltex does not move to the nucleus following Notch activation but instead can interact with Grb2 and modulate the Ras-JNK signalling pathway.

Thus, signal transduction from the Notch receptor can occur via two different pathways both of which are illustrated in FIG. 1. Target genes of the Notch signalling pathway include Deltex, genes of the Hes family (Hes-1 in particular), Enhancer of Split [E(spl)] complex genes, IL-10, CD-23, CD-4 and Dll-1.

Deltex, an intracellular docking protein, replaces Su(H) as it leaves its site of interaction with the intracellular tail of Notch. Deltex is a cytoplasmic protein containing a zinc-finger (Artavanis-Tsakomas et al. (1995) Science 268:225-232; Artavanis-Tsakomas et al. (1999) Science 284:770-776; Osborne B, Miele L. (1999) Immunity 11:653-663 (Osborne)). It interacts with the ankyrin repeats of the Notch intracellular domain. Studies indicate that Deltex promotes Notch pathway activation by interacting with Grb2 and modulating the Ras-JNK signalling pathway (Matsuno et al. (1995) Development 121(8):2633-44; Matsuno K, et al. (1998) Nat. Genet. 19:74-78). Deltex also acts as a docking protein which prevents Su(H) from binding to the intracellular tail of Notch (Matsuno). Thus, Su(H) is released into the nucleus where it acts as a transcriptional modulator. Recent evidence also suggests that, in a vertebrate B-cell system, Deltex, rather than the Su(H) homologue CBF1, is responsible for inhibiting E47 function (Ordentlich et al. (1998) Mol. Cell. Biol. 18:2230-2239 (Ordentlich)). Expression of Deltex is upregulated as a result of Notch activation in a positive feedback loop. The sequence of Homo sapiens Deltex (DTX1) mRNA may be found in GenBank Accession No. AF053700.

Hes-1 (Hairy-enhancer of Split-1) (Takebayashi K. et al. (1994) J Biol Chem 269(7):150-6 (Takebayashi)) is a transcriptional factor with a basic helix-loop-helix structure. It binds to an important functional site in the CD4 silencer leading to repression of CD4 gene expression. Thus, Hes-1 is strongly involved in the determination of T-cell fate. Other genes from the Hes family include Hes-5 (mammalian Enhancer of Split homologue), the expression of which is also upregulated by Notch activation, and Hes-3. Expression of Hes-1 is upregulated as a result of Notch activation. The sequence of Mus musculus Hes-1 can be found in GenBank Accession No. D16464.

The E(spl) gene complex [E(spl)-C] (Leimeister C. et al. (1999) Mech Dev 85(1-2):173-7 (Leimeister)) comprises seven genes of which only E(spl) and Groucho show visible phenotypes when mutant. E(spl) was named after its ability to enhance Split mutations, Split being another name for Notch. Indeed, E(spl)-C genes repress Delta through regulation of achaete-scute complex gene expression. Expression of E(spl) is upregulated as a result of Notch activation.

Interleukin-10 (IL-10) was first characterised in the mouse as a factor produced by Th2 cells which was able to suppress cytokine production by Th1 cells. It was then shown that IL-10 was produced by many other cell types including macrophages, keratinocytes, B cells, Th0 and Th1 cells. It shows extensive homology with the Epstein-Barr bcrf1 gene which is now designated viral IL-10. Although a few immunostimulatory effects have been reported, it is mainly considered as an immunosuppressive cytokine. Inhibition of T cell responses by IL-10 is mainly mediated through a reduction of accessory functions of antigen presenting cells. IL-10 has notably been reported to suppress the production of numerous pro-inflammatory cytokines by macrophages and to inhibit co-stimulatory molecules and MHC class II expression. IL-10 also exerts anti-inflammatory effects on other myeloid cells such as neutrophils and eosinophils. On B cells, IL-10 influences isotype switching and proliferation. More recently, IL-10 was reported to play a role in the induction of regulatory T cells and as a possible mediator of their suppressive effect. Although it is not clear whether it is a direct downstream target of the Notch signalling pathway, its expression has been found to be strongly up-regulated coincident with Notch activation. The mRNA sequence of IL-10 may be found in GenBank ref. No. GI1041812.

CD-23 is the human leukocyte differentiation antigen CD23 (FCE2) which is a key molecule for B-cell activation and growth. It is the low-affinity receptor for IgE. Furthermore, the truncated molecule can be secreted, then functioning as a potent mitogenic growth factor. The sequence for CD-23 may be found in GenBank ref. No. GI1783344.

CTLA4 (cytotoxic T-lymphocyte activated protein 4) is an accessory molecule found on the surface of T-cells which is thought to play a role in the regulation of airway inflammatory cell recruitment and T-helper cell differentiation after allergen inhalation. The promoter region of the gene encoding CTLA4 has CBF1 response elements and its expression is upregulated as a result of Notch activation. The sequence of CTLA4 can be found in GenBank Accession No. L15006.

Dlx-1 (distalless-1) (McGuinness T. Et al (1996) Genomics 35(3):473-85 (McGuiness)) expression is downregulated as a result of Notch activation. Sequences for Dlx genes may be found in GenBank Accession Nos. U51000-3.

CD-4 expression is downregulated as a result of Notch activation. A sequence for the CD-4 antigen may be found in GenBank Accession No. XM006966.

Other genes involved in the Notch signaling pathway, such as Numb, Mastermind and Dsh, and all genes the expression of which is modulated by Notch activation, are included in the scope of this invention.

As described above the Notch receptor family participates in cell-cell signalling events that influence T cell fate decisions. In this signalling NotchIC localises to the nucleus and functions as an activated receptor. Mammalian NotchIC interacts with the transcriptional repressor CBF1. It has been proposed that the NotchIC cdc10/ankyrin repeats are essential for this interaction. Hsieh et al (Hsieh et al. (1996) Molecular & Cell Biology 16(3):952-959) suggests rather that the N-terminal 114 amino acid region of mouse NotchIC contains the CBF1 interactive domain. It is also proposed that NotchIC acts by targeting DNA-bound CBF1 within the nucleus and abolishing CBF1-mediated repression through masking of the repression domain. It is known that Epstein Barr virus (EBV) immortalizing protein EBNA” also utilises CBF1 tethering and masking of repression to upregulate expression of CBF1-repressed B-cell genes. Thus, mimicry of Notch signal transduction is involved in EBV-driven immortalization. Strobl et al (Strobl et al. (2000) J Virol 74(4):1727-35) similarly reports that “EBNA2 may hence be regarded as a functional equivalent of an activated Notch receptor”. Other EBV proteins which fall in this category include BARF0 (Kusano and Raab-Truab (2001) J Virol 75(1):384-395 (Kusano and Raab-Traub)) and LMP2A.

By a “homologue” is meant a gene product that exhibits sequence homology, either amino acid or nucleic acid sequence homology, to any one of the known Notch ligands, for example as mentioned above. Typically, a homologue of a known Notch ligand will be at least 20%, preferably at least 30%, identical at the amino acid level to the corresponding known Notch ligand over a sequnce of at least 10, preferably at least 20, preferably at least 50, suitably at least 100 amino acids, or over the entire length of the Notch ligand. Techniques and software for calculating sequence homology between two or more amino acid or nucleic acid sequences are well known in the art (see for example databases maintained by the National Institutes of Health, available at the National Center for Biotechnology Information website, and Ausubel et al., Current Protocols in Molecular Biology (1995), John Wiley & Sons, Inc.)

Notch ligands identified to date have a diagnostic DSL domain (Delta, S. Serrate, L. Lag2) comprising 20 to 22 amino acids at the amino terminus of the protein and up to 14 or more EGF-like repeats on the extracellular surface. It is therefore preferred that homologues of Notch ligands also comprise a DSL domain at the N-terminus and up to 14 or more EGF-like repeats on the extracellular surface.

In addition, suitable homologues will be capable of binding to a Notch receptor. Binding may be assessed by a variety of techniques known in the art including in vitro binding assays.

Homologues of Notch ligands can be identified in a number of ways, for example by probing genomic or cDNA libraries with probes comprising all or part of a nucleic acid encoding a Notch ligand under conditions of medium to high stringency (for example 0.03M sodium chloride and 0.03M sodium citrate at from about 50° C. to about 60° C.). Alternatively, homologues may also be obtained using degenerate PCR which will generally use primers designed to target sequences within the variants and homologues encoding conserved amino acid sequences. The primers will contain one or more degenerate positions and will be used at stringency conditions lower than those used for cloning sequences with single sequence primers against known sequences.

Polypeptide substances may be purified from mammalian cells, obtained by recombinant expression in suitable host cells or obtained commercially. Alternatively, nucleic acid constructs encoding the polypeptides may be used. As a further example, overexpression of Notch or Notch ligand, such as Delta or Serrate, may be brought about by introduction of a nucleic acid construct capable of activating the endogenous gene, such as the Serrate or Delta gene. In particular, gene activation can be achieved by the use of homologous recombination to insert a heterologous promoter in place of the natural promoter, such as the Serrate or Delta promoter, in the genome of the target cell.

The activating molecule of the present invention may, in an alternative embodiment, be capable of modifying Notch-protein expression or presentation on the cell membrane or signalling pathways. Agents that enhance the presentation of a fully functional Notch-protein on the target cell surface include matrix metalloproteinases such as the product of the Kuzbanian gene of Drosophila (Dkuz et al. (1997) Cell 90: 271-280 (Dkuz)) and other ADAMALYSIN gene family members.

Notch Ligand Domains

As discussed above, Notch ligands typically comprise a number of distinctive domains. Some predicted/potential domain locations for various naturally occurring human Notch ligands (based on amino acid numbering in the precursor proteins) are shown below:

| | | |
|---|---|---|
| Component | Amino acids | Proposed function/domain |

Human Delta 1

| | | |
|---|---|---|
| SIGNAL | 1-17 | SIGNAL |
| CHAIN | 18-723 | DELTA-LIKE PROTEIN 1 |
| DOMAIN | 18-545 | EXTRACELLULAR |
| TRANSMEM | 546-568 | TRANSMEMBRANE |
| DOMAIN | 569-723 | CYTOPLASMIC |
| DOMAIN | 159-221 | DSL |
| DOMAIN | 226-254 | EGF-LIKE 1 |
| DOMAIN | 257-285 | EGF-LIKE 2 |
| DOMAIN | 292-325 | EGF-LIKE 3 |
| DOMAIN | 332-363 | EGF-LIKE 4 |
| DOMAIN | 370-402 | EGF-LIKE 5 |
| DOMAIN | 409-440 | EGF-LIKE 6 |
| DOMAIN | 447-478 | EGF-LIKE 7 |
| DOMAIN | 485-516 | EGF-LIKE 8 |

Human Delta 3

| | | |
|---|---|---|
| DOMAIN | 158-248 | DSL |
| DOMAIN | 278-309 | EGF-LIKE 1 |
| DOMAIN | 316-350 | EGF-LIKE 2 |
| DOMAIN | 357-388 | EGF-LIKE 3 |
| DOMAIN | 395-426 | EGF-LIKE 4 |
| DOMAIN | 433-464 | EGF-LIKE 5 |

Human Delta 4

| | | |
|---|---|---|
| SIGNAL | 1-26 | SIGNAL |
| CHAIN | 27-685 | DELTA-LIKE PROTEIN 4 |
| DOMAIN | 27-529 | EXTRACELLULAR |
| TRANSMEM | 530-550 | TRANSMEMBRANE |
| DOMAIN | 551-685 | CYTOPLASMIC |
| DOMAIN | 155-217 | DSL |
| DOMAIN | 218-251 | EGF-LIKE 1 |
| DOMAIN | 252-282 | EGF-LIKE 2 |
| DOMAIN | 284-322 | EGF-LIKE 3 |
| DOMAIN | 324-360 | EGF-LIKE 4 |
| DOMAIN | 362-400 | EGF-LIKE 5 |
| DOMAIN | 402-438 | EGF-LIKE 6 |
| DOMAIN | 440-476 | EGF-LIKE 7 |
| DOMAIN | 480-518 | EGF-LIKE 8 |

Human Jagged 1

| | | |
|---|---|---|
| SIGNAL | 1-33 | SIGNAL |
| CHAIN | 34-1218 | JAGGED 1 |
| DOMAIN | 34-1067 | EXTRACELLULAR |
| TRANSMEM | 1068-1093 | TRANSMEMBRANE |
| DOMAIN | 1094-1218 | CYTOPLASMIC |
| DOMAIN | 167-229 | DSL |
| DOMAIN | 234-262 | EGF-LIKE 1 |
| DOMAIN | 265-293 | EGF-LIKE 2 |
| DOMAIN | 300-333 | EGF-LIKE 3 |
| DOMAIN | 340-371 | EGF-LIKE 4 |
| DOMAIN | 378-409 | EGF-LIKE 5 |
| DOMAIN | 416-447 | EGF-LIKE 6 |
| DOMAIN | 454-484 | EGF-LIKE 7 |
| DOMAIN | 491-522 | EGF-LIKE 8 |
| DOMAIN | 529-560 | EGF-LIKE 9 |
| DOMAIN | 595-626 | EGF-LIKE 10 |
| DOMAIN | 633-664 | EGF-LIKE 11 |
| DOMAIN | 671-702 | EGF-LIKE 12 |
| DOMAIN | 709-740 | EGE-LIKE 13 |
| DOMAIN | 748-779 | EGF-LIKE 14 |
| DOMAIN | 786-817 | EGF-LIKE 15 |
| DOMAIN | 824-855 | EGF-LIKE 16 |
| DOMAIN | 863-917 | VON WILLEBRAND FACTOR C |

Human Jagged 2

| | | |
|---|---|---|
| SIGNAL | 1-26 | SIGNAL |
| CHAIN | 27-1238 | JAGGED 2 |
| DOMAIN | 27-1080 | EXTRACELLULAR |
| TRANSMEM | 1081-1105 | TRANSMEMBRANE |
| DOMAIN | 1106-1238 | CYTOPLASMIC |
| DOMAIN | 178-240 | DSL |
| DOMAIN | 249-273 | EGF-LIKE 1 |
| DOMAIN | 276-304 | EGF-LIKE 2 |
| DOMAIN | 311-344 | EGF-LIKE 3 |
| DOMAIN | 351-382 | EGF-LIKE 4 |
| DOMAIN | 389-420 | EGF-LIKE 5 |
| DOMAIN | 427-458 | EGF-LIKE 6 |
| DOMAIN | 465-495 | EGF-LIKE 7 |
| DOMAIN | 502-533 | EGF-LIKE 8 |
| DOMAIN | 540-571 | EGF-LIKE 9 |
| DOMAIN | 602-633 | EGF-LIKE 10 |
| DOMAIN | 640-671 | EGF-LIKE 11 |
| DOMAIN | 678-709 | EGF-LIKE 12 |
| DOMAIN | 716-747 | EGF-LIKE 13 |
| DOMAIN | 755-786 | EGF-LIKE 14 |
| DOMAIN | 793-824 | EGF-LIKE 15 |
| DOMAIN | 831-862 | EGF-LIKE 16 |
| DOMAIN | 872-949 | VON WILLEBRAND FACTOR C |

DSL Domain

A typical DSL domain may include most or all of the following consensus amino acid sequence (SEQ ID NO: 26):

Cys Xaa Xaa Xaa Xaa Xaa Xaa Xaa Xaa Cys Xaa Xaa
Xaa Cys Xaa Xaa
Xaa Xaa Xaa Xaa Xaa Xaa Xaa Xaa Xaa Cys Xaa Xaa
Xaa Xaa Xaa Xaa
Xaa Cys Xaa Xaa Xaa Xaa Xaa Xaa Xaa Xaa Cys

Preferably the DSL domain may include most or all of the following consensus amino acid sequence (SEQ ID NO: 27):

Cys Xaa Xaa Xaa ARO ARO Xaa Xaa Xaa Cys Xaa Xaa
Xaa Cys BAS NOP
BAS ACM ACM Xaa ARO NOP ARO Xaa Xaa Cys Xaa Xaa
Xaa NOP Xaa Xaa
Xaa Cys Xaa Xaa NOP ARO Xaa NOP Xaa Xaa Cys

wherein:
ARO is an aromatic amino acid residue, such as tyrosine, phenylalanine, tryptophan or histidine;
NOP is a non-polar amino acid residue such as glycine, alanine, proline, leucine, isoleucine or valine;
BAS is a basic amino acid residue such as arginine or lysine; and
ACM is an acid or amide amino acid residue such as aspartic acid, glutamic acid, asparagine or glutamine.

Preferably the DSL domain may include most or all of the following consensus amino acid sequence (SEQ ID NO: 28):

Cys Xaa Xaa Xaa Tyr Tyr Xaa Xaa Xaa Cys Xaa Xaa
Xaa Cys Arg Pro
Arg Asx Asp Xaa Phe Gly His Xaa Xaa Cys Xaa Xaa
Xaa Gly Xaa Xaa
Xaa Cys Xaa Xaa Gly Trp Xaa Gly Xaa Xaa Cys

(wherein Xaa may be any amino acid and Asx is either aspartic acid or asparagine).

An alignment of DSL domains from Notch ligands from various sources is shown in FIG. 3.

The DSL domain used may be derived from any suitable species, including for example Drosophila, Xenopus, rat, mouse or human. Preferably the DSL domain is derived from a vertebrate, preferably a mammalian, preferably a human Notch ligand sequence.

It will be appreciated that the term “DSL domain” as used herein includes sequence variants, fragments, derivatives and mimetics having activity corresponding to naturally occurring domains.

Suitably, for example, a DSL domain for use in the present invention may have at least 30%, preferably at least 50%, preferably at least 60%, preferably at least 70%, preferably at least 80%, preferably at least 90%, preferably at least 95% amino acid sequence identity to the DSL domain of human Jagged 1.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Jagged 2.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 1.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 3.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 4.

EGF-Like Domain

The EGF-like motif has been found in a variety of proteins, as well as EGF and Notch and Notch ligands, including those involved in the blood clotting cascade (Furie and Furie, 1988, Cell 53: 505-518). For example, this motif has been found in extracellular proteins such as the blood clotting factors IX and X (Rees et al., 1988, EMBO J. 7:2053-2061; Furie and Furie, 1988, Cell 53: 505-518), in other Drosophila genes (Knust et al., 1987 EMBO J. 761-766; Rothberg et al., 1988, Cell 55:1047-1059), and in some cell-surface receptor proteins, such as thrombomodulin (Suzuki et al., 1987, EMBO J. 6:1891-1897) and LDL receptor (Sudhof et al., 1985, Science 228:815-822). A protein binding site has been mapped to the EGF repeat domain in thrombomodulin and urokinase (Kurosawa et al., 1988, J. Biol. Chem 263:5993-5996; Appella et al., 1987, J. Biol. Chem. 262:4437-4440).

As reported by PROSITE a typical EGF domain may include six cysteine residues which have been shown (in EGF) to be involved in disulfide bonds. The main structure is proposed, but not necessarily required, to be a two-stranded beta-sheet followed by a loop to a C-terminal short two-stranded sheet. Subdomains between the conserved cysteines strongly vary in length as shown in the following schematic representation of a typical EGF-like domain: 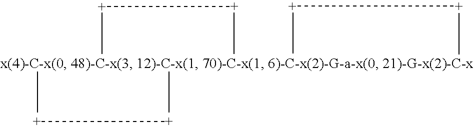
wherein:
‘C’: conserved cysteine involved in a disulfide bond.
‘G’: often conserved glycine
‘a’: often conserved aromatic amino acid
‘x’: any residue

The region between the 5th and 6th cysteine contains two conserved glycines of which at least one is normally present in most EGF-like domains.

The EGF-like domain used may be derived from any suitable species, including for example Drosophila, Xenopus, rat, mouse or human. Preferably the EGF-like domain is derived from a vertebrate, preferably a mammalian, preferably a human Notch ligand sequence.

It will be appreciated that the term “EGF domain” as used herein includes sequence variants, fragments, derivatives and mimetics having activity corresponding to naturally occurring domains.

Suitably, for example, an EGF-like domain for use in the present invention may have at least 30%, preferably at least 50%, preferably at least 60%, preferably at least 70%, preferably at least 80%, preferably at least 90%, preferably at least 95% amino acid sequence identity to an EGF-like domain of human Jagged 1.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Jagged 2.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 1.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 3.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 4.

As a practical matter, whether any particular amino acid sequence is at least X % identical to another sequence can be determined conventionally using known computer programs. For example, the best overall match between a query sequence and a subject sequence, also referred to as a global sequence alignment, can be determined using a program such as the FASTDB computer program based on the algorithm of Brutlag et al. (Comp. App. Biosci. (1990) 6:237-245). In a sequence alignment the query and subject sequences are either both nucleotide sequences or both amino acid sequences. The result of the global sequence alignment is given as percent identity.

The term “Notch ligand N-terminal domain” means the part of a Notch ligand sequence from the N-terminus to the start of the DSL domain. It will be appreciated that this term includes sequence variants, fragments, derivatives and mimetics having activity corresponding to naturally occurring domains.

The term “heterologous amino acid sequence” or “heterologous nucleotide sequence” as used herein means a sequence which is not found in the native Notch ligand or its coding sequence.

Whether an agent can be used for activating or reducing Notch signalling may be determined using suitable screening assays, for example, as described in the examples herein.

Activation of Notch signalling may also be achieved by repressing inhibitors of the Notch signalling pathway. As such, polypeptides for Notch signalling activation will include molecules capable of repressing any Notch signalling inhibitors. Preferably the molecule will be a polypeptide, or a polynucleotide encoding such a polypeptide, that decreases or interferes with the production or activity of compounds that are capable of producing an decrease in the expression or activity of Notch, Notch ligands, or any downstream components of the Notch signalling pathway. In a preferred embodiment, the molecules will be capable of repressing polypeptides of the Toll-like receptor protein family and growth factors such as the bone morphogenetic protein (BMP), BMP receptors and activins, derivatives, fragments, variants and homologues thereof.

The present invention also relates to modification of Notch-protein expression or presentation on the cell membrane or signalling pathways. Agents that enhance the presentation of a fully functional Notch-protein on the lymphocyte or APC surface include matrix metalloproteinases such as the product of the Kuzbanian gene of Drosophila (Dkuz et al (1997) Cell 90: 271-280) and other ADAMALYSIN gene family members.

In more detail, whether a substance can be used for modulating Notch signalling may be determined using suitable screening assays.

Screening assays for the detection of increased Notch, Notch ligand expression and/or processing include:

Notch-Notch ligand expression may be assessed following exposure of isolated cells to test compounds in culture using for example:
(a) at the protein level by specific antibody staining using immunohistochemistry or flow cytometry.
(b) at the RNA level by quantitative—reverse transcriptase-polymerase chain reaction (RT-PCR). RT-PCR may be performed using a control plasmid with in-built standards for measuring endogenous gene expression with primers specific for Notch 1 and Notch 2, Serrate 1 and Serrate 2, Delta 1 and Delta 2 and Delta 3. This construct may be modified as new ligand members are identified.
(c) at the functional level in cell adhesion assays.

Increased Notch ligand or Notch expression should lead to increased adhesion between cells expressing Notch and its ligands. Test cells will be exposed to a particular treatment in culture and radiolabelled or flourescein labelled target cells (transfected with Notch/Notch ligand protein) will be overlayed. Cell mixtures will be incubated at 37° C. for 2 hours. Nonadherent cells will be washed away and the level of adherence measured by the level of radioactivity/immunofluorescence at the plate surface.

Using such methods it is possible to detect compounds or Notch-ligands that affect the expression or processing of a Notch-protein or Notch-ligand. The invention also relates to compounds, or Notch-ligands detectable by these assays methods, and also to their use in the methods of the present invention.

These procedures may also be used to identify particularly effective combinations of substances for use according to the present invention.

Polynucleotides for Notch Signalling Inhibition

Preferably, the nucleic acid sequence for use in the present invention is capable of inhibiting Serrate and Delta, preferably Serrate 1 and Serrate 2 as well as Delta 1, Delta 3 and Delta 4 expression in APCs such as dendritic cells. In particular, the nucleic acid sequence may be capable of inhibiting Serrate expression but not Delta expression in APCs. Alternatively, the nucleic acid sequence for use in the present invention is capable of inhibiting Delta expression in T cells such as CD4+ helper T cells or other cells of the immune system that express Delta (for example in response to stimulation of cell surface receptors). In particular, the nucleic acid sequence may be capable of inhibiting Delta expression but not Serrate expression in T cells. In a particularly preferred embodiment, the nucleic acid sequence is capable of inhibiting Notch ligand expression in both T cells and APC, for example Serrate expression in APCs and Delta expression in T cells.

Polypeptides, Proteins and Amino Acid Sequences

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “protein”.

“Peptide” usually refers to a short amino acid sequence that is 10 to 40 amino acids long, preferably 10 to 35 amino acids.

The amino acid sequence may be prepared and isolated from a suitable source, or it may be made synthetically or it may be prepared by use of recombinant DNA techniques.

Nucleotide Sequences

As used herein, the term “nucleotide sequence” is synonymous with the term “polynucleotide”.

The nucleotide sequence may be DNA or RNA of genomic or synthetic or of recombinant origin. They may also be cloned by standard techniques. The nucleotide sequence may be double-stranded or single-stranded whether representing the sense or antisense strand or combinations thereof.

Longer nucleotide sequences will generally be produced using recombinant means, for example using a PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector. In general, primers will be produced by synthetic means, involving a step wise manufacture of the desired nucleic acid sequence one nucleotide at a time. Techniques for accomplishing this using automated techniques are readily available in the art.

“Polynucleotide” refers to a polymeric form of nucleotides of at least 10 bases in length and up to 5,000 bases or even more, either ribonucleotides or deoxyribonucleotides or a modified form of either type of nucleotide. The term includes single and double stranded forms of DNA.

These may be constructed using standard recombinant DNA methodologies. The nucleic acid may be RNA or DNA and is preferably DNA. Where it is RNA, manipulations may be performed via cDNA intermediates. Generally, a nucleic acid sequence encoding the first region will be prepared and suitable restriction sites provided at the 5′ and/or 3′ ends. Conveniently the sequence is manipulated in a standard laboratory vector, such as a plasmid vector based on pBR322 or pUC19 (see below). Reference may be made to Molecular Cloning by Sambrook et al. (Cold Spring Harbor, 1989) or similar standard reference books for exact details of the appropriate techniques.

Sources of nucleic acid may be ascertained by reference to published literature or databanks such as GenBank. Nucleic acid encoding the desired first or second sequences may be obtained from academic or commercial sources where such sources are willing to provide the material or by synthesising or cloning the appropriate sequence where only the sequence data are available. Generally this may be done by reference to literature sources which describe the cloning of the gene in question.

Alternatively, where limited sequence data is available or where it is desired to express a nucleic acid homologous or otherwise related to a known nucleic acid, exemplary nucleic acids can be characterised as those nucleotide sequences which hybridise to the nucleic acid sequences known in the art.

For some applications, preferably, the nucleotide sequence is DNA. For some applications, preferably, the nucleotide sequence is prepared by use of recombinant DNA techniques (e.g. recombinant DNA). For some applications, preferably, the nucleotide sequence is cDNA. For some applications, preferably, the nucleotide sequence may be the same as the naturally occurring form.

Variants, Derivatives, Analogues, Homologues and Fragments

In addition to the specific amino acid sequences and nucleotide sequences mentioned herein, the present invention also encompasses the use of variants, derivatives, analogues, homologues and fragments thereof.

In the context of the present invention, a variant of any given sequence is a sequence in which the specific sequence of residues (whether amino acid or nucleic acid residues) has been modified in such a manner that the polypeptide or polynucleotide in question retains at least one of its endogenous functions. A variant sequence can be modified by addition, deletion, substitution modification replacement and/or variation of at least one residue present in the naturally-occurring protein.

The term “derivative” as used herein, in relation to proteins or polypeptides of the present invention includes any substitution of, variation of, modification of, replacement of, deletion of and/or addition of one (or more) amino acid residues from or to the sequence providing that the resultant protein or polypeptide retains at least one of its endogenous functions.

The term “analogue” as used herein, in relation to polypeptides or polynucleotides includes any mimetic, that is, a chemical compound that possesses at least one of the endogenous functions of the polypeptides or polynucleotides which it mimics.

Within the definitions of “proteins” useful in the present invention, the specific amino acid residues may be modified in such a manner that the protein in question retains at least one of its endogenous functions, such modified proteins are referred to as “variants”. A variant protein can be modified by addition, deletion and/or substitution of at least one amino acid present in the naturally-occurring protein.

Typically, amino acid substitutions may be made, for example from 1, 2 or 3 to 10 or 20 substitutions provided that the modified sequence retains the required activity or ability. Amino acid substitutions may include the use of non-naturally occurring analogues.

Proteins of use in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity, and/or the amphipathic nature of the residues as long as the transport or modulation function is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

For ease of reference, the one and three letter codes for the main naturally occurring amino acids (and their associated codons) are set out below:

| | 3- | | |
|---|---|---|---|
| Symbol | letter | Meaning | Codons |
| A | Ala | Alanine | GCT, GCC, GCA, GCG |
| B | Asp, | Aspartic, | GAT, GAC, AAT, AAC |
| | Asn | Asparagine | |
| C | Cys | Cysteine | TGT, TGC |
| D | Asp | Aspartic | GAT, GAC |
| E | Glu | Glutamic | GAA, GAG |
| F | Phe | Phenylalanine | TTT, TTC |
| G | Gly | Glycine | GGT, GGC, GGA, GGG |
| H | His | Histidine | CAT, CAC |
| I | Ile | Isoleucine | ATT, ATC, ATA |
| K | Lys | Lysine | AAA, AAG |
| L | Leu | Leucine | TTG, TTA, CTT, CTC, CTA, |
| | | | CTG |
| M | Met | Methionine | ATG |
| N | Asn | Asparagine | AAT, AAC |
| P | Pro | Proline | CCT, CCC, CCA, CCG |
| Q | Gln | Glutamine | CAA, CAG |
| R | Arg | Arginine | CGT, CGC, CGA, CGG, AGA, |
| | | | AGG |
| S | Ser | Serine | TCT, TCC, TCA, TCG, AGT, |
| | | | AGC |
| T | Thr | Threonine | ACT, ACC, ACA, ACG |
| V | Val | Valine | GTT, GTC, GTA, GTG |
| W | Trp | Tryptophan | TGG |
| X | Xxx | Unknown | |
| Y | Tyr | Tyrosine | TAT, TAC |
| Z | Glu, | Glutamic, | GAA, GAG, CAA, CAG |
| | Gln | Glutamine | |
| * | End | Terminator | TAA, TAG, TGA |

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

As used herein, the term “protein” includes single-chain polypeptide molecules as well as multiple-polypeptide complexes where individual constituent polypeptides are linked by covalent or non-covalent means. As used herein, the terms “polypeptide” and “peptide” refer to a polymer in which the monomers are amino acids and are joined together through peptide or disulfide bonds. The terms subunit and domain may also refer to polypeptides and peptides having biological function.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleodtide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Polynucleotide variants will preferably comprise codon optimised sequences. Codon optimisation is known in the art as a method of enhancing RNA stability and therefor gerie expression. The redundancy of the genetic code means that several different codons may encode the same amino-acid. For example, Leucine, Arginine and Serine are each encoded by six different codons. Different organisms show preferences in their use of the different codons. Viruses such as HIV, for instance, use a large number of rare codons. By changing a nucleotide sequence such that rare codons are replaced by the corresponding commonly used mammalian codons, increased expression of the sequences in mammalian target cells can be achieved. Codon usage tables are known in the art for mammalian cells, as well as for a variety of other organisms. Preferably, at least part of the sequence is codon optimised. Even more preferably, the sequence is codon optimised in its entirety.

As used herein, the term “homology” can be equated with “identity”. An homologous sequence will be taken to include an amino acid sequence which may be at least 75, 85 or 90% identical, preferably at least 95 or 98% identical. In particular, homology should typically be considered with respect to those regions of the sequence (such as amino acids at positions 51, 56 and 57) known to be essential for an activity. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

Percent homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefor firstly requires the production of an optimal alignment, taking into consideration gap penalties. A suitable computer program for carrying out such an alignment is the GCG Wisconsin Bestfit package (University of Wisconsin, U.S.A.; Devereux). Examples of other software than can perform sequence comparisons include, but are not limited to, the BLAST package, FASTA (Atschul et al. (1990) J. Mol. Biol. 403-410 (Atschul)) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

The five BLAST programs available at the National Center for Biotechnology Information website (maintained by the National Institutes of Health) perform the following tasks:

blastp—compares an amino acid query sequence against a protein sequence database.

blastn—compares a nucleotide query sequence against a nucleotide sequence database.

blastx—compares the six-frame conceptual translation products of a nucleotide query sequence (both strands) against a protein sequence database.

tblastn—compares a protein query sequence against a nucleotide sequence database dynamically translated in all six reading frames (both strands).

tblastx—compares the six-frame translations of a nucleotide query sequence against the six-frame translations of a nucleotide sequence database.

BLAST uses the following search parameters:

HISTOGRAM—Display a histogram of scores for each search; default is yes. (See parameter H in the BLAST Manual).

DESCRIPTIONS—Restricts the number of short descriptions of matching sequences reported to the number specified; default limit is 100 descriptions. (See parameter V in the manual page).

EXPECT—The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF—Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

ALIGNMENTS—Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

MATRIX—Specify an alternate scoring matrix for BLASTP, BLASTX, TBLASTN and TBLASTX. The default matrix is BLOSUM62 (Henikoff & Henikoff, 1992). The valid alternative choices include: PAM40, PAM120, PAM250 and IDENTITY. No alternate scoring matrices are available for BLASTN; specifying the MATRIX directive in BLASTN requests returns an error response.

STRAND—Restrict a TBLASTN search to just the top or bottom strand of the database sequences; or restrict a BLASTN, BLASTX or TBLASTX search to just reading frames on the top or bottom strand of the query sequence.

FILTER—Mask off segments of the query sequence that have low compositional complexity, as determined by the SEG program of Wootton & Federhen (1993) Computers and Chemistry 17:149-163, or segments consisting of short-periodicity internal repeats, as determined by the XNU program of Claverie & States (1993) Computers and Chemistry 17:191-201, or, for BLASTN, by the DUST program of Tatusov and Lipman (see the National Center for Biotechnology Information website, maintained by the National Institutes of Health). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “NNNNNNNNNNNNN”) and the letter “X” in protein sequences (e.g., “XXXXXXXXX”).

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi—Causes NCBI gi identifiers to be shown in the output, in addition to the accession and/or locus name.

Most preferably, sequence comparisons are conducted using the simple BLAST search algorithm provided at the National Center for Biotechnology Information website (maintained by the National Institutes of Health).

In some aspects of the present invention, no gap penalties are used when determining sequence identity.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

Nucleotide sequences which are homologous to or variants of sequences of use in the present invention can be obtained in a number of ways, for example by probing DNA libraries made from a range of sources. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the reference nucleotide sequence under conditions of medium to high stringency. Similar considerations apply to obtaining species homologues and allelic variants of the amino acid and/or nucleotide sequences useful in the present invention.

Variants and strain/species homologues may also be obtained using degenerate PCR which will use primers designed to target sequences within the variants and homologues encoding conserved amino acid sequences within the sequences of use in the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Variants and strain/species homologues may also be obtained using degenerate PCR which will use primers designed to target sequences within the variants and homologues encoding conserved amino acid sequences within the sequences of use in the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

PCR technology as described e.g. in section 14 of Sambrook et al., 1989, requires the use of oligonucleotide probes that will hybridise to nucleic acid. Strategies for selection of oligonucleotides are described below.

As used herein, a probe is e.g. a single-stranded DNA or RNA that has a sequence of nucleotides that includes between 10 and 50, preferably between 15 and 30 and most preferably at least about 20 contiguous bases that are the same as (or the complement of) an equivalent or greater number of contiguous bases. The nucleic acid sequences selected as probes should be of sufficient length and sufficiently unambiguous so that false positive results are minimised. The nucleotide sequences are usually based on conserved or highly homologous nucleotide sequences or regions of polypeptides. The nucleic acids used as probes may be degenerate at one or more positions.

Preferred regions from which to construct probes include 5′ and/or 3′ coding sequences, sequences predicted to encode ligand binding sites, and the like. For example, either the full-length cDNA clone disclosed herein or fragments thereof can be used as probes. Preferably, nucleic acid probes of the invention are labelled with suitable label means for ready detection upon hybridisation. For example, a suitable label means is a radiolabel. The preferred method of labelling a DNA fragment is by incorporating α³²P dATP with the Klenow fragment of DNA polymerase in a random priming reaction, as is well known in the art. Oligonucleotides are usually end-labelled with γ³²P-labelled ATP and polynucleotide kinase. However, other methods (e.g. non-radioactive) may also be used to label the fragment or oligonucleotide, including e.g. enzyme labelling, fluorescent labelling with suitable fluorophores and biotinylation.

Preferred are such sequences, probes which hybridise under high-stringency conditions.

Alternatively, such nucleotide sequences may be obtained by site directed mutagenesis of characterised sequences. This may be useful where for example silent codon changes are required to sequences to optimise codon preferences for a particular host cell in which the nucleotide sequences are being expressed. Other sequence changes may be desired in order to introduce restriction enzyme recognition sites, or to alter the activity of the polynucleotide or encoded polypeptide.

In general, the terms “variant”, “homologue” or “derivative” in relation to the nucleotide sequence used in the present invention includes any substitution of, variation of, modification of, replacement of, deletion of or addition of one (or more) nucleic acid from or to the sequence providing the resultant nucleotide sequence codes for a target protein or protein for T cell signalling modulation.

As indicated above, with respect to sequence homology, preferably there is at least 75%, more preferably at least 85%, more preferably at least 90% homology to the reference sequences. More preferably there is at least 95%, more preferably at least 98%, homology. Nucleotide homology comparisons may be conducted as described above. A preferred sequence comparison program is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

Hybridisation

The present invention also encompasses nucleotide sequences that are capable of hybridising selectively to the reference sequences, or any variant, fragment or derivative thereof, or to the complement of any of the above. Nucleotide sequences are preferably at least 15 nucleotides in length, more preferably at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction (PCR) technologies.

Nucleotide sequences useful in the invention capable of selectively hybridising to the nucleotide sequences presented herein, or to their complement, will be generally at least 75%, preferably at least 85 or 90% and more preferably at least 95% or 98% homologous to the corresponding nucleotide sequences presented herein over a region of at least 20, preferably at least 25 or 30, for instance at least 40, 60 or 100 or more contiguous nucleotides. Preferred nucleotide sequences of the invention will comprise regions homologous to the nucleotide sequence, preferably at least 80 or 90% and more preferably at least 95% homologous to the nucleotide sequence.

The term “selectively hybridizable” means that the nucleotide sequence used as a probe is used under conditions where a target nucleotide sequence of the invention is found to hybridize to the probe at a level significantly above background. The background hybridization may occur because of other nucleotide sequences present, for example, in the cDNA or genomic DNA library being screened. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm −5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical nucleotide sequences while an intermediate (or low) stringency hybridization can be used to identify or detect similar or related polynucleotide sequences.

In a preferred aspect, the present invention covers nucleotide sequences that can hybridise to the nucleotide sequence of the present invention under stringent conditions (e.g. 65° C. and 0.1×SSC (1×SSC=0.15 M NaCl, 0.015 M Na₃ Citrate pH 7.0). Where the nucleotide sequence of the invention is double-stranded, both strands of the duplex, either individually or in combination, are encompassed by the present invention. Where the nucleotide sequence is single-stranded, it is to be understood that the complementary sequence of that nucleotide sequence is also included within the scope of the present invention.

Stringency of hybridisation refers to conditions under which polynucleic acids hybrids are stable. Such conditions are evident to those of ordinary skill in the field. As known to those of skill in the art, the stability of hybrids is reflected in the melting temperature (Tm) of the hybrid which decreases approximately 1 to 1.5° C. with every 1% decrease in sequence homology. In general, the stability of a hybrid is a function of sodium ion concentration and temperature. Typically, the hybridisation reaction is performed under conditions of higher stringency, followed by washes of varying stringency.

As used herein, high stringency preferably refers to conditions that permit hybridisation of only those nucleic acid sequences that form stable hybrids in 1 M Na+ at 65-68° C. High stringency conditions can be provided, for example, by hybridisation in an aqueous solution containing 6×SSC, 5× Denhardt's, 1% SDS (sodium dodecyl sulphate), 0.1 Na+ pyrophosphate and 0.1 mg/ml denatured salmon sperm DNA as non specific competitor. Following hybridisation, high stringency washing may be done in several steps, with a final wash (about 30 min) at the hybridisation temperature in 0.2-0.1×SSC, 0.1% SDS.

It is understood that these conditions may be adapted and duplicated using a variety of buffers, e.g. formamide-based buffers, and temperatures. Denhardt's solution and SSC are well known to those of skill in the art as are other suitable hybridisation buffers (see, e.g. Sambrook, et al., eds. (1989) Molecular Cloning: A Laboratory Manual, Cold Spring Harbor Laboratory Press, New York or Ausubel, et al., eds. (1990) Current Protocols in Molecular Biology, John Wiley & Sons, Inc.). Optimal hybridisation conditions have to be determined empirically, as the length and the GC content of the hybridising pair also play a role.

Cloning and Expression

Nucleotide sequences which are not 100% homologous to the sequences of the present invention but fall within the scope of the invention can be obtained in a number of ways. Other variants of the sequences described herein may be obtained for example by probing DNA libraries made from a range of sources. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the reference nucleotide sequence under conditions of medium to high stringency. Similar considerations apply to obtaining species homologues and allelic variants of the amino acid and/or nucleotide sequences useful in the present invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Alternatively, such nucleotide sequences may be obtained by site directed mutagenesis of characterised sequences. This may be useful where for example silent codon changes are required to sequences to optimise codon preferences for a particular host cell in which the nucleotide sequences are being expressed. Other sequence changes may be desired in order to introduce restriction enzyme recognition sites, or to alter the activity of the target protein or protein for T cell signalling modulation encoded by the nucleotide sequences.

The nucleotide sequences such as a DNA polynucleotides useful in the invention may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer nucleotide sequences will generally be produced using recombinant means, for example using a PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector

The present invention also relates to vectors which comprise a polynucleotide useful in the present invention, host cells which are genetically engineered with vectors of the invention and the production of polypeptides useful in the present invention by such techniques.

For recombinant production, host cells can be genetically engineered to incorporate expression systems or polynucleotides of the invention. Introduction of a polynucleotide into the host cell can be effected by methods described in many standard laboratory manuals, such as Davis et al and Sambrook et al, such as calcium phosphate transfection, DEAE-dextran mediated transfection, transfection, microinjection, cationic lipid-mediated transfection, electroporation, transduction, scrape loading, ballistic introduction and infection. It will be appreciated that such methods can be employed in vitro or in vivo as drug delivery systems.

Representative examples of appropriate hosts include bacterial cells, such as streptococci, staphylococci, E. coli, streptomyces and Bacillus subtilis cells; fungal cells, such as yeast cells and Aspergillus cells; insect cells such as Drosophila S2 and Spodoptera Sf9 cells; animal cells such as CHO, COS, NSO, HeLa, C127, 3T3, BHK, 293 and Bowes melanoma cells; and plant cells.

A great variety of expression systems can be used to produce a polypeptide useful in the present invention. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al.

For secretion of the translated protein into the lumen of the endoplasmic reticulum, into the periplasmic space or into the extracellular environment, appropriate secretion signals may be incorporated into the expressed polypeptide. These signals may be endogenous to the polypeptide or they may be heterologous signals.

Proteins or polypeptides may be in the form of the “mature” protein or may be a part of a larger protein such as a fusion protein or precursor. For example, it is often advantageous to include an additional amino acid sequence which contains secretory or leader sequences or pro-sequences (such as a HIS oligomer, immunoglobulin Fc, glutathione S-transferase, FLAG etc) to aid in purification. Likewise such an additional sequence may sometimes be desirable to provide added stability during recombinant production. In such cases the additional sequence may be cleaved (e.g. chemically or enzymatically) to yield the final product. In some cases, however, the additional sequence may also confer a desirable pharmacological profile (as in the case of IgFc fusion proteins) in which case it may be preferred that the additional sequence is not removed so that it is present in the final product as administered.

Proteins or polypeptides may be in the form of the “mature” protein or may be a part of a larger protein such as a fusion protein or precursor. For example, it is often advantageous to include an additional amino acid sequence which contains secretory or leader sequences or pro-sequences (such as a HIS oligomer, immunoglobulin Fc, glutathione S-transferase, FLAG etc) to aid in purification. Likewise such an additional sequence may sometimes be desirable to provide added stability during recombinant production. In such cases the additional sequence may be cleaved (e.g. chemically or enzymatically) to yield the final product. In some cases, however, the additional sequence may also confer a desirable pharmacological profile (as in the case of IgFc fusion proteins) in which case it may be preferred that the additional sequence is not removed so that it is present in the final product as administered.

Active agents for use in the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding protein may be employed to regenerate active conformation when the polypeptide is denatured during isolation and/or purification.

Particles and Particle Delivery

In one embodiment, RNAi agents of the present invention may be administered on delivery particles, preferably microparticles, preferably in combination with antigens or antigenic determinants or nucleic acids coding for antigens or antigenic determinants, to modulate immune responses to such antigens or antigenic determinants.

Thus, for example, in one embodiment the present invention provides a delivery particle suitable for administration to a subject to modulate an immune response to an antigen or antigenic determinant which comprises (e.g. is coated or impregnated with):
i) an RNAi agent targeting a component of the Notch signaling pathway; and
ii) an antigen or antigenic determinant or a nucleic acid coding for an antigen or antigenic determinant.

In one embodiment such a particle may comprise (e.g. be coated or impregnated with):
i) an RNAi agent targeting a component of the Notch signaling pathway to increase Notch signalling; and
ii) an autoantigen, bystander antigen, allergen, pathogen antigen or graft antigen or an antigenic determinant thereof or a nucleic acid coding for such an antigen or antigenic determinant;
   Such a particle may be administered to reduce an immune response to said antigen or antigenic determinant.

In an alternative embodiment such a particle may comprise (e.g. be coated or impregnated with):
i) an RNAi agent targeting a component of the Notch signaling pathway to decrease Notch signalling; and
ii) a pathogen antigen or tumour antigen or an antigenic determinant thereof or a nucleic acid coding for such an antigen or antigenic determinant;
   Such a particle may be administered to increase an immune response to said antigen or antigenic determinant, e.g. in a pathogen or tumour vaccine.

A variety of particles and delivery systems may be used in the present invention, including but not limited to, the following:

(i) Biolistic Particle Delivery

In one embodiment, agents according to the present invention may be administered by a needleless or “ballistic” (biolistic) delivery mechanism. A range of such delivery systems are known in the art. One system, developed by Powderject Vaccines, is particularly useful and a variety of suitable forms and embodiments are described, for example, in the following publications, which are incorporated herein by reference:

WO03011380 Silencing Device And Method For Needleless Syringe; WO03011379 Particle Cassette, Method And Kit Therefor; WO02101412 Spray Freeze-Dried Compositions; WO02100380 Production Of Hard, Dense Particles; WO02055139 Needleless Syringe; WO0243774 Nucleic Acid Immunization; WO0219989 Alginate Particle Formulation; WO0207803 Needleless Syringe; WO0193829 Powder Compositions; WO0183528 Nucleic Acid Immunization; WO0168167 Apparatus And Method For Adjusting The Characteristics Of A Needleless Syringe; WO0134185 Induction Of Mucosal Immunity By Vaccination Via The Skin Route; WO0133176 Apparatus And Method For Dispensing Small Quantities Of Particles; WO01015455 Needleless Syringe; WO0063385 Nucleic Acid Immunization; WO0062846 Needleless Syringe; WO0054827 Needleless Syringe; WO0053160 Delivery Of Microparticle Formulations Using Needleless Syringe Device For Sustained-Release Of Bioactive Compounds; WO0044421 Particle Delivery Device; WO0026385 Nucleic Acid Constructs For Genetic Immunization; WO0023592 Minimal Promoters And Uses Thereof; WO0019982 Spray Coated Microparticles For Use In Needleless Syringes; WO9927961 Transdermal Delivery Of Particulate Vaccine Compositions; WO9908689 Mucosal Immunization Using Particle-Mediated Delivery Techniques; WO9901169 Syringe And Capsule Therefor; WO9901168 Drug Particle Delivery; WO9821364 Method And Apparatus For Preparing Sample Cartridges For A Particle Acceleration Device; WO9813470 Gas-Driven Particle Delivery Device; WO9810750 Nucleic Acid Particle Delivery; WO9748485 Method For Providing Dense Particle Compositions For Use In Transdermal Particle Delivery; WO9734652 Needleless Syringe With Therapeutic Agent Particles Entrained In Supersonic Gas Flow.

As described, for example, in 20020165176 A1, particle-mediated methods for delivering such nucleic acid preparations are known in the art. Thus, once prepared and suitably purified, the nucleic acid molecules can be coated onto carrier particles (e.g., core carriers) using a variety of techniques known in the art. Carrier particles are selected from materials which have a suitable density in the range of particle sizes typically used for intracellular delivery from a particle-mediated delivery device. The optimum carrier particle size will, of course, depend on the diameter of the target cells. Alternatively, colloidal gold particles can be used wherein the coated colloidal gold is administered (e.g., injected) into tissue (e.g., skin or muscle) and subsequently taken-up by immune-competent cells.

Suitable particles include metal particles such as, tungsten, gold, platinum and iridium carrier particles. Tungsten and gold particles are preferred. Tungsten particles are readily available in average sizes of 0.5 to 2.0 um in diameter. Gold particles or microcrystalline gold (e.g.; gold powder A1570, available from Engelhard Corp., East Newark, N.J.) may also be used. Gold particles provide uniformity in size (available from Alpha Chemicals in particle sizes of 1-3 um, or available from Degussa, South Plainfield, N.J. in a range of particle sizes including 0.95 um) and low toxicity. Microcrystalline gold provides a diverse particle size distribution, typically in the range of 0.1-5 um. The irregular surface area of microcrystalline gold provides for highly efficient coating with nucleic acids.

A large number of methods are known and have been described for coating or precipitating polynucleotides such as DNA or RNA onto articles such as gold or tungsten particles. Typically such methods combine a predetermined amount of gold or tungsten with plasmid DNA, CaCl₂ and spermidine. The resulting solution is suitably vortexed continually during the coating procedure to ensure uniformity of the reaction mixture. After precipitation of the nucleic acid, the coated particles can for example be transferred to suitable membranes and allowed to dry prior to use, coated onto surfaces of a sample module or cassette, or loaded into a delivery cassette for use in particular particle-mediated delivery instruments.

Following their formation, carrier particles coated with the nucleic acid preparations can be delivered to a subject using particle-mediated delivery techniques.

Various particle acceleration devices suitable for particle-mediated delivery are known in the art, and are all suited for use in the practice of the invention. Current device designs employ an explosive, electric or gaseous discharge to propel coated carrier particles toward target cells. The coated carrier particles can themselves be releasably attached to a movable carrier sheet, or removably attached to a surface along which a gas stream passes, lifting the particles from the surface and accelerating them toward the target. An example of a gaseous discharge device is described in U.S. Pat. No. 5,204,253. An explosive-type device is described in U.S. Pat. No. 4,945,050. One example of an electric discharge-type particle acceleration apparatus is described in U.S. Pat. No. 5,120,657. Another electric discharge apparatus suitable for use herein is described in U.S. Pat. No. 5,149,655. The disclosure of all of these patents is incorporated herein by reference in their entireties.

If desired, these particle acceleration devices can be provided in a preloaded condition containing a suitable dosage of the coated carrier particles comprising the polynucleotide vaccine composition, with or without additional influenza vaccine compositions and/or a selected adjuvant component. The loaded syringe can be packaged in a hermetically sealed container.

The coated particles are administered to the subject to be treated in a manner compatible with the dosage formulation, and in an amount that will be effective to bring about a desired immune response. The amount of the composition to be delivered which, in the case of nucleic acid molecules is generally in the range of from 0.001 to 1000 ug, more preferably 0.01 to 10.0 ug of nucleic acid molecule per dose, depends on the subject to be treated. The exact amount necessary will vary depending on the age and general condition of the individual being immunized and the particular nucleotide sequence or peptide selected, as well as other factors. An appropriate effective amount can be readily determined by one of skill in the art.

The formulated compositions may suitably be prepared as particles using standard techniques, such as by simple evaporation (air drying), vacuum drying, spray drying, freeze drying (lyophilization), spray-freeze drying, spray coating, precipitation, supercritical fluid particle formation, and the like. If desired, the resultant particles can be densified using the techniques described in International Publication No. WO 97/48485, incorporated herein by reference.

These methods can be used to obtain nucleic acid particles having a size ranging from about 0.01 to about 250 um, preferably about 10 to about 150 um, and most preferably about 20 to about 60 um; and a particle density ranging from about 0.1 to about 25 g/cm³, and a bulk density of about 0.5 to about 3.0 g/cm³, or greater.

Single unit dosages or multidose containers, in which the particles may be packaged prior to use, may suitably comprise a hermetically sealed container enclosing a suitable amount of the particles. The particulate compositions can be packaged as a sterile formulation, and the hermetically sealed container can thus be designed to preserve sterility of the formulation until use in the methods of the invention. If desired, the containers can be adapted for direct use in a needleless syringe system. Such containers can take the form of capsules, foil pouches, sachets, cassettes, and the like. Appropriate needleless syringes are described herein above.

The container in which the particles are packaged can further be labeled to identify the composition and provide relevant dosage information. In addition, the container can be labeled with a notice in the form prescribed by a governmental agency, for example the Food and Drug Administration, wherein the notice indicates approval by the agency under Federal law of the manufacture, use or sale of the composition contained therein for human administration.

Following their formation, the particulate composition (e.g., powder) can be delivered transdermally to the subject's tissue using a suitable transdermal delivery technique. Various particle acceleration devices suitable for transdermal delivery of the substance of interest are known in the art, and will find use in the practice of the invention. A particularly preferred transdermal delivery system employs a needleless syringe to fire solid drug-containing particles in controlled doses into and through intact skin and tissue. See, e.g., U.S. Pat. No. 5,630,796 to Bellhouse et al. which describes a needleless syringe (also known as “the PowderJect.RTM. needleless syringe device”). Other needleless syringe configurations are known in the art and are described herein.

Suitably, the particulate compositions will be delivered via a powder injection method, e.g., delivered from a needleless syringe system such as those described in commonly owned International Publication Nos. WO 94/24263, WO 96/04947, WO 96/12513, and WO 96/20022, all of which are incorporated herein by reference. Delivery of particles from such needleless syringe systems is typically practised with particles having an approximate size generally ranging from 0.1 to 250 um, preferably ranging from about 1-70 um. Particles larger than about 250 um can also be delivered from the devices, with the upper limitation being the point at which the size of the particles would cause untoward damage to the skin cells. The actual distance which the delivered particles will penetrate a target surface depends upon particle size (e.g., the nominal particle diameter assuming a roughly spherical particle geometry), particle density, the initial velocity at which the particle impacts the surface, and the density and kinematic viscosity of the targeted skin tissue. In this regard, optimal particle densities for use in needleless injection generally range between about 0.1 and 25 g/cm³, preferably between about 0.9 and 1.5 g/cm³, and injection velocities generally range between about 100 and 3,000 m/sec, or greater. With appropriate gas pressure, particles having an average diameter of 1-70 um can be accelerated through the nozzle at velocities approaching the supersonic speeds of a driving gas flow.

If desired, these needleless syringe systems can be provided in a preloaded condition containing a suitable dosage of the particles comprising the antigen of interest and/or the selected adjuvant. The loaded syringe can be packaged in a hermetically sealed container, which may further be labeled as described above.

Compositions containing a therapeutically effective amount of the powdered molecules described herein can be delivered to any suitable target tissue via the above-described needleless syringes. For example, the compositions can be delivered to muscle, skin, brain, lung, liver, spleen, bone marrow, thymus, heart, lymph, blood, bone cartilage, pancreas, kidney, gall bladder, stomach, intestine, testis, ovary, uterus, rectum, nervous system, eye, gland and connective tissues. For nucleic acid molecules, delivery is preferably to, and the molecules expressed in, terminally differentiated cells; however, the molecules can also be delivered to non-differentiated, or partially differentiated cells such as stem cells of blood and skin fibroblasts.

The powdered compositions are administered to the subject to be treated in a manner compatible with the dosage formulation, and in an amount that will be prophylactically and/or therapeutically effective. The amount of the composition to be delivered, generally in the range of from 0.5 ug/kg to 100 ug/kg of nucleic acid molecule per dose, depends on the subject to be treated. Doses for other pharmaceuticals, such as physiological active peptides and proteins, generally range from about 0.1 ug to about 20 mg, preferably 10 ug to about 3 mg. The exact amount necessary will vary depending on the age and general condition of the individual to be treated, the severity of the condition being treated, the particular preparation delivered, the site of administration, as well as other factors. An appropriate effective amount can be readily determined by one of skill in the art.

(ii) Liposome Particle Delivery

In an alternative embodiment, particles may take the form of lipid complexes and/or liposomes.

For example, lipid-nucleic acid formulations can be formed by combining the nucleic acid with a preformed cationic liposome (see, U.S. Pat. Nos. 4,897,355, 5,264,618, 5,279,833 and 5,283,185). In such methods, the nucleic acid is attracted to the cationic surface charge of the liposome and the resulting complexes are thought to be of the liposome-covered “sandwich-type.”

Liposome-based delivery of polynucleotides is also described, for example, in N. J. Caplen, et al., Liposome-mediated CFTR gene transfer to the nasal epithelium of patients with cystic fibrosis, Nature Medicine, 1(1995) 39; M. Cotten and E. Wagner, Non-viral approaches to gene therapy, Current opinion in biotechnology, (1993) 705-710; A. Singhal and L. Huang, Gene transfer in mammalian cells using liposomes as carriers, in Gene Therapeutics: Methods and Applications of Direct Gene Transfer, J. A. Wolff, Editor. 1994, Birkhauser: Boston; and J. P. Schonfield and C. T. Caskey, Non-viral approaches to gene therapy, Brit. Med. J., 51(1995) 56.

(iii) Delivery of Particles for Uptake by Cells

In an alternative embodiment, particles may be administered for active uptake by cells, for example by phagocytosis, as described for example in U.S. Pat. No. 5,783,567 (Pangaea), which is herein incorporated by reference.

As described, for example, in U.S. Pat. No. 5,783,567, phagocytosis of microparticles by macrophages and other antigen presenting cells (APCs) is an effective means for introducing the nucleic acid into these cells. Phagocytosis by these cells can be increased by maintaining a particle size preferably below about 20 um, and preferably below about 11 um. The type of polymer used in the microparticle can also affect the efficiency of uptake by phagocytic cells, as discussed below.

The microparticles can be delivered directly into the bloodstream (i.e., by intravenous or intraarterial injection or infusion) if uptake by the phagocytic cells of the reticuloendothelial system (RES) is desired. Alternatively, one can target, via subcutaneous injection, take-up by the phagocytic cells of the draining lymph nodes. The microparticles can also be introduced intradermally (i.e., to the APCs of the skin, such as dendritic cells and Langerhans cells). Another useful route of delivery (particularly for DNAs encoding tolerance-inducing polypeptides) is via the gastrointestinal tract, e.g., orally. Alternatively, the microparticles can be introduced into organs such as the lung (e.g., by inhalation of powdered microparticles or of a nebulized or aerosolized solution containing the microparticles), where the particles are picked up by the alveolar macrophages, or may be administered intranasally or buccally.

Once a phagocytic cell phagocytoses the microparticle, the nucleic acid is released into the interior of the cell. Upon release, it can perform its intended function: for example, expression by normal cellular transcription/translation machinery.

Because these microparticles are passively targeted to dendritic cells, macrophages and other types of phagocytic cells, they represent a means for modulating immune function. Macrophages serve as professional APCs, expressing both MHC class I and class II molecules.

Suitable polymeric material may be obtained from commercial sources or can be prepared by known methods. For example, polymers of lactic and glycolic acid can be generated as described in U.S. Pat. No. 4,293,539 or purchased from Aldrich.

Alternatively, or in addition, the polymeric matrix can include, for example, polylactide, polyglycolide, poly(lactide-co-glycolide), polyanhydride, polyorthoester, polycaprolactone, polyphosphazene, proteinaceous polymer, polypeptide, polyester, or polyorthoester.

Polymeric particles containing nucleic acids are suitably prepared using a double emulsion technique, for example, as follows: First, the polymer is dissolved in an organic solvent. A preferred polymer is polylactic-co-glycolic acid (PLGA), with a lactic/glycolic acid weight ratio of 65:35, 50:50, or 75:25. Next, a sample of nucleic acid suspended in aqueous solution is added to the polymer solution and the two solutions are mixed to form a first emulsion. The solutions can be mixed by vortexing or shaking, and in a preferred method, the mixture can be sonicated. Most preferable is any method by which the nucleic acid receives the least amount of damage in the form of nicking, shearing, or degradation, while still allowing the formation of an appropriate emulsion. For example, acceptable results can be obtained with a Vibra-cell model VC-250 sonicator with a ⅛″ microtip probe, at setting #3.

During this process, the polymer forms into minute “microparticles,” each of which contains some of the nucleic acid-containing solution. If desired, one can isolate a small amount of the nucleic acid at this point in order to assess integrity, e.g., by gel electrophoresis.

The first emulsion is then added to an organic solution. The solution can be comprised of, for example, methylene chloride, ethyl acetate, or acetone, preferably containing polyvinyl alcohol (PVA), and most preferably having a 1:100 ratio of the weight of PVA to the volume of the solution. The first emulsion is generally added to the organic solution with stirring in a homogenizer or sonicator. For example, one can use a Silverson Model L4RT homogenizer (⅝″ probe) set at 7000 RPM for about 12 seconds. A 60 second homogenization time would be too harsh at this homogenization speed.

This process forms a second emulsion which is subsequently added to another organic solution with stirring (e.g., in a homogenizer). In a preferred method, the latter solution is 0.05% w/v PVA. The resultant microparticles are washed several times with water to remove the organic compounds. Particles can be passed through sizing screens to selectively remove those larger than the desired size. If the size of the microparticles is not crucial, one can dispense with the sizing step. After washing, the particles can either be used immediately or be lyophilized for storage.

The size distribution of the microparticles prepared by the above method can be determined with a COULTERM™ counter. This instrument provides a size distribution profile and statistical analysis of the particles. Alternatively, the average size of the particles can be determined by visualization under a microscope fitted with a sizing slide or eyepiece.

If desired, the nucleic acid can be extracted from the microparticles for analysis by the following procedure. Microparticles are dissolved in an organic solvent such as chloroform or methylene chloride in the presence of an aqueous solution. The polymer stays in the organic phase, while the DNA goes to the aqueous phase. The interface between the phases can be made more distinct by centrifugation. Isolation of the aqueous phase allows recovery of the nucleic acid. To test for degradation, the extracted nucleic acid can be analyzed by HPLC or gel electrophoresis.

To increase the recovery of nucleic acid, additional organic solvents, such as phenol and chloroform, can be added to the dissolved microparticles, prior to the addition of the aqueous solution. Following addition of the aqueous solution, the nucleic acid enters the aqueous phase, which can easily be partitioned from the organic phase after mixing. For a clean interface between the organic and aqueous phases, the samples should be centrifuged. The nucleic acid is retrieved from the aqueous phase by precipitation with salt and ethanol in accordance with standard methods.

Microparticles containing nucleic acid can be injected into mammals intramuscularly, intravenously, intraarterially, intradermally, intraperitoneally, or subcutaneously, or they can be introduced into the gastrointestinal tract or the respiratory tract, e.g., by inhalation of a solution or powder containing the microparticles. Expression of the nucleic acid may be monitored by an appropriate method.

Vectors for Introduction and Expression of Polynucleotides in Cells

An important aspect of the present invention is the use of delivery agents to introduce selected polynucleotide sequences into cells in vitro and in vivo, followed by expression of the selected gene in the host cell. Thus, the nucleic acids in the particles are typically in the form of vectors that are capable of being expressed in the desired subject host cell. Promoter, enhancer, stress or chemically-regulated promoters, antibiotic-sensitive or nutrient-sensitive regions, as well as therapeutic protein encoding sequences, may be included as required.

As described, for example, in U.S. Pat. No. 5,976,567 (Inex), the expression of natural or synthetic nucleic acids is typically achieved by operably linking a nucleic acid of interest to a promoter (which may be either constitutive or inducible), preferably incorporating the construct into an expression vector, and introducing the vector into a suitable host cell. Typical vectors contain transcription and translation terminators, transcription and translation initiation sequences, and promoters useful for regulation of the expression of the particular nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors may be suitable for replication and integration in prokaryotes, eukaryotes, or preferably both. See, Giliman and Smith (1979), Gene, 8: 81-97; Roberts et al. (1987), Nature, 328: 731-734; Berger and Kimmel, Guide to Molecular Cloning Techniques, Methods in Enzymology, volume 152, Academic Press, Inc., San Diego, Calif. (Berger); Sambrook et al. (1989), MOLECULAR CLONING—A LABORATORY MANUAL (2nd ed.) Vol. 1-3, Cold Spring Harbor Laboratory, Cold Spring Harbor Press, N.Y., (Sambrook); and F. M. Ausubel et al., CURRENT PROTOCOLS IN MOLECULAR BIOLOGY, eds., Current Protocols, a joint venture between Greene Publishing Associates, Inc. and John Wiley & Sons, Inc., (1994 Supplement) (Ausubel). Product information from manufacturers of biological reagents and experimental equipment also provide information useful in known biological methods. Such manufacturers include the SIGMA chemical company (Saint Louis, Mo.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), and Applied Biosystems (Foster City, Calif.), as well as many other commercial sources.

Vectors to which foreign nucleic acids are operably linked may be used to introduce these nucleic acids into host cells and mediate their replication and/or expression. “Cloning vectors” are useful for replicating and amplifying the foreign nucleic acids and obtaining clones of specific foreign nucleic acid-containing vectors. “Expression vectors” mediate the expression of the foreign nucleic acid. Some vectors are both cloning and expression vectors.

In general, the particular vector used to transport a foreign gene into the cell is not particularly critical. Any of the conventional vectors used for expression in the chosen host cell may be used.

An expression vector typically comprises a eukaryotic transcription unit or “expression cassette” that contains all the elements required for the expression of exogenous genes in eukaryotic cells. A typical expression cassette contains a promoter operably linked to the DNA sequence encoding a desired protein and signals required for efficient polyadenylation of the transcript.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated. Suitable promoters include the immediate early promoter from human cytomegalovirus (hCMV) and its associated intron A sequence (see e.g. WO0023592 for a suitable minimal promoter)

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Another suitable enhancer element is the HBV 3′-enhancer and HBV preS2 5′-UTR (see for example GenBank Accession No AF462041). Other enhancer/promoter combinations that are suitable for the present invention include those drived from polyoma virus, human or murine cytomegalovirus, the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In addition to a promoter sequence, the expression cassette should also contain a transcription termination region downstream of the structural gene to provide for efficient termination. The termination region may be obtained from the same source as the promoter sequence or may be obtained from a different source.

If the mRNA encoded by the selected structural gene is be efficiently translated, polyadenylation sequences are also commonly added to the vector construct (e.g. Rabbit B-globin pA: GenBank Accession No V00882). Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40, or a partial genomic copy of a gene already resident on the expression vector. A suitable

In addition to the elements already described, the expression vector of the present invention may typically contain other specialized elements intended to increase the level of expression of cloned nucleic acids or to facilitate the identification of cells that carry the transduced DNA. For instance, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The expression vectors of the present invention will typically contain both prokaryotic sequences that facilitate the cloning of the vector in bacteria as well as one or more eukaryotic transcription units that are expressed only in eukaryotic cells, such as mammalian cells. The prokaryotic sequences are preferably chosen such that they do not interfere with the replication of the DNA in eukaryotic cells.

Selected genes are normally be expressed when the DNA sequence is functionally inserted into a vector. “Functionally inserted” means that it is inserted in proper reading frame and orientation and operably linked to proper regrulatory elements. Typically, a gene will be inserted downstream from a promoter and will be followed by a stop codon, although production as a hybrid protein followed by cleavage may be used, if desired.

Expression vectors containing regulatory elements from eukaryotic viruses such as retroviruses are typically used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A.sup.+, pMTO10/A.sup.+, pMAMneo-5, baculovirus pDSVE, and any other vector allowing expression of proteins under the direction of the SV-40 early promoter, SV-40 later promoter, metallothionein promoter, murine mammary turnor virus promoter, Rous sarcoma virus promoter, polyhedrin promoter, or other promoters shown effective for expression in eukaryotic cells.

While a variety of vectors may be used, it should be noted that viral vectors such as retroviral vectors are useful for modifying eukaryotic cells because of the high efficiency with which the retroviral vectors transfect target cells and integrate into the target cell genome. Additionally, the retroviruses harboring the retroviral vector are capable of infecting cells from a wide variety of tissues.

In addition to the retroviral vectors mentioned above, cells may be lipofected with adeno-associated viral vectors. See, e.g., Methods in Enzymology, Vol. 185, Academic Press, Inc., San Diego, Calif. (D. V. Goeddel, ed.) (1990) or M. Krieger (1990), Gene Transfer and Expression—A Laboratory Manual, Stockton Press, New York, N.Y., and the references cited therein. Adeno associated viruses (AAVs) require helper viruses such as adenovirus or herpes virus to achieve productive infection. In the absence of helper virus functions, AAV integrates (site-specifically) into a host cell's genome, but the integrated AAV genome has no pathogenic effect. The integration step allows the AAV genome to remain genetically intact until the host is exposed to the appropriate environmental conditions (e.g., a lytic helper virus), whereupon it re-enters the lytic life-cycle. Samulski (1993), Current Opinion in Genetic and Development, 3: 74-80, and the references cited therein provides an overview of the AAV life cycle. See also West et al. (1987), Virology, 160: 38-47; Carter et al. (1989), U.S. Pat. No. 4,797,368; Carter et al. (1993), WO 93/24641; Kotin (1994), Human Gene Therapy, 5: 793-801; Muzyczka (1994), J. Clin. Invest., 94: 1351 and Samulski, supra, for an overview of AAV vectors.

Plasmids designed for producing recombinant vaccinia, such as pGS62, (Langford, C. L. et al. (1986), Mol. Cell. Biol., 6: 3191-3199) may also be used. This plasmid consists of a cloning site for insertion of foreign nucleic acids, the P7.5 promoter of vaccinia to direct synthesis of the inserted nucleic acid, and the vaccinia TK gene flanking both ends of the foreign nucleic acid.

Whatever the vector is used, generally the vector is genetically engineered to contain, in expressible form, a gene of interest. The particular gene selected will depend on the intended tretment. Examples of such genes of interest are described below at Section D.3. Insertion of Functional Copy of a Gene, and throughout the specification.

The vectors further usually comprise selectable markers which result in nucleic acid amplification such as the sodium, potassium ATPase, thymidine kinase, aminoglycoside phosphotransferase, hygromycin B phosphotransferase, xanthine-guanine phosphoribosyl transferase, CAD (carbamyl phosphate synthetase, aspartate transcarbamylase, and dihydroorotase), adenosine deaminase, dihydro folate reductase, and asparagine synthetase and ouabain selection. Alternatively, high yield expression systems not involving nucleic acid amplification are also suitable, such as using a bacculovirus vector in insect cells, with the encoding sequence under the direction of the polyhedrin promoter or other strong baculovirus promoters.

Therapeutic Uses

Immunological Indications

In the preferred embodiment the therapeutic effect results from a protein for Notch signalling. A detailed description of the Notch signalling pathway and conditions affected by it may be found in our WO98/20142, WO00/36089 and PCT/GB00/04391.

Diseased or infectious states that may be described as being mediated by T cells include, but are not limited to, any one or more of asthma, allergy, graft rejection, autoimmunity, tumour induced aberrations to the T cell system and infectious diseases such as those caused by Plasmodium species, Microfilariae, Helminths, Mycobacteria, HIV, Cytomegalovirus, Pseudomonas, Toxoplasma, Echinococcus, Haemophilus influenza type B, measles, Hepatitis C or Toxicara. Thus particular conditions that may be treated or prevented which are mediated by T cells include multiple schlerosis, rheumatoid arthritis and diabetes. The present invention may also be used in organ transplantation or bone marrow transplantation.

As indicated above, the present invention is useful in treating immune disorders such as autoimmune diseases or graft rejection such as allograft rejection.

Autoimmune Disease

Examples of disorders that may be treated include a group commonly called autoimmune diseases. The spectrum of autoimmune disorders ranges from organ specific diseases (such as thyroiditis, insulitis, multiple sclerosis, iridocyclitis, uveitis, orchitis, hepatitis, Addison's disease, myasthenia gravis) to systemic illnesses such as rheumatoid arthritis or lupus erythematosus. Other disorders include immune hyperreactivity, such as allergic reactions.

In more detail: Organ-specific autoimmune diseases include multiple sclerosis, insulin dependent diabetes mellitus, several forms of anemia (aplastic, hemolytic), autoimmune hepatitis, thyroiditis, insulitis, iridocyclitis, scleritis, uveitis, orchitis, myasthenia gravis, idiopathic thrombocytopenic purpura, inflammatory bowel diseases (Crohn's disease, ulcerative colitis).

Systemic autoimmune diseases include: rheumatoid arthritis, juvenile arthritis, scleroderma and systemic sclerosis, sjogren's syndrom, undifferentiated connective tissue syndrome, antiphospholipid syndrome, different forms of vasculitis (polyarteritis nodosa, allergic granulomatosis and angiitis, Wegner's granulomatosis, Kawasaki disease, hypersensitivity vasculitis, Henoch-Schoenlein purpura, Behcet's Syndrome, Takayasu arteritis, Giant cell arteritis, Thrombangiitis obliterans), lupus erythematosus, polymyafgia rheumatica, essentiell (mixed) cryoglobulinemia, Psoriasis vulgaris and psoriatic arthritis, diffus fasciitis with or without eosinophilia, polymyositis and other idiopathic inflammatory myopathies, relapsing panniculitis, relapsing polychondritis, lymphomatoid granulomatosis, erythema nodosum, ankylosing spondylitis, Reiter's syndrome, different forms of inflammatory dermatitis.

A more extensive list of disorders includes: unwanted immune reactions and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaeinic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery or organ, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

The present invention is also useful in cancer therapy. The present invention is especially useful in relation to adenocarcinomas such as: small cell lung cancer, and cancer of the kidney, uterus, prostrate, bladder, ovary, colon and breast.

Transplant Rejection

The present invention may be used, for example, for the treatment of organ transplants (e.g. kidney, heart, lung, liver or pancreas transplants), tissue transplants (e.g. skin grafts) or cell transplants (e.g. bone marrow transplants or blood transfusions).

A brief overview of the most common types of organ and tissue transplants is set out below.

1. Kidney Transplants:

Kidneys are the most commonly transplanted organs. Kidneys can be donated by both cadavers and living donors and kidney transplants can be used to treat numerous clinical indications (including diabetes, various types of nephritis and kidney failure). Surgical procedure for kidney transplantation is relatively simple. However, matching blood types and histocompatibility groups is desirable to avoid graft rejection. It is indeed important that a graft is accepted as many patients can become “sensitised” after rejecting a first transplant. Sensitisation results in the formation of antibodies and the activation of cellular mechanisms directed against kidney antigens. Thus, any subsequent graft containing antigens in common with the first is likely to be rejected. As a result, many kidney transplant patients must remain on some form of immunosuppressive treatment for the rest of their lives, giving rise to complications such as infection and metabolic bone disease.

2. Heart Transplantation

Heart transplantation is a very complex and high-risk procedure. Donor hearts must be maintained in such a manner that they will begin beating when they are placed in the recipient and can therefore only be kept viable for a limited period under very specific conditions. They can also only be taken from brain-dead donors. Heart transplants can be used to treat various types of heart disease and/or damage. HLA matching is obviously desirable but often impossible because of the limited supply of hearts and the urgency of the procedure.

3. Lung Transplantation

Lung transplantation is used (either by itself or in combination with heart transplantation) to treat diseases such as cystic fibrosis and acute damage to the lungs (e.g. caused by smoke inhalation). Lungs for use in transplants are normally recovered from brain-dead donors.

4. Pancreas Transplantation

Pancreas transplantation is mainly used to treat diabetes mellitus, a disease caused by malfunction of insulin-producing islet cells in the pancreas. Organs for transplantation can only be recovered from cadavers although it should be noted that transplantation of the complete pancreas is not necessary to restore the function needed to produce insulin in a controlled fashion. Indeed, transplantation of the islet cells alone could be sufficient. Because kidney failure is a frequent complication of advanced diabetes, kidney and pancreas transplants are often carried out simultaneously.

5. Skin Grafting

Most skin transplants are done with autologous tissue. However, in cases of severe burning (for example), grafts of foreign tissue may be required (although it should be noted that these grafts are generally used as biological dressings as the graft will not grow on the host and will have to be replaced at regular intervals). In cases of true allogenic skin grafting, rejection may be prevented by the use of immunosuppressive therapy. However, this leads to an increased risk of infection and is therefore a major drawback in burn victims.

6. Liver Transplantation

Liver transplants are used to treat organ damage caused by viral diseases such as hepititis, or by exposure to harmful chemicals (e.g. by chronic alcoholism). Liver transplants are also used to treat congenital abnormalities. The liver is a large and complicated organ meaning that transplantation initially posed a technical problem. However, most transplants (65%) now survive for more than a year and it has been found that a liver from a single donor may be split and given to two recipients. Although there is a relatively low rate of graft rejection by liver transplant patients, leukocytes within the donor organ together with anti-blood group antibodies can mediate antibody-dependent hemolysis of recipient red blood cells if there is a mismatch of blood groups. In addition, manifestations of GVHD have occurred in liver transplants even when donor and recipient are blood-group compatible.

General (not Necessarily Immunological) Indications

Cell Fate/Cancer Indications

The present invention is also useful in methods for altering the fate of a cell, tissue or organ type by altering Notch pathway function in the cell. Thus, the present application has application in the treatement of malignant and pre-neoplastic disorders. The present invention is especially useful in relation to adenocarcinomas such as: small cell lung cancer, and cancer of the kidney, uterus, prostrate, bladder, ovary, colon and breast. For example, malignancies which may be treatable according to the present invention include acute and chronic leukemias, lymphomas, myelomas, sarcomas such as Fibrosarcoma, myxosarcoma, liposarcoma, lymphangioendotheliosarcoma, angiosarcoma, endotheliosarcoma, chondrosarcoma, osteogenic sarcoma, chordoma, lymphangiosarcoma, synovioma, mesothelioma, leimyosarcoma, rhabdomyosarcoma, colon carcinoma, ovarian cancer, prostate cancer, pancreatic cancer, breasy cancer, squamous cell carcinoma, basal cell carcinoma, adenocarcinoma, sewat gland carcinoma, sebaceous gland carcinoma, papillary carcinoma, papillary adenocarcinomas, cystadenocarcinoma, medullary carcinoma, bronchogenic carcinoma, choriocarcinoma, renal cell carcinoma, hepatoma, bile duct carcinoma seminoma, embryonal carcinoma, cervical cancer, testicular tumour, lung carcinoma, small cell lung carcinoma, bladder carcinoma, epithelial carcinoma, glioma, astrocytoma, ependymoma, pinealoma, hemangioblastoma, acoustic neuoma, medulloblastoma, craniopharyngioma, oligodendroglioma, menangioma, melanoma, neutroblastoma and retinoblastoma.

The present invention may also have application in the treatment of nervous system disorders. Nervous system disorders which may be treated according to the present invention include neurological lesions including traumatic lesions resulting from physical injuries; ischaemic lesions; malignant lesions; infectious lesions such as those caused by HIV, herpes zoster or herpes simplex virus, Lyme disease, tuberculosis or syphilis; degenerative lesions and diseases and demyelinated lesions.

The present invention may be used to treat, for example, diabetes (including diabetic neuropathy, Bell's palsy), systemic lupus erythematosus, sarcoidosis, multiple sclerosis, human immunodeficiency virus-associated myelopathy, transverse myelopathy or various etiologies, progressive multi focal leukoencephalopathy, central pontine myelinolysis, Parkinson's disease, Alzheimer's disease, Huntington's chorea, amyotrophic lateral sclerosis, cerebral infarction or ischemia, spinal cord infarction or ischemia, progressive spinal muscular atrophy, progressive bulbar palsy, primary lateral sclerosis, infantile and juvenile muscular atrophy, progressive bulbar paralysis of childhood (Fazio-Londe syndrome), poliomyelitis and the post polio syndrome, and Hereditary Motorsensory Neuropathy (Charcot-Marie-Tooth Disease).

The present invention may further be useful in the promotion of tissue regeneration and repair. The present invention, therefore, may also be used to treat diseases associated with defective tissue repair and regeneration such as, for example, cirrhosis of the liver, hypertrophic scar formation and psoriasis. The invention may also be useful in the treatment of neutropenia or anemia and in techniques of organ regeneration and tissue engineering.

Antigens

In one embodiment, the constructs/particles of the present invention may be administered in simultaneous, separate or sequential combination with antigens or antigenic determinants (or polynucleotides coding therefor), to modify (increase or decrease) the immune response to such antigens or antigenic determinants.

An antigen suitable for use in the present invention may be any substance that can be recognised by the immune system, and is generally recognised by an antigen receptor. Preferably the antigen used in the present invention is an immunogen. An allergic response occurs when the host is re-exposed to an antigen that it has encountered previously.

The immune response to antigen is generally either cell mediated (T cell mediated killing) or humoral (antibody production via recognition of whole antigen). The pattern of cytokine production by TH cells involved in an immune response can influence which of these response types predominates: cell mediated immunity (TH1) is characterised by high IL-2 and IFNγ but low IL-4 production, whereas in humoral immunity (TH2) the pattern is low IL-2 and IFNy but high IL-4, IL-5 and IL-13. Since the secretory pattern is modulated at the level of the secondary lymphoid organ or cells, then pharmacological manipulation of the specific TH cytokine pattern can influence the type and extent of the immune response generated.

The TH1-TH2 balance refers to the relative representation of the two different forms of helper T cells. The two forms have large scale and opposing effects on the immune system. If an immune response favours TH1 cells, then these cells will drive a cellular response, whereas TH2 cells will drive an antibody-dominated response. The type of antibodies responsible for some allergic reactions is induced by TH2 cells.

The antigen or allergen (or antigenic determinant thereof) used in the present invention may be a peptide, polypeptide, carbohydrate, protein, glycoprotein, or more complex material containing multiple antigenic epitopes such as a protein complex, cell-membrane preparation, whole cells (viable or non-viable cells), bacterial cells or virus/viral component. In particular, it is preferred to use antigens known to be associated with auto-immune diseases such as myelin basic protein (associated with multiple sclerosis), collagen (associated with rheumatoid arthritis), and insulin (diabetes), or antigens associated with rejection of non-self tissue such as MHC antigens or antigenic determinants thereof. Where primed the APCs and/or T cells of the present invention are to be used in tissue transplantation procedures, antigens may be obtained from the tissue donor. Polynucleotides coding for antigens or antigenic determinants which may be expessed in a subject may also be used.

In a further embodiment, such antigens or antigenic determinants or polynucleotides coding for them may be included in or on a matrix/substrate e.g. particle.

Autoantigens and Bystander Antigens

The term “autoantigen” as used herein includes any substance or a component thereof normally found within a mammal that, in an autoimmune disease, becomes a target of attack by the immune system, preferably the primary (or a primary) target of attack. The term also includes antigenic substances that induce conditions having the characteristics of an autoimmune disease when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack. The term includes but is not limited to autoantigens and fragments thereof such as antigenic determinants (epitopes) involved in auto immune attack. In addition, the term includes antigens normally not exposed to the immune system which become exposed in the locus of autoimmune attack as a result of autoimmune tissue destruction, such as heatshock proteins (HSP), which although not necessarily specific to a particular tissue are normally shielded from the immune system.

“Bystander suppression” is suppression at the locus of autoimmune attack of cells that contribute to autoimmune destruction; this suppression is mediated by the release of one or more immunosuppressive factors (including Th2-enhancing cytokines and Th1-inhibiting cytokines) from suppressor/regulatory T-cells elicited by a bystander antigen and recruited to the site where cells contributing to autoimmune destruction are found. The result may be antigen-nonspecific but locally restricted downregulation of the autoimmune responses responsible for tissue destruction.

“Autoimmune disease” includes spontaneous or induced malfunction of the immune system of mammals, including humans, in which the immune system fails to distinguish between foreign immunogenic substances within the mammal and/or autologous substances and, as a result, treats autologous tissues and substances as if they were foreign and mounts an immune response against them.

Autoimmune diseases are characterized by immune responses that are directed against self antigens. These responses are maintained by the persistent activation of self-reactive T lymphocytes. T lymphocytes are specifically activated upon recognition of foreign and/or self antigens as a complex with self Major Histocompatibility Complex (MHC) gene products on the surface of antigen-presenting cells (APC).

A detailed discussion of autoimmune diseases, autoantigens and bystander antigens is included in the textbook “The Autoimmune Diseases” Third Edition, 1998, edited by Rose and Mackay, Academic Press, San Diego, Calif., US (Library of Congress Card Catalog No 98-84368, ISBN 0-12-596923-6), the text of which is hereby incorporated herein by reference.

A non-limiting list of autoimmune diseases and tissue- or organ-specific confirmed or potential bystander antigens and autoantigens of use in the products of the present invention is provided below.

Autoimmune Disorders

Autoimmune disorders include organ specific diseases and systemic illnesses.

In more detail, organ-specific autoimmune diseases include, for example, several forms of anemia (aplastic, hemolytic), autoimmune hepatitis, iridocyclitis, scleritis, uveitis, orchitis and idiopathic thrombocytopenic purpura.

Systemic autoimmune diseases include, for example: undifferentiated connective tissue syndrome, antiphospholipid syndrome, different forms of vasculitis (polyarteritis nodosa, allergic granulomatosis and angiitis), Wegner's granulomatosis, Kawasaki disease, hypersensitivity vasculitis, Henoch-Schoenlein purpura, Behcet's Syndrome, Takayasu arteritis, Giant cell arteritis, Thrombangiitis obliterans, polymyalgia rheumatica, essential (mixed) cryoglobulinemia, psoriasis vulgaris and psoriatic arthritis, diffuse fasciitis with or without eosinophilia, relapsing panniculitis, relapsing polychondritis, lymphomatoid granulomatosis, erythema nodosum, ankylosing spondylitis, Reiter's syndrome and different forms of inflammatory dermatitis.

Autoantigens

Autoantigens may be derived from tissues, proteins etc associated with the disease which give rise to the relevant autoimmune response. For example:

| Autoimmune condition | Source of autoantigens |
|---|---|
| Addison's disease | adrenal cell antigens; 21-hydroxylase, |
| | 17-hydroxylase |
| Alopecia | hair follicle antigens |
| Autoimmune hepatitis | liver cell antigens |
| Autoimmune parotitis | parotid gland antigens |
| Autoimmune haemolytic anemia | red cell membrane proteins; 95-110 kDa membrane protein |
| Chronic active hepatitis | liver cell antigens |
| Goodpasture's syndrome | renal and lung basement membrane antigens; |
| | collagens |
| Guillain-Barre syndrome | nerve cell antigens |
| Hypophysial insufficiency | Hypophyseal antigens |
| Biermer's gastritis | Parietal cell of the stomach; intrinsic |
| | Factor |
| Idiopathic leukopenia | granulocyte antigens |
| Idiopathic thrombocytopenia | platelet membrane proteins; |
| | Glycoprotein IIa/IIIb |
| Isaac's syndrome | voltage-gated potassium channels |
| Lambert-Eaton | synaptogamin in voltage-gated |
| myasthenic syndrome (LEMS) | calcium channels |
| Myocardial infraction | heart cell antigens |
| Paraneoplastic encephalitis | RNA-binding protein (HuD) |
| Pemphigus vulgaris | “PeV antigen complex”; desmoglein (DG) (see e.g. |
| | Eur. J. Cell Biol. 55: 200 (91)) |
| Primary biliary cirrhosis | mitochondrial antigens; dihydrolipoamide |
| | acetyltransferase; pyruvate dehydrogenase |
| | complex 2 (PDC-E2) |
| Progressive systemic sclerosis | DNA topoisomerase; RNA polymerase |
| Spontaneous infertility | Sperm antigens (e.g. post-acrosomal sperm protein |
| | (PASP)) (see e.g. Biol. Reprod. 43: 559 (90)) |
| Uveitis | Ocular antigen, S-antigen, interphotoreceptor |
| | retinoid binding protein |
| | (see e.g. Exp. Eye Res. 56: 463 (93)) |
| Vitiligo | melanocyte antigens |

It will be appreciated that combinations of such autoimmune antigens and autoimmune antigenic determinants and/or polynucleotide sequences coding for them may also be used as appropriate.

An antigen suitable for use in the present invention may be any substance that can be recognised by the immune system, and is generally recognised by an antigen (T-cell) receptor. Preferably the antigen used in the present invention is an immunogen.

The antigen used in the present invention may be a peptide, polypeptide, carbohydrate, protein, glycoprotein, or more complex material containing multiple antigenic epitopes such as a protein complex, cell-membrane preparation, whole cells (viable or non-viable cells), bacterial cells or virus/viral component.

The antigen moiety may be, for example, a synthetic MHC-peptide complex i.e. a fragment of the MHC molecule bearing the antigen groove bearing an element of the antigen. Such complexes have been described in Altman et al. (1996) Science 274: 94-96.

Some preferred autoantigens for use in the products, methods, uses and constructs etc of the present invention include the following:

Goodpasture's Autoantigens and Bystander Antigens

In one embodiment of the present invention the autoantigen or bystander antigen may be a Goodpasture's autoantigen or bystander antigen for use to treat Goodpasture's disease/syndrome.

The term “Goodpasture's autoantigen” as used herein includes any substance or a component thereof normally found within a mammal that, in Goodpasture's disease, becomes a target of attack by the immune system, preferably the primary (or a primary) target of attack. The term also includes antigenic substances that induce conditions having the characteristics of Goodpasture's disease when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “Goodpasture's bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in Goodpasture's disease. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Examples of Goodpasture's autoantigens and Goodpasture's bystander antigens include, but are not limited to collagens in particular, type IV, alpha 3 collagens.

An amino acid sequence for a human collagen, type IV, alpha 3 (Goodpasture antigen) is reported as follows (GenBank Accession No NM 001723; SEQ ID NO: 78):

MSARTAPRPQVLLLPLLLVLLAAAPAASKGCVCKDKGQCFCDGAKGEKGEKGFPGPPGSPGQKGFTGPEGLPGP
QGPKGFPGLPGLTGSKGVRGISGLPGFSGSPGLPGTPGNTGPYGLVGVPGCSGSKGEQGFPGLPGTPGYPGIPG
AAGLKGQKGAPAKGEDIELDAKGDPGLPGAPGPQGLPGPPGFPGPVGPPGPPGFFGFPGAMGPRGPKGHMGERV
IGHKGERGVKGLTGPPGPPGTVIVTLTGPDNRTDLKGEKGDKGAMGEPGPPGPSGLPGESYGSEKGAPGDPGLQ
GKPGKDGVPGFPGSEGVKGNRGFPGLMGEDGIKGQKGDIGPPGFRGPTEYYDTYQEKGDEGTPGPPGPRGARGP
QGPSGPPGVPGSPGSSRPGLRGAPGWPGLKGSKGERGRPGKDAMGTPGSPGCAGSPGLPGSPGPPGPPGDIVFR
KGPPGDHGLPGYLGSPGIPGVDGPKGEPGLLCTQCPYIPGPPGLPGLPGLHGVKGIPGRQGAAGLKGSPGSPGN
TGLPGFPGFPGAQGDPGLKGEKGETLQPEGQVGVPGDPGLRGQPGRKGLDGIPGTLGVKGLPGPKGELALSGEK
GDQGPPGDPGSPGSPGPAGPAGPPGYGPQGEPGLQGTQGVPGAPGPPGEAGPRGELSVSTPVPGPPGPPGPPGH
PGPQGPPGIPGSLGKCGDPGLPGPDGEPGIPGIGFPGPPGPKGDQGFPGTKGSLGCPGKMGEPGLPGKPGLPGA
KGEPAVAMPGGPGTPGFPGERGNSGEHGEIGLPGLPGLPGTPGNEGLDGPRGDPGQPGPPGEQGPPGRCIEGPR
GAQGLPGLNGLKGQQGRRGKTGPKGDPGIPGLDRSGFPGETGSPGIPGHQGEMGPLGQRGYPGNPGILGPPGED
GVIGMMGFPGAIGPPGPPGNPGTPGQRGSPGIPGVKGQRGTPGAKGEQGDKGNPGPSEISHVIGDKGEPGLKGF
AGNPGEKGNRGVPGMPGLKGLKGLPGPAGPPGPRGDLGSTGNPGEPGLRGIPGSMGNMGMPGSKGKRGTLGFPG
RAGRPGLPGIHGLQGDKGEPGYSEGTRPGPPGPTGDPGLPGDMGKKGEMGQPGPPGHLGPAGPEGAPGSPGSPG
LPGKPGPHGDLGFKGIKGLLGPPGIRGPPGLPGFPGSPGPMGIRGDQGRDGIPGPAGEKGETGLLRAPPGPRGN
PGAQGAKGDRGAPGFPGLPGRKGAMGDAGPRGPTGIEGFPGPPGLPGAIIPGQTGNRGPPGSRGSPGAPGPPGP
PGSHVIGIKGDKGSMGHPGPKGPPGTAGDMGPPGRLGAPGTPGLPGPRGDPGFQGFPGVKGEKGNPGFLGSIGP
PGPIGPKGPPGVRGDPGTLKIISLPGSPGPPGTPGEPGMQGEPGPPGPPGNLGPCGPRGKPGKDGKPGTPGPAG
EKGNKGSKGEPESLFHQL

(see also Turner et al, Molecular cloning of the human Goodpasture antigen demonstrates it to be the alpha 3 chain of type IV collagen, J. Clin. Invest. 89 (2), 592-601 (1992))

Further sequences are provided, for example, under GenBank Accession Nos NM_{—}031366.1, NM_{—}031364.1, NM_{—}031363.1, NM_{—}031362.1 and NM_{—}000091.2 (collagen, type IV, alpha 3 (Goodpasture antigen) (COL4A3)) and NM_{—}130778.1 and NM_{—}000494.2 (collagen, type XVII, alpha 1 (COL17A1)).

Renal Autoantigens and Bystander Antigens

In another embodiment the autoantigen or bystander antigen may be a renal autoantigen or renal bystander antigen, for use to treat autoimmune disease of the kidney.

The term “renal autoantigen” as used herein includes any substance or a component thereof normally found within a mammal that, in autoimmune disease of the kidney, becomes a target of attack by the immune system, preferably the primary (or a primary) target of attack. The term also includes antigenic substances that induce conditions having the characteristics of an autoimmune disease of the kidney when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “renal bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the kidney under autoimmune attack in an autoimmune disease of the kidney. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Examples of renal autoantigens and renal bystander antigens include, but are not limited to glomerular basement membrane (GBM) antigens (Goodpasture's antigens as described further above) and tubular basement membrane (TBM) antigens associated with tubulointerstitial nephritis (TIN).

Pemphigus Autoantigens and Bystander Antigens

In an alternative embodiment of the present invention the autoantigen or bystander antigen may be a Pemphigus autoantigen or bystander antigen for use to treat Pemphigus.

The term “Pemphigus autoantigen” as used herein includes any substance or a component thereof normally found within a mammal that, in Pemphigus, becomes a target of attack by the immune system, preferably the primary (or a primary) target of attack. The term also includes antigenic substances that induce conditions having the characteristics of Pemphigus when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “Pemphigus bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in Pemphigus. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Pemphigus includes, for example, pemphigus vulgaris, pemphigus foliaceus and bullous pemphigoid.

Examples of Pemphigus autoantigens and Pemphigus bystander antigens include, but are not limited to desmoglein 1 and desmoglein 3.

An amino acid sequence for a human desmoglein 1 (DSG1) autoantigen protein is reported as follows (GenBank Accession No AF097935; SEQ ID NO: 79):

MDWSFFRVVAVLFIFLVVVEVNSEFRIQVRDYNTKNGTIKWHSIRRQKREWIKFAAACREGEDNSKRNPIAKIH
SDCAANQQVTYRISGVGIDQPPYGIFVINQKTGEINITSIVDREVTPFFIIYCRALNSMGQDLERPLELRVRVL
DINDNPPVFSMATFAGQIEENSNANTLVMILNATDADEPNNLNSKIAFKIIRQEPSDSPMFIINRNTGEIRTMN
NFLDREQYGQYALAVRGSDRDGGADGMSAECECNIKILDVNDNIPYMEQSSYTIEIQENTLNSNLLEIRVIDLD
EEFSANWMAVIFFISGNEGNWFEIEMNERTNVGILKVVKPLDYEAMQSLQLSIGVRNKAEFHHSIMSQYKLKAS
AISVTVLNVIEGPVFRPGSKTYVVTGNMGSNDKVGDFVATDLDTGRPSTTVRYVMGNNPADLLAVDSRTGKLTL
KNKVTKEQYNMLGGKYQGTILSIDDNLQRTCTGTININIQSFGNDDRTNTEPNTKITTNTGRQESTSSTNYDTS
TTSTDSSQVYSSEPGNGAKDLLSDNVHFGPAGIGLLIMGFLVLGLVPFLMICCDCGGAPRSAAGFEPVPECSDG
AIHSWAVEGPQPEPRDITTVIPQIPPDNANIIECIDNSGVYTNEYGGREMQDLGGGERMTGFELTEGVKTSGMP
EICQEYSGTLRRNSMRECREGGLNMNFMESYFCQKAYAYADEDEGRPSNDCLLIYDIEGVGSPAGSVGCCSFIG
EDLDDSFLDTLGPKFKKLADISLGKESYPDLDPSWPPQSTEPVCLPQETEPVVSGHPPISPHFGTTTVISESTY
PSGPGVLHPKPILDPLGYGNVTVTESYTTSDTLKPSVHVHDNRPASNVVVTERVVGPISGADLHGMLEMPDLRD
GSNVIVTERVIAPSSSLPTSLTIHHPRESSNVVVTERVIQPTSGMIGSLSMHPELANAHNVIVTERVVSGAGVT
GISGTTGISGGIGSSGLVGTSMGAGSGALSGAGISGGGIGLSSLGGTASIGHMRSSSDHHFNQTIGSASPSTAR
SRITKYSTVQYSK

(see also Nilles et al, Structural analysis and expression of human desmoglein: a cadherin-like component of the desmosome, J. Cell. Sci. 99 (Pt 4), 809-821 (1991))

An amino acid sequence for a human bullous pemphigoid antigen 1, 230/240 kDa (BPAG1) is reported as follows (GenBank Accession No NM_{—}001723; SEQ ID NO: 80):

MHSSSYSYRSSDSVFSNTTSTRTSLDSNENLLLVHCGPTLINSCISFGSESFDGHRLEMLQQIANRVQRDSVIC
EDKLILAGNALQSDSKRLESGVQFQNEAEIAGYILECENLLRQHVIDVQILIDGKYYQADQLVQRVAKLRDEIM
ALRNECSSVYSKGRILTTEQTKLMISGITQSLNSGFAQTLHPSLTSGLTQSLTPSLTSSSMTSGLSSGMTSRLT
PSVTPAYTPGFPSGLVPNFSSGVEPNSLQTLKLMQIRKPLLKSSLLDQNLTEEEINMKFVQDLLNWVDEMQVQL
DRTEWGSDLPSVESHLENHKNVHRAIEEFESSLKEAKISEIQMTAPLKLTYAEKLHRLESQYAKLLNTSRNQER
HLDTLHNFVSRATNELIWLNEKEEEEVAYDWSERNTNIARKKDYHAELMRELDQKEENIKSVQEIAEQLLLENH
PARLTIEAYRAAMQTQWSWILQLCQCVEQHIKENTAYFEFFNDAKEATDYLRNLKDAIQRKYSCDRSSSIHKLE
DLVQESMEEKEELLQYKSTIANLNGKAKTIIQLKPRNSDCPLKTSIPIKAICDYRQIEITIYKDDECVLANNSH
RAKWKVISPTGNEAMVPSVCFTVPPPNKEAVDLANRIEQQYQNVLTLWHESHINMKSVVSWHYLINEIDRIRAS
NVASIKTMLPGEHQQVLSNLQSRFEDFLEDSQESQVFSGSDITQLEKEVNVCKQYYQELLKSAEREEQEESVYN
LYISEVRNIRLRLENCEDRLIRQIRTPLERDDLHESVFRITEQEKLKKELERLKDDLGTITNKCEEFFSQAAAS
SSVPTLRSELNVVLQNMNQVYSMSSTYIDKLKTVNLVLKNTQAAEALVKLYETKLCEEEAVIADKNNIENLIST
LKQWRSEVDEKRQVFHALEDELQKAKAISDEMFKTYKERDLDFDWHKEKADQLVERWQNVHVQIDNRLRDLEGI
GKSLKYYRDTYHPLDDWIQQVETTQRKIQENQPENSKTLATQLNQQKMLVSEIEMKQSKMDECQKYAEQYSATV
KDYELQTMTYRAMVDSQQKSPVKRRRMQSSADLIIQEFMDLRTRYTALVTLMTQYIKFAGDSLKRLEEEEIKRC
KETSEHGAYSDLLQRQKATVLENSKLTGKISELERMVAELKKQKSRVEEELPKVREAAENELRKQQRNVEDISL
QKIRAESEAKQYRRELETIVREKEAAERELERVRQLTIEAEAKRAAVEENLLNFRNQLEENTFTRRTLEDHLKR
KDLSLNDLEQQKNKLMEELRRKRDNEEELLKLIKQMEKDLAFQKQVAEKQLKEKQKIELEARRKITETQYTCRE
NALPVCPITQATSCRAVTGLQQEHDKQKAEELKQQVDELTAANRKAEQDMRELTYELNALQLEKTSSEEKARLL
KDKLDETNNTLRCLKLELERKDQAEKGYSQQLRELGRQLNQTTGKAEEAMQEASDLKKIKRNYQLELESLNHEK
GKLQREVDRITRAHAVAEKNIQHLNSQIHSFRDEKELERLQICQRKSDHLKEQFEKSHEQLLQNIKAEKENNDK
IQRLNEELEKSNECAEMLKQKVEELTRQNNETKLMMQRIQAESENIVLEKQTIQQRCEALKIQADGFKDQLRST
NEHLHKQTKTEQDFQRKIKCLEEDLAKSQNLVSEFKQKCDQQNIIIQNTKKEVRNLNAELNASKEEKRRGEQKV
QLQQAQVQELNNRLKKVQDELHLKTIEEQMTHRKMVLFQEESGKFKQSAEEFRKKMEKLMESKVITENDISGIR
LDFVSLQQENSRAQENAKLCETNIKELERQLQQYREQMQQGQHMEANHYQKCQKLEDELIAQKREVENLKQKMD
QQIKEHEHQLVLLQCEIQKKSTAKDCTFKPDFEMTVKECQHSGELSSRNTGHLHPTPRSPLLRWTQEPQPLEEK
WQHRVVEQIPKEVQFQPPGAPLEKEKSQQCYSEYFSQTSTELQITFDETNPITRLSEIEKIRDQALNNSRPPVR
YQDNACEMELVKVLTPLEIAKNKQYDMHTEVTTLKQEKNPVPSAEEWMLEGCRASGGLKKGDFLKKGLEPETFQ
NFDGDHACSVRDDEFKFQGLRHTVTARQLVEAKLLDMRTIEQLRLGLKTVEEVQKTLNKFLTKATSIAGLYLES
TKEKISFASAAERIIIDKMVALAFLEAQAATGFIIDPISGQTYSVEDAVLKGVVDPEFRIRLLEAEKAAVGYSY
SSKTLSVFQAMENRMLDRQKGKHILEAQIASGGVIDPVRGIRVPPEIALQQGLLNNAILQFLHEPSSNTRVFPN
PNNKQALYYSELLRMCVFDVESQCFLFPFGERNISNLNVKKTHRISVVDTKTGSELTVYEAFQRNLIEKSIYLE
LSGQQYQWKEAMFFESYGHSSHMLTDTKTGLHFNINEAIEQGTIDKALVKKYQEGLITLTELADSLLSRLVPKK
DLHSPVAGYWLTASGERISVLKASRRNLVDRITALRCLEAQVSTGGIIDPLTGKKYRVAEALHRGLVDEGFAQQ
LRQCELVITGIGHPITNKMMSVVEAVNANIINKEMGIRCLEFQYLTGGLIEPQVHSRLSIEEALQVGIIDVLIA
TKLKDQKSYVRNIICPQTKRKLTYKEALEKADFDFHTGLKLLEVSEPLMTGISSLYYSS

(see also, for example Sawamura et al, Bullous pemphigoid antigen (BPAG1): cDNA cloning and mapping of the gene to the short arm of human chromosome 6, Genomics 8 (4), 722-726 (1990))

Further sequences are provided, for example, under GenBank Accession Nos NM_{—}015548.1, NM_{—}020388.2 and NM_{—}001723.2 (Bullous pemphigoid antigen 1 (230/240 kD) (BPAG1)), M91669.1 (Bullous pemphigoid autoantigen BP180), NM_{—}001942.1 (desmoglein 1 (DSG1)) and NM_{—}001944.1 (desmoglein 3 (pemphigus vulgaris antigen; DSG3))

In one embodiment one or more antigenic determinants may be used in place of a full antigen. For example, some specific class II MHC-associated autoantigen peptide sequences are as follows (see U.S. Pat. No. 5,783,567):

| | |
|---|---|
| Peptide Sequence | Source |

| | | |
|---|---|---|
| LNSKIAFKIVSQEPA | desmoglein 3 | (aa 190-204; |
| | | SEQ ID NO: 81) |
| TPMFLLSRNTGEVRT | desmoglein 3 | (aa 206-220; |
| | | SEQ ID NO: 82)) |

Thyroid Autoantigens and Bystander Antigens

In an alternative embodiment of the present invention the autoantigen or bystander antigen may be a thyroid autoantigen or bystander antigen for use to treat thyroid autoimmune disease.

The term “thyroid autoimmune disease” as used herein includes any condition in which there is an autoimmune reaction to the thyroid or a component thereof. The best known autoimmune diseases of the thyroid include Graves' disease (also known as thyrotoxicosis), Hashimoto's thyroiditis and primary hypothyroidism. Further examples include atrophic autoimmune thyroiditis, primary myxoedema, asymptomatic thyroiditis, postpartal thyroiditis and neonatal hypothyroidism.

Diagnosis is typically based on the detection of autoantibodies in the patient. The three main thyroid autoantigens are the TSH receptor, thyroperoxidase (TPO, also known as microsomal antigen) and thyroglobulin (Tg) (Dawe, K., Hutchings, P., Champion, B., Cooke, A., Roitt, I., “Autoantigens in Thyroid diseases”, Springer Semin. Immunopathol. 14, 285-307, 1993).

The term “thyroid autoantigen” as used herein includes any substance or a component thereof normally found within a mammal that, in a thyroid autoimmune disease, becomes a target of attack by the immune system, preferably the primary (or a primary) target of attack. The term also includes antigenic substances that induce conditions having the characteristics of a thyroid autoimmune disease when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ (usually the thyroid gland) under autoimmune attack and recognized by a substantial percentage (e.g. a majority though not necessarily an absolute majority) of autoimmune attack T-cells.

The term “thyroid bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the thyroid gland under autoimmune attack. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

It will be appreciated that combinations of thyroid autoimmune/bystander antigens and thyroid autoimmune/bystander antigenic determinants and/or polynucleotide sequences coding for them may also be used as appropriate.

Examples of thyroid autoantigens and thyroid bystander antigens include, but are not limited to, the thyroid stimulatory hormone (TSH) receptor (associated in particular with Grave's disease), thyroperoxidase (TPO; associated with Hashimoto's thyroiditis) and thyroglobulin (Tg).

For example, an amino acid sequence for a human thyroid stimulatory hormone receptor (TSHR) is reported as follows (GenBank Accession No M32215; SEQ ID NO: 83):

MRPADLLQLVLLLDLPRDLGGMGCSSPPCECHQEEDFRVTCKDIQRIPSLPPSTQTLKLIETHLRTIPSHAFSN
LPNISRIYVSIDVTLQQLESHSFYNLSKVTHIEIRNTRNLTYIDPDALKELPLLKFLGIFNTGLKMFPDLTKVY
STDIFFILEITDNPYMTSIPVNAFQGLCNETLTLKLYNNGFTSVQGYAFNGTKLDAVYLNKNKYLTVIDKDAFG
GVYSGPSLLDVSQTSVTALPSKGLEHLKELIARNTWTLKKLPLSLSFLHLTRADLSYPSHCCAFKNQKKIRGIL
ESLMCNESSMQSLRQRKSVNALNSPLHQEYEENLGDSIVGYKEKSKFQDTHNNAHYYVFFEEQEDEIIGFGQEL
KNPQEETLQAFDSHYDYTICGDSEDMVCTPKSDEFNPCEDIMGYKFLRIVVWFVSLLALLGNVFVLLILLTSHY
KLNVPRFLMCNLAFADFCMGMYLLLIASVDLYTHSEYYNHAIDWQTGPGCNTAGFFTVFASELSVYTLTVITLE
RWYAITFAMRLDRKIRLRHACAIMVGGWVCCFLLALLPLVGISSYAKVSICLPMDTETPLALAYIVFVLTLNIV
AFVIVCCCYVKIYITVRNPQYNPGDKDTKIAKRMAVLIFTDFICMAPISFYALSAILNKPLITVSNSKILLVLF
YPLNSCANPFLYAIFTKAFQRDVFILLSKFGICKRQAQAYRGQRVPPKNSTDIQVQKVTHEMRQGLHNMEDVYE
LIEKSHLTPKKQGQISEEYMQTVL

An amino acid sequence for a human thyroperoxidase (described as the primary autoantigen in human autoimmune thyroiditis (Hashimoto's thyroiditis) is reported as follows (GenBank Accession No M17755; SEQ ID NO: 84):

MRALAVLSVTLVMACTEAFFPFISRGKELLWGKPEESRVSSVLEESKRLVDTAMYATMQRNLKKRGTLSGAQLL
SFSKLPEPTSGVIARAAEIMETSIQAMKRKVNLKTQQSQHPTDALSEDLLSIIANMSGCLPYMLPPKCPNTCLA
NKYRPITGACNNRDHPRWGASNTALARWLPPVYEDGFSQPRGWNPGFLYNGFPLPPVREVTRHVIQVSNEVVTD
DDRYSDLLMAWGQYIDHDIAFTPQSTSKAAFGGGSDCQMTCENQNPCFPIQLPEEARPAAGTACLPFYRSSAAC
GTGDQGALFGNLSTANPRQQMNGLTSFLDASTVYGSSPALERQLRNWTSAEGLLRVHGRLRDSGRAYLPFVPPR
APAACAPEPGNPGETRGPCFLAGDGRASEVPSLTALHTLWLREHNRLAAALKALNAHWSADAVYQEARKVVGAL
HQIITLRDYIPRILGPEAFQQYVGPYEGYDSTANPTVSNVFSTAAFRFGHATIHPLVRRLDASFQEHPDLPGLW
LHQAFFSPWTLLRGGGLDPLIRGLLARPAKLQVQDQLMNEELTERLFVLSNSSTLDLASINLQRGRDHGLPGYN
EWREFCGLPRLETPADLSTAIASRSVADKILDLYKHPDNIDVWLGGLAENFLPRARTGPLFACLIGKQMKALRD
GDWFWWENSHVFTDAQRRELEKHSLSRVICDNTGLTRVPMDAFQVGKFPEDFESCDSITGMNLEAWRETFPQDD
KCGFPESVENGDFVHCEESGRRVLVYSCRHGYELQGREQLTCTQEGWDFQPPLCKDVNECADGAHPPCHASARC
RNTKGGFQCLCADPYELGDDGRTCVDSGRLPRVTWISMSLAALLIGGFAGLTSTVICRWTRTGTKSTLPISETG
GGTPELRCGKHQAVGTSPQRAAAQDSEQESAGMEGRDTHRLPRAL

5 Wegener's Autoantigens and Bystander Antigens

In an alternative embodiment of the present invention the autoantigen or bystander antigen may be a Wegener's autoantigen or bystander antigen for use to treat Wegener's disease.

The term “Wegener's autoantigen” as used herein includes any substance or a component thereof normally found within a mammal that, in Wegener's disease, becomes a target of attack by the immune system, preferably the primary (or a primary) target of attack. The term also includes antigenic substances that induce conditions having the characteristics of Wegener's disease when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “Wegener's bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in Wegener's disease. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Examples of Wegener's autoantigens and Wegener's bystander antigens include, but are not limited to myeloblastin/proteinase 3.

An amino acid sequence for a Wegener's autoantigen/myeloblastin/proteinase 3 autoantigen is reported as follows (GenBank Accession No M75154; SEQ ID NO: 85):

MAHRPPSPALASVLLALLLSGAARAAEIVGGHEAQPHSRPYMASLQMRGNPGSHFCGGTLIHPSFVLTAPHCLR
DIPQRLVNVVLGAHNVRTQEPTQQHFSVAQVFLNNYDAENKLNDILLIQLSSPANLSASVTSVQLPQQDQPVPH
GTQCLAMGWGRVGAHDPPAQVLQELNVTVVTFFCRPHNICTFVPRRKAGICFGDSGGPLICDGIIQGIDSFVIW
GCATRLFPDFFTRVALYVDWIRSTLRRVEAKGRP

(see also Labbaye et al, Wegener autoantigen and myeloblastin are encoded by a single mRNA, Proc. Natl. Acad. Sci. U.S.A. 88 (20), 9253-9256 (1991))
Autoimmune Anemia Autoantigens and Bystander Antigens

In a further alternative embodiment of the present invention the autoantigen or bystander antigen may be an autoimmune anemia autoantigen or bystander antigen fo use to treat autoimmune anemia.

The term “autoimmune anemia” as used herein includes any disease in which red blood cells (RBCs) or a component thereof come under autoimmune attack. The term includes, for example, autoimmune haemolytic anemia, including both “warm autoantibody type” and “cold autoantibody type”.

The term “autoimmune anemia autoantigen” as used herein includes any substance or a component thereof normally found within a mammal that, in autoimmune anemia, becomes a target of attack by the immune system, preferably the primary (or a primary) target of attack. The term also includes antigenic substances that induce conditions having the characteristics of autoimmune anemia when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “autoimmune anemia bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the red blood cells (RBCs) under autoimmune attack in autoimmune anemia. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Autoimmune anemia includes, in particular, autoimmune hemolytic anemia. Examples of autoimmune hemolytic anemia autoantigens and bystander antigens include, but are not limited to Rhesus (Rh) antigens such as E, e or C, red cell proteins and glycoproteins such as red cell protein band 4.1 and red cell membrane band 3 glycoprotein. Further examples include Wr^{b}, Ena, Ge, A, B and antigens within the Kidd and Kell blood group systems.

Autoimmune Thrombocytopenia Autoantigens and Bystander Antigens

In a further alternative embodiment of the present invention the autoantigen or bystander antigen may be an autoimmune thrombocytopenia autoantigen or bystander antigen for use to treat autoimmune thrombocytopenia.

The term “autoimmune thrombocytopenia autoantigen” as used herein includes any substance or a component thereof normally found within a mammal that, in autoimmune thrombocytopenia, becomes a target of attack by the immune system, preferably the primary (or a primary) target of attack. The term also includes antigenic substances that induce conditions having the characteristics of autoimmune thrombocytopenia when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “autoimmune thrombocytopenia bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the platelets under autoimmune attack in autoimmune thrombocytopenia. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Autoimmune thrombocytopenia includes, in particular, autoimmune thrombocytopenia purpura. Examples of autoimmune thrombocytopenia purpura autoantigens and bystander antigens include, but are not limited to platelet glycoproteins such as GPIIb/IIIa and/or GPIb/IX.

For example, an amino acid sequence for a human platelet glycoprotein IIb (GPIIb) is reported as follows (GenBank Accession No M34480; SEQ ID NO: 86)

MARALCPLQALWLLEWVLLLLGACAAPPAWALNLDPVQLTFYAGPNGSQFGFSLDFHKDSHGRVAIVVGAPRTL
GPSQEETGGVFLCPWRAEGGQCPSLLFDLRDETRNVGSQTLQTFKARQGLGASVVSWSDVIVACAPWQHWNVLE
KTEEAEKTPVGSCFLAQPESGRRAEYSPCRGNTLSRIYVENDFSWDKRYCEAGFSSVVTQAGELVLGAPGGYYF
LGLLAQAPVADIFSSYRPGILLWHVSSQSLSFDSSMPEYFDGYWGYSVAVGEFDGDLNTTEYVVGAPTWSWTLG
AVEILDSYYQRLHRLRAEQMASYFGHSVAVTDVNGDGRHDLLVGAPLYMDSRADRKLAEVGRVYLFLQPRGPHA
LGAPSLLLTGTQLYGRFGSAIAPLGDLDRDGYNDIAVAAPYGGPSGRGQVLVFLGQSEGLRSRPSQVLDSPFPT
GSAFGFSLRGAVDIDDNGYPDLIVGAYGANQVAVYRAQPVVKASVQLLVQDSLNPAVKSCVLPQTKTPVSCFNI
QMCVGATGHNIPQKLSLNAELQLDRQKPRQGRRVLLLGSQQAGTTLDLDLGGKHSPICHTTMAFLRDEADFRDK
LSPIVLSLNVSLPPTEAGMAPAVVLHGDTHVQEQTRIVLDCGEDDVCVPQLQLTASVTGSPLLVGADNVLELQM
DAANEGEGAYEAELAVHLPQGAHYMRALSNVEGFERLICNQKKENETRVVLCELGNPMKKNAQIGIAMLVSVGN
LEEAGESVSFQLQIRSKNSQNPNSKIVLLDVPVRAEAQVELRGNSFPASLVVAAEEGEREQNSLDSWGPKVEHT
YELHNNGPGTVNGLHLSIHLPGQSQPSDLLYILDIQPQGGLQCFPQPPVNPLKVDWGLPIPSPSPIHPAHHKRD
RRQIFLPEPEQPSRLQDPVLVSCDSAPCTVVQCDLQEMARGQRANVTVLAFLWLPSLYQRPLDQFVLQSHAWFN
VSSLPYAVPPLSLPRGEAQVWTQLLRALEERAIPIWWVLVGVLGGLLLLTILVLAMWKVGFFKRMRHTLEEDDE
EGE

An amino acid sequence for a human platelet glycoprotein IIIa (GPIIIa) is reported as follows (GenBank Accession No M35999; SEQ ID NO: 87)

MRARPRPRPLWVTVLALGALAGVGVGGPNICTTRGVSSCQQCLAVSPMCAWCSDEALPLGSPRCDLKENLLKDN
CAPESIEFPVSEARVLEDRPLSDKGSGDSSQVTQVSPQRIALRLRPDDSKNFSIQVRQVEDYPVDIYYLMDLSY
SMKDDLWSIQNLGTKLATQMRKLTSNLRIGFGAFVDKPVSPYMYISPPEALENPCYDMKTTCLPMFGYKHVLTL
TDQVTRFNEEVKKQSVSRNRDAPEGGFDAIMQATVCDEKIGWRNDASHLLVFTTDAKTHIALDGRLAGIVQPND
GQCHVGSDNHYSASTTMDYPSLGLMTEKLSQKNINLIFAVTENVVNLYQNYSELIPGTTVGVLSMDSSNVLQLI
VDAYGKIRSKVELEVRDLPEELSLSFNATCLNNEVIPGLKSCMGLKIGDTVSFSIEAKVRGCPQEKEKSFTIKP
VGFKDSLIVQVTFDCDCACQAQAEPNSHRCNNGNGTFECGVCRCGPGWLGSQCECSEEDYRPSQQDECSPREGQ
PVCSQRGECLCGQCVCHSSDFGKITGKYCECDDFSCVRYKGEMCSGHGQCSCGDCLCDSDWTGYYCNCTTRTDT
CMSSNGLLCSGRGKCECGSCVCIQPGSYGDTCEKCPTCPDACTFKKECVECKKFDRGALHDENTCNRYCRDEIE
SVKELKDTGKDAVNCTYKNEDDCVVRFQYYEDSSGKSILYVVEEPECPKGPDILVVLLSVMGAILLIGLAALLI
WKLLITIHDRKEFAKFEEERARAKWDTANNPLYKEATSTFTNITYRGT

Autoimmune Gastritis Autoantigens and Bystander Antigens

In a further alternative embodiment of the present invention the autoantigen or bystander antigen may be an autoimmune gastritis autoantigen or bystander antigen for use to treat autoimmune gastritis.

The term “autoimmune gastritis” as used herein includes any disease in which gastric tissue or a component thereof comes under autoimmune attack. The term includes, for example, pernicious anemia.

The term “autoimmune gastritis autoantigen” as used herein includes any substance or a component thereof normally found within a mammal that, in autoimmune gastritis, becomes a target of attack by the immune system, preferably the primary (or a primary) target of attack. The term also includes antigenic substances that induce conditions having the characteristics of autoimmune gastritis when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “autoimmune gastritis bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the gastric tissue under autoimmune attack in autoimmune gastritis. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Autoimmune gastritis includes, in particular, pernicious anemia. Examples of autoimmune gastritis autoantigens and bystander antigens include, but are not limited to parietal cell antigens such as gastric H+/K+ ATPase, (100 kDa alpha subunit and 60-90 kDa beta subunit; especially the beta subunit) and intrinsic factor.

For example an amino acid sequence for a human H,K-ATPase beta subunit is reported as follows (GenBank Accession No M75110; SEQ ID NO: 88):

MAALQEKKTCGQRMEEFQRYCWNPDTGQMLGRTLSRWVWISLYYVAFYVVMTGLFALCLYVLMQTVDPYTPDYQ
DQLRSPGVTLRPDVYGEKGLEIVYNVSDNRTWADLTQTLHAFLAGYSPAAQEDSINCTSEQYFFQESFRAPNHT
KFSCKFTADMLQNCSGLADPNFGFEEGKPCFIIKMNRIVKFLPSNGSAPRVDCAFLDQPRELGQPLQVKYYPPN
GTFSLHYFPYYGKKAQPHYSNPLVAAKLLNIPRNAEVAIVCKVMAEHVTFNNPHDPYEGKVEFKLKIEK

(see also GenBank Accession No J05451; human gastric (H+/K+)-ATPase gene and GenBank Accession No M63962; human gastric H,K-ATPase catalytic subunit gene).
Autoimmune Hepatitis Autoantigens and Bystander Antigens

In an alternative embodiment of the present invention the autoantigen or bystander antigen may be an autoimmune hepatitis autoantigen or bystander antigen for use to treat autoimmune hepatitis.

The term “autoimmune hepatitis” as used herein includes any disease in which the liver or a component of the liver comes under autoimmune attack. The term thus includes, for example, primary biliary cirrhosis (PBC) and primary sclerosing cholangitis.

The term “autoimmune hepatitis autoantigen” as used herein includes any substance or a component thereof normally found within a mammal that, in autoimmune hepatitis, becomes a target of attack by the immune system, preferably the primary (or a primary) target of attack. The term also includes antigenic substances that induce conditions having the characteristics of autoimmune hepatitis when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “autoimmune hepatitis bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in autoimmune gastritis. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Examples of autoimmune hepatitis autoantigens and bystander antigens include, but are not limited to cytochrome P450s such as cytochrome P450 2D6, cytochrome P450 2C9 and cytochrome P450 1A2, the asialoglycoprotein receptor (ASGP R) and UDP-glucuronosyltransferases (UGTs).

For example, cDNA encoding human cytochrome P450-2d6 (coding for antigen for AIH Type2a LKM1 antibody) is reported as follows (GenBank Accession No E15820; SEQ ID NO: 89):

| | |
|---|---|
| 1 | atggggctag aagcactggt gcccctggcc atgatagtgg |
| | ccatcttcct gctcctggtg |
| 61 | gacctgatgc accggcgcca acgctgggct gcacgctacc |
| | caccaggccc cctgccactg |
| 121 | cccgggctgg gcaacctgct gcatgtggac ttccagaaca |
| | caccatactg cttcgaccag |
| 181 | ttgcggcgcc gacttcggga cgtgttcagc ctgcanctgg |
| | cctggacgcc ggtggtcgtg |
| 241 | ctcaatgggc tggcggccgt gcgcgaggcg ctggtgaccc |
| | acggcgagga caccgccgac |
| 301 | cgcccgcctg tgcccatcac ccagatcctg ggcttcgggc |
| | cgcgttccca aggggtgttc |
| 361 | ctggcgcgct atgggcccgc gtggcgcgag cagaggcgct |
| | tctccgtctc caccttgcgc |
| 421 | aacttgggcc tgggcaagaa gtcgctggag cagtgggtga |
| | ccgaggaggc ngcctgcctt |
| 481 | tgtgccgcct tcgccaacca ctccggacgc ccctttcgcc |
| | ccaacggtct cttggacaaa |
| 541 | gccgtgagca acgtgatcgc ctccctcacc tgcgggcgcc |
| | gcttcgagta cgacgaccct |
| 601 | cgcttcctca ggctgctgga cctagctcag gagggactga |
| | aggaggagtc gggctttctg |
| 661 | cgcgaggtgc tgaatgctgt ccccgtcctc ctgcatatcc |
| | cngcgctggc tggcaaggtc |
| 721 | ctacgcttcc aaaaggcttt cctgacccag ctggatgagc |
| | tgctaactga gcacaggatg |
| 781 | acctgggacc cagcccagcc cccccgagac ctgactgagg |
| | ccttcctggc agagatggag |
| 841 | aaggccaagg ggaaccctgc gagcagcttc aatgatgaga |
| | acctgcgcat agtggtggct |
| 901 | gacctgttct ctgccgggat ggtgaccacc tcgaccacgc |
| | tggcctgggg cctcctgctc |
| 961 | atgatcctac atccggatgt gcagcgccgt gtccaacagg |
| | agatcgacga cgtgataggg |
| 1021 | caggtgcggc gaccagagat gggtgaccag gctcacatgc |
| | cctacaccac tgccgtgatt |
| 1081 | catgaggtgc agcgctttgg ggacatcgtc cccctgggtg |
| | tgacccatat gacatcccgt |
| 1141 | gacatcgagg tacagggctt cngcatccct aagggaacga |
| | cactcatcac caacctgtca |
| 1201 | tcggtnctga aggatgaggc cgtctgggag aagcccttcc |
| | gcttccaccc cgaacacttc |
| 1261 | ctggatgccc agggccactt tgtgaagccg gaggccttcc |
| | tgcctttctc agcaggccgc |
| 1321 | cgtgcatgcc tcggggagcc cctggcccgc atggagctct |
| | tcctcttctt cacctccctg |
| 1381 | ctgcagcact tcagcttctc ggtgcccact ggacagcccc |
| | ggcccagcca ccatggtgtc |
| 1441 | tttgctttcc tggtgagccc atccccctat gagctttgtg |
| | ctgtgccccg ctagaatggg |
| 1501 | gtacctagtc cccagcctgc tcctagccca gaggctctaa |
| | tgtac |

An amino acid sequence for a human cytochrome P450-1A2 (CYP1A2) is reported as follows (GenBank Accession No AF182274; SEQ ID NO: 90):

MALSQSVPFSATELLLASAIFCLVFWVLKGLRPRVPKGLKSPPEPWGWPLLGHVLTLGKNPHLALSRMSQRYGD
VLQIRIGSTPVLVLSRLDTIRQALVRQGDDFKGRPDLYTSTLITDGQSLTFSTDSGPVWAARRRLAQNALNTFS
IASDPASSSSCYLEEHVSKEAMALISRLQELMAGPGHFDPYNQVVVSVANVIGAMCFGQHFPESSDEMLSLVKN
THEFVETASSGNPLDFFPILRYLPNPALQRFKAFNQRFLWFLQKTVQEHYQDFDKNSVRDITGALFKHSKKGPR
ASGNLIPQEKIVNLVNDVFGAGFDTVTTAISWSLMYLVTKPEIQRKIQKELDTVIGRERRPRLSDRPQLPYLEA
FILETFRHSSFLPFTIPHSTTRDTTLNGFYIPKKCCVFVNQWQVNHDPELWEDPSEFRPERFLTADGTAINKPL
SEKMMLFGMGKRRCIGEVLAKWEIFLFLAILLQQLEFSVPPGVKVDLIPIYGLTMKHARCEHVQARLRFSIN

Examples of primary biliary cirrhosis (PBC) autoantigens and bystander antigens include, but are not limited to mitochondrial antigens such as pyruvate dehydrogenase (E1-alpha decarboxylase, E1-beta decarboxylase and E2 acetyltransferase), branched-chain 2-oxo-acid dehydrogenases and 2-oxoglutarate dehydrogenases.

Autoimmune Vasculitis Autoantigens and Bystander Antigens

In a further alternative embodiment of the present invention the autoantigen or bystander antigen may be an autoimmune vasculitis autoantigen or bystander antigen for use to treat autoimmune vasculitis.

The term “autoimmune vasculitis” as used herein includes any disease in which blood vessels or a component thereof come under autoimmune attack and includes, for example, large vessel vasculitis such as giant cell arteritis and Takayasu's disease, medium-sized vessel vasculitis such as polyarteritis nodosa and Kawasaki disease and small vessel vasculitis such as Wegener's granulomatosis, Churg-Strauss syndrome, microscopic polyangiitis, Henoch Schonlein purpura, essential cryoglobulinaemic vasculitis and cutaneous leukocytoclastic angiitis.

The term “autoimmune vasculitis autoantigen” as used herein includes any substance or a component thereof normally found within a mammal that, in autoimmune vasculitis, becomes a target of attack by the immune system, preferably the primary (or a primary) target of attack. The term also includes antigenic substances that induce conditions having the characteristics of autoimmune vasculitis when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “autoimmune vasculitis bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the blood vessel tissue under autoimmune attack in autoimmune vasculitis. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Examples of vasculitis autoantigens and bystander antigens include, but are not limited to basement membrane antigens (especially the noncollagenous domain of the alpha 3 chain of type IV collagen) and endothelial cell antigens.

Ocular Autoantigens and Bystander Antigens

In a further alternative embodiment of the present invention the autoantigen or bystander antigen may be an ocular autoantigen or bystander antigen for use to treat an autoimmune disease of the eye.

The term “autoimmune disease of the eye” includes any disease in which the eye or a component thereof comes under autoimmune attack. The term thus includes, for example, cicatricial pemphigoid, uveitis, Mooren's ulcer, Reiter's syndrome, Behcet's syndrome, Vogt-Koyanagi-Harada Syndrome, scleritis, lens-induced uveitis, optic neuritis and giant-cell arteritis.

The term “ocular autoantigen” as used herein includes any substance or a component thereof normally found within the eye of a mammal that, in an autoimmune disease of the eye, becomes a target of attack by the immune system, preferably the primary (or a primary) target of attack. The term also includes antigenic substances that induce conditions having the characteristics of autoimmune disease when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “ocular bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the eye under autoimmune attack. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Examples of ocular autoantigens and bystander antigens include, but are not limited to retinal antigens such as ocular antigen, S-antigen, interphotoreceptor retinoid binding protein (see e.g. Exp. Eye Res. 56:463 (93)) in uveitis and alpha crystallin in lens-induced uveitis.

An amino acid sequence for a human retinal S-antigen (48 KDa protein) is reported as follows (GenBank Accession No X12453; SEQ ID NO: 91):

MAASGKTSKSEPNHVIFKKISRDKSVTIYLGNRDYIDHVSQVQPVDGVVLVDPDLVKGKKVYVTLTCAFRYGQE
DVDVIGLTFRRDLYFSRVQVYPPVGAASTPTKLQESLLKKLGSNTYPFLLTFPDYLPCSVMLQPAPQDSGKSCG
VDFEVKAFATDSTDAEEDKIPKKSSVRYLIRSVQHAPLEMGPQPRAEATWQFFMSDKPLHLAVSLNREIYFHGE
PIPVTVTVTNNTEKTVKKIKACVEQVANVVLYSSDYYVKPVAMEEAQEKVPPNSTLTKTLTLLPLLANNRERRG
IALDGKIKHEDTNLASSTIIKEGIDRTVLGILVSYQIKVKLTVSGFLGELTSSEVATEVPFRLMHPQPEDPAKE
SIQDANLVFEEFARHNLKDAGEAEEGKRDKNDADE

An amino acid sequence for a human alpha crystallin is reported as follows (GenBank Accession No U05569; SEQ ID NO: 92):

MDVTIQHPWFKRTLGPFYPSRLFDQFFGEGLFEYDLLPFLSSTISPYYRQSLFRTVLDSGISEVRSDRDKFVIF
LDVKHFSPEDLTVKVQDDFVEIHGKHNERQDDHGYISREFHRRYRLPSNVDQSALSCSLSADGMLTFCGPKIQT
GLDATHAERAIPVSREEKPTSAPSS

Adrenal Autoantigens and Bystander Antigens

In a further alternative embodiment of the present invention the autoantigen or bystander antigen may be an adrenal autoantigen or bystander antigen for use to treat adrenal autoimmune disease.

The term “adrenal autoimmune disease” as used herein includes any disease in which the adrenal gland or a component thereof comes under autoimmune attack. The term includes, for example, Addison's disease.

The term “adrenal autoantigen” as used herein includes any substance or a component thereof normally found within a mammal that, in adrenal autoimmune disease, becomes a target of attack by the immune system, preferably the primary (or a primary) target of attack. The term also includes antigenic substances that induce conditions having the characteristics of adrenal autoimmune disease when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “adrenal bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the adrenal gland under autoimmune attack in adrenal autoimmune disease. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction. Examples of adrenal autoantigens and bystander antigens include, but are not limited to adrenal cell antigens such as the adrenocorticotropic hormone receptor (ACTH receptor) and enzymes such as 21-hydroxylase and 17-hydroxylase.

For example, an amino acid sequence for a human steroid 17-alpha-hydroxylase is reported as follows (GenBank Accession No NM_{—}000102; SEQ ID NO: 93):

MWELVALLLLTLAYLFWPKRRCPGAKYPKSLLSLPLVGSLPFLPRHGHMHNNFFKLQKKYGPIYSVRMGTKTTV
IVGHHQLAKEVLIKKGKDFSGRPQMATLDIASNNRKGIAFADSGAHWQLHRRLAMATFALFKDGDQKLEKIICQ
EISTLCDMLATHNGQSIDISFPVFVAVTNVISLICFNTSYKNGDPELNVIQNYNEGIIDNLSKDSLVDLVPWLK
IFPNKTLEKLKSHVKIRNDLLNKILENYKEKFRSDSITNMLDTLMQAKMNSDNGNAGPDQDSELLSDNHILTTI
GDIFGAGVETTTSVVKWTLAFLLHNPQVKKKLYEEIDQNVGFSRTPTISDRNRLLLLEATIREVLRLRPVAPML
IPHKANVDSSIGEFAVDKGTEVIINLWALHHNEKEWHQPDQFMPERFLNPAGTQLISPSVSYLPFGAGPRSCIG
EILARQELFLIMAWLLQRFDLEVPDDGQLPSLEGIPKVVFLIDSFKVKIKVRQAWREAQAEGST

(see also Krohn et al: Identification by molecular cloning of an autoantigen associated with Addison's disease as steroid 17 alpha-hydroxylase, Lancet 339 (8796), 770-773 (1992))
Cardiovascular Autoantigens and Bystander Antigens

In a further alternative embodiment of the present invention the autoantigen or bystander antigen may be a cardiac autoantigen or bystander antigen for use to treat cardiac autoimmune disease.

The term “cardiac autoimmune disease” as used herein includes any disease in which the heart or a component thereof comes under autoimmune attack. The term includes, for example, autoimmune myocarditis, dilated cardiomyopathy, autoimmune rheumatic fever and Chagas' disease.

The term “cardiac autoantigen” as used herein includes any substance or a component thereof normally found within a mammal that, in cardiac autoimmune disease, becomes a target of attack by the immune system, preferably the primary (or a primary) target of attack. The term also includes antigenic substances that induce conditions having the characteristics of cardiac autoimmune disease when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “cardiac bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the heart tissue under autoimmune attack in cardiac autoimmune disease. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Examples of cardiac autoantigens and bystander antigens include, but are not limited to heart muscle cell antigens such as mysosin; laminin, beta-1 adrenergic receptors, adenine nucleotide translocator (ANT) protein and branched-chain ketodehydrogenase (BCKD).

Scleroderma/Polymyositis Autoantigens and Bystander Antigens

In an alternative embodiment of the present invention the autoantigen or bystander antigen may be a scleroderma or myositis autoantigen or bystander antigen for use to treat scleroderma or myositis.

The term “myositis/scleroderma autoantigen” as used herein includes any substance or a component thereof normally found within a mammal that, in myositis (particularly in dermatomyositis or polymyositis) or scleroderma, becomes a target of attack by the immune system, preferably the primary (or a primary) target of attack. The term also includes antigenic substances that induce conditions having the characteristics of myositis (particularly in dermatomyositis or polymyositis) or scleroderma when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “myositis/scleroderma bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in myositis (particularly in dermatomyositis or polymyositis) or scleroderma. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

As described, for example, in U.S. Pat. No. 5,862,360, scleroderma, or systemic sclerosis, is characterized by deposition of fibrous connective tissue in the skin, and often in many other organ systems. It may be accompanied by vascular lesions, especially in the skin, lungs, and kidneys. The course of this disease is variable, but it is usually slowly progressive. Scleroderma may be limited in scope and compatible with a normal life span. Systemic involvement, however, can be fatal.

Scleroderma may be classified as either diffuse or limited, on the basis of the extent of skin and internal organ involvement. The diffuse form is characterized by thickening and fibrosis of skin over the proximal extremities and trunk. The heart, lungs, kidneys, and gastrointestinal tract below the esophagus are often involved. Limited scleroderma is characterized by cutaneous involvement of the hands and face. Visceral involvement occurs less commonly. The limited form has a better prognosis than the diffuse form, except when pulmonary hypertension is present.

Antinuclear antibodies are found in over 95 percent of patients with scleroderma. Specific antinuclear antibodies have been shown to be directed to topoisomerase I, centromere proteins, RNA polymerases, or nucleolar components. Different antibodies are associated with particular clinical patterns of scleroderma. For example, antibodies to topoisomerase I (Scl-70) and to RNA polymerases (usually RNA polymerase III) are seen in patients with diffuse scleroderma. Antibodies to nuclear ribonucleoprotein (NRNP) are associated with diffuse and limited scleroderma.

Patients with scleroderma typically show autoreactivity against centrosomes (Tuffanelli, et al., Arch. Dermatol., 119:560-566, 1983). Centrosomes are essential structures that are highly conserved, from plants to mammals, and are important for various cellular processes. Centrosomes play a crucial role in cell division and its regulation. Centrosomes organize the mitotic spindle for separating chromosomes during cell division, thus ensuring genetic fidelity. In most cells, the centrosome includes a pair of centrioles that lie at the center of a dense, partially filamentous matrix, the pericentriolar material (PCM). The microtubule cytoskeleton is anchored to the centrosome or some other form of microtubule organizing center (MTOC), which is thought to serve as a site of microtubule nucleation.

As discussed in U.S. Pat. No. 6,160,107 the idiopathic inflammatory myopathies polymyositis, dermatomyositis and the related overlap syndromes disorder, such as polymyositis-scleroderma overlap, are inflammatory myopathies that are characterized by chronic muscle inflammation and proximal muscle weakness. The muscle inflammation causes muscle tenderness, muscle weakness, and ultimately muscle atrophy and fibrosis (see, for example, Plotz, et al. Annals of Internal Med. 111: 143-157(1989)). Also associated with the muscle inflammation are elevated serum levels of aldolase, creatine kinase, transaminases, such as alanine aminotransferase and aspartate aminotransferase, and lactic dehydrogenase. Other systems besides muscle can be affected by these conditions, resulting in arthritis, Raynaud's phenomenon, and interstitial lung disease. Clinically, polymyositis and dermatomyositis are distinguished by the presence of a characteristic rash in patients with dermatomyositis. Differences in the myositis of these conditions can be distinguished in some studies of muscle pathology.

Autoantibodies can be detected in about 90% of patients with polymyositis and dermatomyositis (Reichlin and Arnett, Arthritis and Rheum. 27: 1150-1156 (1984)). Sera from about 60% of these patients form precipitates with bovine thymus extracts on Ouchterlony immunodiffusion (ID), while sera from other patients stain tissue culture substrates, such as HEp-2 cells, by indirect immunofluorescence (IIF) (see, e.g., Targoff and Reichlin Arthritis and Rheum. 28: 796-803 (1985); Nishikai and Reichlin Arthritis and Rheum. 23: 881-888 (1980); Reichlin, et al., J. Clin. Immunol. 4:40-44 (1984)). There are numerous precipitating autoantibody specificities in myositis patients, but each individual antibody specificity occurs in only a fraction of the patients.

Many autoantibodies associated with myositis or myositis-overlap syndromes have been defined, and, in some cases, the antibodies have been identified. These include antibodies that are present in other disorders and also disease-specific antibodies (see, e.g., (Targoff and Reichlin Mt. Sinai J. of Med. 55: 487-493 (1988)). For example, a group of myositis-associated autoantibodies have been identified which are directed at cytoplasmic proteins that are related to tRNA and protein synthesis, particularly aminoacyl-tRNA synthetases. These include anti-Jo-1, which is the most common autoantibody associated with myositis autoimmune disorders (about 20% of such patients (Nishikai, et al. Arthritis Rheum. 23: 881-888 (1980)) and which is directed against histidyl-tRNA synthetase; anti-PL-7, which is directed against threonyl-tRNA synthetase; and anti-PL12, which is directed against alanyl-tRNA synthetase. Anti-U1 RNP, which is frequently found in patients with SLE, may also be found in mixed connective tissue disease, overlap syndromes involving myositis, or in some cases of myositis alone. This antibody reacts with proteins that are uniquely present on the U1 small nuclear ribonucleoprotein, which is one of the nuclear RNPs that are involved in splicing mRNA. Autoantibodies such as anti-Sm, anti-Ro/SSA, and anti-La/SSB, that are usually associated with other conditions, are sometimes found in patients with overlap syndromes. Anti-Ku has been found in myositis-scleroderma overlap syndrome and in SLE. The Ku antigen is a DNA binding protein complex with two polypeptide components, both of which have been cloned.

Anti Jo-1 and other anti-synthetases are disease specific. Other myositis-associated antibodies are anti-PM-Scl, which is present in about 5-10% of myositis patients, many of whom have polymyositis-scleroderma overlap, and anti-Mi-2, which is present in about 8% of myositis patients, almost exclusively in dermatomyositis. Mi-2 is found in high titer in about 20% of all dermatomyositis patients and in low titer in less than 5% of polymyositis patients (see, e.g., Targoff and Reichlin, Mt. Sinai J. of Med. 55: 487-493 (1988)).

Anti-Mi was first described by Reichlin and Mattioli, Clin. Immunol. and Immunopathol. 5: 12-20 (1976)). A complement-fixation reaction was used to detect it and, in that study, patients with dermatomyositis, polymyositis and polymyositis overlap syndromes had positive reactions. The prototype or reference serum, from patient Mi, forms two precipitin lines on immunodiffusion (ID) with calf thymus antigens, Mi-1 and Mi-2. Mi-1, which has been purified from bovine thymus nuclear extracts (Nishikai, et al. Mol. Immunol. 17: 1129-141 (1980)) is rarely found in other sera and is not myositis specific (Targoff, et al., Clin. Exp. Immunol. 53: 76-82 (1983)).

Anti-Mi-2 was found to be a myositis-specific autoantibody by Targoff, et al. Arthritis and Rheum. 28: 796-803 (1985). Furthermore, all patients with the antibody have the dermatomyositis rash.

Bovine thymus Mi-2 antigen was originally found to be a nuclear protein that separates in SDS polyacrylamide (SDS-PAGE) gels into two bands with apparent molecular weights of 53 kilodaltons (hereinafter kDa) and 61 KDa, respectively. Recently, additional higher molecular weight bands have been found. The bovine thymus antigenic activity is destroyed by SDS-PAGE and is trypsin sensitive, but not RNAse sensitive (Targroff et al. Arthritis and Rheum. 28: 796-803 (1985)).

Anti-PM-1 was first identified as an antibody found in 61% of dermatomyositis/polymyositis patients, including patients; with polymyositis-scleroderma overlap (Wolfe, et al. J. Clin. Invest. 59: 176-178 (1977)). PM-1 was subsequently shown to be more than one antibody. The unique specificity component of PM-1 was later named PM-Scl (Reichlin, et al. J. Clin. Immunol. 4: 40-44 (1984)). Anti-PM-Scl is found in the sera of about 5-10% of myositis patients, but is most commonly associated with polymyositis-scleroderma overlap syndrome. It also occurs in patients with polymyositis or dermatomyositis alone or in patients with scleroderma without myositis.

Anti-PM-Scl antibody immunoprecipitates a complex from HeLa cell extracts of at least eleven polypeptides that have molecular weights ranging from about 20 to 110 kDa (see, Reimer, et al., J. Immunol. 137:3802-3808 (1986). The antigen is trypsin-sensitive, occurs in nucleoli (see, e.g., Targoff and Reichlin Arthritis Rheum. 28: 226-230 (1985)) and is believed to be a preribosomal particle.

In an abstract, Bluthner, et al., First Int. Workshop on the Mol. and Cell Biology of Autoantibodies and Autoimmunity in Heidelberg (Springer-Verlag Jul. 27-29, 1989) report that sera from patients suffering from polymyositis/scleroderma-overlap syndrome (PM/Scl) recognize two major nucleolar proteins of 95 and 75 kDa molecular weight in Western blots of a Hela cell extract. They also report that cDNA that encodes a 20 kDa protein reactive with autoantibodies eluting from the 95 kDa PM-Scl HeLa antigen subunit has been cloned from a HeLa cDNA library. The sequence of the cloned DNA has not as yet been reported.

It will be appreciated that combinations of myositis/scleroderma autoimmune/bystander antigens and myositis/scleroderma autoimmune/bystander antigenic determinants and/or polynucleotide sequences coding for them may also be used as appropriate.

Examples of myositis/scleroderma autoantigens and myositis/scleroderma bystander antigens include, but are not limited to, Jo-1 (his-tRNA synthetase), PM-Scl, Mi-2, Ku, PL-7 (thr-tRNA synthetase), PL-12 (ala-tRNA-synthetase), SRP (signal recognition particle), Anti-nRNP (U1 small nuclear RNP), Ro/SS-A, and La/SS-B.

For example, an amino acid sequence for a human 100 kD Pm-Scl autoantigen protein (PM/Scl-100a) is reported as follows (GenBank Accession No L01457; SEQ ID NO: 94):

MAPPSTREPRVLSATSATKSDGEMVLPGFPDADSFVKFALGSVVAVTKASGGLPQFGDEYDFYRSFPGFQAFCE
TQGDRLLQCMSRVMQYHGCRSNIKDRSKVTELEDKFDLLVDANDVILERVGILLDEASGVNKNQQPVLPAGLQV
PKTVVSSWNRKAAEYGKKAKSETFRLLHAKNIIRPQLKFREKIDNSNTPFLPKIFIKPNAQKPLPQALSKERRE
RPQDRPEDLDVPPALADFIHQQRTQQVEQDMFAHPYQYELNHFTPADAVLQKPQPQLYRPIEETPCHFISSLDE
LVELNEKLLNCQEFAVDLEHHSYRSFLGLTCLMQISTRTEDFIIDTLELRSDMYILNESLTDPAIVKVFHGADS
DIEWLQKDFGLYVVNMFDTHQAARLLNLGRHSLDHLLKLYCNVDSNKQYQLADWRIRPLPEEMLSYARDDTHYL
LYIYDKMRLEMWERGNGQPVQLQVVWQRSRDICLKKFIKPIFTDESYLELYRKQKKHLNTQQLTAFQLLFAWRD
KTARREDESYGYVLPNHMMLKIAEELPKEPQGIIACCNPVPPLVRQQINEMHLLIQQAREMPLLKSEVAAGVKK
SGPLPSAERLENVLFGPHDCSHAPPDGYPIIPTSGSVPVQKQASLFPDEKEDNLLGTTCLIATAVITLFNEPSA
EDSKKGPLTVAQKKAQNIMESFENPFRMISNRWKLAQVQVQKDSKEAVKKKAAEQTAAREQAKEACKAAAEQAI
SVRQQVVLENAAKKRERATSDPRTTEQKQEKKRLKISKKPKDPEPPEKEFTPYDYSQSDFKAFAGNSKSKVSSQ
FDPNKQTPSGKKCIAAKKIKQSVGNKSMSFPTGKSDRGFRYNWPQR

(see also Gee et al, Cloning of a complementary DNA coding for the 100-kD antigenic protein of the PM-Scl autoantigen, J. Clin. Invest. 90 (2), 559-570 (1992))

An amino acid sequence for a human 100 kD Pm-Scl autoantigen protein (PM/Scl-100b) is reported as follows (GenBank Accession No X66113; SEQ ID NO: 95):

MAPPSTREPRVLSATSATKSDGEMVLPGFPDADSFVKFALGSVVAVTKASGGLPQFGDEYDFYRSFPGFQAFCE
TQGDRLLQCMSRVMQYHGCRSNIKDRSKVTELEDKFDLLVDANDVILERVGILLDEASGVNKNQQPVLPAGLQV
PKTVVSSWNRKAAEYGKKAKSETFRLLHAKNIIRPQLKFREKIDNSNTPFLPKIFIKPNAQKPLPQALSKERRE
RPQDRPEDLDVPPALADFIHQQRTQQVEQDMFAHPYQYELNHFTPADAVLQKPQPQLYRPIEETPCHFISSLDE
LVELNEKLLNCQEFAVDLEHHSYRSFLGLTCLMQISTRTEDFIIDTLELRSDMYILNESLTDPAIVKVFHGADS
DIEWLQKDFGLYVVNMFDTHQAARLLNLGRHSLDHLLKLYCNVDSNKQYQLADWRIRPLPEEMLSYARDDTHYL
LYIYDKMRLEMWERGNGQPVQLQVVWQRSRDICLKKFIKPIFTDESYLELYRKQKKHLNTQQLTAFQLLFAWRD
KTARREDESYGYVLPNHMMLKIAEELPKEPQGIIACCNPVPPLVRQQINEMHLLIQQAREMPLLKSEVAAGVKK
SGPLPSAERLENVLFGPHDCSHAPPDGYPIIPTSGSVPVQKQASLFPDEKEDNLLGTTCLIATAVITLFNEPSA
EDSKKGPLTVAQKKAQNIMESFENPFRMFLPSLGHRAPVSQAAKFDPSTKIYEISNRWKLAQVQVQKDSKEAVK
KKAAEQTAAREQAKEACKAAAEQAISVRQQVVLENAAKKRERATSDPRTTEQKQEKKRLKISKKPKDPEPPEKE
FTPYDYSQSDFKAFAGNSKSKVSSQFDPNKQTPSGKKCIAAKKIKQSVGNKSMSFPTGKSDRGFRYNWPQR

(see also Bluthner and Bautz, Cloning and characterization of the cDNA coding for a polymyositis-scleroderma overlap syndrome-related nucleolar 100-kD protein, J. Exp. Med. 176 (4), 973-980 (1992))

An amino acid sequence for a human75 kD Pm-Scl autoantigen protein (PM/Scl-75a) is reported as follows (GenBank Accession No M58460; SEQ ID NO: 96):

MAAPAFEPGRQSDLLVKLNRLMERCLRNSKCIDTESLCVVAGEKVWQIRVDLHLLNHDGNIIDAASIAAIVALC
HFRRPDVSVQGDEVTLYTPEERDPVPLSIHHMPICVSFAFFQQGTYLLVDPNEREERVMDGLLVIAMNKHREIC
TIQSSGGIMLLKDQVLRCSKIAGVKVAEITELILKALENDQKVRKEGGKFGFAESIANQRITAFKMEKAPIDTS
DVEEKAEEIIAEAEPPSEVVSTPVLWTPGTAQIGEGVENSWGDLEDSEKEDDEGGGDQAIILDGIKMDTGVEVS
DIGSQDAPIILSDSEEEEMIILEPDKNPKKIRTQTTSAKQEKAPSKKPVKRRKKKRAAN

(see also Alderuccio et al, Molecular characterization of an autoantigen of PM-Scl in the polymyositis/scleroderma overlap syndrome: a unique and complete human cDNA encoding an apparent 75-kD acidic protein of the nucleolar complex, J. Exp. Med. 173 (4), 941-952 (1991))

An amino acid sequence for a human 75 kD Pm-Scl autoantigen protein (PM/Scl-75b) is reported as follows (GenBank Accession No U09215; SEQ ID NO: 97):

MAAPAFEPGRQSDLLVKLNRLMERCLRNSKCIDTESLCVVAGEKVWQIRVDLHLLNHDGNIIDAASIAAIVALC
HFRRPDVSVQGDEVTLYTPEERDPVPLSIHHMPICVSFAFFQQGTYLLVDPNEREERVMDGLLVIAMNKHREIC
TIQSSGGIMLLKDQVLRCSKIAGVKVAEITELILKALENDQKVRKEGGKFGFAESIANQRITAFKMEKAPIDTS
DVEEKAEEIIAEAEPPSEVVSTPVLWTPGTAQIGEGVENSWGDLEDSEKEDDEGGGDQAIILDGIKMDTGVEVS
DIGSQELGFHHVGQTGLEFLTSDAPIILSDSEEEEMIILEPDKNPKKIRTQTTSAKQEKAPSKKPVKRRKKKRA
AN

An amino acid sequence for a Jo-1 (histidyl-tRNA synthetase) autoantigen protein is reported as follows (GenBank Accession No Z11518; SEQ ID NO: 98):

MAERAALEELVKLQGERVRGLKQQKASAELIEEEVAKLLKLKAQLGPDESKQKFVLKTPKGTRDYSPRQMAVRE
KVFDVIIRCFKRHGAEVIDTPVFELKETLMGKYGEDSKLIYDLKDQGGELLSLRYDLTVPFARYLAMNKLTNIK
RYHIAKVYRRDNPAMTRGRYREFYQCDFDIAGNFDPMIPDAECLKIMCEILSSLQIGDFLVKVNDRRILDGMFA
ICGVSDSKFRTICSSVDKLDKVSWEEVKNEMVGEKGLAPEVADRIGDYVQQHGGVSLVEQLLQDPKLSQNKQAL
EGLGDLKLLFEYLTLFGIDDKISFDLSLARGLDYYTGVIYEAVLLQTPAQAGEEPLGVGSVAAGGRYDGLVGMF
DPKGRKVPCVGLSIGVERIFSIVEQRLEALEEKIRTTETQVLVASAQKKLLEERLKLVSELWDAGIKAELLYKK
NPKLLNQLQYCEEAGIPLVAIIGEQELKDGVIKLRSVTSREEVDVRREDLVEEIKRRTGQPLCIC

(see also Raben et al, Human histidyl-tRNA synthetase: recognition of amino acid signature regions in class 2a aminoacyl-tRNA synthetases, Nucleic Acids Res. 20 (5), 1075-1081 (1992))

An amino acid sequence for a PL-7 (threonyl-tRNA synthetase) autoantigen protein is reported as follows (GenBank Accession No M63180; SEQ ID NO: 99):

MGGEEKPIGAGEEKQKEGGKKKNKEGSGDGGRAELNPWPEYIYTRLEMYNILKAEHDSILAEKAEKDSKPIKVT
LPDGKQVDAESWKTTPYQIACGISQGLADNTVIAKVNNVVWDLDRPLEEDCTLELLKFEDEEAQAVYWHSSAHI
MGEGMERVYGGCLCYGPPIENGFYYDMYLEEGGVSSNDFSSLEALCKKIIKEKQAFERLEVKKETLLAMFKYNK
FKCRILNEKVNTPTTTVYRCGPLIDLCRGPHVRHTGKIKALKIHKNSSTYWEGKADMETLQRIYGISFPDPKML
KEWEKFQEEAKNRDHRKIGRDQELYFFHELSPGSCFFLPKGVYIYNALIEFIRSEYRKRGFQEVVTPNIFNSRL
WMTSGHWQHYSENMFSFEVEKELFALKPMNCPGHSLMFDHRPRSWRELPLRLADFGGLHRNELSGALTGLTRVR
RFQQDDAHIFCAMEQIEDEIKGCLDFLRTVYSVFGFSFKLNLSTRPEKFLGDIEVWDQAEKQLENSLNEFGEKW
ELNSGDGAFYGPKIDIQIKDAIGRYHQCATIQLDFQLPIRFNLTYVSHDGEDKKRPVIVHRAILGSVERMIAIL
TENYGGKLAPFWLSPRQVMVVPVGPTCDEYAQNVRQQFHDAKFMADIDLDPGCTLNKKIRNAQLAQYNFILVVG
EKEKITGTVNIRTRDNKVHGERTISETIERLQQLKEFRSKQAEEEF

(See also Cruzen et al, Nucleotide and deduced amino acid sequence of human threonyl-tRNA synthetase reveals extensive homology to the Escherichia coli and yeast enzymes, J. Biol. Chem. 266 (15), 9919-9923 (1991))

An amino acid sequence for a PL-12 (alanyl-tRNA synthetase) autoantigen protein is reported as follows (GenBank Accession No D32050; SEQ ID NO: 100):

MDSTLTASEIRQRFIDFFKRNEHTYVHSSATIPLDDPTLLFANAGMNQFKPIFLNTIDPSHPMAKLSRAANTQK
CIRAGGKQNDLDDVGKDVYHHTFFEMLGSWSFGDYFKELACKMALELLTQEFGIPIERLYVTYFGGDEAAGLEA
DLECKQIWQNLGLDDTKILPGNMKDNFWEMGDTGPCGPCSEIHYDRIGGRDAAHLVNQDDPNVLEIWNLVFIQY
NREADGILKPLPKKSIDTGMGLERLVSVLQNKMSNYDTDLFVPYFEAIQKGTGARPYTGKVGAEDADGIDMAYR
VLADHARTITVALADGGRPDNTGRGYVLRRILRRAVRYAHEKLNASRGFFATLVDVVVQSLGDAFPELKKDPDM
VKDIINEEEVQFLKTLSRGRRILDRKIQSLGDSKTIPGDTAWLLYDTYGFPVDLTGLIAEEKGLVVDMDGFEEE
RKLAQLKSQGKGAGGEDLIMLDIYAIEELRARGLEVTDDSPKYNYHLDSSGSYVFENTVATVMALRREKMFVEE
VSTGQECGVVLDKTCFYAEQGGQIYDEGYLVKVDDSSEDKTEFTVKNAQVRGGYVLHIGTIYGDLKVGDQVWLF
IDEPRRRPIMSNHTATHILNFALRSVLGEADQKGSLVAPDRLRFDFTAKGAMSTQQIKKAEEIANEMIEAAKAV
YTQDCPLAAAKAIQGLRAVFDETYPDPVRVVSIGVPVSELLDDPSGPAGSLTSVEFCGGTHLRNSSHAGAFVIV
TEEAIAKGIRRIVAVTGAEAQKALRKAESLKKCLSVMEAKVKAQTAPNKDVQREIADLGEALATAVIPQWQKDE
LRETLKSLKKVMDDLDRASKADVQKRVLEKTKQFIDSNPNQPLVILEMESGASAKALNEALKLFKMHSPQTSAM
LFTVDNEAGKITCLCQVPQNAANRGLKASEWVQQVSGLMDGKGGGKDVSAQATGKNVGCLQEALQLATSFAQLR
LGDVKN

An amino acid sequence for an EJ (glycyl-tRNA synthetase) autoantigen protein is reported as follows (GenBank Accession No U09587; SEQ ID NO: 101):

MDGAGAEEVLAPLRLAVRQQGDLVRKLKEDKAPQVDVDKAVAELKARKRVLEAKELALQPKDDIVDRAKMEDTL
KRRFFYDQAFAIYGGVSGLYDFGPVGCALKNNIIQTWRQHFTQEEQILEIDCTMLTPEPVLKTSGHVDKFADFM
VKDVKNGECFRADHLLKAHLQKLMSDKKCSVEKKSEMESVLAQLDNYGQQELADLFVNYNVKSPITGNDLSPPV
SFNLMFKTFIGPGGNMPGYLRPETAQGIFLNFKRLLEFNQGKLPFAAAQIGNSFRNEISPRSGLIRVREFTMAE
IEHFVDPSEKDHPKFQNVADLHLYLYSAKAQVSGQSARKMRLGDAVEQGVINNTVLGYFIGRIYLYLTKVGISP
DKLRFRQHMENEMAHYACDCWDAESKTSYGWIEIVGCADRSCYDLSCHARATKVPLVAEKPLKEPKTVNVVQFE
PSKGAIGKAYKKDAKLVMEYLAICDECYITEMEMLLNEKGEFTIETEGKTFQLTKDMINVKRFQKTLYVEEVVP
NVIEPSFGLGRIMYTVFEHTFHVREGDEQRTFFSFPAVVAPFKCSVLPLSQNQEFMPFVKELSEALTRHGVSHK
VDDSSGSIGRRYARTDEIGVAFGVTIDFDTVNKTPHTATLRDRDSMRQIRAEISELPSIVQDLANGNITWADVE
ARYPLFEGQETGKKETIEE

Further sequences are provided, for example, under GenBank Accession Nos AF241268.1, AF353396.1 (scleroderma-associated autoantigen); NM_{—}005033.1 (polymyositis/scleroderma autoantigen 1 (75 kDa) (PMSCL1)); XM_{—}001527.4, NM_{—}002685.1 (polymyositis/scleroderma autoantigen 2 (100 kDa) (PMSCL2)).

Nervous system Autoantigens and Bystander Antigens

In an alternative embodiment of the present invention the autoantigen or bystander antigen may be a nervous system autoantigen or bystander antigen for use to treat an autoimmune disease of the nervous system.

The term “autoimmune disease of the nervous system” includes any disease in which nervous tissue or a component thereof comes under autoimmune attack. The term includes, for example central nervous system diseases having an autoimmune etiology such as multiple sclerosis (MS), perivenous encephalomyelitis, autoimmune myelopathies, paraneoplastic cerebellar degeneration, paraneoplastic limbic (cortical) degeneration, stiff man syndrome, choreas (such as Sydenham's chorea), stroke, focal epilepsy and migraine; and peripheral nervous system diseases having an autoimmune etiology such as Guillain-Barre syndrome, Miller Fisher syndrome, chronic inflammatory demyelinating neuropathy, multifocal motor neuropathy with conduction block, demyelinating neuropathy associated with anti-myelin-associated glycoprotein antibodies, paraneoplastyic sensory neuropathy, POEMS, dorsal root ganglion neuronitis, acute panautonomic neuropathy and brachial neutritis.

The term “nervous system autoantigen” as used herein includes any nervous system substance or a component thereof normally found within a mammal that, in an autoimmune disease of the nervous system, becomes a target of attack by the immune system, preferably the primary (or a primary) target of attack. The term also includes antigenic substances that induce conditions having the characteristics of an autoimmune disease of the nervous system when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “nervous system bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in an autoimmune disease of the nervous system. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Preferably the nervous system autoantigen or nervous system bystander antigen is an MS autoantigen or MS bystander antigen.

The term “MS autoantigen” as used herein includes any nervous system substance or a component thereof normally found within a mammal that, in multiple sclerosis (MS), becomes a target of attack by the immune system, preferably the primary (or a primary) target of attack. The term also includes antigenic substances that induce conditions having the characteristics of MS when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “MS bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of nervous tissue under autoimmune attack in MS. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

It will be appreciated that combinations of nervous system autoimmune/bystander antigens and nervous system autoimmune/bystander antigenic determinants and/or polynucleotide sequences coding for them may also be used as appropriate.

Examples of nervous system autoantigens and nervous system bystander antigens include, but are not limited to, myelin basic protein (MBP), DM20, central nervous system white matter; proteolipid protein (PLP); myelin oligodendrocyte-associated protein (MOG), myelin associated glycoprotein (MAG), alpha B-crystallin (see e.g. J. Chromatog. Biomed. Appl. 526:535 (90))

The protein components of myelin proteins, including myelin basic protein (MBP) I proteolipid protein (PLP), myelin-associated glycoprotein (MAG) and myelin oligodendrocyte glycoprotein (MOG), are of particular interest. The suppression of T cell responsiveness to these antigens may be used to prevent or treat demyelinating diseases.

Proteolipid is a major constituent of myelin, and is known to be involved in demyelinating diseases (see, for example Greer et al. (1992) J. Immunol. 149: 783-788 and Nicholson (1997) Proc. Natl. Acad. Sci. USA 94: 9279-9284).

The integral membrane protein PLP is a dominant autoantigen of myelin.

Determinants of PLP antigenicity have been identified in several mouse strains, and includes residues 139-151 (Tuohy et al. (1989) J. Immunol. 142: 1523-1527), residues 103-116 (Tuohy et al. (1988) J. Immunol. 141: 1126-1130), residues 215-232 (Endoh et al. (1990) Int. Arch. Allerqv Appl. Immunol. 92: 433-438), residues 43-64 (Whitham et al (1991) J. Immunol. 147: 3803-3808) and residues 178-191 (Greer, et al. (1992) J. Immunol. 149: 783-788). Immunization with native PLP or with synthetic peptides corresponding to PLP epitopes induces experimental allergic encephalomyelitis (EAE). Analogues of PLP peptides generated by amino acid substitution can prevent EAE induction and progression (Kuchroo et al. (1994) J. Immunol. 153: 3326-3336, Nicholson et al. (1997) Proc. Natal. Acad. Sci. USA 94:9279-9284).

An amino acid sequence for a human proteolipid protein is reported as follows (GenBank Accession No M27110; SEQ ID NO: 102):

MGLLECCARCLVGAPFASLVATGLCFFGVALFCGCGHEALTGTEKLIETYFSKNYQDYEYLINVIHAFQYVIYG
TASFFFLYGALLLAEGFYTTGAVRQIFGDYKTTICGKGLSATVTGGQKGRGSRGQHQAHSLERVCTCLGKWLGH
PDKFVGITYALTVVWLLVFACSAVPVYIYFNTWTTCQSIAFPSKTSASIGSLCADARMYGVLPWNAFPGKVCGS
NLLSICKTAEFQMTFHLFIAAFVGAAATLVSLLTFMIAATYNFAVLKLMGRGTKF

MBP is an extrinsic myelin protein that has been studied extensively. At least 26 MBP epitopes have been reported (Meinl et al (1993) J. Clin. Invest. 92: 2633-2643). Of particular interest are residues 1-11, 59-76 and 87-99. Analogues of MBP peptides generated by truncation have been shown to reverse EAE (Karin et al (1998) J. Immunol. 160: 5188-5194). DNA encoding polypeptide fragments may comprise coding sequences for immunogenic epitopes, e. g. myelin basic protein p84-102, more particularly myelin basic protein p87-99, VHFFKNIVTPRTP (p87-99), or the truncated 7-mer peptide FKNIVTP. The sequences of myelin basic protein exon 2, including the immunodominant epitope bordered by amino acids 59-85, are also of interest. For examples, see Sakai et al. (1988) J Neuroimmunol 19: 21-32; Baxevanis et al (1989) J Neuroimmunol 22: 23-30; Ota et al (1990) Nature 346: 183-187; Martin et al (1992) J Immunol. 148: 1350-1366, Valli et al (1993) J Clin In 91: 616. The immunodominant MBP (84102) peptide has been found to bind with high affinity to DRB1*1501 and DRB5*0101 molecules of the disease-associated DR2 haplotype. Overlapping but distinct peptide segments were important for binding to these molecules; hydrophobic residues (Val189 and Phe92) in the MBP (88-95) segment for peptide binding to DRB1*1501 molecules; hydrophobic and charged residues (Phe92, Lys93) in the MBP (89-101/102) sequence contributed to DRB5*0101 binding.

An amino acid sequence for a human myelin basic protein (MBP) is reported as follows (GenBank Accession No M13577; SEQ ID NO: 103):

MASQKRPSQRHGSKYLATASTMDHARHGFLPRHRDTGILDSIGRFFGGDRGAPKRGSGKDSHHPARTAHYGSLP
QKSHGRTQDENPVVHFFKNIVTPRTPPPSQGKGRGLSLSRFSWGAEGQRPGFGYGGRASDYKSAHKGFKGVDAQ
GTLSKIFKLGGRDSRSGSPMARR

The transmembrane glycoprotein MOG is a minor component of myelin that has been shown to induce EAE. Immunodominant MOG epitopes that have been identified in several mouse strains include residues 1-22, 35-55, 64-96 (deRosbo et al. (1998) J. Autoimmunity 11: 287-299, deRosbo ef al. (1995) Eur J Immunol. 25: 985-993) and 41-60 (Leadbetter et al (1998) J Immunol 161: 504-512).

An amino acid sequence for a human myelin/oligodendrocyte glycoprotein (MOG) protein (25.1 kD) is reported as follows (GenBank Accession No U64564; SEQ ID NO: 104):

MASLSRPSLPSCLCSFLLLLLLQVSSSYAGQFRVIGPRHPIRALVGDEVE
LPCRISPGKNATGMEVGWYRPPFSRVVHLYRNGKDQDGDQAPEYRGRTEL
LKDAIGEGKVTLRIRNVRFSDEGGTCFFRDHSYQEEAAMELKVEDPFYWV
SPGVLVLLAVLPVLLLQITVGLVFLCLQYRLRGKLRAEIENLHRTFDPHF
LRVPCWKITLFVIVPVLGPLVALIICYNWLHRRLAGQFLEELRNPF

An amino acid sequence for a human myelin-associated glycoprotein (MAG) is reported as follows (GenBank Accession No M29273; SEQ ID NO: 105):

MIFLTALPLFWIMISASRGGHWGAWMPSSISAFEGTCVSIPCRFDFPDEL
RPAVVHGVWYFNSPYPKNYPPVVFKSRTQVVHESFQGRSRLLGDLGLRNC
TLLLSNVSPELGGKYYFRGDLGGYNQYTFSEHSVLDIVNTPNIVVPPEVV
AGTEVEVSCMVPDNCPELRPELSWLGHEGLGEPAVLGRLREEGTWVQVSL
LHFVPTREANGHRLGCQASFPNTTLQFEGYASMDVKYPPVIVEEMNSSVE
AIEGSHVSLLCGADSNPPPLLTWMRDGTVLREAVAESLLLELEEVTPAED
GVYACLAENAYGQDNRTVGLSVMYAPWKPTVNGTMVAVEGETVSILCSTQ
SNPDPILTIFKEKQILSTVIYESELQLELPAVSPEDDGEYWCVAENQYGQ
RATAFNLSVEFAPVLLLESHCAAARDTVQCLCVVKSNPEPSVAFELPSRN
VTVNESEREFVYSERSGLVLTSILTLRGQAQAPPRVICTARNLYGAKSLE
LPFQGAHRLMWAKIGPVGAVVAFAILIAIVCYITQTRRKKNVTESPSFSA
GDNPPVLFSSDFRISGAPEKYESERRLGSERRLLGLRGEPPELDLSYSHS
DLGKRPTKDSYTLTEELAEYAEIRVK

In one embodiment one or more antigenic determinants may be used in place of a full antigen. For example, some specific class II MHC-associated autoantigen peptide sequences are as follows (see U.S. Pat. No. 5,783,567):

Peptide Sequence Source

| | |
|---|---|
| GRTQDENPVVHFFKNIVTPRTPP | MPB (aa 80-102; |
| | SEQ ID NO: 106) |
| AVYVYIYFNTWTTCQFIAFPFK | PLP (aa 170-191; |
| | SEQ ID NO: 107) |
| SQRHGSKYLATASTMDHARHG | MBP (aa 7-27; |
| | SEQ ID NO: 108) |
| RDTGILDSIGRFFGGDRGAP | MBP (aa 33-52; |
| | SEQ ID NO: 109) |
| QKSHGRTQDENPVVHFFKNI | MBP (aa 74-93; |
| | SEQ ID NO: 110) |
| DENPVVHFFKNIVT | MBP (aa 84-97; |
| | SEQ ID NO: 111) |
| ENPVVHFFKNIVTPR | MBP (aa 85-99; |
| | SEQ ID NO: 112) |
| HFFKNIVTPRTPP | MBP (aa 90-102; |
| | SEQ ID NO: 113) |
| KGFKGVDAQGTLSK | MBP (aa 139-152; |
| | SEQ ID NO: 114) |
| VDAQGTLSKIFKLGGRDSRS | MBP (aa 144-163; |
| | SEQ ID NO: 115) |

Autoimmune Arthritis Autoantigens and Bystander Antigens

In an alternative embodiment of the present invention the autoantigen or bystander antigen may be autoimmune arthritis autoantigen or bystander antigen for use to treat autoimmune arthritis.

The term “autoimmune arthritis autoantigen” as used herein includes any substance or a component thereof normally found within a mammal that, in autoimmune arthritis (especially rheumatoid arthritis (RA)), becomes a target of attack by the immune system, preferably the primary (or a primary) target of attack. The term also includes antigenic substances that induce conditions having the characteristics of autoimmune arthritis when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “autoimmune arthritis bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in autoimmune arthritis, especially rheumatoid arthritis (RA). The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

The term “autoimmune arthritis” includes rheumatoid arthritis, juvenile arthritis, psoriatic arthritis, spondylo arthritis, relapsing polychondritis and other connective tissue diseases having an autoimmune disease component.

It will be appreciated that combinations of RA autoimmune/bystander antigens and RA autoimmune/bystander antigenic determinants and/or polynucleotide sequences coding for them may also be used as appropriate.

Some examples of RA autoantigens and RA bystander antigens include, but are not limited to, antigens from connective tissue, collagen (especially types I, II, III, IX, and XI), heat shock proteins and immunoglobulin Fc domains (see, e.g. J. Immunol. Methods 121:21 9 (89) and 151:177 (92)).

Collagen is a family of fibrous proteins that have been classified into a number of structurally and genetically distinct types (Stryer, L. Biochemistry, 2nd Edition, W. H. Freeman & Co., 1981, pp. 184-199). Type I collagen is the most prevalent form and is found inter alia, in skin, tendons, cornea and bones and consists of two subunits of alpha1(I) collagen and one subunit of a different sequence termed alpha2. Other types of collagen, including type II collagen, have three identical subunits or chains, each consisting of about 1,000 amino acids. Type II collagen (“CII”) is the type of collagen found inter alia, in cartilage, the interverbebral disc and the vitreous body. Type II collagen contains three alpha1(II) chains (alpha1(II)₃). Type III collagen is found inter alia, in blood vessels, the cardiovascular system and fetal skin and contains three alpha1(III) chains (alpha1(III)₃). Type IV collagen is localized, inter alia, in basement membranes and contains three alpha 1 (IV) chains (alpha1(IV)₃).

Diabetes Autoantigens and Bystander Antigens

In an alternative embodiment of the present invention the autoantigen or bystander antigen may be a diabetes autoantigen or bystander antigen for use to treat autoimmune diabetes.

The term “autoimmune diabetes” as used herein includes all forms of diabetes having an autoimmune component, and, in particular, Type I diabetes (also known as juvenile diabetes or insulin-dependent diabetes mellitus; IDDM). Type I diabetes is a disease that affects mainly children and young adults. The clinical features of the disease are caused by an insufficiency in the body's own insulin production due to a significant or even total reduction in of insulin production. It has been found that this type of diabetes is an autoimmune disease (cf. Castano, L. and G. S. Eisenbirth (1990) Type I diabetes: A chronic autoimmune disease of human, mouse and rat. Annu. Rev. Immunol. 8:647-679).

All cells of the immune system play a more or less important role. The B lymphocytes produce autoantibodies, whereas the monocytes/macrophages are probably involved in the induction of autoimmunity as antigen presenting cells. It is understood that T lymphocytes play a major role as effector cells in the destruction reaction. Like most autoimmune diseases type I diabetes arises because the tolerance of the T cells towards the body's own tissue (“self”) is lost. In particular, loss of tolerance towards pancreatic beta cells will result in the destruction thereof and diabetes will arise.

It is reported that about 30% to 40% of diabetic children will eventually develop nephropathy requiring dialysis and transplantation (see U.S. Pat. No. 5,624,895) Other significant complications include cardiovascular disease, stroke, blindness and gangrene. Moreover, diabetes mellitus accounts for a significant proportion of morbidity and mortality among dialysis and transplant patients.

Onset of Type I diabetes mellitus normally results from a well-characterized insulitis. During this condition, the inflammatory cells are typically directed against the beta cells of the pancreatic islets. It has been demonstrated that a large proportion of the infiltrating T lymphocytes produced during Type I diabetes mellitus are CD8-positive cytotoxic cells, which confirms the cytotoxic activity of the cellular infiltrate. CD4-positive lymphocytes are also present, the majority of which are helper T cells (Bottazzo et at., 1985, New England Journal of Medicine, 313, 353-359). The infiltrating cells also include lymphocytes or B cells that produce immunoglobulin-G (IgG) which suggest that these antibody-producing cells infiltrate the pancreatic islets (Glerchmann et at., 1987, Immunology Today, 8, 167-170).

The term “diabetes autoantigen” as used herein includes any substance or a component thereof normally found within a mammal that, in autoimmune diabetes, becomes a target of attack by the immune system, preferably the primary (or a primary) target of attack. The term also includes antigenic substances that induce conditions having the characteristics of autoimmune diabetes when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “diabetes bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue (usually the pancreas) under autoimmune attack. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

It will be appreciated that combinations of diabetes autoimmune/bystander antigens and diabetes autoimmune/bystander antigenic determinants and/or polynucleotide sequences coding for them may also be used as appropriate.

Examples of diabetes autoantigens and bystander antigens include, but are not limited to, pancreatic beta cell (Type I) antigens, insulins, insulin receptors, insulin associated antigens (IA-w), glucagons, amylins, gamma amino decarboxylases (GADs) and heat shock proteins (HSPs), carboxypeptidases, peripherins and gangliosides. Some of these are discussed in more detail below.

a) Preproinsulin

Human insulin mRNA is translated as a 110 amino acid single chain precursor called preproinsulin, and removal of its signal peptide during insertion into the endoplasmic reticulum generates proinsulin. Proinsulin consists of three domains: an amino-terminal B chain, a carboxy-terminal A chain and a connecting peptide in the middle known as the C peptide. Within the endoplasmic reticulum, proinsulin is exposed to several specific endopeptidases which excise the C peptide, thereby generating the mature form of insulin which consists of the A and B chain. Insulin and free C peptide are packaged in the Golgi into secretory granules which accumulate in the cytoplasm. The preproinsulin peptide sequence is reported as follows:

| | |
|---|---|
| MALWMRLLPL LALLALWGPD PAAAFVNQHL | (SEQ ID NO: 116) |
| CGSHLVEALY LVCGERGFFY TPKTRREAED | |
| LQVGQVELGG GPGAGSLQPL ALEGSLQKRG | |
| IVEQCCTSIC SLYQLENYCN | |

The insulin A chain includes amino acids 90-110 of this sequence. The B chain includes amino acids 25-54. The connecting sequence (amino acids 55-89) includes a pair of basic amino acids at either end. Proteolytic cleavage of proinsulin at these dibasic sequences liberates the insulin molecule and free C peptide, which includes amino acids 57-87. The human preproinsulin or an immunologically active fragment thereof, e. g., B chain or an immunogenic fragment thereof, e. g., amino acids 33-47 (corresponding to residues 9-23 of the B-chain), are useful as autoantigens in the methods and compositions described herein.

b) GAD65

Gad65 is a primary beta-cell antigen involved in the autoimmune response leading to insulin dependent diabetes mellitus (Christgau et al. (1991) J Biol Chem. 266 (31): 21257-64). The presence of autoantibodies to GAD65 is used as a method of diagnosis of type I diabetes. Gad65 is a 585 amino acid protein with a sequence (SEQ ID NO: 117) reported as follows:

MASPGSGFWS FGSEDGSGDS ENPGTARAWC QVAQKFTGGI
GNKLCALLYG DAEKPAESGG SQPPRAAARK AACACDQKPC
SCSKVDVNYA FLHATDLLPA CDGERPTLAF LQDVMNILLQ
YVVKSFDRST KVIDFHYPNE LLQEYNWELA DQPQNLEEIL
MHCQTTLKYA IKTGHPRYFN QLSTGLDMVG LAADWLTSTA
NTNMFTYEIA PVFVLLEYVT LKKMREIIGW PGGSGDGIFS
PGGAISNMYA MMIARFKMFP EVKEKGMAAL PRLIAFTSEH
SHFSLKKGAA ALGIGTDSVI LIKCDERGKM IPSDLERRIL
EAKQKGFVPF LVSATAGTTV YGAFDPLLAV ADICKKYKIW
MHVDAAWGGG LLMSRKHKWK LSGVERANSV TWNPHKMMGV
PLQCSALLVR EEGLMQNCNQ MHASYLFQQD KHYDLSYDTG
DKALQCGRHV DVFKLWLMWR AKGTTGFEAH VDKCLELAEY
LYNIIKNREG YEMVFDGKPQ HTNVCFWYIP PSLRTLEDNE
ERMSRLSKVA PVIKARMMEY GTTMVSYQPL GDKVNFFRMV
ISNPAATHQD IDLFIEEIER LGQDL

c) Islet Tyrosine Phosphatase IA-2

IA-2/ICA512, a member of the protein tyrosine phosphatase family, is another major autoantigen in type 1 diabetes (Lan et al. DNA Cell Biol 13 : 505-514, 1994). It is reported that 70% of diabetic patients have autoantibodies to IA-2, which may appear years before the development of clinical disease. The IA-2 molecule is 979 amino acids in length and consists of an intracellular, transmembrane, and extracellular domain (Rabin et al. (1994) J. Immunol. 152 (6), 3183-3188). Autoantibodies are typically directed to the intracellular domain, e. g., amino acids 600-979 and fragments thereof (Zhang et al. (1997) Diabetes 46: 40-43 ; Xie et al. (1997) J Immunol 159: 3662-3667). The amino acid sequence of IA-2 (SEQ ID NO: 118) is reported as follows:

MRRPRRPGGL GGSGGLRLLL CLLLLSSRPG GCSAVSAHGC
LFDRRLCSHL EVCIQDGLFG QCQVGVGQAR PLLQVTSPVL
QRLQGVLRQL MSQGLSWHDD LTQYVISQEM ERIPRLRPPE
PRPRDRSGLA PKRPGPAGEL LLQDIPTGSA PAAQHRLPQP
PVGKGGAGAS SSLSPLQAEL LPPLLEHLLL PPQPPHPSLS
YEPALLQPYL FHQFGSRDGS RVSEGSPGMV SVGPLPKAEA
PALFSRTALK GIFGDHPGHS YGDLPGPSPA QLFQDSGLLY
LAQELPAPSR ARVPRLPEQG SSSRAEDSPE GYEKEGLGDR
GEKPASPAVQ PDAALQRLAA VLAGYGVELR QLTPEQLSTL
LTLLQLLPKG AGRNPGGVVN VGADIKKTME GPVEGRDTAE
LPARTSPMPG HPTASPTSSE VQQVPSPVSS EPPKAARPPV
TPVLLEKKSP LGQSQPTVAG QPSARPAAEE YGYIVTDQKP
LSLAAGVKLL EILAEHVHMS SGSFINISVV GPALTFRIRH
NEQNLSLADV TQQAGLVKSE LEAQTGLQIL QTGVGQREEA
AAVLPQTAHS TSPMRSVLLT LVALAGVAGL LVALAVALCV
RQHARQQDKE RLAALGPEGA HGDTTFEYQD LCRQHMATKS
LFNRAEGPPE PSRVSSVSSQ FSDAAQASPS SHSSTPSWCE
EPAQANMDIS TGHMILAYME DHLRNRDRLA KEQQALCAYQ
AEPNTCATAQ GEGNIKKNRH PDFLPYDHAR IKLKVESSPS
RSDYINASPI IEHDPRMPAY IATQPGLSHT IADFWQMVWE
SGCTVIVMLT PLVEDGVKQC DRYWPDEGAS LYHVYEVNLV
SEHIWCEDFL VRSFYLKNVQ TQETRTLTQF HFLSWPAEGT
PASTRPLLDF RRKVNKCYRG RSCPIIVHCS DGAGRTGTYI
LIDMVLNRMA KGVKEIDIAA TLEHVRDQRP GLVRSKDQFE
FALTAVAEEV NAILKALPQ

d) ICA12

ICA12 (Kasimiotis et al. (2000) Diabetes 49 (4): 555-61; GenBank Accession No. AAD16237) is one of a number of islet cell autoantigens associated with diabetes. The amino acid sequence (SEQ ID NO: 119) of ICA12 is reported as follows:

MSMRSPISAQ LALDGVGTMV NCTIKSEEKK EPCHEAPQGS
ATAAEPQPGD PARASQDSAD PQAPAQGNFR GSWDCSSPEG
NGSPEPKRPG ASEAASGSQE KLDFNRNLKE VVPAIEKLLS
SDWKERFLGR NSMEAKDVKG TQESLAEKEL QLLVMIHQLS
TLRDQLLTAH SEQKNMAAML FEKQQQQMEL ARQQQEQIAK
QQQQLIQQQH KINLLQQQIQ QVNMPYVMIP AFPPSHQPLP
VTPDSQLALP IQPIPCKPVE YPLQLLHSPP APVVKRPGAM
ATHHPLQEPS QPLNLTAKPK APELPNTSSS PSLKMSSCVP
RPPSHGGPTR DLQSSPPSLP LGFLGEGDAV TKAIQDARQL
LHSHSGALDG SPNTPFRKDL ISLDSSPAKE RLEDGCVHPL
EEAMLSCDMD GSRHFPESRN SSHIKRPMNA FMVWAKDERR
KILQAFPDMH NSSISKILGS RWKSMTNQEK QPYYEEQARL
SRQHLEKYPD YKYKPRPKRT CIVEGKRLRV GEYKALMRTR
RQDARQSYVI PPQAGQVQMS SSDVLYPRAA GMPLAQPLVE
HYVPRSLDPN MPVIVNTCSL REEGEGTDDR HSVADGEMYR
YSEDEDSEGE EKSDGELVVL TD

e) ICA69

ICA69 is another autoantigen associated with type 1 diabetes (Pietropaolo et al. J Clin Invest 1993; 92: 359-371). An amino acid sequence (SEQ ID NO: 120) of ICA69 is reported as follows:

MSGHKCSYPW DLQDRYAQDK SVVNKMQQRY WETKQAFIKA
TGKKEDEHVV ASDADLDAKL ELFHSIQRTC LDLSKAIVLY
QKRICFLSQE ENELGKFLRS QGFQDKTRAG KMMQATGKAL
CFSSQQRLAL RNPLCRFHQE VETFRHRAIS DTWLTVNRME
QCRTEYRGAL LWMKDVSQEL DPDLYKQMEK FRKVQTQVRL
AKKNFDKLKM DVCQKVDLLG ASRCNLLSHM LATYQTTLLH
FWEKTSHTMA AIHESFKGYQ PYEFTTLKSL QDPMKKLVEK
EEKKKINQQE STDAAVQEPS QLISLEEENQ RKESSSFKTE
DGKSILSALD KGSTHTACSG PIDELLDMKS EEGACLGPVA
GTPEPEGADK DDLLLLSEIF NASSLEEGEF SKEWAAVFGD
GQVKEPVPTM ALGEPDPKAQ TGSGFLPSQL LDQNMKDLQA
SLQEPAKAAS DLTAWFSLFA DLDPLSNPDA VGKTDKEHEL
LNA

f) Glima 38

Glima 38 is a 38 kDa islet cell membrane autoantigen which is specifically immunoprecipitated with sera from a subset of prediabetic individuals and newly diagnosed type 1 diabetic patients. Glima 38 is an amphiphilic membrane glycoprotein, specifically expressed in islet and neuronal cell lines, and thus shares the neuroendocrine expression patterns of GAD65 and IA2 (Aanstoot et al. J Clin Invest. 1996 Jun. 15; 97 (12): 2772-2783).

g) Heat Shock Protein 60 (HSP60)

HSP60, e. g., an immunologically active fragment of HSP60, e. g., p277 (see Elias et al., Eur Jlmmunol 1995 25 (10): 2851-7), can also be used as an autoantigen in the methods and compositions described herein. Other useful epitopes of HSP 60 are described, for example, in U.S. Pat. No. 6,110,746.

h) Carboxypeptidase H

Carboxypeptidase H has been identified as an autoantigen, e. g., in pre-type 1 diabetes patients (Castano et al. (1991) J Clin Endocrinol Metab 73 (6): 1197-201; Alcalde et al. J Autoimmun. 1996 August; 9 (4): 525-8.). Therefore, carboxypeptidase H or immunologically reactive fragments thereof (e. g., the 136-amino acid fragment of carboxypeptidase-H described in Castano, supra) can be used in the methods and compositions described herein.

i) Peripherin

Peripherin is a 58 KDa diabetes autoantigen identified in nod mice (Boitard et al. (1992) Proc Natl Acad Sci USA 89 (1): 172-6). A human peripherin sequence (SEQ ID NO: 121) is reported as follows:

MSHHPSGLRA GFSSTSYRRT FGPPPSLSPG AFSYSSSSRF
SSSRLLGSAS PSSSVRLGSF RSPRAGAGAL LRLPSERLDF
SMAEALNQEF LATRSNEKQE LQELNDRFAN FIEKVRFLEQ
QNAALRGELS QARGQEPARA DQLCQQELRE LRRELELLGR
ERDRVQVERD GLAEDLAALK QRLEEETRKR EDAEHNLVLF
RKDVDDATLS RLELERKIES LMDEIEFLKK LHEEELRDLQ
VSVESQQVQQ VEVEATVKPE LTAALRDIRA QYESIAAKNL
QEAEEWYKSK YADLSDAANR NHEALRQAKQ EMNESRRQIQ
SLTCEVDGLR GTNEALLRQL RELEEQFALE AGGYQAGAAR
LEEELRQLKE EMARHLREYQ ELLNVKMALD IEIATYRKLL
EGEESRISVP VHSFASLNIK TTVPEVEPPQ DSHSRKTVLI
KTIETRNGEQ VVTESQKEQR SELDKSSAHS Y

j) Gangliosides

Gangliosides can also be useful autoantigens in the methods and compositions described herein. Gangliosides are sialic acid-containing glycolipids which are formed by a hydrophobic portion, the ceramide, and a hydrophilic part, i. e. the oligosaccharide chain. Gangliosides are expressed, inter alia, in cytosol membranes of secretory granules of pancreatic islets. Auto-antibodies to gangliosides have been described in type 1 diabetes, e.g., GM1-2 ganglioside is an islet autoantigen in diabetes autoimmunity and is expressed by human native (3 cells (Dotta et al. Diabetes. 1996 September; 45 (9): 1193-6). Gangliosides GT3, GD3 and GM-1 are also the target of autoantibodies associated with autoimmune diabetes (reviewed in Dionisi et al. Aim Ist

Super Sanita 1997; 33 (3): 433-5). Ganglioside GM3 participates in the pathological conditions of insulin resistance (Tagami et al. J Biol Chem 2001 Nov. 13; online publication ahead of print).

Further sequences are provided, for example, under GenBank Accession Nos U26593.1, BC008640.1, NM_{—}022308.1, NM_{—}022307.1, NM_{—}004968.1, AF146363.1, AF147807.1, AH008870.1, U37183.1, U38260.1, AH005787.1, U71264.1, U71263.1, U71262.1, U71261.1, U71260.1, U71259.1, U71258.1, U71257.1, U71256.1, U71255.1, U71254.1, U71253.1, U71252.1, U01882.1, U17989.1 (diabetes mellitus type I autoantigen (ICAp69)), X62899.2 (islet cell antigen 512), A28076.1 (islet GAD sequence (HIGAD-FL)) and AF098915.1 (type 1 diabetes autoantigen ICA12).

Myasthenia Gravis Autoantigens and Bystander Antigens

In an alternative embodiment of the present invention the autoantigen or bystander antigen may be a Myasthenia Gravis autoantigen or bystander antigen for use to treat Myasthenia Gravis.

The term “Myasthenia Gravis autoantigen” as used herein includes any substance or a component thereof normally found within a mammal that, in Myasthenia Gravis, becomes a target of attack by the immune system, preferably the primary (or a primary) target of attack. The term also includes antigenic substances that induce conditions having the characteristics of Myasthenia Gravis when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “Myasthenia Gravis bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in Myasthenia Gravis. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

It will be appreciated that combinations of Myasthenia Gravis autoimmune/bystander antigens and Myasthenia Gravis autoimmune/bystander antigenic determinants and/or polynucleotide sequences coding for them may also be used as appropriate.

Some examples of Myasthenia Gravis autoantigens and Myasthenia Gravis bystander antigens include, but are not limited to, acetyl choline receptors and components thereof, preferably human acetyl choline receptors and components thereof (see e.g. Eur. J. Pharm. 172:231(89)).

An amino acid sequence for a human gravin (A kinase (PRKA) anchor protein) autoantigen is reported as follows (GenBank Accession No M96322; SEQ ID NO: 122):

DCQAKSTPVIVSATTKKGLSSDLEGEKTTSLKWKSDEVDEQVACQEVKVS
VAIEEDLEPENGILELETKSSKLVQNIIQTAVDQFVRTEETATEMLTSEL
QTQAHMIKADSQDAGQETEKEGEEPQASAQDETPITSAKEESESTAVGQA
HSDISKDMSEASEKTMTVEVEGSTVNDQQLEEVVLPSEEEGGGAGTKSVP
EDDGHALLAERIEKSLVEPKEDEKGDDVDDPENQNSALADTDASGGLTKE
SPDTNGPKQKEKEDAQEVELQEGKVHSESDKAITPQAQEELQKQERESAK
SELTES

An amino acid sequence for a human cholinergic receptor (gamma subunit) autoantigen is reported as follows (GenBank Accession No NM_{—}005199; SEQ ID NO: 123):

MHGGQGPLLLLLLLAVCLGGTQRNLRNQEERLLADLMQNYDPNLRPAERD
SDVVNVSLKLTLTNLISLNEREEALTTNVWIEMQWCDYRLRWDPRDYEGL
WVLRVPSTMVWRPDIVLENNVDGVFEVALYCNVLVSPDGCIYWLPPAIFR
SACSISVTYFPFDWQNCSLIFQSQTYSTNEIDLQLSQEDGQTIEWIFIDP
EAFTENGEWAIQHRPAKMLLDPAAPAQEAGHQKVVFYLLIQRKPLFYVIN
IIAPCVLISSVAILIHFLPAKAGGQKCTVAINVLLAQTVFLFLVAKKVPE
TSQAVPLISKYLTFLLVVTILIVVNAVVVLNVSLRSPHTHSMARGVRKVF
LRLLPQLLRMHVRPLAPAAVQDTQSRLQNGSSGWSITTGEEVALCLPRSE
LLFQQWQRQGLVAAALEKLEKGPELGLSQFCGSLKQAAPAIQACVEACNL
IACARHQQSHFDNGNEEWFLVGRVLRVCFLAMLSLFICGTAGIFLMAHYN
RVPALPFPGDPRPYLPSPD

An amino acid sequence for a human cholinergic receptor (alpha subunit) autoantigen is reported as follows (GenBank Accession No S77094; SEQ ID NO: 124):

MEPWPLLLLFSLCSAGLVLGSEHETRLVAKLFKDYSSVVRPVEDHRQVVE
VTVGLQLIQLINVDEVNQIVTTNVRLKQQWVDYNLKWNPDDYGGVKKIHI
PSEKIWRPDLVLYNNADGDFAIVKFTKVLLQYTGHITWTPPAIFKSYCEI
IVTHFPFDEQNCSMKLGTWTYDGSVVAINPESDQPDLSNFMESGEWVIKE
SRGWKHSVTYSCCPDTPYLDITYHFVMQRLPLYFIVNVIIPCLLFSFLTG
LVFYLPTDSGEKMTLSISVLLSLTVFLLVIVELIPSTSSAVPLIGKYMLF
TMVFVIASIIITVIVINTHHRSPSTHVMPNWVRKVFIDTIPNIMFFSTMK
RPSREKQDKKIFTEDIDISDISGKPGPPPMGFHSPLIKHFEVKSAIEGIK
YIAETMKSDQESNNAAAEWKYVAMVMDHILLGVFMLVCIIGTLAVFAGRL
IELNQQG

(see also Gattenlohner et al, Cloning of a cDNA coding for the acetylcholine receptor alpha-subunit from a thymoma associated with myasthenia gravis, Thymus 23 (2), 103-113 (1994))

Purified acetylcholine receptor can be isolated, for example, by the method of Mcintosh et al. J Neuroimmunol. 25: 75, 1989.

In an alternative embodiment one or more antigenic determinants may be used in place of a full antigen. For example, some specific class II MHC-associated autoantigen peptide sequences are as follows (see U.S. Pat. No. 5,783,567):

Peptide Sequence Source

| | |
|---|---|
| TVGLQLIQLINVDEVNQIVTTNVRLK | AChR alpha (aa 32-67; |
| | SEQ ID NO: 125) |
| QQWVDYNLKW | (SEQ ID NO: 126) |
| QIVTTNVRLKQQWVDYNLKW | AChR alpha (aa 48-67; |
| | SEQ ID NO: 127) |
| QWVDYNL | AChR alpha (aa 59-65; |
| | SEQ ID NO: 128) |
| GGVKKIHIPSEKIWRPDL | AChR alpha (aa 73-90; |
| | SEQ ID NO: 129) |
| AIVKFTKVLLQY | AChR alpha (aa 101-112; |
| | SEQ ID NO: 130) |
| WTPPAIFKSYCEIIVTHFPF | AChR alpha (aa 118-137; |
| | SEQ ID NO: 131) |
| MKLGTWTYDGSVV | AChR alpha (aa 144-156; |
| | SEQ ID NO: 132) |
| MKLGIWTYDGSVV | AChR alpha (aa 144-157; |
| | SEQ ID NO: 133) |
| analog (I-148) | |
| WTYDGSVVA | AChR alpha (aa 149-157; |
| | SEQ ID NO: 134) |
| SCCPDTPYLDITYHFVM | AChR alpha (aa 191-207; |
| | SEQ ID NO: 135) |
| DTPYLDITYHFVMQRLPL | AChR alpha (aa 195-212; |
| | SEQ ID NO: 136) |
| FIVNVIIPCLLFSFLTGLVFY | AChR alpha (aa 214-234; |
| | SEQ ID NO: 137) |
| LLVIVELIPSTSS | AChR alpha (aa 257-269; |
| | SEQ ID NO: 138) |
| STHVMPNWVRKVFIDTIPN | AChR alpha (aa 304-322; |
| | SEQ ID NO: 139) |
| NWVRKVFIDTIPNIMFFS | AChR alpha (aa 310-327; |
| | SEQ ID NO: 140) |
| IPNIMFFSTMKRPSREKQ | AChR alpha (aa 320-337; |
| | SEQ ID NO: 141) |
| AAAEWKYVAMVMDHIL | AChR alpha (aa 395-410; |
| | SEQ ID NO: 142) |
| IIGTLAVFAGRLIELNQQG | AChR alpha (aa 419-437; |
| | SEQ ID NO: 143) |
| GQTIEWIFIDPEAFTENGEW | AChR gamma (aa 165-184; |
| | SEQ ID NO: 144) |
| MAHYNRVPALPFPGDPRPYL | AChR gamma (aa 476-495; |
| | SEQ ID NO: 145) |

SLE Autoantigens and SLE Bystander Antigens

In an alternative embodiment of the present invention the autoantigen or bystander antigen may be a Systemic Lupus Erythematosus (SLE) autoantigen or bystander antigen for use to treat SLE.

The term “SLE autoantigen” as used herein includes any substance or a component thereof normally found within a mammal that, in Systemic Lupus Erythematosus (SLE), becomes a target of attack by the immune system, preferably the primary (or a primary) target of attack. The term also includes antigenic substances that induce conditions having the characteristics of an autoimmune disease when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “SLE bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in SLE. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction, such as heatshock proteins (HSP), which although not necessarily specific to a particular tissue are normally shielded from the immune system.

It will be appreciated that combinations of SLE autoimmune/bystander antigens and SLE autoimmune/bystander antigenic determinants and/or polynucleotide sequences coding for them may also be used as appropriate.

Some examples of SLE autoantigens and SLE bystander antigens include, but are not limited to, ds-DNA, chromatin, histones, nucleolar antigens, soluble RNA protein particles (such as U1RNP, Sm, Ro/SSA and La/SSB) erythrocyte antigens and platelet antigens. Examples of proteins include, for example, the human Ku and La antigens.

For example, an amino acid sequence for a human lupus p70 (Ku) autoantigen protein is reported as follows (GenBank Accession No J04611; SEQ ID NO: 146):

MSGWESYYKTEGDEEAEEEQEENLEASGDYKYSGRDSLIFLVDASKAMFE
SQSEDELTPFDMSIQCIQSVYISKIISSDRDLLAVVFYGTEKDKNSVNFK
NIYVLQELDNPGAKRILELDQFKGQQGQKRFQDMMGHGSDYSLSEVLWVC
ANLFSDVQFKMSHKRIMLFTNEDNPHGNDSAKASRARTKAGDLRDTGIFL
DLMHLKKPGGFDISLFYRDIISIAEDEDLRVHFEESSKLEDLLRKVRAKE
TRKRALSRLKLKLNKDIVISVGIYNLVQKALKPPPIKLYRETNEPVKTKT
RTFNTSTGGLLLPSDTKRSQIYGSRQIILEKEETEELKRFDDPGLMLMGF
KPLVLLKKHHYLRPSLFVYPEESLVIGSSTLFSALLIKCLEKEVAALCRY
TPRRNIPPYFVALVPQEEELDDQKIQVTPPGFQLVFLPFADDKRKMPFTE
KIMATPEQVGKMKAIVEKLRFTYRSDSFENPVLQQHFRNLEALALDLMEP
EQAVDLTLPKVEAMNKRLGSLVDEFKELVYPPDYNPEGKVTKRKHDNEGS
GSKRPKVEYSEEELKTHISKGTLGKFTVPMLKEACRAYGLKSGLKKQELL
EALTKHFQD

(see also Reeves, W. H. and Sthoeger, Z. M., Molecular cloning of cDNA encoding the p70 (Ku) lupus autoantigen, J. Biol. Chem. 264 (9), 5047-5052 (1989))

An amino acid sequence for a human lupus p80 (Ku) autoantigen protein is reported as follows (GenBank Accession No J04977; SEQ ID NO: 147):

MVRSGNKAAVVLCMDVGFTMSNSIPGIESPFEQAKKVITMFVQRQVFAEN
KDEIALVLFGTDGTDNPLSGGDQYQNITVHRHLMLPDFDLLEDIESKIQP
GSQQADFLDALIVSMDVIQHETIGKKFEKRHIEIFTDLSSRFSKSQLDII
IHSLKKCDISLQFFLPFSLGKEDGSGDRGDGPFRLGGHGPSFPLKGITEQ
QKEGLEIVKMVMISLEGEDGLDEIYSFSESLRKLCVFKKIERHSIHWPCR
LTIGSNLSIRIAAYKSILQERVKKTWTVVDAKTLKKEDIQKETVYCLNDD
DETEVLKEDIIQGFRYGSDIVPFSKVDEEQMKYKSEGKCFSVLGFCKSSQ
VQRRFFMGNQVLKVFAARDDEAAAVALSSLIHALDDLDMVAIVRYAYDKR
ANPQVGVAFPHIKHNYECLVYVQLPFMEDLRQYMFSSLKNSKKYAPTEAQ
LNAVDALIDSMSLAKKDEKTDTLEDLFPTTKIPNPRFQRLFQCLLHRALH
PREPLPPIQQHIWNMLNPPAEVTTKSQIPLSKIKTLFPLIEAKKKDQVTA
QEIFQDNHEDGPTAKKLKTEQGGAHFSVSSLAEGSVTSVGSVNPAENFRV
LVKQKKASFEEASNQLINHIEQFLDTNETPYFMKSIDCIRAFREEAIKFS
EEQRFNNFLKALQEKVEIDQLNHFWEIVVQDGITLITKEEASGSSVTAEE
AKKFLAPKDKPSGDTAAVFEEGGDVDDLLDMI

(see also Yaneva, M., Wen, J., Ayala, A. and Cook, R., cDNA-derived amino acid sequence of the 86-kDa subunit of the Ku antigen, J. Biol. Chem. 264 (23), 13407-13411 (1989))

An amino acid sequence for a human La protein/SS-B antigen is reported as follows (GenBank Accession No J04205 M11108; SEQ ID NO: 148):

MAENGDNEKMAALEAKICHQIEYYFGDFNLPRDKFLKEQIKLDEGWVPLE
IMIKFNRLNRLTTDFNVIVEALSKSKAELMEISEDKTKIRRSPSKPLPEV
TDEYKNDVKNRSVYIKGFPTDATLDDIKEWLEDKGQVLNIQMRRTLHKAF
KGSIFVVFDSIESAKKFVETPGQKYKETDLLILFKDDYFAKKNEERKQNK
VEAKLRAKQEQEAKQKLEEDAEMKSLEEKIGCLLKFSGDLDDQTCREDLH
ILFSNHGEIKWIDFVRGAKEGIILFKEKAKEALGKAKDANNGNLQLRNKE
VTWEVLEGEVEKEALKKIIEDQQESLNKWKSKGRRFKGKGKGNKAAQPGS
GKGKVQFQGKKTKFASDDEHDEHDENGATGPVKRAREETDKEEPASKQQK
TENGAGDQ

(see also Chambers et al, Genomic structure and amino acid sequence domains of the human La autoantigen, J. Biol. Chem. 263 (34), 18043-18051 (1988))
Bowel Autoantigens and Bystander Antigens

In an alternative embodiment of the present invention the autoantigen or bystander antigen may be a bowel autoantigen or bystander antigen for use to treat an autoimmune disease of the bowel.

The term “autoimmune disease of the bowel” as used herein includes any disease in which the bowel or a component of the bowel comes under autoimmune attack. The main autoimmune diseases of the bowel are inflammatory bowel disease (IBD) and celiac (also known as coeliac) disease.

Inflammatory bowel disease (IBD) is the term generally applied to four diseases of the bowel, namely Crohn's disease, ulcerative colitis, indeterminate colitis, and infectious colitis.

Ulcerative colitis is a chronic inflammatory disease mainly affecting the large intestine. The course of the disease may be continuous or relapsing, mild or severe. The earliest lesion is typically an inflammatory infiltration with abscess formation at the base of the crypts of Lieberkuhn. Coalescence of these distended and ruptured crypts tends to separate the overlying mucosa from its blood supply, leading to ulceration. Signs and symptoms of the disease include cramping, lower abdominal pain, rectal bleeding, and frequent, loose discharges consisting mainly of blood, pus, and mucus with scanty fecal particles. A total colectomy may be required for acute severe or chronic, unremitting ulcerative colitis.

Crohn's disease (also known as regional enteritis or ulcerative ileitis) is also a chronic inflammatory disease of unknown etiology but, unlike ulcerative colitis, it can affect any part of the bowel. The most prominent feature of the disease is the granular, reddish-purple edematous thickening of the bowel wall. With the development of inflammation, these granulomas often lose their circumscribed borders and integrate with the surrounding tissue. Diarrhea and obstruction of the bowel are the predominant clinical features. As with ulcerative colitis, the course of the disease may be continuous or relapsing, mild or severe but, unlike ulcerative colitis, it is not curable by resection of the involved segment of bowel. Many patients with Crohn's disease require surgery at some point, but subsequent relapse is common and continuous medical treatment is usual.

Celiac disease (CD) is a disease of the intestinal mucosa and is usually identified in infants and children. Celiac disease is associated with an inflammation of the mucosa, which causes malabsorption. Individuals with celiac disease are intolerant to the protein gluten, which is present in foods such as wheat, rye and barley. When exposed to gluten, the immune system of an individual with celiac disease responds by attacking the lining of the small intestine.

The term “bowel autoantigen” as used herein includes any substance or a component thereof normally found within a mammal that, in an autoimmune disease of the bowel, becomes a target of attack by the immune system, preferably the primary (or a primary) target of attack. The term also includes antigenic substances that induce conditions having the characteristics of an autoimmune disease of the gut when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “bowel bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the bowel under autoimmune attack in an autoimmune disease of the bowel. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction. It will be appreciated that combinations of bowel autoimmune/bystander antigens and bowel autoimmune/bystander antigenic determinants and/or polynucleotide sequences coding for them may also be used as appropriate.

Examples of bowel autoantigens and bystander antigens include, but are not limited to, gliadins and tissue transglutaminase (tTG) (associated with celiac disease; see Marsh, Nature Medicine 1997; 7:725-6) and tropomyosins, in particular tropomyosin isoform 5, (associated with ulcerative colitis).

Allergens and Antigenic Determinants thereof

In an alternative embodiment of the present invention the antigen or bystander antigen may be a an allergen or bystander antigen for use to treat an allergic condition.

The term “allergen” as used herein means any substance which can induce an allergic response, especially a type I hypersensitive response. Typical allergens include, but are not limited to, pollens, molds, foods, animal danders or their excretions, smuts and insects, their venoms or their excretions. Allergens may, for example, be natural or synthetic organic molecules such as peptides/proteins, polysaccharides or lipids. They may be administered singly or as a mixture. Allergens may be chemically or physically modified. Such modified allergens, or allergen derivatives, are known in the art. Examples include, but are not limited to, peptide fragments, conjugates or polymerized allergen derivatives. Thus, the term “allergen” as used herein includes naturally occurring (native) allergens as well as any biologically active fragment, derivative, homologue or variant thereof or any antigenic determinant or epitope (especially immunodominant epitope) thereof or any polynucleotide coding for an allergen (including any biologically active fragment, derivative, homologue or variant) or antigenic determinant or epitope (especially immunodominant epitope) thereof.

The amount of allergen to be administered can be determined empirically and depends on the sensitivity of the individual as well as the desired clinical result. Generally, a regimen of desensitization initially involves the periodic administration of smaller amounts of allergen, which level is increased over the course of the regimen until a predetermined (planned) upper limit is reached or the individual can tolerate exposure to such allergen without a significant adverse allergic response. The particular regimen often is tailored to individual patient needs. The embodiment and potential advantage of the present invention is that it may be possible to meaningfully decrease the level of allergens administered and/or the number of injections and, thereby, the length of the desensitization regimen. Further, with a meaningful decrease of the level (dose) of allergen administered to particularly sensitive individuals, there is a possible diminished risk of severe allergic reaction to the administration of the allergen.

The progress of immunotherapy can be monitored by any clinically acceptable diagnostic tests. Such tests are well known in the art and include symptom levels and requirement levels for ancillary therapy recorded in a daily diary, as well as skin testing and in vitro serological tests for specific IgE antibody and/or specific IgG antibody.

The present invention may be used for preventing and treating all forms of allergy and allergic disorder, including without limitation: ophthalmic allergic disorders, including allergic conjunctivitis, vernal conjunctivitis, vernal keratoconjunctivitis, and giant papillary conjunctivitis; nasal allergic disorders, including allergic rhinitis and sinusitis; otic allergic disorders, including eustachian tube itching; allergic disorders of the upper and lower airways, including intrinsic and extrinsic asthma; allergic disorders of the skin, including dermatitis, eczema and urticaria; and allergic disorders of the gastrointestinal tract.

Any form of allergen (including any biologically active fragment, derivative, homologue or variant) or antigenic determinant or epitope (especially immunodominant epitope) thereof or any polynucleotide coding for an allergen (including any biologically active fragment, derivative, homologue or variant) or antigenic determinant or epitope (especially immunodominant epitope) thereof may be used, including but not limited to mite allergens etc and antigenic determinants or epitopes (especially immunodominant epitopes) thereof.

In addition, it will be appreciated that modulation of an immune response to one particular antigen or antigenic determinant may also modulate responses to other similar antigens and antigenic determinants by operation of a “bystander effect” and/or by so-called epitope spreading or linked suppression.

An antigen suitable for use in the present invention may be any substance that can be recognised by the immune system, and is generally recognised by an antigen (T-cell) receptor. Preferably the antigen used in the present invention is an immunogen.

The immune response to antigen is generally either cell mediated (T cell mediated killing) or humoral (antibody production via recognition of whole antigen). The pattern of cytokine production by TH cells involved in an immune response can influence which of these response types predominates: cell mediated immunity (TH1) is characterised by high IL-2 and IFNγ but low IL-4 production, whereas in humoral immunity (TH2) the pattern is low IL-2 and IFNγ but high IL-4, IL-5 and IL-13. Since the secretory pattern is modulated at the level of the secondary lymphoid organ or cells, then pharmacological manipulation of the specific TH cytokine pattern can influence the type and extent of the immune response generated.

The TH1-TH2 balance refers to the relative representation of the two different forms of helper T cells. The two forms have large scale and opposing effects on the immune system. If an immune response favours TH1 cells, then these cells will drive a cellular response, whereas TH2 cells will drive an antibody-dominated response. The type of antibodies responsible for some allergic reactions is induced by TH2 cells.

The antigen used in the present invention may be a peptide, polypeptide, carbohydrate, protein, glycoprotein, or more complex material containing multiple antigenic epitopes such as a protein complex, cell-membrane preparation, whole cells (viable or non-viable cells), bacterial cells or virus/viral component.

The antigen moiety may be, for example, a synthetic MHC-peptide complex i.e. a fragment of the MHC molecule bearing the antigen groove bearing an element of the antigen. Such complexes have been described in Altman et al. (1996) Science 274: 94-96.

Graft (Transplant) Antigens

The term “graft antigen” as used herein means an antigen or antigenic determinant from a graft which is at least partly responsible for immune response against the graft, and in extreme cases contributes to graft rejection. Typically, a graft antigen will be a Type I or Type II MHC antigen (especially HLA antigen) present on cells of the graft.

Vaccines and Cancer Vaccines

The active RNAi agents of the present invention may for example be used in therapeutic and prophylactic vaccine compositions such as cancer and pathogen vaccines.

Vaccine Compositions

RNAi agents according to the present invention which inhibit Notch signalling (e.g. as determined by use of assays as described herein) may be employed in vaccine compositions (such as pathogen or cancer vaccines) to protect or treat a mammal susceptible to, or suffering from disease, by means of administering said vaccine via a mucosal route, such as the oral/bucal/intestinal/vaginal/rectal or nasal route. Such administration may for example be in a droplet, spray, or dry powdered form. Nebulised or aerosolised vaccine formulations may also be used where appropriate.

Enteric formulations such as gastro resistant capsules and granules for oral administration, suppositories for rectal or vaginal administration may also be used. The present invention may also be used to enhance the immunogenicity of antigens applied to the skin, for example by intradermal, transdermal or transcutaneous delivery. In addition, the adjuvants of the present invention may be parentally delivered, for example by intramuscular or subcutaneous administration.

Depending on the route of administration, a variety of administration devices may be used. For example, for intranasal administration a spray device such as the commercially available Accuspray (Becton Dickinson) may be used.

Preferred spray devices for intranasal use are devices for which the performance of the device is not dependent upon the pressure applied by the user. These devices are known as pressure threshold devices. Liquid is released from the nozzle only when a threshold pressure is attained. These devices make it easier to achieve a spray with a regular droplet size. Pressure threshold devices suitable for use with the present invention are known in the art and are described for example in WO 91/13281 and EP 311 863 B. Such devices are commercially available from Pfeiffer GmbH.

For certain vaccine formulations, other vaccine components may be included in the formulation. For example the adjuvant formulations of the present invention may also comprise a bile acid or derivative of cholic acid. Suitably the derivative of cholic acid is a salt thereof, for example a sodium salt thereof. Examples of bile acids include cholic acid itself, deoxycholic acid, chenodeoxy colic acid, lithocholic acid, taurodeoxycholate ursodeoxycholic acid, hyodeoxycholic acid and derivatives like glyco-, tauro-, amidopropyl-1-propanesulfonic- and amidopropyl-2-hydroxy-1-propanesulfonic-derivatives of the above bile acids, or N,N-bis(3DGluconoamidopropyl)deoxycholamide.

Suitably, an adjuvant formulation of the present invention may be in the form of an aqueous solution or a suspension of non-vesicular forms. Such formulations are convenient to manufacture, and also to sterilise (for example by terminal filtration through a 450 or 220 nm pore membrane).

Suitably, the route of administration may be via the skin, intramuscular or via a mucosal surface such as the nasal mucosa. When the admixture is administered via the nasal mucosa, the admixture may for example be administered as a spray. The methods to enhance an immune response may be either a priming or boosting dose of the vaccine.

The term “adjuvant” as used herein includes an agent having the ability to enhance the immune response of a vertebrate subject's immune system to an antigen or antigenic determinant.

The term “immune response” includes any response to an antigen or antigenic determinant by the immune system of a subject. Immune responses include for example humoral immune responses (e. g. production of antigen-specific antibodies) and cell-mediated immune responses (e. g. lymphocyte proliferation).

The term “cell-mediated immune response” includes the immunological defence provided by lymphocytes, such as the defence provided by T cell lymphocytes when they come into close proximity with their victim cells.

When “lymphocyte proliferation” is measured, the ability of lymphocytes to proliferate in response to specific antigen may be measured. Lymphocyte proliferation includes B cell, T-helper cell or CTL cell proliferation.

Compositions of the present invention may be used to formulate vaccines containing antigens derived from a wide variety of sources. For example, antigens may include human, bacterial, or viral nucleic acid, pathogen derived antigen or antigenic preparations, host-derived antigens, including GnRH and IgE peptides, recombinantly produced protein or peptides, and chimeric fusion proteins.

Preferably the vaccine formulations of the present invention contain an antigen or antigenic composition capable of eliciting an immune response against a human pathogen. The antigen or antigens may, for example, be peptides/proteins, polysaccharides and lipids and may be derived from pathogens such as viruses, bacteria and parasites/fungi as follows:

Viral Antigens

Viral antigens or antigenic determinants may be derived, for example, from:

Cytomegalovirus (especially Human, such as gB or derivatives thereof); Epstein Barr virus (such as gp350); flaviviruses (e. g. Yellow Fever Virus, Dengue Virus, Tick-borne encephalitis virus, Japanese Encephalitis Virus); hepatitis virus such as hepatitis B virus (for example Hepatitis B Surface antigen such as the PreS1, PreS2 and S antigens described in EP-A-414 374; EP-A-0304 578, and EP-A-198474), hepatitis A virus, hepatitis C virus and hepatitis E virus; HIV-1, (such as tat, nef, gp120 or gp160); human herpes viruses, such as gD or derivatives thereof or Immediate Early protein such as ICP27 from HSV1 or HSV2; human papilloma viruses (for example HPV6, 11, 16, 18); Influenza virus (whole live or inactivated virus, split influenza virus, grown in eggs or MDCK cells, or Vero cells or whole flu virosomes (as described by Gluck, Vaccine, 1992, 10, 915-920) or purified or recombinant proteins thereof, such as NP, NA, HA, or M proteins); measles virus; mumps virus; parainfluenza virus; rabies virus; Respiratory Syncytial virus (such as F and G proteins); rotavirus (including live attenuated viruses); smallpox virus; Varicella Zoster Virus (such as gpI, II and IE63); and the HPV viruses responsible for cervical cancer (for example the early proteins E6 or E7 in fusion with a protein D carrier to form Protein D-E6 or E7 fusions from HPV 16, or combinations thereof; or combinations of E6 or E7 with L2 (see for example WO 96/26277).

Bacterial Antigens

Bacterial antigens or antigenic determinants may be derived, for example, from: Bacillus spp., including B. anthracis (e.g. botulinum toxin); Bordetella spp, including B. pertussis (for example pertactin, pertussis toxin, filamenteous hemagglutinin, adenylate cyclase, fimbriae); Borrelia spp., including B. burgdorferi (e.g. OspA, OspC, DbpA, DbpB), B. garinii (e.g. OspA, OspC, DbpA, DbpB), B. afzelii (e.g. OspA, OspC, DbpA, DbpB), B. andersonii (e.g. OspA, OspC, DbpA, DbpB), B. hermsii; Campylobacter spp, including C. jejuni (for example toxins, adhesins and invasins) and C. coli; Chlamydia spp., including C. trachomatis (e.g. MOMP, heparin-binding proteins), C. pneumonie (e.g. MOMP, heparin-binding proteins), C. psittaci; Clostridium spp., including C. tetani (such as tetanus toxin), C. botulinum (for example botulinum toxin), C. difficile (e.g. clostridium toxins A or B); Corynebacterium spp., including C. diphtheriae (e.g. diphtheria toxin); Ehrlichia spp., including E. equi and the agent of the Human Granulocytic Ehrlichiosis; Rickettsia spp, including R.rickettsii; Enterococcus spp., including E. faecalis, E. faecium; Escherichia spp, including enterotoxic E. coli (for example colonization factors, heat-labile toxin or derivatives thereof, or heat-stable toxin), enterohemorragic E. coli, enteropathogenic E. coli (for example shiga toxin-like toxin); Haemophilus spp., including H. influenzae type B (e.g. PRP), non-typable H. influenzae, for example OMP26, high molecular weight adhesins, P5, P6, protein D and lipoprotein D, and fimbrin and fimbrin derived peptides (see for example U.S. Pat. No. 5,843,464); Helicobacter spp, including H. pylori (for example urease, catalase, vacuolating toxin); Pseudomonas spp, including P. aeruginosa; Legionella spp, including L. pneumophila; Leptospira spp., including L. interrogans; Listeria spp., including L. monocytogenes; Moraxella spp, including M catarrhalis, also known as Branhamella catarrhalis (for example high and low molecular weight adhesins and invasins); Morexella Catarrhalis (including outer membrane vesicles thereof, and OMP106 (see for example W097/41731)); Mycobacterium spp., including M. tuberculosis (for example ESAT6, Antigen 85A, -B or -C), M. bovis, M. leprae, M. avium, M. paratuberculosis, M. smegmatis; Neisseria spp, including N. gonorrhea and N. meningitidis (for example capsular polysaccharides and conjugates thereof, transferrin-binding proteins, lactoferrin binding proteins, PilC, adhesins); Neisseria mengitidis B (including outer membrane vesicles thereof, and NspA (see for example WO 96/29412); Salmonella spp, including S. typhi, S. paratyphi, S. choleraesuis, S. enteritidis; Shigella spp, including S. sonnei, S. dysenteriae, S. flexnerii; Staphylococcus spp., including S. aureus, S. epidermidis; Streptococcus spp, including S. pneumonie (e.g. capsular polysaccharides and conjugates thereof, PsaA, PspA, streptolysin, choline-binding proteins) and the protein antigen Pneumolysin (Biochem Biophys Acta, 1989, 67, 1007; Rubins et al., Microbial Pathogenesis, 25, 337-342), and mutant detoxified derivatives thereof (see for example WO 90/06951; WO 99/03884); Treponema spp., including T. pallidum (e.g. the outer membrane proteins), T. denticola, T. hyodysenteriae; Vibrio spp, including V. cholera (for example cholera toxin); and Yersinia spp, including Y. enterocolitica (for example a Yop protein), Y. pestis, Y. pseudotuberculosis.

Parasite/Fungal Antigens

Parasitic/fungal antigens or antigenic determinants may be derived, for example, from:

Babesia spp., including B. microti; Candida spp., including C. albicans; Cryptococcus spp., including C. neoformans; Entamoeba spp., including E. histolytica; Giardia spp., including; G. lamblia; Leshmania spp., including L. major; Plasmodium faciparum (MSP1, AMA1, MSP3, EBA, GLURP, RAP1, RAP2, Sequestrin, PfEMP1, Pf332, LSA1, LSA3, STARP, SALSA, PfEXP1, Pfs25, Pfs28, PFS27/25, Pfs16, Pfs48/45, Pfs230 and their analogues in Plasmodium spp.); Pneumocystis spp., including P. carinii; Schisostoma spp., including S. mansoni; Trichomonas spp., including T. vaginalis; Toxoplasma spp., including T. gondii (for example SAG2, SAG3, Tg34); Trypanosoma spp., including T. cruzi.

Approved/licensed vaccines include, for example anthrax vaccines such as Biothrax (BioPort Corp); tuberculosis (BCG) vaccines such as TICE BCG (Organon Teknika Corp) and Mycobax (Aventis Pasteur, Ltd); diphtheria & tetanus toxoid and acellular pertussis (DTP) vaccines such as Tripedia (Aventis Pasteur, Inc), Infanrix (GlaxoSmithKline), and DAPTACEL (Aventis Pasteur, Ltd); Haemophilus b conjugate vaccines (e.g. diphtheria CRM197 protein conjugates such as HibTITER from Lederle Lab Div, American Cyanamid Co; meningococcal protein conjugates such as PedvaxHIB from Merck & Co, Inc; and tetanus toxoid conjugates such as ActHIB from Aventis Pasteur, SA); Hepatitis A vaccines such as Havrix (GlaxoSmithKline) and VAQTA (Merck & Co, Inc); combined Hepatitis A and Hepatitis B (recombinant) vaccines such as Twinrix (GlaxoSmithKline); recombinant Hepatitis B vaccines such as Recombivax HB (Merck & Co, Inc) and Engerix-B (GlaxoSmithKline); influenza virus vaccines such as Fluvirin (Evans Vaccine), FluShield (Wyeth Laboratories, Inc) and Fluzone (Aventis Pasteur, Inc); Japanese Encephalitis virus vaccine such as JE-Vax (Research Foundation for Microbial Diseases of Osaka University); Measles virus vaccines such as Attenuvax (Merck & Co, Inc); measles and mumps virus vaccines such as M-M-Vax (Merck & Co, Inc); measles, mumps, and rubella virus vaccines such as M-M-R II (Merck & Co, Inc); meningococcal polysaccharide vaccines (Groups A, C, Y and W-135 combined) such as Menomune-A/C/Y/W-135 (Aventis Pasteur, Inc); mumps virus vaccines such as Mumpsvax (Merck & Co, Inc); pneumococcal vaccines such as Pneumovax (Merck & Co, Inc) and Pnu-Imune (Lederle Lab Div, American Cyanamid Co); Pneumococcal 7-valent conjugate vaccines (e.g. diphtheria CRM197 Protein conjugates such as Prevnar from Lederle Lab Div, American Cyanamid Co); poliovirus vaccines such as Poliovax (Aventis Pasteur, Ltd); poliovirus vaccines such as IPOL (Aventis Pasteur, SA); rabies vaccines such as Imovax (Aventis Pasteur, SA) and RabAvert (Chiron Behring GmbH & Co); rubella virus vaccines such as Meruvax II (Merck & Co, Inc); Typhoid Vi polysaccharide vaccines such as TYPHIM Vi (Aventis Pasteur, SA); Varicella virus vaccines such as Varivax (Merck & Co, Inc) and Yellow Fever vaccines such as YF-Vax (Aventis Pasteur, Inc).

Cancer/Tumour Antigens

The term “cancer antigen or antigenic determinant” or “tumour antigen or antigenic determinant” as used herein preferably means an antigen or antigenic determinant which is present on (or associated with) a cancer cell and not typically on normal cells, or an antigen or antigenic determinant which is present on cancer cells in greater amounts than on normal (non-cancer) cells, or an antigen or antigenic determinant which is present on cancer cells in a different form than that found on normal (non-cancer) cells.

Cancer antigens include, for example (but without limitation):

beta chain of human chorionic gonadotropin (hCG beta) antigen, carcinoembryonic antigen, EGFRvIII antigen, Globo H antigen, GM2 antigen, GP100 antigen, HER2/neu antigen, KSA antigen, Le (y) antigen, MUCI antigen, MAGE 1 antigen, MAGE 2 antigen, MUC2 antigen, MUC3 antigen, MUC4 antigen, MUC5AC antigen, MUC5B antigen, MUC7 antigen, PSA antigen, PSCA antigen, PSMA antigen, Thompson-Friedenreich antigen (TF), Tn antigen, sTn antigen, TRP 1 antigen, TRP 2 antigen, tumor-specific immunoglobulin variable region and tyrosinase antigen.

It will be appreciated that in accordance with this aspect of the present invention antigens and antigenic determinants may be used in many different forms. For example, antigens or antigenic determinants may be present as isolated proteins or peptides (for example in so-called “subunit vaccines”) or, for example, as cell-associated or virus-associated antigens or antigenic determinants (for example in either live or killed pathogen strains). Live pathogens will preferably be attenuated in known manner. Alternatively, antigens or antigenic determinants may be generated in situ in the subject by use of a polynucleotide coding for an antigen or antigenic determinant (as in so-called “DNA vaccination”, although it will be appreciated that the polynucleotides which may be used with this approach are not limited to DNA, and may also include RNA and modified polynucleotides as discussed above).

Reducing Immune Pathology

Immune pathology may result from an inappropriate or excessive host response. Non-specific immune responses such as inflammation provide an essential first line of host defense against many pathogens. Normally this is a transient phase, which terminates once the host has acquired specific effector mechanisms such as antibodies or cytotoxic T lymphocytes. In some cases however, especially with chronic pathogenic infections, these non-specific responses may persist to an inappropriate or excessive degree, without significantly reducing the infection, and may then become counterproductive. In these situations, it may be appropriate to reduce the inappropriate immune reponse to relieve symptoms in the patient. The infection may then clear over time or where necessary may be further treated for example with anti-infective drugs.

Thus, in a further aspect of the invention there is provided a product comprising i) an RNAi agent for increasing Notch signalling; and ii) a pathogen antigen or antigenic determinant, or a polynucleotide coding for a pathogen antigen or antigenic determinant; as a combined preparation for simultaneous, contemporaneous, separate or sequential use for reduction of an immune response to said pathogen antigen or antigenic determinant.

In a further aspect there is provided a method for reducing an immune response to a pathogen antigen or antigenic determinant in a mammal comprising simultaneously, contemporaneously, separately or sequentially administering in vivo, in either order:
i) an effective amount of an RNAi agent for increasing Notch signalling; and
ii) an effective amount of a pathogen antigen or antigenic determinant, or a polynucleotide coding for a pathogen antigen or antigenic determinant.
   Assays

Whether a substance can be used for modulating Notch signalling may be determined using suitable screening assays, for example, as described in the Examples herein.

For example, Notch signalling can be monitored either through protein assays or through nucleic acid assays. Activation of the Notch receptor leads to the proteolytic cleavage of its cytoplasmic domain and the translocation thereof into the cell nucleus. The “detectable signal” referred to herein may be any detectable manifestation attributable to the presence of the cleaved intracellular domain of Notch. Thus, increased Notch signalling can be assessed at the protein level by measuring intracellular concentrations of the cleaved Notch domain. Activation of the Notch receptor also catalyses a series of downstream reactions leading to changes in the levels of expression of certain well defined genes. Thus, increased Notch signalling can be assessed at the nucleic acid level by say measuring intracellular concentrations of specific mRNAs. In one preferred embodiment of the present invention, the assay is a protein assay. In another preferred embodiment of the present invention, the assay is a nucleic acid assay.

The advantage of using a nucleic acid assay is that they are sensitive and that small samples can be analysed.

The intracellular concentration of a particular mRNA, measured at any given time, reflects the level of expression of the corresponding gene at that time. Thus, levels of mRNA of downstream target genes of the Notch signalling pathway can be measured in an indirect assay of the T-cells of the immune system. In particular, an increase in levels of Deltex, Hes-1 and/or IL-10 mRNA may, for instance, indicate induced anergy while an increase in levels of Dll-1 or IFN-γ mRNA, or in the levels of mRNA encoding cytokines such as IL-2, IL-5 and IL-13, may indicate improved responsiveness.

Various nucleic acid assays are known. Any convention technique which is known or which is subsequently disclosed may be employed. Examples of suitable nucleic acid assay are mentioned below and include amplification, PCR, RT-PCR, RNase protection, blotting, spectrometry, reporter gene assays, gene chip arrays and other hybridization methods.

In particular, gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately labelled probe. Those skilled in the art will readily envisage how these methods may be modified, if desired.

PCR was originally developed as a means of amplifying DNA from an impure sample. The technique is based on a temperature cycle which repeatedly heats and cools the reaction solution allowing primers to anneal to target sequences and extension of those primers for the formation of duplicate daughter strands. RT-PCR uses an RNA template for generation of a first strand cDNA with a reverse transcriptase. The cDNA is then amplified according to standard PCR protocol. Repeated cycles of synthesis and denaturation result in an exponential increase in the number of copies of the target DNA produced. However, as reaction components become limiting, the rate of amplification decreases until a plateau is reached and there is little or no net increase in PCR product. The higher the starting copy number of the nucleic acid target, the sooner this “end-point” is reached.

Real-time PCR uses probes labeled with a fluorescent tag or fluorescent dyes and differs from end-point PCR for quantitative assays in that it is used to detect PCR products as they accumulate rather than for the measurement of product accumulation after a fixed number of cycles. The reactions are characterized by the point in time during cycling when amplification of a target sequence is first detected through a significant increase in fluorescence.

The ribonuclease protection (RNase protection) assay is an extremely sensitive technique for the quantitation of specific RNAs in solution. The ribonuclease protection assay can be performed on total cellular RNA or poly(A)-selected mRNA as a target. The sensitivity of the ribonuclease protection assay derives from the use of a complementary in vitro transcript probe which is radiolabeled to high specific activity. The probe and target RNA are hybridized in solution, after which the mixture is diluted and treated with ribonuclease (RNase) to degrade all remaining single-stranded RNA. The hybridized portion of the probe will be protected from digestion and can be visualized via electrophoresis of the mixture on a denaturing polyacrylamide gel followed by autoradiography. Since the protected fragments are analyzed by high resolution polyacrylamide gel electrophoresis, the ribonuclease protection assay can be employed to accurately map mRNA features. If the probe is hybridized at a molar excess with respect to the target RNA, then the resulting signal will be directly proportional to the amount of complementary RNA in the sample.

Gene expression may also be detected using a reporter system. Such a reporter system may comprise a readily identifiable marker under the control of an expression system, e.g. of the gene being monitored. Fluorescent markers, which can be detected and sorted by FACS, are preferred. Especially preferred are GFP and luciferase. Another type of preferred reporter is cell surface markers, i.e. proteins expressed on the cell surface and therefore easily identifiable.

In general, reporter constructs useful for detecting Notch signalling by expression of a reporter gene may be constructed according to the general teaching of Sambrook et al (1989). Typically, constructs according to the invention comprise a promoter by the gene of interest, and a coding sequence encoding the desired reporter constructs, for example of GFP or luciferase. Vectors encoding GFP and luciferase are known in the art and available commercially.

Sorting of cells, based upon detection of expression of genes, may be performed by any technique known in the art, as exemplified above. For example, cells may be sorted by flow cytometry or FACS. For a general reference, see Flow Cytometry and Cell Sorting: A Laboratory Manual (1992) A. Radbruch (Ed.), Springer Laboratory, New York.

Flow cytometry is a powerful method for studying and purifying cells. It has found wide application, particularly in immunology and cell biology: however, the capabilities of the FACS can be applied in many other fields of biology. The acronym F.A.C.S. stands for Fluorescence Activated Cell Sorting, and is used interchangeably with “flow cytometry”. The principle of FACS is that individual cells, held in a thin stream of fluid, are passed through one or more laser beams, causing light to be scattered and fluorescent dyes to emit light at various frequencies. Photomultiplier tubes (PMT) convert light to electrical signals, which are interpreted by software to generate data about the cells. Sub-populations of cells with defined characteristics can be identified and automatically sorted from the suspension at very high purity (˜100%).

FACS can be used to measure gene expression in cells transfected with recombinant DNA encoding polypeptides. This can be achieved directly, by labelling of the protein product, or indirectly by using a reporter gene in the construct. Examples of reporter genes are β-galactosidase and Green Fluorescent Protein (GFP). β-galactosidase activity can be detected by FACS using fluorogenic substrates such as fluorescein digalactoside (FDG). FDG is introduced into cells by hypotonic shock, and is cleaved by the enzyme to generate a fluorescent product, which is trapped within the cell. One enzyme can therefore generate a large amount of fluorescent product. Cells expressing GFP constructs will fluoresce without the addition of a substrate. Mutants of GFP are available which have different excitation frequencies, but which emit fluorescence in the same channel. In a two-laser FACS machine, it is possible to distinguish cells which are excited by the different lasers and therefore assay two transfections at the same time.

Alternative means of cell sorting may also be employed. For example, the invention comprises the use of nucleic acid probes complementary to mRNA. Such probes can be used to identify cells expressing polypeptides individually, such that they may subsequently be sorted either manually, or using FACS sorting. Nucleic acid probes complementary to mRNA may be prepared according to the teaching set forth above, using the general procedures as described by Sambrook et al (1989) supra.

In a preferred embodiment, the invention comprises the use of an antisense nucleic acid molecule, complementary to a mRNA, conjugated to a fluorophore which may be used in FACS cell sorting.

Methods have also been described for obtaining information about gene expression and identity using so-called gene chip arrays or high density DNA arrays (Chee M. et al. (1996) Science 274:601-614 (Chee)). These high density arrays are particularly useful for diagnostic and prognostic purposes. Use may also be made of In vivo Expression Technology (IVET) (Camilli et al. (1994) Proc Natl Acad Sci USA 91:2634-2638 (Camilli)). IVET identifies genes up-regulated during say treatment or disease when compared to laboratory culture.

The advantage of using a protein assay is that Notch activation can be directly measured. Assay techniques that can be used to determine levels of a polypeptide are well known to those skilled in the art. Such assay methods include radioimmunoassays, competitive-binding assays, Western Blot analysis, antibody sandwich assays, antibody detection, FACS and ELISA assays.

As described above the modulator of Notch signalling may also be an immune cell which has been treated to modulate expression or interaction of Notch, a Notch ligand or the Notch signalling pathway. Such cells may readily be prepared, for example, as described in WO 00/36089 in the name of Lorantis Ltd, the text of which is herein incorporated by reference.

Antigen Presenting Cells

Where required, antigen-presenting cells (APCs) may be “professional” antigen presenting cells or may be another cell that may be induced to present antigen to T cells. Alternatively a APC precursor may be used which differentiates or is activated under the conditions of culture to produce an APC. An APC for use in the ex vivo methods of the invention is typically isolated from a tumour or peripheral blood found within the body of a patient. Preferably the APC or precursor is of human origin. However, where APCs are used in preliminary in vitro screening procedures to identify and test suitable nucleic acid sequences, APCs from any suitable source, such as a healthy patient, may be used.

APCs include dendritic cells (DCs) such as interdigitating DCs or follicular DCs, Langerhans cells, PBMCs, macrophages, B-lymphocytes, or other cell types such as epithelial cells, fibroblasts or endothelial cells, activated or engineered by transfection to express a MHC molecule (Class I or II) on their surfaces. Precursors of APCs include CD34⁺ cells, monocytes, fibroblasts and endothelial cells. The APCs or precursors may be modified by the culture conditions or may be genetically modified, for instance by transfection of one or more genes encoding proteins which play a role in antigen presentation and/or in combination of selected cytokine genes which would promote to immune potentiation (for example IL-2, IL-12, IFN-γ, TNF-α, IL-18 etc.). Such proteins include MHC molecules (Class I or Class II), CD80, CD86, or CD40. Most preferably DCs or DC-precursors are included as a source of APCs.

Dendritic cells (DCs) can be isolated/prepared by a number of means, for example they can either be purified directly from peripheral blood, or generated from CD34⁺ precursor cells for example after mobilisation into peripheral blood by treatment with GM-CSF, or directly from bone marrow. From peripheral blood, adherent precursors can be treated with a GM-CSF/IL-4 mixture (Inaba K, et al. (1992) J. Exp. Med. 175: 1157-1167 (Inaba)), or from bone marrow, non-adherent CD34⁺ cells can be treated with GM-CSF and TNF-a (Caux C, et al. (1992) Nature 360: 258-261 (Caux)). DCs can also be routinely prepared from the peripheral blood of human volunteers, similarly to the method of Sallusto and Lanzavecchia (Sallusto F and Lanzavecchia A (1994) J. Exp. Med. 179: 1109-1118) using purified peripheral blood mononucleocytes (PBMCs) and treating 2 hour adherent cells with GM-CSF and IL-4. If required, these may be depleted of CD19⁺ B cells and CD3⁺, CD2⁺ T cells using magnetic beads (Coffin R S, et al. (1998) Gene Therapy 5: 718-722 (Coffin)). Culture conditions may include other cytokines such as GM-CSF or IL-4 for the maintenance and, or activity of the dendritic cells or other antigen presenting cells.

Thus, it will be understood that the term “antigen presenting cell or the like” are used herein is not intended to be limited to APCs. The skilled man will understand that any vehicle capable of presenting to the T cell population may be used, for the sake of convenience the term APCs is used to refer to all these. As indicated above, preferred examples of suitable APCs include dendritic cells, L cells, hybridomas, fibroblasts, lymphomas, macrophages, B cells or synthetic APCs such as lipid membranes.

T Cells

Where required, T cells from any suitable source, such as a healthy patient, may be used and may be obtained from blood or another source (such as lymph nodes, spleen, or bone marrow). They may optionally be enriched or purified by standard procedures. The T cells may be used in combination with other immune cells, obtained from the same or a different individual. Alternatively whole blood may be used or leukocyte enriched blood or purified white blood cells as a source of T cells and other cell types. It is particularly preferred to use helper T cells (CD4⁺). Alternatively other T cells such as CD8⁺ cells may be used. It may also be convenient to use cell lines such as T cell hybridomas.

Exposure of Agent to APCs and T Cells

T cells/APCs may be cultured as described above. The APCs/T cells may be incubated/exposed to active agents. For example, they may be prepared for administration to a patient or incubated with T cells in vitro (ex vivo).

Where treated ex-vivo, modified cells of the present invention are preferably administered to a host by direct injection into the lymph nodes of the patient. Typically from 10⁴ to 10⁸ treated cells, preferably from 10⁵ to 10⁷ cells, more preferably about 10⁶ cells are administered to the patient. Preferably, the cells will be taken from an enriched cell population.

As used herein, the term “enriched” as applied to the cell populations of the invention refers to a more homogeneous population of cells which have fewer other cells with which they are naturally associated. An enriched population of cells can be achieved by several methods known in the art. For example, an enriched population of T-cells can be obtained using immunoaffinity chromatography using monoclonal antibodies specific for determinants found only on T-cells.

Enriched populations can also be obtained from mixed cell suspensions by positive selection (collecting only the desired cells) or negative selection (removing the undesirable cells). The technology for capturing specific cells on affinity materials is well known in the art (Wigzel, et al., J. Exp. Med., 128:23, 1969; Mage, et al., J. Immunol. Meth., 15:47, 1977; Wysocki, et al., Proc. Natl. Acad. Sci. U.S.A., 75:2844, 1978; Schrempf-Decker, et al., J. Immunol Meth., 32:285, 1980; Muller-Sieburg, et al., Cell, 44:653, 1986).

Monoclonal antibodies against antigens specific for mature, differentiated cells have been used in a variety of negative selection strategies to remove undesired cells, for example, to deplete T-cells or malignant cells from allogeneic or autologous marrow grafts, respectively (Gee, et al., J.N.C.I. 80:154, 1988). Purification of human hematopoietic cells by negative selection with monoclonal antibodies and immunomagnetic microspheres can be accomplished using multiple monoclonal antibodies (Griffin, et al., Blood, 63:904, 1984).

Procedures for separation of cells may include magnetic separation, using antibodycoated magnetic beads, affinity chromatography, cytotoxic agents joined to a monoclonal antibody or used in conjunction with a monoclonal antibody, for example, complement and cytotoxins, and “panning” with antibodies attached to a solid matrix, for example, plate, or other convenient technique. Techniques providing accurate separation include fluorescence activated cell sorters, which can have varying degrees of sophistication, for example, a plurality of color channels, low angle and obtuse light scattering detecting channels, impedance channels, etc.

It will be appreciated that in one embodiment the therapeutic agents used in the present invention may be administered directly to patients in vivo. Alternatively or in addition, the agents may be administered to cells such as T cells and/or APCs in an ex vivo manner. For example, leukocytes such as T cells or APCs may be obtained from a patient or donor in known manner, treated/incubated ex vivo in the manner of the present invention, and then administered to a patient. In addition, it will be appreciated that a combination of routes of administration may be employed if desired. For example, where appropriate one component (such as the modulator of Notch signalling) may be administered ex-vivo and the other may be administered in vivo, or vice versa.

Introduction of Nucleic Acid Sequences into APCs and T-Cells

T-cells and APCs as described above are cultured in a suitable culture medium such as DMEM or other defined media, optionally in the presence of fetal calf serum.

Polypeptide substances may be administered to T-cells and/or APCs by introducing nucleic acid constructs/viral vectors encoding the polypeptide into cells under conditions that allow for expression of the polypeptide in the T-cell and/or APC. Similarly, nucleic acid constructs encoding antisense constructs may be introduced into the T-cells and/or APCs by transfection, viral infection or viral transduction.

In a preferred embodiment, nucleotide sequences will be operably linked to control sequences, including promoters/enhancers and other expression regulation signals. The term “operably linked” means that the components described are in a relationship permitting them to function in their intended manner. A regulatory sequence “operably linked” to a coding sequence is peferably ligated in such a way that expression of the coding sequence is achieved under condition compatible with the control sequences.

The promoter is typically selected from promoters which are functional in mammalian cells, although prokaryotic promoters and promoters functional in other eukaryotic cells may be used. The promoter is typically derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of a-actin, b-actin, tubulin) or, alternatively, a tissue-specific manner (such as promoters of the genes for pyruvate kinase). Tissue-specific promoters specific for lymphocytes, dendritic cells, skin, brain cells and epithelial cells within the eye are particularly preferred, for example the CD2, CD11c, keratin 14, Wnt-1 and Rhodopsin promoters respectively. Preferably the epithelial cell promoter SPC is used. They may also be promoters that respond to specific stimuli, for example promoters that bind steroid hormone receptors. Viral promoters may also be used, for example the Moloney murine leukaemia virus long terminal repeat (MMLV LTR) promoter, the rous sarcoma virus (RSV) LTR promoter or the human cytomegalovirus (CMV) I.E. promoter.

It may also be advantageous for the promoters to be inducible so that the levels of expression of the heterologous gene can be regulated during the life-time of the cell. Inducible means that the levels of expression obtained using the promoter can be regulated.

Any of the above promoters may be modified by the addition of further regulatory sequences, for example enhancer sequences. Chimeric promoters may also be used comprising sequence elements from two or more different promoters.

Alternatively (or in addition), the regulatory sequences may be cell specific such that the gene of interest is only expressed in cells of use in the present invention. Such cells include, for example, APCs and T-cells.

If required, a small aliquot of cells may be tested for up-regulation of Notch signalling activity as described above. The cells may be prepared for administration to a patient or incubated with T-cells in vitro (ex vivo).

Tolerisation Assays

Any of the assays described above (see “Assays”) can be adapted to monitor or to detect reduced reactivity and tolerisation in immune cells for use in clinical applications. Such assays will involve, for example, detecting increased Notch-ligand expression or activity in host cells or monitoring Notch cleavage in donor cells. Further methods of monitoring immune cell activity are set out below.

Immune cell activity may be monitored by any suitable method known to those skilled in the art. For example, cytotoxic activity may be monitored. Natural killer (NK) cells will demonstrate enhanced cytotoxic activity after activation. Therefore any drop in or stabilisation of cytotoxicity will be an indication of reduced reactivity.

Once activated, leukocytes express a variety of new cell surface antigens. NK cells, for example, will express transferrin receptor, HLA-DR and the CD25 IL-2 receptor after activation. Reduced reactivity may therefore be assayed by monitoring expression of these antigens.

Hara et al. Human T-cell Activation: III, Rapid Induction of a Phosphorylated 28 kD/32 kD Disulfide linked Early Activation Antigen (EA-1) by 12-0-tetradecanoyl Phorbol-13-Acetate, Mitogens and Antigens, J. Exp. Med., 164:1988 (1986), and Cosulich et al. Functional Characterization of an Antigen (MLR3) Involved in an Early Step of T-Cell Activation, PNAS, 84:4205 (1987), have described cell surface antigens that are expressed on T-cells shortly after activation. These antigens, EA-1 and MLR3 respectively, are glycoproteins having major components of 28 kD and 32 kD. EA-1 and MLR3 are not HLA class II antigens and an MLR3 Mab will block IL-1 binding. These antigens appear on activated T-cells within 18 hours and can therefore be used to monitor immune cell reactivity.

Additionally, leukocyte reactivity may be monitored as described in EP 0325489, which is incorporated herein by reference. Briefly this is accomplished using a monoclonal antibody (“Anti-Leu23”) which interacts with a cellular antigen recognised by the monoclonal antibody produced by the hybridoma designated as ATCC No. HB-9627.

Anti-Leu 23 recognises a cell surface antigen on activated and antigen stimulated leukocytes. On activated NK cells, the antigen, Leu 23, is expressed within 4 hours after activation and continues to be expressed as late as 72 hours after activation. Leu 23 is a disulfide-linked homodimer composed of 24 kD subunits with at least two N-linked carbohydrates.

Because the appearance of Leu 23 on NK cells correlates with the development of cytotoxicity and because the appearance of Leu 23 on certain T-cells correlates with stimulation of the T-cell antigen receptor complex, Anti-Leu 23 is useful in monitoring the reactivity of leukocytes.

Further details of techniques for the monitoring of immune cell reactivity may be found in:
‘The Natural Killer Cell’ Lewis C. E. and J. O'D. McGee 1992. Oxford University Press;
Trinchieri G. ‘Biology of Natural Killer Cells’ Adv. Immunol. 1989 vol 47 pp 187-376;
‘Cytokines of the Immune Response’ Chapter 7 in “Handbook of Immune Response Genes”.
Mak T. W. and J. J. L. Simard 1998, which are incorporated herein by reference.
   Preparation of Primed APCs and Lymphocytes

According to one aspect of the invention immune cells may be used to present antigens or allergens and/or may be treated with active agents. Thus, for example, Antigen Presenting Cells (APCs) may be cultured in a suitable culture medium such as DMEM or other defined media, optionally in the presence of a serum such as fetal calf serum. Optimum cytokine concentrations may be determined by titration. One or more active agents are then typically added to the culture medium together with the antigen of interest. The antigen may be added before, after or at substantially the same time as the substance(s). Cells are typically incubated with the substance(s) and antigen for at least one hour, preferably at least 3 hours, suitably at least 9, 12, 24, 48 or 36 or more hours at 37° C. If required, a small aliquot of cells may be tested for modulated target gene expression as described above. Alternatively, cell activity may be measured by the inhibition of T cell activation by monitoring surface markers, cytokine secretion or proliferation as described in WO98/20142. APCs transfected with a nucleic acid construct directing the expression of, for example Serrate, may be used as a control.

As discussed above, polypeptide substances may be administered to APCs by introducing nucleic acid constructs/viral vectors encoding the polypeptide into cells under conditions that allow for expression of the polypeptide in the APC. Similarly, nucleic acid constructs encoding antigens may be introduced into the APCs by transfection, viral infection or viral transduction.

Preparation of Regulatory T Cells (and B Cells) Ex Vivo

The techniques described below are described in relation to T cells, but are equally applicable to B cells. The techniques employed are essentially identical to those described for APCs alone except that T cells are generally co-cultured with the APCs. However, it may be preferred to prepare primed APCs first and then incubate them with T cells. For example, once the primed APCs have been prepared, they may be pelleted and washed with PBS before being resuspended in fresh culture medium. This has the advantage that if, for example, it is desired to treat the T cells with a different substance(s), then the T cell will not be brought into contact with the different substance(s) used with the APC. Once primed APCs have been prepared, it is not always necessary to administer any substances to the T cell since the primed APC is itself capable of promoting immunotolerance leading to increased Notch or Notch ligand expression in the T cell, presumably via Notch/Notch ligand interactions between the primed APC and T cell.

Incubations will typically be for at least 1 hour, preferably at least 3, 6, 12, 24, 48 or 36 or more hours, in suitable culture medium at 37° C. The progress of Notch signalling may be determined for a small aliquot of cells using the methods described above. Induction of immunotolerance may be determined, for example, by subsequently challenging T cells with antigen and measuring IL-2 production compared with control cells not exposed to APCs.

Primed T cells or B cells may also be used to induce immunotolerance in other T cells or B cells in the absence of APCs using similar culture techniques and incubation times.

Alternatively, T cells may be cultured and primed in the absence of APCs by use of APC substitutes such as anti-TCR antibodies (e.g. anti-CD3) with or without antibodies to costimulatory molecules (e.g. anti-CD28) or alternatively T cells may be activated with MHC-peptide complexes (e.g. tetramers).

Induction of immunotolerance may be determined by subsequently challenging T cells with antigen and measuring IL-2 production compared with control cells not exposed to APCs.

T cells or B cells which have been primed in this way may be used according to the invention to promote or increase immunotolerance in other T cells or B cells.

Administration

Suitably the active agents are administered in combination with a pharmaceutically acceptable carrier or diluent. The pharmaceutically acceptable carrier or diluent may be, for example, sterile isotonic saline solutions, or other isotonic solutions such as phosphate-buffered saline. The conjugates of the present invention may be admixed with any suitable binder(s), lubricant(s), suspending agent(s), coating agent(s), solubilising agent(s). It is also preferred to formulate the compound in an orally active form.

Pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

For some applications, active agents may be administered orally in the form of tablets containing excipients such as starch or lactose, or in capsules or ovules either alone or in admixture with excipients, or in the form of elixirs, solutions or suspensions containing flavouring or colouring agents.

Alternatively or in addition, active agents may be administered by inhalation, intranasally or in the form of aerosol, or in the form of a suppository or pessary, or they may be applied topically in the form of a lotion, solution, cream, ointment or dusting powder. An alternative means of transdermal administration is by use of a skin patch. For example, they can be incorporated into a cream consisting of an aqueous emulsion of polyethylene glycols or liquid paraffin. They can also be incorporated, at a concentration of between 1 and 10% by weight, into an ointment consisting of a white wax or white soft paraffin base together with such stabilisers and preservatives as may be required.

Active agents such as polynucleotides and proteins/polypeptides may also be administered by viral or non-viral techniques. Viral delivery mechanisms include but are not limited to adenoviral vectors, adeno-associated viral (AAV) vectors, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof. The routes for such delivery mechanisms include but are not limited to mucosal, nasal, oral, parenteral, gastrointestinal, topical, or sublingual routes. Active agents may be adminstered by conventional DNA delivery techniques, such as DNA vaccination etc., or injected or otherwise delivered with needleless systems, such as ballistic delivery on particles coated with the DNA for delivery to the epidermis or other sites such as mucosal surfaces.

Typically, the physician will determine the actual dosage which will be most suitable for an individual patient and it will vary with the age, weight and response of the particular patient. The above dosages are exemplary of the average case. There can, of course, be individual instances where higher or lower dosage ranges are merited, and such are within the scope of this invention.

In general, a therapeutically effective oral or intravenous dose is likely to range from 0.01 to 50 mg/kg body weight of the subject to be treated, preferably 0.1 to 20 mg/kg. The conjugate may also be administered by intravenous infusion, at a dose which is likely to range from 0.001-10 mg/kg/hr.

Tablets or capsules of the conjugates may be administered singly or two or more at a time, as appropriate. It is also possible to administer the conjugates in sustained release formulations.

Active agents may also be injected parenterally, for example intracavernosally, intravenously, intramuscularly or subcutaneously

For parenteral administration, active agents may be used in the form of a sterile aqueous solution which may contain other substances, for example enough salts or monosaccharides to make the solution isotonic with blood.

For buccal or sublingual administration, agents may be administered in the form of tablets or lozenges which can be formulated in a conventional manner.

For oral, parenteral, buccal and sublingual administration to subjects (such as patients), the dosage level of active agents and their pharmaceutically acceptable salts and solvates may typically be from 10 to 500 mg (in single or divided doses). Thus, and by way of example, tablets or capsules may contain from 5 to 100 mg of active agent for administration singly, or two or more at a time, as appropriate.

The routes of administration and dosages described are intended only as a guide since a skilled practitioner will be able to determine readily the optimum route of administration and dosage for any particular patient depending on, for example, the age, weight and condition of the patient.

The term treatment or therapy as used herein should be taken to encompass diagnostic and prophylatic applications.

The treatment of the present invention includes both human and veterinary applications.

Where treated ex-vivo, modified cells of the present invention are preferably administered to a host by direct injection into the lymph nodes of the patient. Typically from 10⁴ to 10⁸ treated cells, preferably from 10⁵ to 10⁷ cells, more preferably about 10⁶ cells are administered to the patient. Preferably, the cells will be taken from an enriched cell population.

As used herein, the term “enriched” as applied to the cell populations of the invention refers to a more homogeneous population of cells which have fewer other cells with which they are naturally associated. An enriched population of cells can be achieved by several methods known in the art. For example, an enriched population of T-cells can be obtained using immunoaffinity chromatography using monoclonal antibodies specific for determinants found only on T-cells.

Enriched populations can also be obtained from mixed cell suspensions by positive selection (collecting only the desired cells) or negative selection (removing the undesirable cells). The technology for capturing specific cells on affinity materials is well known in the art (Wigzel, et al., J. Exp. Med., 128:23, 1969; Mage, et al., J. Immunol. Meth., 15:47, 1977; Wysocki, et al., Proc. Natl. Acad. Sci. U.S.A., 75:2844, 1978; Schrempf-Decker, et al., J. Immunol Meth., 32:285, 1980; Muller-Sieburg, et al., Cell, 44:653, 1986).

Monoclonal antibodies against antigens specific for mature, differentiated cells have been used in a variety of negative selection strategies to remove undesired cells, for example, to deplete T-cells or malignant cells from allogeneic or autologous marrow grafts, respectively (Gee, et al., J.N.C.I. 80:154, 1988). Purification of human hematopoietic cells by negative selection with monoclonal antibodies and immunomagnetic microspheres can be accomplished using multiple monoclonal antibodies (Griffin, et al., Blood, 63:904, 1984).

Procedures for separation of cells may include magnetic separation, using antibodycoated magnetic beads, affinity chromatography, cytotoxic agents joined to a monoclonal antibody or used in conjunction with a monoclonal antibody, for example, complement and cytotoxins, and “panning” with antibodies attached to a solid matrix, for example, plate, or other convenient technique. Techniques providing accurate separation include fluorescence activated cell sorters, which can have varying degrees of sophistication, for example, a plurality of color channels, low angle and obtuse light scattering detecting channels, impedance channels, etc.

Combination Treatments

By “simultaneously” is meant that the active agents are administered at substantially the same time, and preferably together in the same formulation.

By “contemporaneously” it is meant that the active agents are administered closely in time, e.g., one agent is administered within from about one minute to within about one day before or after another. Any contemporaneous time is useful. However, it will often be the case that when not administered simultaneously, the agents will be administered within about one minute to within about eight hours, and preferably within less than about one to about four hours. When administered contemporaneously, the agents are preferably administered at the same site on the animal. The term “same site” includes the exact location, but can be within about 0.5 to about 15 centimeters, preferably from within about 0.5 to about 5 centimeters.

The term “separately” as used herein means that the agents are administered at an interval, for example at an interval of about a day to several weeks or months. The active agents may be administered in either order.

The term “sequentially” as used herein means that the agents are administered in sequence, for example at an interval or intervals of minutes, hours, days or weeks. If appropriate the active agents may be administered in a regular repeating cycle.

It will be appreciated that in one embodiment the therapeutic agents used in the present invention may be administered directly to patients in vivo. Alternatively or in addition, the agents may be administered to cells such as T cells and/or APCs in an ex vivo manner. For example, leukocytes such as T cells or APCs may be obtained from a patient or donor in known manner, treated/incubated ex vivo in the manner of the present invention, and then administered to a patient. In addition, it will be appreciated that a combination of routes of administration may be employed if desired. For example, where appropriate one component (such as the modulator of Notch signalling) may be administered ex-vivo and the other may be administered in vivo, or vice versa.

Chemical Cross-Linking

Chemically coupled sequences can be prepared from individual proteins sequences and coupled using known chemically coupling techniques. The conjugate can be assembled using conventional solution- or solid-phase peptide synthesis methods, affording a fully protected precursor with only the terminal amino group in deprotected reactive form. This function can then be reacted directly with a protein for T cell signalling modulation or a suitable reactive derivative thereof. Alternatively, this amino group may be converted into a different functional group suitable for reaction with a cargo moiety or a linker. Thus, e.g. reaction of the amino group with succinic anhydride will provide a selectively addressable carboxyl group, while further peptide chain extension with a cysteine derivative will result in a selectively addressable thiol group. Once a suitable selectively addressable functional group has been obtained in the delivery vector precursor, a protein for T cell signalling modulation or a derivative thereof may be attached through e.g. amide, ester, or disulphide bond formation. Cross-linking reagents which can be utilized are discussed, for example, in Neans, G. E. and Feeney, R. E., Chemical Modification of Proteins, Holden-Day, 1974, pp. 39-43.

As discussed above the target protein and protein for T cell signalling modulation may be linked directly or indirectly via a cleavable linker moiety. Direct linkage may occur through any convenient functional group on the protein for T cell signalling modulation such as a hydroxy, carboxy or amino group. Indirect linkage which is preferable, will occur through a linking moiety. Suitable linking moieties include bi- and multi-functional alkyl, aryl, aralkyl or peptidic moieties, alkyl, aryl or aralkyl aldehydes acids esters and anyhdrides, sulphydryl or carboxyl groups, such as maleimido benzoic acid derivatives, maleimido proprionic acid derivatives and succinimido derivatives or may be derived from cyanuric bromide or chloride, carbonyldiimidazole, succinimidyl esters or. sulphonic halides and the like. The functional groups on the linker moiety used to form covalent bonds between linker and protein for T cell signalling modulation on the one hand, as well as linker and target protein on the other hand, may be two or more of, e.g., amino, hydrazino, hydroxyl, thiol, maleimido, carbonyl, and carboxyl groups, etc. The linker moiety may include a short sequence of from 1 to 4 amino acid residues that optionally includes a cysteine residue through which the linker moiety bonds to the target protein.

Various preferred features and embodiments of the present invention will now be described in more detail by way of non-limiting Examples.

### EXAMPLE 1

Knockdown of Human Delta 1 Expression

The knockdown of overexpressed and stably expressed hDelta in CHO and CHO-Delta cells respectively was studied using 3 siRNAs. The sequences of the oligos used were:

| | |
|---|---|
| No. 1 sense | |
| GGAGUUGUCAACAAGAAGtt | (SEQ ID NO: 29) |
| No. 1 antisense | |
| CUUCUUGUUGACGAACUCCtg | (SEQ ID NO: 30) |
| Target sequence- | |
| AAGGAGTTCGTCAACAAGAAG | (SEQ ID NO: 31) |
| No. 2 sense | |
| GGACCUGAACUACUGCACAtt | (SEQ ID NO: 32) |
| No. 2 antisense | |
| UGUGCAGUAGUUCAGGUCCtg | (SEQ ID NO: 33) |
| Target sequence- | |
| AAGGACCTGAACTACTGCACA | (SEQ ID NO: 34) |
| No. 3 sense | |
| GGUGUAAAAUGGAAGUGAGtt | (SEQ ID NO: 35) |
| No. 3 antisense | |
| CUCACUUCCAUUUUACACCtc | (SEQ ID NO: 36) |
| Target sequence- | |
| AAGGTGTAAAATGGAAGTGAG | (SEQ ID NO: 37) |

A cDNA clone spanning the complete coding sequence of the human Delta1 gene (see, e.g. GenBank Accession No AF003522) was isolated from a cDNA library using a PCR-based screening strategy and cloned into the mammalian expression vector pcDNA3.1-V5-HisA (Invitrogen) without a stop codon, to generate the plasmid pcDNA3.1 FL hDelta1 V5-His. When expressed in mammalian cells, this plasmid expresses the full-length human Delta1 protein with V5 and His tags at the 3′ end of the intracellular domain.

Chinese Hamster Ovary (CHO) cells (CHO-K1) were transfected with pcDNA3.1 FL hDelta1 V5-His using Lipofectamine 2000 transfection reagent to create CHO cells expressing human Delta1 and maintained in DMEM plus 10% (HI)FCS plus glutamine plus P/S.

24 hours later the cells were further transfected with 100 nM final concentration Delta1 siRNAs (or Luc siRNA as control; Luc siRNA target sequence was GATTATGTCCGGTTATGTA (SEQ ID NO: 38), sense oligo was GAUUAUGUCCGGUUAUGUAtt (SEQ ID NO: 39), antisense oligo was UACAUAACCGGACAUAAUCtt (SEQ ID NO: 40)) using siPORT amine transfection reagent (Ambion). An additional 24 hours later the cells were plated onto CHO-N2 reporter cells (CHO cells expressing full length human Notch2 and a CBF1-luciferase reporter construct as described in WO 03/012441, Lorantis, e.g. see Example 7 therein) at 5×10⁵ cells/ml in the presence of 10 mM LiCl, in duplicate and incubated overnight.

100 ul Supernatant was then removed from all wells and 100 μl of SteadyGlo™ luciferase assay reagent (Promega) was added and the cells were left at room temperature for 5 minutes. The mixture was pipetted up and down 4 times to ensure cell lysis and contents from each well were transferred into a white 96-well OptiPlate™ (Packard). Luminescence was measured in a TopCount™ counter (Packard).

Level of gene knockdown is shown by percentage activity, where luciferase expression seen with the Luc siRNA control is shown as 100% activity. Results are shown in FIG. 10.

Cells were also harvested into sample buffer and an equal concentration loaded onto 12% SDS-PAGE gel and Western blotted with anti-V5 HRP. Results are shown in FIG. 11.

A clear knockdown in gene expression is seen when cells were transfected with Delta siRNA with both CHO-N2 (luciferase) assay and Western Blot.

### EXAMPLE 2

Knockdown in CHO-Delta Cells

Stable CHO-Delta cells were transfected with Delta 1 siRNAs as in Example 1 at a final concentration of 100 nM or with GAPDH siRNA (Ambion) as a control, using siPORT amine transfection reagent. 24 hours post-transfection the cells were harvested and plated onto CHO-N2 cells at 5×10⁵ cells/ml. After overnight incubation, luciferase expression was measured using a Steady-Glo luciferase assay as described in Example 1. Level of gene knockdown is shown by percentage activity, where luciferase expression seen with the GAPDH siRNA control is shown as 100% activity. Results are shown in FIG. 12.

Knockdown in expression was seen with all 3 Delta1 siRNAs.

### EXAMPLE 3

Knockdown of hDelta 1 Expression in Jurkat Cells

Jurkat cells (Clone E6.1) were co-transfected with pcDNA3.1 FL hdelta1 V5-His (4 ug) and Delta1 siRNAs (as in Example 1) were added to a final concentration of 500 nM using an Amaxa Nucleofector™ Kit (Amaxa Inc, MD, US) using cell line nucleofector solution V and protocol S18. 6 hours and 24 hours after transfection cells were harvested into sample buffer and an equal concentration of protein was loaded onto 12% SDS-PAGE and Western blotted with anti-V5 HRP. Results are shown in FIG. 13.

Knockdown in gene expression is clearly seen when cells were transfected with Delta1 siRNAs.

### EXAMPLE 4

Knockdown of Endogenous Delta1 Expression in Human CD4+ T Cells

Human peripheral blood mononuclear cells (PBMC) were purified from blood using Ficoll-Paque separation medium (Pharmacia). Briefly, 28 ml of blood were overlaid on 21 ml of Ficoll-Paque separation medium and centrifuged at 18-20° C. for 40 minutes at 400 g. PBMC were recovered from the interface and washed 3 times before use for CD4+ T cell purification.

Human CD4+ T cells were isolated by positive selection using anti-CD4 microbeads from Miltenyi Biotech according to the manufacturer's instructions.

The CD4+ T cells were transfected with a final concentration of 100 nM Delta1 siRNAs 30 numbers 1 & 2 using an Amaxa Nucleofector™ Kit (Amaxa Inc, MD, US) with Human CD4 Nucleofector solution and protocol U14. Transfected cells were seeded at 400,000 cells/well on plates coated with 10 ug/ml anti-CD3 antibody (clone UCHT1, BD Biosciences) plus 2 ug/ml soluble anti-CD28 antibody (clone CD28.2, BD Biosciences.

The supernatants were harvested 72 hours post-transfection at 37° C./5% CO₂/humidified atmosphere and cytokine (IL2, IL-5, IL-10 & IFNγ) production was evaluated by ELISA using BD OptEIA kit for IL-10 (Catalog No 555157), R&D Duoset IL-5 (Catalog No DY205), R&D Duoset IFNg (Catalog No DY285) and R&D biotinylated and monoclonal anti-IL-2 antibodies (Catalog No BAF202 and MAB602) for IL-2. Results for IL-2, IL-5 and IFN-g are shown in FIGS. 14-16 respectively.

### EXAMPLE 5

Knockdown of Human Jagged 1 Levels

Initial experiments examined the knockdown of overexpressed Jagged 1 in CHO cells, using 3 siRNAs with sequences as follows:

| | |
|---|---|
| No. 1 sense- | |
| GGUCCUAUACGUUGUUGUtt | (SEQ ID NO: 41) |
| No. 1 antisense- | |
| ACAAGCAACGUAUAGGACCtc | (SEQ ID NO: 42) |
| Target sequence- | |
| GGTCCTATACGTTGTTGT | (SEQ ID NO: 43) |
| No. 2 sense- | |
| GGCGUGGGAUUCCAGUAAUtt | (SEQ ID NO: 44) |
| No. 2 antisense- | |
| AUUACUGGAAUCCCACGCCtc | (SEQ ID NO: 45) |
| Target sequence- | |
| GGCGTGGGATTCCAGTAAT | (SEQ ID NO: 46) |
| No. 3 sense- | |
| GGAAAUCAAAGUGCUAUUAtt | (SEQ ID NO: 47) |
| No. 3 antisense- | |
| UAAUAGCACUUUGAUUUCCtc | (SEQ ID NO: 48) |
| Target sequence- | |
| GGAAATCAAAGTGCTATTA | (SEQ ID NO: 49) |

Knockdown in CHO Cells

A cDNA clone spanning the complete coding sequence of the human Jagged1 gene (see, e.g. GenBank Accession No AF028593) was isolated from a cDNA library using a PCR-based screening strategy and cloned into the mammalian expression vector pcDNA3.1-V5-HisA (Invitrogen) without a stop codon, to generate the plasmid pcDNA3.1 FL hjagged1 V5-His. When expressed in mammalian cells, this plasmid expresses the full-length human Jagged1 protein with V5 and His tags at the 3′ end of the intracellular domain.

CHO cells were transfected with pcDNA3.1 FL hjagged1 V5-His using Lipofectamine 2000 transfection reagent and 24 hours later were further transfected with 100 nM final concentration Jagged1 siRNAs or Luc siRNA (as siRNA control), using siPORT Amine transfection reagent. An additional 24 hours later cells were plated onto CHO-N2 cells at 5×10⁵ cells/ml in the presence of 10 mM LiCl, in duplicate. After overnight incubation cells were assayed for luciferase expression by Steady-Glo luciferase assay as described in EXAMPLE 1. Level of gene knockdown is shown by percentage activity, where luciferase expression seen with the Luc siRNA control is shown as 100% activity. Results are shown in FIG. 17.

Cells were also harvested into sample buffer and an equal concentration loaded onto 8% SDS-PAGE gel and Western blotted with anti-V5 HRP.

Knockdown in expression can be seen with all the Jagged siRNAs in the CHO-N2 assay and in a Western Blot (FIG. 18) with anti-V5 HRP. Probing the blot with anti-AKT confirms equal protein loading.

### EXAMPLE 6

Knockdown in Jurkats

Jurkat cells were co-transfected with pcDNA3.1 FL Jagged1 V5-His and 500 nM final concentration of Jagged 1 siRNAs using an Amaxa Nucleofector™ Kit (Amaxa Inc, MD, US) with Cell Line Nucleofector solution V and using manufacturer's protocol S18. Cells were harvested 6 & 24 hours post-transfection and equal protein concentrations were loaded onto 8% SDS-Page and Western blotted with anti-V5 HRP. Results are shown in FIG. 19.

Knockdown in gene expression can be seen, despite the fact that protein transfer was poor, probably due to the large size of Jagged 1.

### EXAMPLE 7

Delta1 Knockdown in Dendritic Cells (DCs)

Monocyte derived DCs were purified from Buffy coats using CD14+ beads (Miltenyi Biotec) and incubated for 6 days in RPMI+10% FBS,+ glutamine, pen/strep (standard amounts) and 2-ME supplemented with IL-4 and GMCSF 50 ng/ml. Cells were matured on day 6 with TNFα 20 ng/ml, 24 h. Cells were harvested, counted and resuspended in Amaxa Dendritic cell nucleofection solution at 2×10⁶ cells/100 ul solution. Cells were transfected with 100 nM final concentration human Delta1 siRNA (sequence as above) using Amaxa protocol U-02 and immediately resuspended in 1 ml pre-warmed media as above, supplemented with IL-4 and GMCSF as above.

Cells were harvested 24 h post-transfection and assayed for Delta1 expression (relative to 18S rRNA expression) by RT-PCR using a Lightcycler™ RT-PCR instrument according to the manufacturer's directions (Roche). Results are shown in FIG. 20.

### EXAMPLE 8

Delta1 Knockdown in a Mixed Lymphocyte Reaction (MLR)

Monocyte-derived DCs were purified from Buffy coats using CD14+ beads (Miltenyi Biotec) and incubated for 6 days in RPMI+10% FBS,+ glutamine, pen/strep (standard amounts) and 2-ME supplemented with IL-4 and GMCSF 50 ng/ml. Cells were matured on day 6 with TNFα 20 ng/ml, 24 h. Cells were harvested, counted and resuspended in Amaxa Dendritic cell nucleofection solution at 2×10⁶ cells/100 ul solution. Cells were transfected with 500 nM final concentration human Delta1 siRNA (sequence as above) using Amaxa protocol U-02 and immediately resuspended in 1 ml pre-warmed media as above, supplemented with IL-4 and GMCSF as above, incubated 24 h.

DCs were then washed 2×PBS and resuspended to top concentration of 4×10⁵ cells/ml in RPMI (as above except using 5% human serum (Sigma) rather than 10% FBS). Syngeneic and allogeneic CD4+ cells (purified typically from buffy coats using CD4+ beads, Miltenyi Biotec) were resuspended to 4×10⁶ cells/ml in RPMI+5% human serum (as above).

CD4+ cells were seeded at 4×10⁵ cells/well in 100 ul media (as above) in 96-well round bottomed plates. DCs added in 100 ul media at a ratio of 10:1, 20:1, 40:1 (CD4 to DC, ratio, constant CD4) and no DCs—all in triplicate. Cells were incubated for 5 days at 37° C., 5% CO₂, supernatants were then harvested and assayed by ELISA for cytokine (IFNγ) levels. Results are shown in FIG. 21.

As can be seen, hDelta siRNA in DCs promoted activation of CD4+ T-cells in this primary human mixed lymphocyte reaction (MLR). Knockdown of Delta 1 expression resulted in increased IFNγ in a ratio-dependent manner i.e more DCs corresponded to greater IFNγ expression.

### EXAMPLE 9

Jagged2 Knockdown in DCs

Monocyte derived DCs were purified from Buffy coats using CD14+0 beads (Miltenyi Biotec) and incubated for 6 days in RPMI+10% FBS,+ glutamine, pen/strep (standard amounts) and 2-ME supplemented with IL-4 and GMCSF 50 ng/ml. Cells were matured on day 6 with TNFα 20 ng/ml, 24 h. Cells were harvested, counted and resuspended in Amaxa Dendritic cell nucleofection solution at 2×10⁶ cells/100 ul solution. Cells were transfected with 500 nM final concentration human Jagged2 siRNA using Amaxa protocol U-02 and immediately resuspended in 1 ml pre-warmed media as above, supplemented with IL-4 and GMCSF as above.

Jagged2 siRNA sequences used were as follows:

| | |
|---|---|
| No. 1 sense | |
| 5′ GGAGUGCAAGGAAGCUGUGtt 3′ | (SEQ ID NO: 50) |
| No. 1 antisense | |
| 5′ CACAGCUUCCUUGCACUCCtt 3′ | (SEQ ID NO: 51) |
| No. 1 target sequence | |
| 5′ GGAGTGCAAGGAAGCTGTGtt 3′ | (SEQ ID NO: 52) |
| No. 2 sense | |
| 5′ GGAAGCUGUGUGUAAACAAtt 3′ | (SEQ ID NO: 53) |
| No. 2 antisense | |
| 5′ UUGUUUACACACAGCUUCCtt 3′ | (SEQ ID NO: 54) |
| No. 2 target sequence | |
| 5′ GGAAGCTGTGTGTAAACAAtt 3′ | (SEQ ID NO: 55) |

Cells were harvested 24 h post-transfection and assayed for Jagged2 expression (relative to 18S rRNA expression) by RT-PCR using a Lightcycler™ RT-PCR instrument according to the manufacturer's directions (Roche). Results are shown in FIG. 22.

### EXAMPLE 10

Effect of POFUT siRNA Knockdown in CD4s

CD4+ T-cells were purified using CD4+ beads, (Miltenyi Biotec). Cells were resuspended in Amaxa T cell nucleofection solution, 5×10⁶ cells/100 ul. Cells were transfected with 100 nM final concentration human POFUT-1 siRNA using Amaxa protocol U-14 and immediately resuspended in 1 ml pre-warmed media.

Human POFUT-1 siRNA sequences used were as follows:

| | |
|---|---|
| No. 1-sense | |
| 5′ GGAUUUCAUGGAGAAGCUGTT 3′ | (SEQ ID NO: 56) |
| antisense | |
| 5′ CAGCUUCUCCAUGAAAUCCTC 3′ | (SEQ ID NO: 57) |
| No. 2-sense | |
| 5′ GGCAUUUCCUUCAGUGCUUTT 3′ | (SEQ ID NO: 58) |
| antisense | |
| 5′ AAGCAUGAAGGAAAUGCCTG 3′ | (SEQ ID NO: 59) |
| No. 3-sense | |
| 5′ GCUGCUAAACCGUACCUUGTT 3′ | (SEQ ID NO: 60) |
| antisense | |
| 5′ CAAGGUACGGUUUAGCAGCTT 3′ | (SEQ ID NO: 61) |

A “scrambled” control used was for No.2 sequence as follows:

| | |
|---|---|
| Sense | |
| 5′ CUUAUUCUCGUUAUCCGGUtt 3′ | (SEQ ID NO: 62) |
| Antisense | |
| 5′ ACCGGAUAACGAGAAUAAGtt 3′ | (SEQ ID NO: 63) |

Cells were incubated 48 h on anti-CD3 coated wells (10 ug/ml) with 2 ug/ml soluble anti-CD28. Samples were harvested 24 h and 48 h post-transfection and assayed for POFUT expression (relative to 18S rRNA expression) by RT-PCR using a Lightcycler™ RT-PCR instrument according to the manufacturer's directions (Roche). Results are shown in FIG. 23.

### EXAMPLE 11

CBF-1 Knockdown in Jurkat Reporter Cells

Jurkat cells were co-transfected with CBF-VP16 (a construct comprising full-length human CBF1 sequence with a VP16 activation domain fused in frame to the 3′ end in a pSG5 expression vector (Stratagene); VP16 recruits transcriptional activators to the protein to drive transcription in the absence of Notch-IC), CBF-Luc reporter construct (fusion construct with luciferase expressed downstream of CBF-1; e.g. see WO 03/012441, Lorantis Ltd, Example 7) and 3 different CBF-specific siRNAs.

The human CBF-1 siRNA sequences used were as follows:

| | |
|---|---|
| No. 1-sense | |
| 5′ GGUUUUUUUGCCCACCUCCTT 3′ | (SEQ ID NO: 64) |
| antisense | |
| 5′ GGAGGUGGGCAAAAAAACCTT 3′ | (SEQ ID NO: 65) |
| No. 2-sense | |
| 5′ GGAAAGAACUAUUGCACAGTT 3′ | (SEQ ID NO: 66) |
| antisense | |
| 5′ CUGUGCAAUAGUUCUUUCCTT 3′ | (SEQ ID NO: 67) |
| No. 3-sense | |
| 5′ GGAGGUAAUUUUCAUGCCATT 3′ | (SEQ ID NO: 68) |
| antisense | |
| 5′ UGGCAUGAAAAUUACCUCCTT 3′ | (SEQ ID NO: 69) |

“Scrambled” controls were used for the No. 1 and No.2 sequences as follows:

| | |
|---|---|
| Control for No. 1: | |
| sense | |
| 5′ UUCUCCGUUCGUCCACGUUtt 3′ | (SEQ ID NO: 70) |
| antisense | |
| 5′ AACGUGGACGAACGGAGAAtt 3′ | (SEQ ID NO: 71) |
| Control for No. 2: | |
| sense | |
| 5′ AAGUGACAGUACGAUAGACtt 3′ | (SEQ ID NO: 72) |
| antisense | |
| 5′ GUCUAUCGUACUGUCACUUtt 3′ | (SEQ ID NO: 73) |

Knockdown of CBF-1expression results in reduced luciferase expression which was then measured by adding SteadyGlo™ luciferase assay reagent (Promega) and measuring Luminescence in a TopCount™ counter (Packard))

Results are shown in FIG. 24.

### EXAMPLE 12

Mouse Delta1 Knockdown in CHO Reporter Cells

CHO cells were co-transfected with a mDelta-Luc fusion reporter construct (with Luciferase expressed downstream of mdelta—therefore knockdown of Delta expression results in reduced luciferase expression which is then measured as described in Example 11 above) and two different mouse Delta 1 siRNAs.

Mouse Delta 1 siRNA sequences used were as follows:

| | |
|---|---|
| No. 1-sense | |
| 5′ GGAGUUCGUCAACAAGAAGTT 3′ | (SEQ ID NO: 74) |
| antisense | |
| 5′ CUUCUUGUUGACGAACUCCTG 3′ | (SEQ ID NO: 75) |
| No. 2-sense | |
| 5′ GGACCUUCUUUCGCGUAUGTT 3′ | (SEQ ID NO: 76) |
| antisense | |
| 5′ CAUACGCGAAAGAAGGUCCTG 3′ | (SEQ ID NO: 77) |

Results are shown in FIG. 25. Duplicate transfections were assayed in triplicate—hence 2 bars per siRNA type are shown. Firefly luciferase was normalised vs. constitutive Renilla luciferase.

The data shows that both mouse Delta1 siRNAs (same sequences as human Delta1siRNAs) were specific for mouse Delta, number 3 is a control included to show specificity of knockdown as this siRNA was against a region of mDelta not present in the construct.

### EXAMPLE 13

Knockdown in Reporter Cells (Co-transfection Method)

Reporter plasmids (as above) and siRNAs were transfected into CHO cells using Lipofectamine 2000™. Cells were seeded in DMEM supplemented with 10% v/v FBS, 50 iu/ml Penicillin, 50 μg/ml Streptomycin, 2 mM L-glutamine 24 h prior to transfection (6×10⁵ cells/well for 6-well plates, 5×10⁴ cells/well for 24-well plates). Complexes of 100 nM siRNA (Delta1, Jagged1 and Jagged2, sequences as above) with Lipofectamine 2000, and reporter plasmid DNA with Lipofectamine 2000 were prepared in Optimem™, incubated for 20 min at room temperature and then added to cells in DMEM supplemented with 10% v/v FBS, 2 mM L-glutamine. After 4 h incubation a further 3.5 ml or 500 μl (6-well and 24-well respectively) DMEM supplemented with 10% v/v FBS, 2 mM L-glutamine was added and cells incubated a further 24 h.

Knockdown was measured by adding SteadyGlo™ luciferase assay reagent (Promega) and measuring Luminescence in a TopCount™ counter (Packard), as described above.

In each case case appropriate “scrambled” siRNAs were included as controls. Results and corresponding Western blots are shown in FIG. 26.

### EXAMPLE 14

Ligand Knockdown in CD4+ T-Cells

CD4+ T-cells were purified using CD4+ beads (Miltenyi Biotec). Cells were resuspended in Amaxa T cell nucleofection solution, 5×10⁶ cells/100 ul. Cells were transfected with 100 nM final concentration human Notch ligand siRNA (sequences as above) using Amaxa protocol U-14 and immediately resuspended in 1 ml pre-warmed media.

Cells were incubated 48 h on anti-CD3 coated wells (10 ug/ml) with 2 ug/ml soluble anti-CD28. Samples were harvested 24 h and 48 h post-transfection and assayed for ligand expression (relative to 18S rRNA expression) by RT-PCR using a Lightcycler™ RT-PCR instrument according to the manufacturer's directions (Roche). Results are shown in FIG. 27.

### EXAMPLE 15

MLR—Jagged 1 and Jagged2 Knockdown in DCs

The MLR method of Example 8 above was repeated with use of Jagged1 and Jagged2 siRNAs (sequences as above) in place of Delta1 siRNA. Activation of CD4+ T-cells was observed with use of both Jagged1 and Jagged2 siRNA. Results are shown in FIG. 28.

The invention is further described by the following numbered paragraphs:

1. An RNAi agent which targets a component of a human Notch signalling pathway other than presenilin1 or presenilin2 by RNA interference to reduce expression of said component.

2. An RNAi agent as described in paragraph 1 which comprises an interfering ribonucleic acid (RNA).

3. An RNAi agent as described in paragraph 2 in the form of a siRNA.

4. An RNAi agent as described in paragraph 2 in the form of a shRNA.

5. An RNAi agent as described in paragraph 1 which comprises a transcription template of an interfering ribonucleic acid.

6. An RNAi agent as described in paragraph 5 wherein said transcription template comprises a DNA sequence.

7. An RNAi agent as described in paragraph 6 wherein said DNA sequence encodes a shRNA.

8. An RNAi agent as described in any of the preceding paragraphs wherein said RNAi agent targets a Notch ligand to reduce expression of thereof.

9. An RNAi agent as described in any of paragraphs 1 to 7 wherein said RNAi agent targets Delta to reduce expression thereof.

10. An RNAi agent as described in paragraph 9 wherein said RNAi agent targets Delta1, Delta3 or Delta4 to reduce expression thereof.

11. An RNAi agent as described in paragraph 10 wherein said RNAi agent targets Delta1 to reduce expression thereof.

12. An RNAi agent as described in paragraph 11 wherein the Delta1 target sequence comprises a sequence of about 19-22 nucleic acids of human Delta1.

13. An RNAi agent as described in any of paragraphs 1 to 7 wherein said RNAi agent targets Jagged to reduce expression thereof.

14. An RNAi agent as described in paragraph 13 wherein said RNAi agent targets Jagged 1 or Jagged 2 to reduce expression thereof.

15. An RNAi agent as described in paragraph 14 wherein said RNAi agent targets Jagged 1 to reduce expression thereof.

16. An RNAi agent as described in paragraph 15 wherein the Jagged1 target sequence comprises a sequence of about 19-22 nucleic acids of human Jagged1.

17. An RNAi agent as described in any of paragraphs 1 to 7 wherein said RNAi agent targets expression of Notch to reduce expression thereof

18. An RNAi agent as described in paragraph 17 wherein said RNAi agent targets Notch 1, 2, 3 or 4 to reduce expression thereof.

19. An RNAi agent as described in paragraph 18 wherein said RNAi agent targets Notch IC to reduce expression thereof.

20. An RNAi agent as described in any of paragraphs 1 to 7 wherein said RNAi agent targets a Fringe to reduce expression thereof.

21. An RNAi agent as described in any of paragraphs 1 to 7 wherein said RNAi agent targets a Notch IC protease complex component to reduce expression thereof.

22. An RNAi agent as described in any of paragraphs 1 to 7 wherein said RNAi agent targets a Notch Ubiquitin ligase to reduce expression thereof.

23. An RNAi agent as described in any of paragraphs 1 to 7 wherein said RNAi agent targets Deltex to reduce expression thereof.

24. An RNAi agent as described in any of paragraphs 1 to 7 wherein said RNAi agent targets a member of the HES family of basic helix-loop-helix transcriptional regulators, or a CSL transcriptional cofactor to reduce expression thereof.

25. An RNAi agent as described in any of the preceding paragraphs which targets Notch signalling in immune cells.

26. An RNAi agent as described in paragraph 25 which targets Notch signalling in T-cells, B-cells or APCs.

27. A method for treating a disease or disorder by modulating Notch signalling by RNA interference.

28. A method for treating an immune disease or disorder by modulating Notch signalling by RNA interference.

29. A method for modulating an immune response by modulating Notch signalling by RNA interference.

30. A method for treating a disease or disorder associated with Notch signaling comprising reducing expression of a component of the Notch signaling pathway in a target cell of a mammal, said method comprising administering to said mammal an effective amount of an RNAi agent specific for said component to reduce expression thereof.

31. A method as described in paragraph 30 wherein said RNAi agent comprises an interfering ribonucleic acid.

32. A method as described in paragraph 31 wherein said interfering ribonucleic acid comprises a siRNA.

33. A method as described in paragraph 31 wherein said interfering ribonucleic acid comprises a shRNA.

34. A method as described in paragraph 30 wherein said RNAi agent comprises a transcription template of an interfering ribonucleic acid.

35. A method as described in paragraph 34 wherein said transcription template comprises a DNA sequence.

36. A method as described in paragraph 35 wherein said DNA sequence encodes a shRNA.

37. A method as described in any of paragraphs 30 to 36 wherein said RNAi agent targets a Notch ligand to reduce expression thereof.

38. A method as described in any of paragraphs 30 to 36 wherein said RNAi agent targets Delta to reduce expression thereof.

39. A method as described in paragraph 38 wherein said RNAi agent targets Delta 1, 3 or 4 to reduce expression thereof.

40. A method as described in paragraph 39 wherein said RNAi agent targets Delta 1 to reduce expression thereof.

41. A method as described in paragraph 40 wherein the Delta1 target sequence comprises a sequence of about 19-22 nucleic acids of human Delta1.

42. A method as described in paragraph 39 wherein said RNAi agent targets Jagged to reduce expression thereof.

43. A method as described paragraph 42 wherein said RNAi agent targets Jagged 1 or 2 to reduce expression thereof.

44. A method as described in paragraph 43 wherein said RNAi agent targets Jagged 1 to reduce expression thereof.

45. A method as described in paragraph 44 wherein the Jagged1 target sequence comprises a sequence of about 19-22 nucleic acids of human Jagged1.

46. A method as described in any of paragraphs 30 to 36 wherein said RNAi agent targets Notch to reduce expression thereof.

47. A method as described in paragraph 46 wherein said RNAi agent targets Notch 1, 2, 3 or 4 to reduce expression thereof.

48. A method as described in paragraph 46 wherein said RNAi agent targets Notch IC to reduce expression thereof.

49. A method as described in any of paragraphs 30 to 36 wherein said RNAi agent targets Fringe to reduce expression thereof.

50. A method as described in any of paragraphs 30 to 36 wherein said RNAi agent targets a component of a Notch IC protease complex to reduce expression thereof

51. A method as described in any of paragraphs 30 to 36 wherein said RNAi agent targets a Notch Ubiquitin ligase to reduce expression thereof

52. A method as described in any of paragraphs 30 to 36 wherein said RNAi agent targets Deltex to reduce expression thereof

53. A method as described in any of paragraphs 30 to 36 wherein said RNAi agent targets a member of the HES family of basic helix-loop-helix transcriptional regulators, or a CSL transcriptional cofactor, to reduce expression thereof

54. A method as described in any of paragraphs 30 to 53 wherein said RNAi agent targets Notch signalling in immune cells

55. A method as described in paragraph 54 wherein said RNAi agent targets Notch signalling in T-cells, B-cells or APCs

56. The use of RNA interference to modulate Notch signaling for treatment of a disease or disorder.

57. The use of RNA interference to modulate Notch signaling for treatment of an immune disease or disorder.

58. The use of an RNAi agent targeting a component of the Notch signalling pathway to reduce an immune response.

59. The use of an RNAi agent targeting a component of the Notch signalling pathway to increase an immune response.

60. A use as described in any of paragraphs 56 to 59 wherein said RNAi agent comprises an interfering ribonucleic acid.

61. A use as described in paragraph 60 wherein said interfering ribonucleic acid comprises a siRNA.

62. A use as described in paragraph 60 wherein said interfering ribonucleic acid comprises a shRNA.

63. A use as described in any of paragraphs 56 to 59 wherein said RNAi agent comprises a transcription template of an interfering ribonucleic acid.

64. A use as described in paragraph 63 wherein said transcription template comprises a DNA sequence.

65. A use as described in paragraph 64 wherein said DNA sequence encodes a shRNA.

66. A use as described in any of paragraphs 56 to 65 wherein said RNAi agent targets a Notch ligand to reduce expression thereof.

67. A use as described in paragraph 66 wherein said RNAi agent targets Delta to reduce expression thereof.

68. A use as described in paragraph 67 wherein said RNAi agent targets Delta 1, 3 or 4 to reduce expression thereof.

69. A use as described in paragraph 68 wherein said RNAi agent targets Delta 1 to reduce expression thereof.

70. A use as described in paragraph 69 wherein the Delta1 target sequence comprises a sequence of about 19-22 nucleic acids of human Delta1.

71. A use as described in any of paragraphs 56 to 65 wherein said RNAi agent targets Jagged to reduce expression thereof.

72. A use as described in paragraph 71 wherein said RNAi agent targets Jagged 1 or 2 to reduce expression thereof.

73. A use as described in paragraph 72 wherein said RNAi agent targets Jagged 1 to reduce expression thereof.

74. A use as described in paragraph 73 wherein the Jagged1 target sequence comprises a sequence of about 19-22 nucleic acids of human Jagged1.

75. A use as described in any of paragraphs 56 to 65 wherein said RNAi agent targets Notch to reduce expression thereof.

76. A use as described in paragraph 75 wherein said RNAi agent targets Notch 1, 2, 3 or 4 to reduce expression thereof.

77. A use as described in paragraph 76 wherein said RNAi agent targets Notch IC to reduce expression thereof.

78. A use as described in any of paragraphs 56 to 65 wherein said RNAi agent targets Fringe to reduce expression thereof.

79. A use as described in any of paragraphs 56 to 65 wherein said RNAi agent targets a member of a Notch IC protease complex to reduce expression thereof

80. A use as described in any of paragraphs 56 to 65 wherein said RNAi agent targets Notch Ubiquitin ligase to reduce expression thereof

81. A use as described in any of paragraphs 56 to 65 wherein said RNAi agent targets Deltex to reduce expression thereof

82. A use as described in any of paragraphs 56 to 65 wherein said RNAi agent targets a member of the HES family of basic helix-loop-helix transcriptional regulators, or a CSL transcriptional cofactor to reduce expression thereof

83. A use as described in any of paragraphs 56 to 82 wherein said RNAi agent targets Notch signaling in immune cells

84. A use as described in paragraph 83 wherein said RNAi agent targets Notch signalling in T-cells, B-cells or APCs

85. A pharmaceutical composition for modulation of Notch signaling comprising an RNAi agent which downregulates expression of a component of the Notch signaling pathway by RNA interference.

86. A pharmaceutical composition for treatment of an immune disease or disorder comprising an RNAi agent which downregulates expression of a component of the Notch signaling pathway by RNA interference.

87. A pharmaceutical composition for modulation of an immune response comprising an RNAi agent which downregulates expression of a component of the Notch signaling pathway by RNA interference.

88. A pharmaceutical composition comprising:
i) an RNAi agent targeting a component of the Notch signaling pathway; and
ii) an antigen or antigenic determinant or a nucleic acid coding for an antigen or antigenic determinant;
   as a combined preparation for simultaneous, separate or sequential administration for the modulation of an immune response.

89. A pharmaceutical composition comprising:
i) an RNAi agent targeting a component of the Notch signaling pathway; and
ii) an antigen or antigenic determinant or a nucleic acid coding for an antigen or antigenic determinant;
   as a combined preparation for simultaneous, separate or sequential administration for the modulation of an immune response to said antigen or antigenic determinant.

90. A pharmaceutical composition comprising:
i) an RNAi agent targeting a component of the Notch signaling pathway; and
ii) an antigen or antigenic determinant or a nucleic acid coding for an antigen or antigenic determinant;
   as a combined preparation for simultaneous, separate or sequential administration for reducing an immune response to said antigen or antigenic determinant.

91. A pharmaceutical composition as described in any of paragraphs 85 to 90 wherein the antigen is an allergen, autoantigen, pathogen antigen or graft antigen.

92. A pharmaceutical composition comprising:
i) an RNAi agent targeting a component of the Notch signaling pathway; and
ii) an antigen or antigenic determinant or a nucleic acid coding for an antigen or antigenic determinant;
   as a combined preparation for simultaneous, separate or sequential administration for increasing an immune response to said antigen or antigenic determinant.

93. A pharmaceutical composition as described in paragraph 92 wherein the antigen is a pathogen or cancer antigen.

94. A pharmaceutical composition as described in any of paragraphs 85 to 93 wherein said RNAi agent comprises an interfering ribonucleic acid.

95. A pharmaceutical composition as described in paragraph 94 wherein said interfering ribonucleic acid comprises a siRNA.

96. A pharmaceutical composition as described in paragraph 94 wherein said interfering ribonucleic acid comprises a shRNA.

97. A pharmaceutical composition as described in any of paragraphs 85 to 93 wherein said RNAi agent comprises a transcription template of an interfering ribonucleic acid.

98. A pharmaceutical composition as described in paragraph 97 wherein said transcription template comprises a DNA sequence.

99. A pharmaceutical composition as described in paragraph 98 wherein said DNA sequence encodes a shRNA.

100. A pharmaceutical composition as described in any of paragraphs 85 to 99 wherein said RNAi agent targets a Notch ligand to reduce expression thereof.

101. A pharmaceutical composition as described in paragraph 100 wherein said RNAi agent targets Delta to reduce expression thereof.

102. A pharmaceutical composition as described in paragraph 101 wherein said RNAi agent targets Delta 1, 3 or 4 to reduce expression thereof.

103. A pharmaceutical composition as described in paragraph 102 wherein said RNAi agent targets Delta 1 to reduce expression thereof.

104. A pharmaceutical composition as described in paragraph 102 wherein the Delta1 target sequence comprises a sequence of about 19-22 nucleic acids of human Delta1.

105. A pharmaceutical composition as described in any of paragraphs 85 to 99 wherein said RNAi agent targets Jagged to reduce expression thereof.

106. A pharmaceutical composition as described in paragraph 105 wherein said RNAi agent targets Jagged 1 or 2 to reduce expression thereof.

107. A pharmaceutical composition as described in paragraph 106 wherein said RNAi agent targets Jagged 1 to reduce expression thereof.

108. A pharmaceutical composition as described in paragraph 107 wherein the Jagged1 target sequence comprises a sequence of about 19-22 nucleic acids of human Jagged1.

109. A pharmaceutical composition as described in any of paragraphs 85 to 99 wherein said RNAi agent targets Notch to reduce expression thereof.

110. A pharmaceutical composition as described in any of paragraphs 85 to 99 wherein said RNAi agent targets Notch 1, 2, 3 or 4 to reduce expression thereof.

111. A pharmaceutical composition as described in any of paragraphs 85 to 99 wherein said RNAi agent targets Notch IC to reduce expression thereof.

112. A pharmaceutical composition as described in any of paragraphs 85 to 99 wherein said RNAi agent targets Fringe to reduce expression thereof.

113. A pharmaceutical composition as described in any of paragraphs 85 to 99 wherein said RNAi agent targets a member of a Notch IC protease complex to reduce expression thereof

114. A pharmaceutical composition as described in any of paragraphs 85 to 99 wherein said RNAi agent targets Notch Ubiquitin ligase to reduce expression thereof

115. A pharmaceutical composition as described in any of paragraphs 85 to 99 wherein said RNAi agent targets Deltex to reduce expression thereof

116. A pharmaceutical composition as described in any of paragraphs 85 to 99 wherein said RNAi agent targets a member of the HES family of basic helix-loop-helix transcriptional regulators, or a CSL transcriptional cofactor to reduce expression thereof

117. A pharmaceutical composition as described in any of paragraphs 85 to 116 wherein said RNAi agent targets Notch signalling in immune cells

118. A pharmaceutical composition as described in paragraph 117 wherein said RNAi agent targets Notch signalling in T-cells, B-cells or APCs

119. A cancer vaccine composition comprising an RNAi agent targeting a component of the Notch signalling pathway which is effective to reduce Notch signalling.

120. A pathogen vaccine composition comprising an RNAi agent targeting a component of the Notch signalling pathway which is effective to reduce Notch signalling.

121. A vaccine composition as described in paragraph 119 or paragraph 120 wherein said RNAi agent comprises an interfering ribonucleic acid.

122. A vaccine composition as described in paragraph 121 wherein said interfering ribonucleic acid is in the form of a siRNA.

123. A vaccine composition as described in paragraph 121 wherein said interfering ribonucleic acid is in the form of a shRNA.

124. A vaccine composition as described in paragraph 119 or paragraph 120 wherein said RNAi agent comprises a transcription template of an interfering ribonucleic acid.

125. A vaccine composition as described in paragraph 124 wherein said transcription template comprises a DNA sequence.

126. A vaccine composition as described in paragraph 125 wherein said DNA sequence encodes a shRNA.

127. A vaccine composition as described in any of paragraphs 119 to 126 wherein said RNAi agent targets a Notch ligand to reduce expression thereof.

128. A vaccine composition as described in paragraph 127 wherein said RNAi agent targets Delta to reduce expression thereof.

129. A vaccine composition as described in paragraph 128 wherein said RNAi agent targets Delta 1, 3 or 4 to reduce expression thereof.

130. A vaccine composition as described in paragraph 129 wherein said RNAi agent targets Delta 1 to reduce expression thereof.

131. A vaccine composition as described in paragraph 129 wherein the Delta1 target sequence comprises a sequence of about 19-22 nucleic acids of human Delta1

132. A vaccine composition as described in paragraph 127 wherein said RNAi agent targets Jagged to reduce expression thereof.

133. A vaccine composition as described in paragraph 132 wherein said RNAi agent targets Jagged 1 or 2 to reduce expression thereof.

134. A vaccine composition as described in paragraph 133 wherein said RNAi agent targets Jagged 1 to reduce expression thereof.

135. A vaccine composition as described in paragraph 134 wherein the Jagged1 target sequence comprises a sequence of about 19-22 nucleic acids of human Jagged1.

136. A vaccine composition as described in any of paragraphs 119 to 126 wherein said RNAi agent targets Notch to reduce expression thereof.

137. A vaccine composition as described in paragraph 136 wherein said RNAi agent targets Notch 1, 2, 3 or 4 to reduce expression thereof.

138. A vaccine composition as described in paragraph 136 or paragraph 137 wherein said RNAi agent targets Notch IC to reduce expression thereof.

139. A vaccine composition as described in any of paragraphs 119 to 126 wherein said RNAi agent targets Fringe to reduce expression thereof.

140. A vaccine composition as described in any of paragraphs 119 to 126 wherein said RNAi agent targets a member of a Notch IC protease complex to reduce expression thereof.

141. A vaccine composition as described in any of paragraphs 119 to 126 wherein said RNAi agent targets Notch Ubiquitin ligase to reduce expression thereof.

142. A vaccine composition as described in any of paragraphs 119 to 126 wherein said RNAi agent targets Deltex to reduce expression thereof.

143. A vaccine composition as described in any of paragraphs 119 to 126 wherein said RNAi agent targets a member of the HES family of basic helix-loop-helix transcriptional regulators, or a CSL transcriptional cofactor, to reduce expression thereof.

144. A vaccine composition as described in any of paragraphs 119 to 143 wherein said RNAi agent targets Notch signalling in immune cells.

145. A vaccine composition as described in paragraph 144 wherein said RNAi agent targets Notch signalling in T-cells, B-cells or APCs.

146. A vector coding for an RNAi agent as described in any of paragraphs 1 to 26.

147. A vector comprising:
a. a first polynucleotide sequence coding for an RNAi agent as described in any of paragraphs 1 to 26; and
b. a second polynucleotide sequence coding for an antigen or antigenic determinant.

148. A vector as described in paragraph 146 or paragraph 147 wherein the antigen is an autoantigen, allergen, pathogen antigen or graft antigen or antigenic determinant thereof.

149. A vector as described in paragraph 146 or paragraph 147 wherein the antigen is a pathogen or tumour antigen or antigenic determinant thereof.

150. A vector as described in any of paragraphs 146 to 149 in the form of an expression vector.

151. A pharmaceutical composition comprising a vector as described in any of paragraphs 146 to 149.

### REFERENCES (INCORPORATED HEREIN BY REFERENCE)

Artavanis-Tsakonas S, et al. (1995) Science 268:225-232.
Artavanis-Tsakonas S, et al. (1999) Science 284:770-776.
Brucker K, et al. (2000) Nature 406:411-415.
Camilli et al. (1994) Proc Natl Acad Sci USA 91:2634-2638.
Caux C, et al. (1992) Nature 360: 258-261.
Chee M. et al. (1996) Science 274:601-614.
Dkuz et al. (1997) Cell 90: 271-280.
Hemmati-Brivanlou and Melton (1997) Cell 88:13-17.
Hicks C, et al. (2000) Nat. Cell. Biol. 2:515-520.
Hoyne G. F. et al (1999) Int Arch Allergy Immunol 118:122-124.
Hoyne et al. (2000) Immunology 100:281-288.
Hoyne G. F. et al (2000) Intl Immunol 12:177-185.
Hoyne, G. et al. (2001) Immunological Reviews 182:215-227.
Hsieh et al. (1996) Molecular & Cell Biology 16(3):952-959.
Iemura et al. (1998) PNAS 95:9337-9345.
Irvine K D (1999) Curr. Opin. Genet. Devel. 9:434-441.
Ju B J, et al. (2000) Nature 405:191-195.
Kusano and Raab-Truab (2001) J Virol 75(1):384-395.
Leimeister C. et al. (1999) Mech Dev 85(1-2):173-7.
Li et al. (1998) Immunity 8(1):43-55.
Lieber, T. et al. (1993) Genes Dev 7(10):1949-65.
Lu, F. M. et al. (1996) Proc Natl Acad Sci 93(11):5663-7.
Matsuno K, et al. (1998) Nat. Genet. 19:74-78.
Matsuno, K. et al. (1995) Development 121(8):2633-44.
McGuinness T. Et al (1996) Genomics 35(3):473-85.
Medhzhitov et al. (1997) Nature 388:394-397.
Moloney D J, et al. (2000) Nature 406:369-375.
Munro S, Freeman M. (2000) Curr. Biol. 10:813-820.
Ordentlich et al. (1998) Mol. Cell. Biol. 18:2230-2239.
Osborne B, Miele L. (1999) Immunity 11:653-663.
Panin V M, et al. (1997) Nature 387:908-912.
Sallusto F and Lanzavecchia A (1994) J. Exp. Med. 179: 1109-1118.
Sasai et al. (1994) Cell 79:779-790.
Schroeter, E. H. et al. (1998) Nature 393(6683):382-6.
Strobl et al. (2000) J Virol 74(4):1727-35.
Struhl G, Adachi A. Cell 93(4):649-60.
Takebayashi K. et al. (1994) J Biol Chem 269(7):150-6.
Tamura K, et al. (1995) Curr. Biol. 5:1416-1423.
Valenzuela et al. (1995) J. Neurosci. 15:6077-6084.
Weinmaster G. (2000) Curr. Opin. Genet. Dev. 10:363-369.
Wilson and Hemmati-Brivanlou (1997) Neuron 18:699-710.
Zhao et al. (1995) J. Immunol. 155:3904-3911.

1. An RNAi agent that targets a component of a human Notch signalling pathway other than presenilin1 or presenilin2 by RNA interference to reduce expression of said component.

2. The RNAi agent as claimed in claim 1, which comprises an interfering ribonucleic acid (RNA).

3. The RNAi agent as claimed in claim 2, wherein the interfering RNA is a siRNA.

4. The RNAi agent as claimed in claim 2, wherein the interfering RNA is a shRNA.

5. The RNAi agent as claimed in claim 1, which comprises a transcription template of an interfering ribonucleic acid.

6. The RNAi agent as claimed in claim 5, wherein said transcription template comprises a DNA sequence.

7. The RNAi agent as claimed in claim 6, wherein said DNA sequence encodes a shRNA.

8. The RNAi agent as claimed in claim 1, wherein the component of a human Notch signalling pathway is Notch ligand.

9. The RNAi agent as claimed in claim 1, wherein the component of a human Notch signalling pathway is selected from the group consisting of Delta, Jagged, Notch, Fringe, Notch IC protease complex, Notch ubiquitin ligase, Deltex, a member of the HES family of basic helix-loop-helix transcriptional regulators and a CSL transcriptional cofactor.

10. The RNAi agent as claimed in claim 9, the component of a human Notch signalling pathway is Delta 1, Delta 3 or Delta 4.

11. The RNAi agent as claimed in claim 10, the component of a human Notch signalling pathway is Delta 1.

12. The RNAi agent as claimed in claim 11, wherein said RNAi agent targets a sequence of about 19-22 nucleic acids of human Delta 1.

13. The RNAi agent as claimed in claim 9, wherein the component of a human Notch signalling pathway is Jagged 1 or Jagged 2.

14. The RNAi agent as claimed in claim 13, wherein the component of a human Notch signalling pathway is Jagged 1.

15. The RNAi agent as claimed in claim 14, wherein said RNAi agent targets a sequence of about 19-22 nucleic acids of human Jagged 1.

16. The RNAi agent as claimed in claim 9, wherein the component of a human Notch signalling pathway is Notch 1, 2, 3 or 4.

17. The RNAi agent as claimed in claim 16, wherein the component of a human Notch signalling pathway is Notch IC.

18. The RNAi agent as claimed in claim 1, which targets Notch signalling in immune cells.

19. The RNAi agent as claimed in claim 18, wherein the immune cells are T-cells, B-cells or APCs.

20. A method for treating a disease or disorder by modulating Notch signalling by RNA interference.

21. The method of claim 20, wherein the disease or disorder is an immune disease or disorder.

22. A method for modulating an immune response by modulating Notch signalling by RNA interference.

23. A method for treating a disease or disorder associated with Notch signaling comprising reducing expression of a component of the Notch signaling pathway in a target cell of a mammal, said method comprising administering to said mammal an effective amount of an RNAi agent specific for said component to reduce expression thereof.

24. The method as claimed in claim 23, wherein said RNAi agent comprises an interfering ribonucleic acid.

25. The method as claimed in claim 24, wherein said interfering ribonucleic acid comprises a siRNA.

26. The method as claimed in claim 24, wherein said interfering ribonucleic acid comprises a shRNA.

27. The method as claimed in claim 23, wherein said RNAi agent comprises a transcription template of an interfering ribonucleic acid.

28. The method as claimed in claim 27, wherein said transcription template comprises a DNA sequence.

29. The method as claimed in claim 28, wherein said DNA sequence encodes a shRNA.

30. The method as claimed in claim 23, wherein the component of a human Notch signalling pathway is Notch ligand.

31. The method as claimed in claim 23, wherein the component of a human Notch signalling pathway is selected from the group consisting of Delta, Jagged, Notch, Fringe, Notch IC protease complex, Notch ubiquitin ligase, Deltex, a member of the HES family of basic helix-loop-helix transcriptional regulators and a CSL transcriptional cofactor.

32. The method as claimed in claim 31, wherein the component of a human Notch signalling pathway is Delta 1, 3 or 4.

33. The method as claimed in claim 32, wherein the component of a human Notch signalling pathway is Delta 1.

34. The method as claimed in claim 33, wherein said RNAi agent targets a sequence of about 19-22 nucleic acids of human Delta 1.

35. The method as claimed claim 31, wherein the component of a human Notch signalling pathway is Jagged 1 or 2.

36. The method as claimed in claim 35, wherein the component of a human Notch signalling pathway is Jagged 1.

37. The method as claimed in claim 36, wherein said RNAi agent targets a sequence of about 19-22 nucleic acids of human Jagged 1.

38. The method as claimed in claim 31, wherein the component of a human Notch signalling pathway is Notch 1, 2, 3 or 4.

39. The method as claimed in claim 31, wherein the component of a human Notch signalling pathway is Notch IC to reduce expression thereof.

40. The method as claimed in claim 31, wherein said RNAi agent targets Notch signalling in immune cells

41. The method as claimed in claim 40, wherein the immune cells are T-cells, B-cells or APCs

42. A composition comprising the RNAi agent as claimed in claim 1.


SEQUENCE LISTING (151 sequences)

SEQ ID NO: 1 - protein, 63 aa, Drosophila sp.
WKTNKSESQYTSLEYDFRVTCDLNYYGSGCAKFCRPRDDSFGHSTCSETGEIICLTGWQGDYC

SEQ ID NO: 2 - protein, 63 aa, Homo sapiens
WSQDLHSSGRTDLKYSYRFVCDEHYYGEGCSVFCRPRDDAFGHFTCGERGEKVCNPGWKGPYC

SEQ ID NO: 3 - protein, 63 aa, Mus sp.
WSQDLHSSGRTDLRYSYRFVCDEHYYGEGCSVFCRPRDDAFGHFTCGDRGEKMCDPGWKGQYC

SEQ ID NO: 4 - protein, 63 aa, Rattus sp.
WSQDLHSSGRTDLRYSYRFVCDEHYYGEGCSVFCRPRDDAFGHFTCGERGEKMCDPGWKGQYC

SEQ ID NO: 5 - protein, 63 aa, Mus sp.
WRTDEQNDTLTRLSYSYRVICSDNYYGESCSRLCKKRDDHFGHYECQPDGSLSCLPGWTGKYC

SEQ ID NO: 6 - protein, 63 aa, Homo sapiens
WLLDEQTSTLTRLRYSYRVICSDNYYGDNCSRLCKKRNDHFGHYVCQPDGNLSCLPGWTGEYC

SEQ ID NO: 7 - protein, 63 aa, Rattus sp.
WQTLKQNTGIAHFEYQIRVTCDDHYYGFGCNKFCRPRDDFFGHYACDQNGNKTCMEGWMGPEC

SEQ ID NO: 8 - protein, 63 aa, Mus sp.
WQTLKQNTGIAHFEYQIRVTCDDHYYGFGCNKFCRPRDDFFGHYACDQNGNKTCMEGWMGPDC

SEQ ID NO: 9 - protein, 63 aa, Homo sapiens
WQTLKQNTGVAHFEYQIRVTCDDYYYGFGCNKFCRPRDDFFGHYACDQNGNKTCMEGWMGREC

SEQ ID NO: 10 - protein, 63 aa, Gallus sp.
WQTLKHNTGAAHFEYQIRVTCAEHYYGFGCNKFCRPRDDFFTHHTCDQNGNKTCLEGWTGPEC

SEQ ID NO: 11 - protein, 63 aa, Gallus sp.
WKTLQFNGPVANFEVQIRVKCDENYYSALCNKFCGPRDDFVGHYTCDQNGNKACMEGWMGEEC

SEQ ID NO: 12 - protein, 63 aa, Mus sp.
WKSLHFSGHVAHLELQIRVRCDENYYSATCNKFCRPRNDFFGHYTCDQYGNKACMDGWMGKEC

SEQ ID NO: 13 - protein, 63 aa, Homo sapiens; identical to SEQ ID NO: 12

SEQ ID NO: 14 - protein, 63 aa, Rattus sp.; identical to SEQ ID NO: 12

SEQ ID NO: 15 - protein, 63 aa, Homo sapiens; identical to SEQ ID NO: 12

SEQ ID NO: 16 - protein, 63 aa, Drosophila sp.
WKTLDHIGRNARITYRVRVQCAVTYYNTTCTTFCRPRDDQFGHYACGSEGQKLCLNGWQGVNC

SEQ ID NO: 17 - protein, 723 aa, Homo sapiens [sequence omitted]

SEQ ID NO: 18 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 19 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 20 - protein, 1218 aa, Homo sapiens
MRSPRTRGRSGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDFLVPLLSSVLTVAWICCLVTAFYWCLRKRRKPGSHTHSASEDNTTNNVREQLNQIKNPIEKHGANTVPIKDYENKNSKMSKIRTHNSEVEEDDMDKHQQKARFAKQPAYTLVDREEKPPNGTPTKHPNWTNKQDNRDLESAQSLNRMEYIV

SEQ ID NO: 21 - protein, 1238 aa, Homo sapiens [sequence omitted]

SEQ ID NO: 22 - DNA, 3162 bp, Homo sapiens
gaattccattttaagttatacaaaactgattaccataagtgcggtcgactgcttttatttttacgttgtgtgtgttggaaaaatgctaaaacatcagtctacaattctatatattgttattaaagattaatccaaccagcaacccaaggacatataagcgatttccactattgcatcagagcactcggcaggaaaggcctagccacggggaacattagaagctacagaagcattgcagagaagagaagatcccccgcgcgtccgccgctgttctaaggagagaagtgggggccccccaggctcgcgcgtggagcgaagcagcatgggcagtcggtgcgcgctggccctggcggtgctctcggccttgctgtgtcaggtctggagctctggggtgttcgaactgaagctgcaggagttcgtcaacaagaaggggctgctggggaaccgcaactgctgccgcgggggcgcggggccaccgccgtgcgcctgccggaccttcttccgcgtgtgcctcaagcactaccaggccagcgtgtcccccgagccgccctgcacctacggcagcgccgtcacccccgtgctgggcgtcgactccttcagtctgcccgacggcgggggcgccgactccgcgttcagcaaccccatccgcttccccttcggcttcacctggccgggcaccttctctctgattattgaagctctccacacagattctcctgatgacctcgcaacagaaaacccagaaagactcatcagccgcctggccacccagaggcacctgacggtgggcgaggagtggtcccaggacctgcacagcagcggccgcacggacctcaagtactcctaccgcttcgtgtgtgacgaacactactacggagagggctgctccgttttctgccgtccccgggacgatgccttcggccacttcacctgtggggagcgtggggagaaagtgtgcaaccctggctggaaagggccctactgcacagagccgatctgcctgcctggatgtgatgagcagcatggattttgtgacaaaccaggggaatgcaagtgcagagtgggctggcagggccggtactgtgacgagtgtatccgctatccaggctgtctccatggcacctgccagcagccctggcagtgcaactgccaggaaggctgggggggccttttctgcaaccaggacctgaactactgcacacaccataagccctgcaagaatggagccacctgcaccaacacgggccaggggagctacacttgctcttgccggcctgggtacacaggtgccacctgcgagctggggattgacgagtgtgaccccagcccttgtaagaacggagggagctgcacggatctcgagaacagctactcctgtacctgcccacccggcttctacggcaaaatctgtgaattgagtgccatgacctgtgcggacggcccttgctttaacgggggtcggtgctcagacagccccgatggagggtacagctgccgctgccccgtgggctactccggcttcaactgtgagaagaaaattgactactgcagctcttcaccctgttctaatggtgccaagtgtgtggacctcggtgatgcctacctgtgccgctgccaggccggcttctcggggaggcactgtgacgacaacgtggacgactgcgcctcctccccgtgcgccaacgggggcacctgccgggatggcgtgaacgacttctcctgcacctgcccgcctggctacacgggcaggaactgcagtgcccccgtcagcaggtgcgagcacgcaccctgccacaatggggccacctgccacgagaggggccacggctatgtgtgcgagtgtgcccgaggctacgggggtcccaactgccagttcctgctccccgagctgcccccgggcccagcggtggtggacctcactgagaagctagagggccagggcgggccattcccctgggtggccgtgtgcgccggggtcatccttgtcctcatgctgctgctgggctgtgccgctgtggtggtctgcgtccggctgaggctgcagaagcaccggcccccagccgacccctgccggggggagacggagaccatgaacaacctggccaactgccagcgtgagaaggacatctcagtcagcatcatcggggccacgcagatcaagaacaccaacaagaaggcggacttccacggggaccacagcgccgacaagaatggcttcaaggcccgctacccagcggtggactataacctcgtgcaggacctcaagggtgacgacaccgccgtcagggacgcgcacagcaagcgtgacaccaagtgccagccccagggctcctcaggggaggagaaggggaccccgaccacactcaggggtggagaagcatctgaaagaaaaaggccggactcgggctgttcaacttcaaaagacaccaagtaccagtcggtgtacgtcatatccgaggagaaggatgagtgcgtcatagcaactgaggtgtaaaatggaagtgagatggcaagactcccgtttctcttaaaataagtaaaattccaaggatatatgccccaacgaatgctgctgaagaggagggaggcctcgtggactgctgctgagaaaccgagttcagaccgagcaggttctcctcctgaggtcctcgacgcctgccgacagcctgtcgcggcccggccgcctgcggcactgccttccgtgacgtcgccgttgcactatggacagttgctcttaagagaatatatatttaaatgggtgaactgaattacgcataagaagcatgcactgcctgagtgtatattttggattcttatgagccagtcttttcttgaattagaaacacaaacactgcctttattgtcctttttgatacgaagatgtgctttttctagatggaaaagatgtgtgttattttttggatttgtaaaaatatttttcatgatatctgtaaagcttgagtattttgtgatgttcgttttttataatttaaattttggtaaatatgtacaaaggcacttcgggtctatgtgactatatttttttgtatataaatgtatttatggaatattgtgcaaatgttatttgagttttttactgttttgttaatgaagaaattcctttttaaaatatttttccaaaataaattttatgaggaattc

SEQ ID NO: 23 - DNA, 5455 bp, Homo sapiens
aggtggaagaggagggggagcgtctcaaagaagcgatcagaataataaaaggaggccgggctctttgccttctggaacgggccgctcttgaaagggcttttgaaaagtggtgttgttttccagtcgtgcatgctccaatcggcggagtatattagagccgggacgcggcggccgcaggggcagcggcgacggcagcaccggcggcagcaccagcgcgaacagcagcggcggcgtcccgagtgcccgcggcgcgcggcgcagcgatgcgttccccacggacgcgcggccggtccgggcgccccctaagcctcctgctcgccctgctctgtgccctgcgagccaaggtgtgtggggcctcgggtcagttcgagttggagatcctgtccatgcagaacgtgaacggggagctgcagaacgggaactgctgcggcggcgcccggaacccgggagaccgcaagtgcacccgcgacgagtgtgacacatacttcaaagtgtgcctcaaggagtatcagtcccgcgtcacggccggggggccctgcagcttcggctcagggtccacgcctgtcatcgggggcaacaccttcaacctcaaggccagccgcggcaacgaccgcaaccgcatcgtgctgcctttcagtttcgcctggccgaggtcctatacgttgcttgtggaggcgtgggattccagtaatgacaccgttcaacctgacagtattattgaaaaggcttctcactcgggcatgatcaaccccagccggcagtggcagacgctgaagcagaacacgggcgttgcccactttgagtatcagatccgcgtgacctgtgatgactactactatggctttggctgcaataagttctgccgccccagagatgacttctttggacactatgcctgtgaccagaatggcaacaaaacttgcatggaaggctggatgggccccgaatgtaacagagctatttgccgacaaggctgcagtcctaagcatgggtcttgcaaactcccaggtgactgcaggtgccagtacggctggcaaggcctgtactgtgataagtgcatcccacacccgggatgcgtccacggcatctgtaatgagccctggcagtgcctctgtgagaccaactggggcggccagctctgtgacaaagatctcaattactgtgggactcatcagccgtgtctcaacgggggaacttgtagcaacacaggccctgacaaatatcagtgttcctgccctgaggggtattcaggacccaactgtgaaattgctgagcacgcctgcctctctgatccctgtcacaacagaggcagctgtaaggagacctccctgggctttgagtgtgagtgttccccaggctggaccggccccacatgctctacaaacattgatgactgttctcctaataactgttcccacgggggcacctgccaggacctggttaacggatttaagtgtgtgtgccccccacagtggactgggaaaacgtgccagttagatgcaaatgaatgtgaggccaaaccttgtgtaaacgccaaatcctgtaagaatctcattgccagctactactgcgactgtcttcccggctggatgggtcagaattgtgacataaatattaatgactgccttggccagtgtcagaatgacgcctcctgtcgggatttggttaatggttatcgctgtatctgtccacctggctatgcaggcgatcactgtgagagagacatcgatgaatgtgccagcaacccctgtttgaatgggggtcactgtcagaatgaaatcaacagattccagtgtctgtgtcccactggtttctctggaaacctctgtcagctggacatcgattattgtgagcctaatccctgccagaacggtgcccagtgctacaaccgtgccagtgactatttctgcaagtgccccgaggactatgagggcaagaactgctcacacctgaaagaccactgccgcacgaccccctgtgaagtgattgacagctgcacagtggccatggcttccaacgacacacctgaaggggtgcggtatatttcctccaacgtctgtggtcctcacgggaagtgcaagagtcagtcgggaggcaaattcacctgtgactgtaacaaaggcttcacgggaacatactgccatgaaaatattaatgactgtgagagcaacccttgtagaaacggtggcacttgcatcgatggtgtcaactcctacaagtgcatctgtagtgacggctgggagggggcctactgtgaaaccaatattaatgactgcagccagaacccctgccacaatgggggcacgtgtcgcgacctggtcaatgacttctactgtgactgtaaaaatgggtggaaaggaaagacctgccactcacgtgacagtcagtgtgatgaggccacgtgcaacaacggtggcacctgctatgatgagggggatgcttttaagtgcatgtgtcctggcggctgggaaggaacaacctgtaacatagcccgaaacagtagctgcctgcccaacccctgccataatgggggcacatgtgtggtcaacggcgagtcctttacgtgcgtctgcaaggaaggctgggaggggcccatctgtgctcagaataccaatgactgcagccctcatccctgttacaacagcggcacctgtgtggatggagacaactggtaccggtgcgaatgtgccccgggttttgctgggcccgactgcagaataaacatcaatgaatgccagtcttcaccttgtgcctttggagcgacctgtgtggatgagatcaatggctaccggtgtgtctgccctccagggcacagtggtgccaagtgccaggaagtttcagggagaccttgcatcaccatggggagtgtgataccagatggggccaaatgggatgatgactgtaatacctgccagtgcctgaatggacggatcgcctgctcaaaggtctggtgtggccctcgaccttgcctgctccacaaagggcacagcgagtgccccagcgggcagagctgcatccccatcctggacgaccagtgcttcgtccacccctgcactggtgtgggcgagtgtcggtcttccagtctccagccggtgaagacaaagtgcacctctgactcctattaccaggataactgtgcgaacatcacatttacctttaacaaggagatgatgtcaccaggtcttactacggagcacatttgcagtgaattgaggaatttgaatattttgaagaatgtttccgctgaatattcaatctacatcgcttgcgagccttccccttcagcgaacaatgaaatacatgtggccatttctgctgaagatatacgggatgatgggaacccgatcaaggaaatcactgacaaaataatcgatcttgttagtaaacgtgatggaaacagctcgctgattgctgccgttgcagaagtaagagttcagaggcggcctctgaagaacagaacagatttccttgttcccttgctgagctctgtcttaactgtggcttggatctgttgcttggtgacggccttctactggtgcctgcggaagcggcggaagccgggcagccacacacactcagcctctgaggacaacaccaccaacaacgtgcgggagcagctgaaccagatcaaaaaccccattgagaaacatggggccaacacggtccccatcaaggattatgagaacaagaactccaaaatgtctaaaataaggacacacaattctgaagtagaagaggacgacatggacaaacaccagcagaaagcccggtttgccaagcagccggcgtacacgctggtagacagagaagagaagccccccaacggcacgccgacaaaacacccaaactggacaaacaaacaggacaacagagacttggaaagtgcccagagcttaaaccgaatggagtacatcgtatagcagaccgcgggcactgccgccgctaggtagagtctgagggcttgtagttctttaaactgtcgtgtcatactcgagtctgaggccgttgctgacttagaatccctgtgttaatttaagttttgacaagctggcttacactggcaatggtagtttctgtggttggctgggaaatcgagtgccgcatctcacagctatgcaaaaagctagtcaacagtaccctggttgtgtgtccccttgcagccgacacggtctcggatcaggctcccaggagcctgcccagccccctggtctttgagctcccacttctgccagatgtcctaatggtgatgcagtcttagatcatagttttatttatatttattgactcttgagttgtttttgtatattggttttatgatgacgtacaagtagttctgtatttgaaagtgcctttgcagctcagaaccacagcaacgatcacaaatgactttattatttattttttttaattgtatttttgttgttgggggaggggagactttgatgtcagcagttgctggtaaaatgaagaatttaaagaaaaaaatgtcaaaagtagaactttgtatagttatgtaaataattcttttttattaatcactgtgtatatttgatttattaacttaataatcaagagccttaaaacatcattcctttttatttatatgtatgtgtttagaattgaaggtttttgatagcattgtaagcgtatggctttatttttttgaactcttctcattacttgttgcctataagccaaaattaaggtgtttgaaaatagtttattttaaaacaataggatgggcttctgtgcccagaatactgatggaatttttttgtacgacgtcagatgtttaaaacaccttctatagcatcacttaaaacacgttttaaggactgactgaggcagtttgaggattagtttagaacaggtttttttgtttgtttgttttttgtttttctgctttagacttgaaaagagacaggcaggtgatctgctgcagagcagtaagggaacaagttgagctatgacttaacatagccaaaatgtgagtggttgaatatgattaaaaatatcaaattaattgtgtgaacttggaagcacaccaatcttactttgtaaattctgatttcttttcaccattcgtacataatactgaaccacttgtagatttgattttttttttaatctactgcatttagggagtattctaataagctagttgaatacttgaaccataaaatgtccagtaagatcactgtttagatttgccatagagtacactgcctgccttaagtgaggaaatcaaagtgctattacgaagttcaagatcaaaaaggcttataaaacagagtaatcttgttggttcaccattgagaccgtgaagatactttgtattgtcctattagtgttatatgaacatacaaatgcatctttgatgtgttgttcttggcaataaattttgaaaagtaatatttattaaatttttttgtatgaaaaaaaaaaaaaaaaaaaa

SEQ ID NO: 24 - DNA, 4974 bp, Homo sapiens
ctcatgcatatgcaggtgcgcgggtgacgaatgggcgagcgagctgtcagtctcgttccgaacttgttggctgcggtgccgggagcgcgggcgcgcagagcccgaggccgggacccgctgccttcaccgccgccgccgtcgccgccgggtgggagccgggccgggcagccggagcgcggccgccagcgagccggagctgccgccgcccctgcacgcccgccgcccaggcccgcgcgccggacgctgcgctcgaccccgcccgcgccgccgccgccgccgcctctgccgctgccgctgcctctgcgggcgctcggagggcgggcgggcgctgggaggccggcgcggcggctgggagccgggcgcgggcggcggcggcggggccgggcgggcgggtcgcgggggcaatgcgggcgcagggccgggggcgccttccccggcggctgctgctgctgctggcgctctgggtgcaggcggcgcggcccatgggctatttcgagctgcagctgagcgcgctgcggaacgtgaacggggagctgctgagcggcgcctgctgtgacggcgacggccggacaacgcgcgcggggggctgcggccacgacgagtgcgacacgtacgtgcgcgtgtgccttaaggagtaccaggccaaggtgacgcccacggggccctgcagctacggccacggcgccacgcccgtgctgggcggcaactccttctacctgccgccggcgggcgctgcgggggaccgagcgcgggcgcgggcccgggccggcggcgaccaggacccgggcctcgtcgtcatccccttccagttcgcctggccgcgctcctttaccctcatcgtggaggcctgggactgggacaacgataccaccccgaatgaggagctgctgatcgagcgagtgtcgcatgccggcatgatcaacccggaggaccgctggaagagcctgcacttcagcggccacgtggcgcacctggagctgcagatccgcgtgcgctgcgacgagaactactacagcgccacttgcaacaagttctgccggccccgcaacgactttttcggccactacacctgcgaccagtacggcaacaaggcctgcatggacggctggatgggcaaggagtgcaaggaagctgtgtgtaaacaagggtgtaatttgctccacgggggatgcaccgtgcctggggagtgcaggtgcagctacggctggcaagggaggttctgcgatgagtgtgtcccctaccccggctgcgtgcatggcagttgtgtggagccctggcagtgcaactgtgagaccaactggggcggcctgctctgtgacaaagacctgaactactgtggcagccaccacccctgcaccaacggaggcacgtgcatcaacgccgagcctgaccagtaccgctgcacctgccctgacggctactcgggcaggaactgtgagaaggctgagcacgcctgcacctccaacccgtgtgccaacgggggctcttgccatgaggtgccgtccggcttcgaatgccactgcccatcgggctggagcgggcccacctgtgcccttgacatcgatgagtgtgcttcgaacccgtgtgcggccggtggcacctgtgtggaccaggtggacggctttgagtgcatctgccccgagcagtgggtgggggccacctgccagctggacgccaatgagtgtgaagggaagccatgccttaacgctttttcttgcaaaaacctgattggcggctattactgtgattgcatcccgggctggaagggcatcaactgccatatcaacgtcaacgactgtcgcgggcagtgtcagcatgggggcacctgcaaggacctggtgaacgggtaccagtgtgtgtgcccacggggcttcggaggccggcattgcgagctggaacgagacaagtgtgccagcagcccctgccacagcggcggcctctgcgaggacctggccgacggcttccactgccactgcccccagggcttctccgggcctctctgtgaggtggatgtcgacctttgtgagccaagcccctgccggaacggcgctcgctgctataacctggagggtgactattactgcgcctgccctgatgactttggtggcaagaactgctccgtgccccgcgagccgtgccctggcggggcctgcagagtgatcgatggctgcgggtcagacgcggggcctgggatgcctggcacagcagcctccggcgtgtgtggcccccatggacgctgcgtcagccagccagggggcaacttttcctgcatctgtgacagtggctttactggcacctactgccatgagaacattgacgactgcctgggccagccctgccgcaatgggggcacatgcatcgatgaggtggacgccttccgctgcttctgccccagcggctgggagggcgagctctgcgacaccaatcccaacgactgccttcccgatccctgccacagccgcggccgctgctacgacctggtcaatgacttctactgtgcgtgcgacgacggctggaagggcaagacctgccactcacgcgagttccagtgcgatgcctacacctgcagcaacggtggcacctgctacgacagcggcgacaccttccgctgcgcctgcccccccggctggaagggcagcacctgcgccgtcgccaagaacagcagctgcctgcccaacccctgtgtgaatggtggcacctgcgtgggcagcggggcctccttctcctgcatctgccgggacggctgggagggtcgtacttgcactcacaataccaacgactgcaaccctctgccttgctacaatggtggcatctgtgttgacggcgtcaactggttccgctgcgagtgtgcacctggcttcgcggggcctgactgccgcatcaacatcgacgagtgccagtcctcgccctgtgcctacggggccacgtgtgtggatgagatcaacgggtatcgctgtagctgcccacccggccgagccggcccccggtgccaggaagtgatcgggttcgggagatcctgctggtcccggggcactccgttcccacacggaagctcctgggtggaagactgcaacagctgccgctgcctggatggccgccgtgactgcagcaaggtgtggtgcggatggaagccttgtctgctggccggccagcccgaggccctgagcgcccagtgcccactggggcaaaggtgcctggagaaggccccaggccagtgtctgcgaccaccctgtgaggcctggggggagtgcggcgcagaagagccaccgagcaccccctgcctgccacgctccggccacctggacaataactgtgcccgcctcaccttgcatttcaaccgtgaccacgtgccccagggcaccacggtgggcgccatttgctccgggatccgctccctgccagccacaagggctgtggcacgggaccgcctgctggtgttgctttgcgaccgggcgtcctcgggggccagtgccgtggaggtggccgtgtccttcagccctgccagggacctgcctgacagcagcctgatccagggcgcggcccacgccatcgtggccgccatcacccagcgggggaacagctcactgctcctggctgtcaccgaggtcaaggtggagacggttgttacgggcggctcttccacaggtctgctggtgcctgtgctgtgtggtgccttcagcgtgctgtggctggcgtgcgtggtcctgtgcgtgtggtggacacgcaagcgcaggaaagagcgggagaggagccggctgccgcgggaggagagcgccaacaaccagtgggccccgctcaaccccatccgcaaccccatcgagcggccggggggccacaaggacgtgctctaccagtgcaagaacttcacgccgccgccgcgcagggcggacgaggcgctgcccgggccggccggccacgcggccgtcagggaggatgaggaggacgaggatctgggccgcggtgaggaggactccctggaggcggagaagttcctctcacacaaattcaccaaagatcctggccgctcgccggggaggccggcccactgggcctcaggccccaaagtggacaaccgcgcggtcaggagcatcaatgaggcccgctacgccggcaaggagtaggggcggctgccagctgggccgggacccagggccctcggtgggagccatgccgtctgccggacccggaggccgaggccatgtgcatagtttctttattttgtgtaaaaaaaccaccaaaaacaaaaaccaaatgtttattttctacgtttctttaaccttgtataaattattcagtaactgtcaggctgaaaacaatggagtattctcggatagttgctatttttgtaaagtttccgtgcgtggcactcgctgtatgaaaggagagagcaaagggtgtctgcgtcgtcaccaaatcgtagcgtttgttaccagaggttgtgcactgtttacagaatcttccttttattcctcactcgggtttctctgtggctccaggccaaagtgccggtgagacccatggctgtgttggtgtggcccatggctgttggtgggacccgtggctgatggtgtggcctgtggctgtcggtgggactcgtggctgtcaatgggacctgtggctgtcggtgggacctacggtggtcggtgggaccctggttattgatgtggccctggctgccggcacggcccgtggctgttgacgcacctgtggttgttagtggggcctgaggtcatcggcgtggcccaaggccggcaggtcaacctcgcgcttgctggccagtccaccctgcctgccgtctgtgcttcctcctgcccagaacgcccgctccagcgatctctccactgtgctttcagaagtgcccttcctgctgcgaagttctcccatcctgggacggcggcagtattgaagctcgtgacaagtgccttcacacagaaccctcggaactgtccacgcgttccgtgggaacaaggggtt

SEQ ID NO: 25 - DNA, 2058 bp, Homo sapiens
atggcggcagcgtcccggagcgcctctggctgggcgctactgctgctggtggcactttggcagcagcgcgcggccggctccggcgtcttccagctgcagctgcaggagttcatcaacgagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacagtgaaaagccagagtgtcggatatcagcgatatgctcccccagggactccatgtaccagtctgtgtgtttgatatcagaggagaggaatgaatgtgtcattgccacggaggtataa

SEQ ID NO: 26 - protein, 43 aa, Artificial Sequence
  Description of Artificial Sequence Synthetic peptide
  MOD_RES (2)..(9): Variable amino acid
  MOD_RES (11)..(13): Variable amino acid
  MOD_RES (15)..(25): Variable amino acid
  MOD_RES (27)..(33): Variable amino acid
  MOD_RES (35)..(42): Variable amino acid
CXXXXXXXXCXXXCXXXXXXXXXXXCXXXXXXXCXXXXXXXXC

SEQ ID NO: 27 - protein, 43 aa, Artificial Sequence; identical to SEQ ID NO: 26
  Description of Artificial Sequence Synthetic peptide
  MOD_RES (2)..(4): Variable amino acid
  MOD_RES (5)..(6): Aromatic amino acid
  MOD_RES (7)..(9): Variable amino acid
  MOD_RES (11)..(13): Variable amino acid
  MOD_RES (15): Basic amino acid
  MOD_RES (16): Non-polar amino acid
  MOD_RES (17): Basic amino acid
  MOD_RES (18)..(19): Acid or amide amino acid
  MOD_RES (20): Variable amino acid
  MOD_RES (21): Aromatic amino acid
  MOD_RES (22): Non-polar amino acid
  MOD_RES (23): Aromatic amino acid
  MOD_RES (24)..(25): Variable amino acid
  MOD_RES (27)..(29): Variable amino acid
  MOD_RES (30): Non-polar amino acid
  MOD_RES (31)..(33): Variable amino acid
  MOD_RES (35)..(36): Variable amino acid
  MOD_RES (37): Non-polar amino acid
  MOD_RES (38): Aromatic amino acid
  MOD_RES (39): Variable amino acid
  MOD_RES (40): Non-polar amino acid
  MOD_RES (41)..(42): Variable amino acid

SEQ ID NO: 28 - protein, 43 aa, Artificial Sequence
  Description of Artificial Sequence Synthetic peptide
  MOD_RES (2)..(4): Variable amino acid
  MOD_RES (7)..(9): Variable amino acid
  MOD_RES (11)..(13): Variable amino acid
  MOD_RES (20): Variable amino acid
  MOD_RES (24)..(25): Variable amino acid
  MOD_RES (27)..(29): Variable amino acid
  MOD_RES (31)..(33): Variable amino acid
  MOD_RES (35)..(36): Variable amino acid
  MOD_RES (39): Variable amino acid
  MOD_RES (41)..(42): Variable amino acid
CXXXYYXXXCXXXCRPRBDXFGHXXCXXXGXXXCXXGWXGXXC

SEQ ID NO: 29 - DNA, 20 bp, Artificial Sequence
  Description of Combined DNA/RNA Molecule Synthetic oligonucleotide
  Description of Artificial Sequence Synthetic oligonucleotide
ggaguugucaacaagaagtt

SEQ ID NO: 30 - DNA, 21 bp, Artificial Sequence
  Description of Combined DNA/RNA Molecule Synthetic oligonucleotide
  Description of Artificial Sequence Synthetic oligonucleotide
cuucuuguugacgaacucctg

SEQ ID NO: 31 - DNA, 21 bp, Artificial Sequence
  Description of Artificial Sequence Synthetic oligonucleotide
aaggagttcgtcaacaagaag

SEQ ID NO: 32 - DNA, 21 bp, Artificial Sequence
  Description of Combined DNA/RNA Molecule Synthetic oligonucleotide
  Description of Artificial Sequence Synthetic oligonucleotide
ggaccugaacuacugcacatt

SEQ ID NO: 33 - DNA, 21 bp, Artificial Sequence
  Description of Combined DNA/RNA Molecule Synthetic oligonucleotide
  Description of Artificial Sequence Synthetic oligonucleotide
ugugcaguaguucaggucctg

SEQ ID NO: 34 - DNA, 21 bp, Artificial Sequence
  Description of Artificial Sequence Synthetic oligonucleotide
aaggacctgaactactgcaca

SEQ ID NO: 35 - DNA, 21 bp, Artificial Sequence
  Description of Combined DNA/RNA Molecule Synthetic oligonucleotide
  Description of Artificial Sequence Synthetic oligonucleotide
gguguaaaauggaagugagtt

SEQ ID NO: 36 - DNA, 21 bp, Artificial Sequence
  Description of Combined DNA/RNA Molecule Synthetic oligonucleotide
  Description of Artificial Sequence Synthetic oligonucleotide
cucacuuccauuuuacacctc

SEQ ID NO: 37 - DNA, 21 bp, Artificial Sequence
  Description of Artificial Sequence Synthetic oligonucleotide
aaggtgtaaaatggaagtgag

SEQ ID NO: 38 - DNA, 19 bp, Artificial Sequence
  Description of Artificial Sequence Synthetic oligonucleotide
gattatgtccggttatgta

SEQ ID NO: 39 - DNA, 21 bp, Artificial Sequence
  Description of Combined DNA/RNA Molecule Synthetic oligonucleotide
  Description of Artificial Sequence Synthetic oligonucleotide
gauuauguccgguuauguatt

SEQ ID NO: 40 - DNA, 21 bp, Artificial Sequence
  Description of Combined DNA/RNA Molecule Synthetic oligonucleotide
  Description of Artificial Sequence Synthetic oligonucleotide
uacauaaccggacauaauctt

SEQ ID NO: 41 - DNA, 20 bp, Artificial Sequence
  Description of Combined DNA/RNA Molecule Synthetic oligonucleotide
  Description of Artificial Sequence Synthetic oligonucleotide
gguccuauacguuguugutt

SEQ ID NO: 42 - DNA, 21 bp, Artificial Sequence
  Description of Combined DNA/RNA Molecule Synthetic oligonucleotide
  Description of Artificial Sequence Synthetic oligonucleotide
acaagcaacguauaggacctc

SEQ ID NO: 43 - DNA, 18 bp, Artificial Sequence
  Description of Artificial Sequence Synthetic oligonucleotide
ggtcctatacgttgttgt

SEQ ID NO: 44 - DNA, 21 bp, Artificial Sequence
  Description of Combined DNA/RNA Molecule Synthetic oligonucleotide
  Description of Artificial Sequence Synthetic oligonucleotide
ggcgugggauuccaguaautt

SEQ ID NO: 45 - DNA, 21 bp, Artificial Sequence
  Description of Combined DNA/RNA Molecule Synthetic oligonucleotide
  Description of Artificial Sequence Synthetic oligonucleotide
auuacuggaaucccacgcctc

SEQ ID NO: 46 - DNA, 19 bp, Artificial Sequence
  Description of Artificial Sequence Synthetic oligonucleotide
ggcgtgggattccagtaat

SEQ ID NO: 47 - DNA, 21 bp, Artificial Sequence
  Description of Combined DNA/RNA Molecule Synthetic oligonucleotide
  Description of Artificial Sequence Synthetic oligonucleotide
ggaaaucaaagugcuauuatt

SEQ ID NO: 48 - DNA, 21 bp, Artificial Sequence
  Description of Combined DNA/RNA Molecule Synthetic oligonucleotide
  Description of Artificial Sequence Synthetic oligonucleotide
uaauagcacuuugauuucctc

SEQ ID NO: 49 - DNA, 19 bp, Artificial Sequence
  Description of Artificial Sequence Synthetic oligonucleotide
ggaaatcaaagtgctatta

SEQ ID NO: 50 - DNA, 21 bp, Artificial Sequence
  Description of Combined DNA/RNA Molecule Synthetic oligonucleotide
  Description of Artificial Sequence Synthetic oligonucleotide
ggagugcaaggaagcugugtt

SEQ ID NO: 51 - DNA, 21 bp, Artificial Sequence
  Description of Combined DNA/RNA Molecule Synthetic oligonucleotide
  Description of Artificial Sequence Synthetic oligonucleotide
cacagcuuccuugcacucctt

SEQ ID NO: 52 - DNA, 21 bp, Artificial Sequence
  Description of Artificial Sequence Synthetic oligonucleotide
ggagtgcaaggaagctgtgtt

SEQ ID NO: 53 - DNA, 21 bp, Artificial Sequence
  Description of Combined DNA/RNA Molecule Synthetic oligonucleotide
  Description of Artificial Sequence Synthetic oligonucleotide
ggaagcuguguguaaacaatt

SEQ ID NO: 54 - DNA, 21 bp, Artificial Sequence
  Description of Combined DNA/RNA Molecule Synthetic oligonucleotide
  Description of Artificial Sequence Synthetic oligonucleotide
uuguuuacacacagcuucctt

SEQ ID NO: 55 - DNA, 21 bp, Artificial Sequence
  Description of Artificial Sequence Synthetic oligonucleotide
ggaagctgtgtgtaaacaatt

SEQ ID NO: 56 - DNA, 21 bp, Artificial Sequence
  Description of Combined DNA/RNA Molecule Synthetic oligonucleotide
  Description of Artificial Sequence Synthetic oligonucleotide
ggauuucauggagaagcugtt

SEQ ID NO: 57 - DNA, 21 bp, Artificial Sequence
  Description of Combined DNA/RNA Molecule Synthetic oligonucleotide
  Description of Artificial Sequence Synthetic oligonucleotide
cagcuucuccaugaaaucctc

SEQ ID NO: 58 - DNA, 21 bp, Artificial Sequence
  Description of Combined DNA/RNA Molecule Synthetic oligonucleotide
  Description of Artificial Sequence Synthetic oligonucleotide
ggcauuuccuucagugcuutt

SEQ ID NO: 59 - DNA, 20 bp, Artificial Sequence
  Description of Combined DNA/RNA Molecule Synthetic oligonucleotide
  Description of Artificial Sequence Synthetic oligonucleotide
aagcaugaaggaaaugcctg

SEQ ID NO: 60 - DNA, 21 bp, Artificial Sequence
  Description of Combined DNA/RNA Molecule Synthetic oligonucleotide
  Description of Artificial Sequence Synthetic oligonucleotide
gcugcuaaaccguaccuugtt

SEQ ID NO: 61 - DNA, 21 bp, Artificial Sequence
  Description of Combined DNA/RNA Molecule Synthetic oligonucleotide
  Description of Artificial Sequence Synthetic oligonucleotide
caagguacgguuuagcagctt

SEQ ID NO: 62 - DNA, 21 bp, Artificial Sequence
  Description of Combined DNA/RNA Molecule Synthetic oligonucleotide
  Description of Artificial Sequence Synthetic oligonucleotide
cuuauucucguuauccggutt

SEQ ID NO: 63 - DNA, 21 bp, Artificial Sequence
  Description of Combined DNA/RNA Molecule Synthetic oligonucleotide
  Description of Artificial Sequence Synthetic oligonucleotide
accggauaacgagaauaagtt

SEQ ID NO: 64 - DNA, 21 bp, Artificial Sequence
  Description of Combined DNA/RNA Molecule Synthetic oligonucleotide
  Description of Artificial Sequence Synthetic oligonucleotide
gguuuuuuugcccaccucctt

SEQ ID NO: 65 - DNA, 21 bp, Artificial Sequence
  Description of Combined DNA/RNA Molecule Synthetic oligonucleotide
  Description of Artificial Sequence Synthetic oligonucleotide
ggaggugggcaaaaaaacctt

SEQ ID NO: 66 - DNA, 21 bp, Artificial Sequence
  Description of Combined DNA/RNA Molecule Synthetic oligonucleotide
  Description of Artificial Sequence Synthetic oligonucleotide
ggaaagaacuauugcacagtt

SEQ ID NO: 67 - DNA, 21 bp, Artificial Sequence
  Description of Combined DNA/RNA Molecule Synthetic oligonucleotide
  Description of Artificial Sequence Synthetic oligonucleotide
cugugcaauaguucuuucctt

SEQ ID NO: 68 - DNA, 21 bp, Artificial Sequence
  Description of Combined DNA/RNA Molecule Synthetic oligonucleotide
  Description of Artificial Sequence Synthetic oligonucleotide
ggagguaauuuucaugccatt

SEQ ID NO: 69 - DNA, 21 bp, Artificial Sequence
  Description of Combined DNA/RNA Molecule Synthetic oligonucleotide
  Description of Artificial Sequence Synthetic oligonucleotide
uggcaugaaaauuaccucctt

SEQ ID NO: 70 - DNA, 21 bp, Artificial Sequence
  Description of Combined DNA/RNA Molecule Synthetic oligonucleotide
  Description of Artificial Sequence Synthetic oligonucleotide
uucuccguucguccacguutt

SEQ ID NO: 71 - DNA, 21 bp, Artificial Sequence
  Description of Combined DNA/RNA Molecule Synthetic oligonucleotide
  Description of Artificial Sequence Synthetic oligonucleotide
aacguggacgaacggagaatt

SEQ ID NO: 72 - DNA, 21 bp, Artificial Sequence
  Description of Combined DNA/RNA Molecule Synthetic oligonucleotide
  Description of Artificial Sequence Synthetic oligonucleotide
aagugacaguacgauagactt

SEQ ID NO: 73 - DNA, 21 bp, Artificial Sequence
  Description of Combined DNA/RNA Molecule Synthetic oligonucleotide
  Description of Artificial Sequence Synthetic oligonucleotide
gucuaucguacugucacuutt

SEQ ID NO: 74 - DNA, 21 bp, Artificial Sequence
  Description of Combined DNA/RNA Molecule Synthetic oligonucleotide
  Description of Artificial Sequence Synthetic oligonucleotide
ggaguucgucaacaagaagtt

SEQ ID NO: 75 - DNA, 21 bp, Artificial Sequence
  Description of Combined DNA/RNA Molecule Synthetic oligonucleotide
  Description of Artificial Sequence Synthetic oligonucleotide
cuucuuguugacgaacucctg

SEQ ID NO: 76 - DNA, 21 bp, Artificial Sequence
  Description of Combined DNA/RNA Molecule Synthetic oligonucleotide
  Description of Artificial Sequence Synthetic oligonucleotide
ggaccuucuuucgcguaugtt

SEQ ID NO: 77 - DNA, 21 bp, Artificial Sequence
  Description of Combined DNA/RNA Molecule Synthetic oligonucleotide
  Description of Artificial Sequence Synthetic oligonucleotide
cauacgcgaaagaaggucctg

SEQ ID NO: 78 - protein, 1424 aa, Homo sapiens
MSARTAPRPQVLLLPLLLVLLAAAPAASKGCVCKDKGQCFCDGAKGEKGEKGFPGPPGSPGQKGFTGPEGLPGPQGPKGFPGLPGLTGSKGVRGISGLPGFSGSPGLPGTPGNTGPYGLVGVPGCSGSKGEQGFPGLPGTPGYPGIPGAAGLKGQKGAPAKGEDIELDAKGDPGLPGAPGPQGLPGPPGFPGPVGPPGPPGFFGFPGAMGPRGPKGHMGERVIGHKGERGVKGLTGPPGPPGTVIVTLTGPDNRTDLKGEKGDKGAMGEPGPPGPSGLPGESYGSEKGAPGDPGLQGKPGKDGVPGFPGSEGVKGNRGFPGLMGEDGIKGQKGDIGPPGFRGPTEYYDTYQEKGDEGTPGPPGPRGARGPQGPSGPPGVPGSPGSSRPGLRGAPGWPGLKGSKGERGRPGKDAMGTPGSPGCAGSPGLPGSPGPPGPPGDIVFRKGPPGDHGLPGYLGSPGIPGVDGPKGEPGLLCTQCPYIPGPPGLPGLPGLHGVKGIPGRQGAAGLKGSPGSPGNTGLPGFPGFPGAQGDPGLKGEKGETLQPEGQVGVPGDPGLRGQPGRKGLDGIPGTLGVKGLPGPKGELALSGEKGDQGPPGDPGSPGSPGPAGPAGPPGYGPQGEPGLQGTQGVPGAPGPPGEAGPRGELSVSTPVPGPPGPPGPPGHPGPQGPPGIPGSLGKCGDPGLPGPDGEPGIPGIGFPGPPGPKGDQGFPGTKGSLGCPGKMGEPGLPGKPGLPGAKGEPAVAMPGGPGTPGFPGERGNSGEHGEIGLPGLPGLPGTPGNEGLDGPRGDPGQPGPPGEQGPPGRCIEGPRGAQGLPGLNGLKGQQGRRGKTGPKGDPGIPGLDRSGFPGETGSPGIPGHQGEMGPLGQRGYPGNPGILGPPGEDGVIGMMGFPGAIGPPGPPGNPGTPGQRGSPGIPGVKGQRGTPGAKGEQGDKGNPGPSEISHVIGDKGEPGLKGFAGNPGEKGNRGVPGMPGLKGLKGLPGPAGPPGPRGDLGSTGNPGEPGLRGIPGSMGNMGMPGSKGKRGTLGFPGRAGRPGLPGIHGLQGDKGEPGYSEGTRPGPPGPTGDPGLPGDMGKKGEMGQPGPPGHLGPAGPEGAPGSPGSPGLPGKPGPHGDLGFKGIKGLLGPPGIRGPPGLPGFPGSPGPMGIRGDQGRDGIPGPAGEKGETGLLRAPPGPRGNPGAQGAKGDRGAPGFPGLPGRKGAMGDAGPRGPTGIEGFPGPPGLPGAIIPGQTGNRGPPGSRGSPGAPGPPGPPGSHVIGIKGDKGSMGHPGPKGPPGTAGDMGPPGRLGAPGTPGLPGPRGDPGFQGFPGVKGEKGNPGFLGSIGPPGPIGPKGPPGVRGDPGTLKIISLPGSPGPPGTPGEPGMQGEPGPPGPPGNLGPCGPRGKPGKDGKPGTPGPAGEKGNKGSKGEPESLFHQL

SEQ ID NO: 79 - protein, 1049 aa, Homo sapiens
MDWSFFRVVAVLFIFLVVVEVNSEFRIQVRDYNTKNGTIKWHSIRRQKREWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNTKITTNTGRQESTSSTNYDTSTTSTDSSQVYSSEPGNGAKDLLSDNVHFGPAGIGLLIMGFLVLGLVPFLMICCDCGGAPRSAAGFEPVPECSDGAIHSWAVEGPQPEPRDITTVIPQIPPDNANIIECIDNSGVYTNEYGGREMQDLGGGERMTGFELTEGVKTSGMPEICQEYSGTLRRNSMRECREGGLNMNFMESYFCQKAYAYADEDEGRPSNDCLLIYDIEGVGSPAGSVGCCSFIGEDLDDSFLDTLGPKFKKLADISLGKESYPDLDPSWPPQSTEPVCLPQETEPVVSGHPPISPHFGTTTVISESTYPSGPGVLHPKPILDPLGYGNVTVTESYTTSDTLKPSVHVHDNRPASNVVVTERVVGPISGADLHGMLEMPDLRDGSNVIVTERVIAPSSSLPTSLTIHHPRESSNVVVTERVIQPTSGMIGSLSMHPELANAHNVIVTERVVSGAGVTGISGTTGISGGIGSSGLVGTSMGAGSGALSGAGISGGGIGLSSLGGTASIGHMRSSSDHHFNQTIGSASPSTARSRITKYSTVQYSK

SEQ ID NO: 80 - protein, 2649 aa, Homo sapiens
MHSSSYSYRSSDSVFSNTTSTRTSLDSNENLLLVHCGPTLINSCISFGSESFDGHRLEMLQQIANRVQRDSVICEDKLILAGNALQSDSKRLESGVQFQNEAEIAGYILECENLLRQHVIDVQILIDGKYYQADQLVQRVAKLRDEIMALRNECSSVYSKGRILTTEQTKLMISGITQSLNSGFAQTLHPSLTSGLTQSLTPSLTSSSMTSGLSSGMTSRLTPSVTPAYTPGFPSGLVPNFSSGVEPNSLQTLKLMQIRKPLLKSSLLDQNLTEEEINMKFVQDLLNWVDEMQVQLDRTEWGSDLPSVESHLENHKNVHRAIEEFESSLKEAKISEIQMTAPLKLTYAEKLHRLESQYAKLLNTSRNQERHLDTLHNFVSRATNELIWLNEKEEEEVAYDWSERNTNIARKKDYHAELMRELDQKEENIKSVQEIAEQLLLENHPARLTIEAYRAAMQTQWSWILQLCQCVEQHIKENTAYFEFFNDAKEATDYLRNLKDAIQRKYSCDRSSSIHKLEDLVQESMEEKEELLQYKSTIANLMGKAKTIIQLKPRNSDCPLKTSIPIKAICDYRQIEITIYKDDECVLANNSHRAKWKVISPTGNEAMVPSVCFTVPPPNKEAVDLANRIEQQYQNVLTLWHESHINMKSVVSWHYLINEIDRIRASNVASIKTMLPGEHQQVLSNLQSRFEDFLEDSQESQVFSGSDITQLEKEVNVCKQYYQELLKSAEREEQEESVYNLYISEVRNIRLRLENCEDRLIRQIRTPLERDDLHESVFRITEQEKLKKELERLKDDLGTITNKCEEFFSQAAASSSVPTLRSELNVVLQNMNQVYSMSSTYIDKLKTVNLVLKNTQAAEALVKLYETKLCEEEAVIADKNNIENLISTLKQWRSEVDEKRQVFHALEDELQKAKAISDEMFKTYKERDLDFDWHKEKADQLVERWQNVHVQIDNRLRDLEGIGKSLKYYRDTYHPLDDWIQQVETTQRKIQENQPENSKTLATQLNQQKMLVSEIEMKQSKMDECQKYAEQYSATVKDYELQTMTYRAMVDSQQKSPVKRRRMQSSADLIIQEFMDLRTRYTALVTLMTQYIKFAGDSLKRLEEEEIKRCKETSEHGAYSDLLQRQKATVLENSKLTGKISELERMVAELKKQKSRVEEELPKVREAAENELRKQQRNVEDISLQKIRAESEAKQYRRELETIVREKEAAERELERVRQLTIEAEAKRAAVEENLLNFRNQLEENTFTRRTLEDHLKRKDLSLNDLEQQKNKLMEELRRKRDNEEELLKLIKQMEKDLAFQKQVAEKQLKEKQKIELEARRKITEIQYTCRENALPVCPITQATSCRAVTGLQQEHDKQKAEELKQQVDELTAANRKAEQDMRELTYELNALQLEKTSSEEKARLLKDKLDETNNTLRCLKLELERKDQAEKGYSQQLRELGRQLNQTTGKAEEAMQEASDLKKIKRNYQLELESLNHEKGKLQREVDRITRAHAVAEKNIQHLNSQIHSFRDEKELERLQICQRKSDHLKEQFEKSHEQLLQNIKAEKENNDKIQRLNEELEKSNECAEMLKQKVEELTRQNNETKLMMQRIQAESENIVLEKQTIQQRCEALKIQADGFKDQLRSTNEHLHKQTKTEQDFQRKIKCLEEDLAKSQNLVSEFKQKCDQQNIIIQNTKKEVRNLNAELNASKEEKRRGEQKVQLQQAQVQELNNRLKKVQDELHLKTIEEQMTHRKMVLFQEESGKFKQSAEEFRKKMEKLMESKVITENDISGIRLDFVSLQQENSRAQENAKLCETNIKELERQLQQYREQMQQGQHMEANHYQKCQKLEDELIAQKREVENLKQKMDQQIKEHEHQLVLLQCEIQKKSTAKDCTFKPDFEMTVKECQHSGELSSRNTGHLHPTPRSPLLRWTQEPQPLEEKWQHRVVEQIPKEVQFQPPGAPLEKEKSQQCYSEYFSQTSTELQITFDETNPITRLSEIEKIRDQALNNSRPPVRYQDNACEMELVKVLTPLEIAKNKQYDMHTEVTTLKQEKNPVPSAEEWMLEGCRASGGLKKGDFLKKGLEPETFQNFDGDHACSVRDDEFKFQGLRHTVTARQLVEAKLLDMRTIEQLRLGLKTVEEVQKTLNKFLTKATSIAGLYLESTKEKISFASAAERIIIDKMVALAFLEAQAATGFIIDPISGQTYSVEDAVLKGVVDPEFRIRLLEAEKAAVGYSYSSKTLSVFQAMENRMLDRQKGKHILEAQIASGGVIDPVRGIRVPPEIALQQGLLNNAILQFLHEPSSNTRVFPNPNNKQALYYSELLRMCVFDVESQCFLFPFGERNISNLNVKKTHRISVVDTKTGSELTVYEAFQRNLIEKSIYLELSGQQYQWKEAMFFESYGHSSHMLTDTKTGLHFNINEAIEQGTIDKALVKKYQEGLITLTELADSLLSRLVPKKDLHSPVAGYWLTASGERISVLKASRRNLVDRITALRCLEAQVSTGGIIDPLTGKKYRVAEALHRGLVDEGFAQQLRQCELVITGIGHPITNKMMSVVEAVNANIINKEMGIRCLEFQYLTGGLIEPQVHSRLSIEEALQVGIIDVLIATKLKDQKSYVRNIICPQTKRKLTYKEALEKADFDFHTGLKLLEVSEPLMTGISSLYYSS

SEQ ID NO: 81 - protein, 15 aa, Artificial Sequence
  Description of Artificial Sequence Synthetic peptide
LNSKIAFKIVSQEPA

SEQ ID NO: 82 - protein, 15 aa, Artificial Sequence
  Description of Artificial Sequence Synthetic peptide
TPMFLLSRNTGEVRT

SEQ ID NO: 83 - protein, 764 aa, Homo sapiens [sequence omitted]

SEQ ID NO: 84 - protein, 933 aa, Homo sapiens [sequence omitted]

SEQ ID NO: 85 - protein, 256 aa, Homo sapiens
MAHRPPSPALASVLLALLLSGAARAAEIVGGHEAQPHSRPYMASLQMRGNPGSHFCGGTLIHPSFVLTAPHCLRDIPQRLVNVVLGAHNVRTQEPTQQHFSVAQVFLNNYDAENKLNDILLIQLSSPANLSASVTSVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRRVEAKGRP

SEQ ID NO: 86 - protein, 1039 aa, Homo sapiens [sequence omitted]

SEQ ID NO: 87 - protein, 788 aa, Homo sapiens [sequence omitted]

SEQ ID NO: 88 - protein, 291 aa, Homo sapiens
MAALQEKKTCGQRMEEFQRYCWNPDTGQMLGRTLSRWVWISLYYVAFYVVMTGLFALCLYVLMQTVDPYTPDYQDQLRSPGVTLRPDVYGEKGLEIVYNVSDNRTWADLTQTLHAFLAGYSPAAQEDSINCTSEQYFFQESFRAPNHTKFSCKFTADMLQNCSGLADPNFGFEEGKPCFIIKMNRIVKFLPSNGSAPRVDCAFLDQPRELGQPLQVKYYPPNGTFSLHYFPYYGKKAQPHYSNPLVAAKLLNIPRNAEVAIVCKVMAEHVTFNNPHDPYEGKVEFKLKIEK

SEQ ID NO: 89 - DNA, 1545 bp, Homo sapiens
  modified_base (216): a, c, g, t, unknown or other
  modified_base (471): a, c, g, t, unknown or other
  modified_base (702): a, c, g, t, unknown or other
  modified_base (1162): a, c, g, t, unknown or other
  modified_base (1206): a, c, g, t, unknown or other
atggggctagaagcactggtgcccctggccatgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccagttgcggcgccgacttcgggacgtgttcagcctgcanctggcctggacgccggtggtcgtgctcaatgggctggcggccgtgcgcgaggcgctggtgacccacggcgaggacaccgccgaccgcccgcctgtgcccatcacccagatcctgggcttcgggccgcgttcccaaggggtgttcctggcgcgctatgggcccgcgtggcgcgagcagaggcgcttctccgtctccaccttgcgcaacttgggcctgggcaagaagtcgctggagcagtgggtgaccgaggaggcngcctgcctttgtgccgccttcgccaaccactccggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgctgaatgctgtccccgtcctcctgcatatcccngcgctggctggcaaggtcctacgcttccaaaaggctttcctgacccagctggatgagctgctaactgagcacaggatgacctgggacccagcccagcccccccgagacctgactgaggccttcctggcagagatggagaaggccaaggggaaccctgcgagcagcttcaatgatgagaacctgcgcatagtggtggctgacctgttctctgccgggatggtgaccacctcgaccacgctggcctggggcctcctgctcatgatcctacatccggatgtgcagcgccgtgtccaacaggagatcgacgacgtgatagggcaggtgcggcgaccagagatgggtgaccaggctcacatgccctacaccactgccgtgattcatgaggtgcagcgctttggggacatcgtccccctgggtgtgacccatatgacatcccgtgacatcgaggtacagggcttcngcatccctaagggaacgacactcatcaccaacctgtcatcggtnctgaaggatgaggccgtctgggagaagcccttccgcttccaccccgaacacttcctggatgcccagggccactttgtgaagccggaggccttcctgcctttctcagcaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaatggggtacctagtccccagcctgctcctagcccagaggctctaatgtac

SEQ ID NO: 90 - protein, 516 aa, Homo sapiens [sequence omitted]

SEQ ID NO: 91 - protein, 405 aa, Homo sapiens [sequence omitted]

SEQ ID NO: 92 - protein, 173 aa, Homo sapiens
MDVTIQHPWFKRTLGPFYPSRLFDQFFGEGLFEYDLLPFLSSTISPYYRQSLFRTVLDSGISEVRSDRDKFVIFLDVKHFSPEDLTVKVQDDFVEIHGKHNERQDDHGYISREFHRRYRLPSNVDQSALSCSLSADGMLTFCGPKIQTGLDATHAERAIPVSREEKPTSAPSS

SEQ ID NO: 93 - protein, 508 aa, Homo sapiens
MWELVALLLLTLAYLFWPKRRCPGAKYPKSLLSLPLVGSLPFLPRHGHMHNNFFKLQKKYGPIYSVRMGTKTTVIVGHHQLAKEVLIKKGKDFSGRPQMATLDIASNNRKGIAFADSGAHWQLHRRLAMATFALFKDGDQKLEKIICQEISTLCDMLATHNGQSIDISFPVFVAVTNVISLICFNTSYKNGDPELNVIQNYNEGIIDNLSKDSLVDLVPWLKIFPNKTLEKLKSHVKIRNDLLNKILENYKEKFRSDSITNMLDTLMQAKMNSDNGNAGPDQDSELLSDNHILTTIGDIFGAGVETTTSVVKWTLAFLLHNPQVKKKLYEEIDQNVGFSRTPTISDRNRLLLLEATIREVLRLRPVAPMLIPHKANVDSSIGEFAVDKGTEVIINLWALHHNEKEWHQPDQFMPERFLNPAGTQLISPSVSYLPFGAGPRSCIGEILARQELFLIMAWLLQRFDLEVPDDGQLPSLEGIPKVVFLIDSFKVKIKVRQAWREAQAEGST

SEQ ID NO: 94 - protein, 860 aa, Homo sapiens
MAPPSTREPRVLSATSATKSDGEMVLPGFPDADSFVKFALGSVVAVTKASGGLPQFGDEYDFYRSFPGFQAFCETQGDRLLQCMSRVMQYHGCRSNIKDRSKVTELEDKFDLLVDANDVILERVGILLDEASGVNKNQQPVLPAGLQVPKTVVSSWNRKAAEYGKKAKSETFRLLHAKNIIRPQLKFREKIDNSNTPFLPKIFIKPNAQKPLPQALSKERRERPQDRPEDLDVPPALADFIHQQRTQQVEQDMFAHPYQYELNHFTPADAVLQKPQPQLYRPIEETPCHFISSLDELVELNEKLLNCQEFAVDLEHHSYRSFLGLTCLMQISTRTEDFIIDTLELRSDMYILNESLTDPAIVKVFHGADSDIEWLQKDFGLYVVNMFDTHQAARLLNLGRHSLDHLLKLYCNVDSNKQYQLADWRIRPLPEEMLSYARDDTHYLLYIYDKMRLEMWERGNGQPVQLQVVWQRSRDICLKKFIKPIFTDESYLELYRKQKKHLNTQQLTAFQLLFAWRDKTARREDESYGYVLPNHMMLKIAEELPKEPQGIIACCNPVPPLVRQQINEMHLLIQQAREMPLLKSEVAAGVKKSGPLPSAERLENVLFGPHDCSHAPPDGYPIIPTSGSVPVQKQASLFPDEKEDNLLGTTCLIATAVITLFNEPSAEDSKKGPLTVAQKKAQNIMESFENPFRMISNRWKLAQVQVQKDSKEAVKKKAAEQTAAREQAKEACKAAAEQAISVRQQVVLENAAKKRERATSDPRTTEQKQEKKRLKISKKPKDPEPPEKEFTPYDYSQSDFKAFAGNSKSKVSSQFDPNKQTPSGKKCIAAKKIKQSVGNKSMSFPTGKSDRGFRYNWPQR

SEQ ID NO: 95 - protein, 885 aa, Homo sapiens
MAPPSTREPRVLSATSATKSDGEMVLPGFPDADSFVKFALGSVVAVTKASGGLPQFGDEYDFYRSFPGFQAFCETQGDRLLQCMSRVMQYHGCRSNIKDRSKVTELEDKFDLLVDANDVILERVGILLDEASGVNKNQQPVLPAGLQVPKTVVSSWNRKAAEYGKKAKSETFRLLHAKNIIRPQLKFREKIDNSNTPFLPKIFIKPNAQKPLPQALSKERRERPQDRPEDLDVPPALADFIHQQRTQQVEQDMFAHPYQYELNHFTPADAVLQKPQPQLYRPIEETPCHFISSLDELVELNEKLLNCQEFAVDLEHHSYRSFLGLTCLMQISTRTEDFIIDTLELRSDMYILNESLTDPAIVKVFHGADSDIEWLQKDFGLYVVNMFDTHQAARLLNLGRHSLDHLLKLYCNVDSNKQYQLADWRIRPLPEEMLSYARDDTHYLLYIYDKMRLEMWERGNGQPVQLQVVWQRSRDICLKKFIKPIFTDESYLELYRKQKKHLNTQQLTAFQLLFAWRDKTARREDESYGYVLPNHMMLKIAEELPKEPQGIIACCNPVPPLVRQQINEMHLLIQQAREMPLLKSEVAAGVKKSGPLPSAERLENVLFGPHDCSHAPPDGYPIIPTSGSVPVQKQASLFPDEKEDNLLGTTCLIATAVITLFNEPSAEDSKKGPLTVAQKKAQNIMESFENPFRMFLPSLGHRAPVSQAAKFDPSTKIYEISNRWKLAQVQVQKDSKEAVKKKAAEQTAAREQAKEACKAAAEQAISVRQQVVLENAAKKRERATSDPRTTEQKQEKKRLKISKKPKDPEPPEKEFTPYDYSQSDFKAFAGNSKSKVSSQFDPNKQTPSGKKCIAAKKIKQSVGNKSMSFPTGKSDRGFRYNWPQR

SEQ ID NO: 96 - protein, 355 aa, Homo sapiens
MAAPAFEPGRQSDLLVKLNRLMERCLRNSKCIDTESLCVVAGEKVWQIRVDLHLLNHDGNIIDAASIAAIVALCHFRRPDVSVQGDEVTLYTPEERDPVPLSIHHMPICVSFAFFQQGTYLLVDPNEREERVMDGLLVIAMNKHREICTIQSSGGIMLLKDQVLRCSKIAGVKVAEITELILKALENDQKVRKEGGKFGFAESIANQRITAFKMEKAPIDTSDVEEKAEEIIAEAEPPSEVVSTPVLWTPGTAQIGEGVENSWGDLEDSEKEDDEGGGDQAIILDGIKMDTGVEVSDIGSQDAPIILSDSEEEEMIILEPDKNPKKIRTQTTSAKQEKAPSKKPVKRRKKKRAAN

SEQ ID NO: 97 - protein, 372 aa, Homo sapiens
MAAPAFEPGRQSDLLVKLNRLMERCLRNSKCIDTESLCVVAGEKVWQIRVDLHLLNHDGNIIDAASIAAIVALCHFRRPDVSVQGDEVTLYTPEERDPVPLSIHHMPICVSFAFFQQGTYLLVDPNEREERVMDGLLVIAMNKHREICTIQSSGGIMLLKDQVLRCSKIAGVKVAEITELILKALENDQKVRKEGGKFGFAESIANQRITAFKMEKAPIDTSDVEEKAEEIIAEAEPPSEVVSTPVLWTPGTAQIGEGVENSWGDLEDSEKEDDEGGGDQAIILDGIKMDTGVEVSDIGSQELGFHHVGQTGLEFLTSDAPIILSDSEEEEMIILEPDKNPKKIRTQTTSAKQEKAPSKKPVKRRKKKRAAN

SEQ ID NO: 98 - protein, 509 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 99 - protein, 712 aa, Homo sapiens
MGGEEKPIGAGEEKQKEGGKKKNKEGSGDGGRAELNPWPEYIYTRLEMYNILKAEHDSILAEKAEKDSKPIKVTLPDGKQVDAESWKTTPYQIACGISQGLADNTVIAKVNNVVWDLDRPLEEDCTLELLKFEDEEAQAVYWHSSAHIMGEGMERVYGGCLCYGPPIENGFYYDMYLEEGGVSSNDFSSLEALCKKIIKEKQAFERLEVKKETLLAMFKYNKFKCRILNEKVNTPTTTVYRCGPLIDLCRGPHVRHTGKIKALKIHKNSSTYWEGKADMETLQRIYGISFPDPKMLKEWEKFQEEAKNRDHRKIGRDQELYFFHELSPGSCFFLPKGVYIYNALIEFIRSEYRKRGFQEVVTPNIFNSRLWMTSGHWQHYSENMFSFEVEKELFALKPMNCPGHSLMFDHRPRSWRELPLRLADFGGLHRNELSGALTGLTRVRRFQQDDAHIFCAMEQIEDEIKGCLDFLRTVYSVFGFSFKLNLSTRPEKFLGDIEVWDQAEKQLENSLNEFGEKWELNSGDGAFYGPKIDIQIKDAIGRYHQCATIQLDFQLPIRFNLTYVSHDGEDKKRPVIVHRAILGSVERMIAILTENYGGKLAPFWLSPRQVMVVPVGPTCDEYAQNVRQQFHDAKFMADIDLDPGCTLNKKIRNAQLAQYNFILVVGEKEKITGTVNIRTRDNKVHGERTISETIERLQQLKEFRSKQAEEEF

SEQ ID NO: 100 - protein, 968 aa, Homo sapiens
MDSTLTASEIRQRFIDFFKRNEHTYVHSSATIPLDDPTLLFANAGMNQFKPIFLNTIDPSHPMAKLSRAANTQKCIRAGGKQNDLDDVGKDVYHHTFFEMLGSWSFGDYFKELACKMALELLTQEFGIPIERLYVTYFGGDEAAGLEADLECKQIWQNLGLDDTKILPGNMKDNFWEMGDTGPCGPCSEIHYDRIGGRDAAHLVNQDDPNVLEIWNLVFIQYNREADGILKPLPKKSIDTGMGLERLVSVLQNKMSNYDTDLFVPYFEAIQKGTGARPYTGKVGAEDADGIDMAYRVLADHARTITVALADGGRPDNTGRGYVLRRILRRAVRYAHEKLNASRGFFATLVDVVVQSLGDAFPELKKDPDMVKDIINEEEVQFLKTLSRGRRILDRKIQSLGDSKTIPGDTAWLLYDTYGFPVDLTGLIAEEKGLVVDMDGFEEERKLAQLKSQGKGAGGEDLIMLDIYAIEELRARGLEVTDDSPKYNYHLDSSGSYVFENTVATVMALRREKMFVEEVSTGQECGVVLDKTCFYAEQGGQIYDEGYLVKVDDSSEDKTEFTVKNAQVRGGYVLHIGTIYGDLKVGDQVWLFIDEPRRRPIMSNHTATHILNFALRSVLGEADQKGSLVAPDRLRFDFTAKGAMSTQQIKKAEEIANEMIEAAKAVYTQDCPLAAAKAIQGLRAVFDETYPDPVRVVSIGVPVSELLDDPSGPAGSLTSVEFCGGTHLRNSSHAGAFVIVTEEAIAKGIRRIVAVTGAEAQKALRKAESLKKCLSVMEAKVKAQTAPNKDVQREIADLGEALATAVIPQWQKDELRETLKSLKKVMDDLDRASKADVQKRVLEKTKQFIDSNPNQPLVILEMESGASAKALNEALKLFKMHSPQTSAMLFTVDNEAGKITCLCQVPQNAANRGLKASEWVQQVSGLMDGKGGGKDVSAQATGKNVGCLQEALQLATSFAQLRLGDVKN

SEQ ID NO: 101 - protein, 685 aa, Homo sapiens
MDGAGAEEVLAPLRLAVRQQGDLVRKLKEDKAPQVDVDKAVAELKARKRVLEAKELALQPKDDIVDRAKMEDTLKRRFFYDQAFAIYGGVSGLYDFGPVGCALKNNIIQTWRQHFIQEEQILEIDCTMLTPEPVLKTSGHVDKFADFMVKDVKNGECFRADHLLKAHLQKLMSDKKCSVEKKSEMESVLAQLDNYGQQELADLFVNYNVKSPITGNDLSPPVSFNLMFKTFIGPGGNMPGYLRPETAQGIFLNFKRLLEFNQGKLPFAAAQIGNSFRNEISPRSGLIRVREFTMAEIEHFVDPSEKDHPKFQNVADLHLYLYSAKAQVSGQSARKMRLGDAVEQGVINNTVLGYFIGRIYLYLTKVGISPDKLRFRQHMENEMAHYACDCWDAESKTSYGWIEIVGCADRSCYDLSCHARATKVPLVAEKPLKEPKTVNVVQFEPSKGAIGKAYKKDAKLVMEYLAICDECYITEMEMLLNEKGEFTIETEGKTFQLTKDMINVKRFQKTLYVEEVVPNVIEPSFGLGRIMYTVFEHTFHVREGDEQRTFFSFPAVVAPFKCSVLPLSQNQEFMPFVKELSEALTRHGVSHKVDDSSGSIGRRYARTDEIGVAFGVTIDFDTVNKTPHTATLRDRDSMRQIRAEISELPSIVQDLANGNITWADVEARYPLFEGQETGKKETIEE

SEQ ID NO: 102 - protein, 277 aa, Homo sapiens [sequence omitted]

SEQ ID NO: 103 - protein, 171 aa, Homo sapiens
MASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 104 - protein, 247 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLVFLCLQYRLRGKLRAEIENLHRTFDPHFLRVPCWKITLFVIVPVLGPLVALIICYNWLHRRLAGQFLEELRNPF

SEQ ID NO: 105 - protein, 626 aa, Homo sapiens
MIFLTALPLFWIMISASRGGHWGAWMPSSISAFEGTCVSIPCRFDFPDELRPAVVHGVWYFNSPYPKNYPPVVFKSRTQVVHESFQGRSRLLGDLGLRNCTLLLSNVSPELGGKYYFRGDLGGYNQYTFSEHSVLDIVNTPNIVVPPEVVAGTEVEVSCMVPDNCPELRPELSWLGHEGLGEPAVLGRLREDEGTWVQVSLLHFVPTREANGHRLGCQASFPNTTLQFEGYASMDVKYPPVIVEMNSSVEAIEGSHVSLLCGADSNPPPLLTWMRDGTVLREAVAESLLLELEEVTPAEDGVYACLAENAYGQDNRTVGLSVMYAPWKPTVNGTMVAVEGETVSILCSTQSNPDPILTIFKEKQILSTVIYESELQLELPAVSPEDDGEYWCVAENQYGQRATAFNLSVEFAPVLLLESHCAAARDTVQCLCVVKSNPEPSVAFELPSRNVTVNESEREFVYSERSGLVLTSILTLRGQAQAPPRVICTARNLYGAKSLELPFQGAHRLMWAKIGPVGAVVAFAILIAIVCYITQTRRKKNVTESPSFSAGDNPPVLFSSDFRISGAPEKYESERRLGSERRLLGLRGEPPELDLSYSHSDLGKRPTKDSYTLTEELAEYAEIRVK

SEQ ID NO: 106 - protein, 23 aa, Homo sapiens
GRTQDENPVVHFFKNIVTPRTPP

SEQ ID NO: 107 - protein, 22 aa, Homo sapiens
AVYVYIYFNTWTTCQFIAFPFK

SEQ ID NO: 108 - protein, 21 aa, Homo sapiens
SQRHGSKYLATASTMDHARHG

SEQ ID NO: 109 - protein, 20 aa, Homo sapiens
RDTGILDSIGRFFGGDRGAP

SEQ ID NO: 110 - protein, 20 aa, Homo sapiens
QKSHGRTQDENPVVHFFKNI

SEQ ID NO: 111 - protein, 14 aa, Homo sapiens
DENPVVHFFKNIVT

SEQ ID NO: 112 - protein, 15 aa, Homo sapiens
ENPVVHFFKNIVTPR

SEQ ID NO: 113 - protein, 13 aa, Homo sapiens
HFFKNIVTPRTPP

SEQ ID NO: 114 - protein, 14 aa, Homo sapiens
KGFKGVDAQGTLSK

SEQ ID NO: 115 - protein, 20 aa, Homo sapiens
VDAQGTLSKIFKLGGRDSRS

SEQ ID NO: 116 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 117 - protein, 585 aa, Homo sapiens
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 118 - protein, 979 aa, Homo sapiens
MRRPRRPGGLGGSGGLRLLLCLLLLSSRPGGCSAVSAHGCLFDRRLCSHLEVCIQDGLFGQCQVGVGQARPLLQVTSPVLQRLQGVLRQLMSQGLSWHDDLTQYVISQEMERIPRLRPPEPRPRDRSGLAPKRPGPAGELLLQDIPTGSAPAAQHRLPQPPVGKGGAGASSSLSPLQAELLPPLLEHLLLPPQPPHPSLSYEPALLQPYLFHQFGSRDGSRVSEGSPGMVSVGPLPKAEAPALFSRTASKGIFGDHPGHSYGDLPGPSPAQLFQDSGLLYLAQELPAPSRARVPRLPEQGSSSRAEDSPEGYEKEGLGDRGEKPASPAVQPDAALQRLAAVLAGYGVELRQLTPEQLSTLLTLLQLLPKGAGRNPGGVVNVGADIKKTMEGPVEGRDTAELPARTSPMPGHPTASPTSSEVQQVPSPVSSEPPKAARPPVTPVLLEKKSPLGQSQPTVAGQPSARPAAEEYGYIVTDQKPLSLAAGVKLLEILAEHVHMSSGSFINISVVGPALTFRIRHNEQNLSLADVTQQAGLVKSELEAQTGLQILQTGVGQREEAAAVLPQTAHSTSPMRSVLLTLVALAGVAGLLVALAVALCVRQHARQQDKERLAALGPEGAHGDTTFEYQDLCRQHMATKSLFNRAEGPPEPSRVSSVSSQFSDAAQASPSSHSSTPSWCEEPAQANMDISTGHMILAYMEDHLRNRDRLAKEWQALCAYQAEPNTCATAQGEGNIKKNRHPDFLPYDHARIKLKVESSPSRSDYINASPIIEHDPRMPAYIATQGPLSHTIADFWQMVWESGCTVIVMLTPLVEDGVKQCDRYWPDEGASLYHVYEVNLVSEHIWCEDFLVRSFYLKNVQTQETRTLTQFHFLSWPAEGTPASTRPLLDFRRKVNKCYRGRSCPIIVHCSDGAGRTGTYILIDMVLNRMAKGVKEIDIAATLEHVRDQRPGLVRSKDQFEFALTAVAEEVNAILKALPQ

SEQ ID NO: 119 - protein, 622 aa, Homo sapiens
MSMRSPISAQLALDGVGTMVNCTIKSEEKKEPCHEAPQGSATAAEPQPGDPARASQDSADPQAPAQGNFRGSWDCSSPEGNGSPEPKRPGASEAASGSQEKLDFNRNLKEVVPAIEKLLSSDWKERFLGRNSMEAKDVKGTQESLAEKELQLLVMIHQLSTLRDQLLTAHSEQKNMAAMLFEKQQQQMELARQQQEQIAKQQQQLIQQQHKINLLQQQIQQVNMPYVMIPAFPPSHQPLPVTPDSQLALPIQPIPCKPVEYPLQLLHSPPAPVVKRPGAMATHHPLQEPSQPLNLTAKPKAPELPNTSSSPSLKMSSCVPRPPSHGGPTRDLQSSPPSLPLGFLGEGDAVTKAIQDARQLLHSHSGALDGSPNTPFRKDLISLDSSPAKERLEDGCVHPLEEAMLSCDMDGSRHFPESRNSSHIKRPMNAFMVWAKDERRKILQAFPDMHNSSISKILGSRWKSMTNQEKQPYYEEQARLSRQHLEKYPDYKYKPRPKRTCIVEGKRLRVGEYKALMRTRRQDARQSYVIPPQAGQVQMSSSDVLYPRAAGMPLAQPLVEHYVPRSLDPNMPVIVNTCSLREEGEGTDDRHSVADGEMYRYSEDEDSEGEEKSDGELVVLTD

SEQ ID NO: 120 - protein, 483 aa, Homo sapiens [sequence omitted]

SEQ ID NO: 121 - protein, 471 aa, Homo sapiens
MSHHPSGLRAGFSSTSYRRTFGPPPSLSPGAFSYSSSSRFSSSRLLGSASPSSSVRLGSFRSPRAGAGALLRLPSERLDFSMAEALNQEFLATRSNEKQELQELNDRFANFIEKVRFLEQQNAALRGELSQARGQEPARADQLCQQELRELRRELELLGRERDRVQVERDGLAEDLAALKQRLEEETRKREDAEHNLVLFRKDVDDATLSRLELERKIESLMDEIEFLKKLHEEELRDLQVSVESQQVQQVEVEATVKPELTAALRDIRAQYESIAAKNLQEAEEWYKSKYADLSDAANRNHEALRQAKQEMNESRRQIQSLTCEVDGLRGTNEALLRQLRELEEQFALEAGGYQAGAARLEEELRQLKEEMARHLREYQELLNVKMALDIEIATYRKLLEGEESRISVPVHSFASLNIKTTVPEVEPPQDSHSRKTVLIKTIETRNGEQVVTESQKEQRSELDKSSAHSY

SEQ ID NO: 122 - protein, 306 aa, Homo sapiens
DCQAKSTPVIVSATTKKGLSSDLEGEKTTSLKWKSDEVDEQVACQEVKVSVAIEEDLEPENGILELETKSSKLVQNIIQTAVDQFVRTEETATEMLTSELQTQAHMIKADSQDAGQETEKEGEEPQASAQDETPITSAKEESESTAVGQAHSDISKDMSEASEKTMTVEVEGSTVNDQQLEEVVLPSEEEGGGAGTKSVPEDDGHALLAERIEKSLVEPKEDEKGDDVDDPENQNSALADTDASGGLTKESPDTNGPKQKEKEDAQEVELQEGKVHSESDKAITPQAQEELQKQERESAKSELTES

SEQ ID NO: 123 - protein, 520 aa, Homo sapiens
MHGGQGPLLLLLLLAVCLGGTQRNLRNQEERLLADLMQNYDPNLRPAERDSDVVNVSLKLTLTNLISLNEREEALTTNVWIEMQWCDYRLRWDPRDYEGLWVLRVPSTMVWRPDIVLENNVDGVFEVALYCNVLVSPDGCIYWLPPAIFRSACSISVTYFPFDWQNCSLIFQSQTYSTNEIDLQLSQEDGQTIEWIFIDPEAFTENGEWAIQHRPAKMLLDPAAPAQEAGHQKVVFYLLIQRKPLFYVINIIAPCVLISSVAILIHFLPAKAGGQKCTVAINVLLAQTVFLFLVAKKVPETSQAVPLISKYLTFLLVVTILIVVNAVVVLNVSLRSPHTHSMARGVRKVFLRLLPQLLRMHVRPLAPAAVQDTQSRLQNGSSGWSITTGEEVALCLPRSELLFQQWQRQGLVAAALEKLEKGPELGLSQFCGSLKQAAPAIQACVEACNLIACARHQQSHFDNGNEEWFLVGRVLDRVCFLAMLSLFICGTAGIFLMAHYNRVPALPFPGDPRPYLPSPD

SEQ ID NO: 124 - protein, 457 aa, Homo sapiens [sequence omitted]

SEQ ID NO: 125 - protein, 26 aa, Homo sapiens
TVGLQLIQLINVDEVNQIVTTNVRLK

SEQ ID NO: 126 - protein, 10 aa, Homo sapiens
QQWVDYNLKW

SEQ ID NO: 127 - protein, 20 aa, Homo sapiens
QIVTTNVRLKQQWVDYNLKW

SEQ ID NO: 128 - protein, 7 aa, Homo sapiens
QWVDYNL

SEQ ID NO: 129 - protein, 18 aa, Homo sapiens
GGVKKIHIPSEKIWRPDL

SEQ ID NO: 130 - protein, 12 aa, Homo sapiens
AIVKFTKVLLQY

SEQ ID NO: 131 - protein, 20 aa, Homo sapiens
WTPPAIFKSYCEIIVTHFPF

SEQ ID NO: 132 - protein, 13 aa, Homo sapiens
MKLGTWTYDGSVV

SEQ ID NO: 133 - protein, 13 aa, Homo sapiens
MKLGIWTYDGSVV

SEQ ID NO: 134 - protein, 9 aa, Homo sapiens
WTYDGSVVA

SEQ ID NO: 135 - protein, 17 aa, Homo sapiens
SCCPDTPYLDITYHFVM

SEQ ID NO: 136 - protein, 18 aa, Homo sapiens
DTPYLDITYHFVMQRLPL

SEQ ID NO: 137 - protein, 21 aa, Homo sapiens
FIVNVIIPCLLFSFLTGLVFY

SEQ ID NO: 138 - protein, 13 aa, Homo sapiens
LLVIVELIPSTSS

SEQ ID NO: 139 - protein, 19 aa, Homo sapiens
STHVMPNWVRKVFIDTIPN

SEQ ID NO: 140 - protein, 18 aa, Homo sapiens
NWVRKVFIDTIPNIMFFS

SEQ ID NO: 141 - protein, 18 aa, Homo sapiens
IPNIMFFSTMKRPSREKQ

SEQ ID NO: 142 - protein, 16 aa, Homo sapiens
AAAEWKYVAMVMDHIL

SEQ ID NO: 143 - protein, 19 aa, Homo sapiens
IIGTLAVFAGRLIELNQQG

SEQ ID NO: 144 - protein, 20 aa, Homo sapiens
GQTIEWIFIDPEAFTENGEW

SEQ ID NO: 145 - protein, 20 aa, Homo sapiens
MAHYNRVPALPFPGDPRPYL

SEQ ID NO: 146 - protein, 609 aa, Homo sapiens
MSGWESYYKTEGDEEAEEEQEENLEASGDYKYSGRDSLIFLVDASKAMFESQSEDELTPFDMSIQCIQSVYISKIISSDRDLLAVVFYGTEKDKNSVNFKNIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLMHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPMLKEACRAYGLKSGLKKQELLEALTKHFQD

SEQ ID NO: 147 - protein, 732 aa, Homo sapiens
MVRSGNKAAVVLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISLQFFLPFSLGKEDGSGDRGDGPFRLGGHGPSFPLKGITEQQKEGLEIVKMVMISLEGEDGLDEIYSFSESLRKLCVFKKIERHSIHWPCRLTIGSNLSIRIAAYKSILQERVKKTWTVVDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDMVAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAKKLKTEQGGAHFSVSSLAEGSVTSVGSVNPAENFRVLVKQKKASFEEASNQLINHIEQFLDTNETPYFMKSIDCIRAFREEAIKFSEEQRFNNFLKALQEKVEIKQLNHFWEIVVQDGITLITKEEASGSSVTAEEAKKFLAPKDKPSGDTAAVFEEGGDVDDLLDMI

SEQ ID NO: 148 - protein, 408 aa, Homo sapiens
MAENGDNEKMAALEAKICHQIEYYFGDFNLPRDKFLKEQIKLDEGWVPLEIMIKFNRLNRLTTDFNVIVEALSKSKAELMEISEDKTKIRRSPSKPLPEVTDEYKNDVKNRSVYIKGFPTDATLDDIKEWLEDKGQVLNIQMRRTLHKAFKGSIFVVFDSIESAKKFVETPGQKYKETDLLILFKDDYFAKKNEERKQNKVEAKLRAKQEQEAKQKLEEDAEMKSLEEKIGCLLKFSGDLDDQTCREDLHILFSNHGEIKWIDFVRGAKEGIILFKEKAKEALGKAKDANNGNLQLRNKEVTWEVLEGEVEKEALKKIIEDQQESLNKWKSKGRRFKGKGKGNKAAQPGSGKGKVQFQGKKTKFASDDEHDEHDENGATGPVKRAREETDKEEPASKQQKTENGAGDQ

SEQ ID NO: 149 - protein, 175 aa, Artificial Sequence
  Description of Artificial Sequence Synthetic peptide
  MOD_RES (1)..(4): Variable amino acid
  MOD_RES (6)..(53): Variable amino acid and this region may encompass 0 to 48 residues
  MOD_RES (55)..(66): Variable amino acid and this region may encompass 3 to 12 residues
  MOD_RES (68)..(137): Variable amino acid and this region may encompass 1 to 70 residues
  MOD_RES (139)..(144): Variable amino acid and this region may encompass 1 to 6 residues
  MOD_RES (146)..(147): Variable amino acid
  MOD_RES (149): Aromatic amino acid
  MOD_RES (150)..(170): Variable amino acid and this region may encompass 0 to 21 residues
  MOD_RES (172)..(173): Variable amino acid
  MOD_RES (175): Variable amino acid
XXXXCXXXXXXXXXXXXXXXXXXXXXXXXXXXXXXXXXXXXXXXXXXXXXXXXCXXXXXXXXXXXXCXXXXXXXXXXXXXXXXXXXXXXXXXXXXXXXXXXXXXXXXXXXXXXXXXXXXXXXXXXXXXXXXXXXXXXCXXXXXXCXXGXXXXXXXXXXXXXXXXXXXXXXGXXCX

SEQ ID NO: 150 - protein, 13 aa, Homo sapiens
VHFFKNIVTPRTP

SEQ ID NO: 151 - protein, 7 aa, Homo sapiens
FKNIVTP